(19) United States
(12) Patent Application Publication
(10) Pub. No.: US 2025/0304996 A1
(43) Pub. Date: Oct. 2, 2025

(54) PSEUDOTYPED LENTIVIRAL VECTORS

(71) Applicant: IMPERIAL COLLEGE INNOVATIONS LIMITED, LONDON (GB)

(72) Inventors: Stephen HYDE, Oxford (GB); Kamran MIAH, Oxford (GB); Deborah GILL, Oxford (GB); Yue DU, Oxford (GB)

(21) Appl. No.: 18/722,500

(22) PCT Filed: Dec. 21, 2022
(86) PCT No.: PCT/GB2022/053351

§ 371 Date: Jun. 20, 2024

(30) Foreign Application Priority Data
Dec. 21, 2021 (GB) 2118685.3

(51) Int. Cl. C12N 15/86; A61K 35/76; A61K 38/16; C07K 14/005

(52) U.S. Cl. CPC C12N 15/86; A61K 35/76; A61K 38/162; C07K 14/005; C12N 2740/15022; C12N 2740/15043; C12N 2740/16022; C12N 2740/16023; C12N 2740/16043; C12N 2740/16045; C12N 2770/20022; C12N 2770/20034; C12N 2800/22; C12N 2830/42; C12N 2830/50

(57) ABSTRACT

The present invention relates to pseudotyped lentiviral vectors, particularly to pseudotyped with a modified severe acute respiratory syndrome coronavirus 2 (SARS-CoV-2) spike protein, as well as related constructs, methods and therapeutic indications.

### FIELD OF THE INVENTION

The present invention relates to pseudotyped lentiviral vectors, particularly those pseudotyped with a modified severe acute respiratory syndrome coronavirus 2 (SARS-CoV-2) spike protein, as well as related constructs, methods and therapeutic indications.

### BACKGROUND TO THE INVENTION

The Coronavirus Disease 2019 (COVID-19) pandemic is caused by the severe acute respiratory syndrome coronavirus 2 (SARS-CoV-2) and remains and ongoing public health issue. This is largely due to the continued identification and emergence of novel mutations within the genome of SARS-CoV-2 arising in infected COVID-19 patients across the globe. Continued global genomic surveillance efforts of the SARS-CoV-2 sequences have resolved thousands of mutations, many of which can be examined further to predict variants of interest early in their emergence. Of particular importance are the rising numbers of independent variants under investigation (VUIs) or of concern (VOCs) discovered globally (e.g. SARS-CoV-2 lineages from the U.K. (B.1.1.7, Alpha), South Africa (B.1.351, Beta); B.1.1.529, (Omicron), Brazil (P.1, Gamma), and India (B.1.617.2, Delta)); the latter are defined as such for their increased tendencies in transmission, and/or disease severity, treatment resistance, escape from neutralizing antibodies (nAbs) including those originally elicited by anti-SARS-CoV-2 Spike (S) glycoprotein vaccine efforts to varying degrees.

With the emergence of VOCs comes an increased need for continued cross-examination of these new variants, especially against existing nAbs, vaccine strategies, and therapeutics. However, handling authentic SARS-CoV-2 (a risk group 3 pathogen) requires high biosafety level (BSL) 3 containment facilities—the access and availability of which are limited. Therefore, there remains an unprecedented need for safe, improved, and easily accessible SARS-CoV-2 equivalent resources to support related research. To help facilitate this need pseudotyping viral vectors to generate pseudovirions (PSVs) has been a widely adopted practice to model infectious and pathogenic viruses. Minimal and essential components for viral infection, commonly the viral envelope glycoprotein(s) that contribute to viral entry into target cells, are expressed on membranes of defunct viral vectors. These viral mimics are relatively safer to handle and supersedes the need for high BSL facilities required to handle live and authentic pathogens. The SARS-CoV-2 membrane S glycoprotein, which mediates viral attachment to target cells by binding to the human angiotensin converting enzyme 2 (hACE2) and employs the human serine protease (hTMPRSS2) to prime membrane fusion between virus and target, has been readily repurposed to generate useful SARS-CoV-2 PSVs. In these cases the SARS-CoV-2 S protein is assumingly assembled as a functional trimer in the acquired envelope of viral vectors. A key advantage of this feature is the resultant PSV adopts similar target cell entry mechanisms of its pathogenic SARS-CoV-2 parent. Their utilization in the current SARS-CoV-2/COVID-19 pandemic has helped drive forward and accelerate efforts in vaccine development and therapeutics. However, well established SARS-CoV-2 S PSVs are associated with numerous problems and disadvantages, limiting their utility.

In particular, current SARS-CoV-2 PSVs are based on pseudotyping γ-retroviruses, rhabdoviruses, or first and second generation rHIV1 LVs. These platforms are still restricted to high BSL 2 settings in order to operate. Pseudotyped γ-retroviruses are further challenged by their limited transduction proficiencies of non-dividing cells, and in turn restricts their utility as a PSV resource including in SARS-CoV-2-related research. Despite the current repertoire of SARS-CoV-2 PSVs, there remains an ongoing global demand for high titre, accessible, and easy to handle SARS-CoV-2 PSV resource to robustly cross-examine continuingly emerging and rampant SARS-CoV-2 variants. This is especially true for VOCs that present an additional level of threat to public health given their capacity to evade well established immunity from prior infections with the Wuhan Hu-1 or the G614 strains, and first-generation vaccine efforts to varying degrees. Further, first-generation vaccines designs are based on the SARS-CoV-2 S glycoprotein from the Wuhan Hu-1 isolate, which is now no longer the dominant and pandemic-defining SARS-CoV-2 strain in circulation. This design feature continually raises concern for how efficacious a given SARS-CoV-2 vaccine strategy is to current and novel emerging VOCs. There is therefore an ongoing and pressing need to cross-examine SARS-CoV-2 variants and representative PSVs under more controlled and standardized research conditions. By extension this would enable standardized examination of the robustness and quality of SARS-CoV-2-related nAbs, vaccine efficacies, and therapeutics.

In addition, better animal and in vitro models would greatly assist in the research and development of prophylaxes, therapeutics, and vaccine strategies. Currently available mouse models for authentic and non-mouse-adapted SARS-CoV-2 infection are difficult to engineer with significant costs and animal wastage associated with supplying hACE2 transgenic or CRISPR/Cas9-mediated knock-in animals as significant primary examples. Even simplifying the humanization and sensitization process of mouse models to SARS-CoV-2, for example by introducing hACE2 to the murine lungs and airway in trans by replication incompetent Adenoviral (AdV), recombinant Adenovirus-Associated Viral (rAAV), or LV vectors, results in non-physiologically relevant biodistribution of hACE2 in the murine lungs as dictated by the tropism of the selected gene transfer vector. Additionally, the complexity of challenging animal models with multiple vectors (assumingly at least two different viral-based materials are necessary—i. the vector of choice for gene transfer to achieve hACE2-humanization, and ii. the challenging PSV or authentic SARS-CoV-2) can be alleviated by mACE2 co-permissive SARS-CoV-2.

The present invention seeks to overcome one or more of these problems. In particular, it is an object of the present invention to provide lentiviral vectors pseudotyped with modified SARS-CoV-2 spike proteins that (i) are not associated with current SARS-CoV-2 PSVs, and (ii) are rodent-adapted, particularly mouse-adapted, and thus are capable of transducing rodent/mouse cells without encountering the issues associated with conventional SARS-CoV-2 mouse models.

### SUMMARY OF THE INVENTION

The present inventors have produced mouse-adapted S-LV vectors with improved function and functional titres, and thus offer advantages in terms of SARS-CoV-2 modelling, and pre-clinical (in vitro and animal models). The S-LV of the invention also retain the ability to transduce cells via hACE2, and so potentially bridge the gap between pre-clinical and clinical application. In more detail, as exemplified herein, the inventors pseudotyped third generation self-inactivating (SIN) HIV1 lentiviral vectors and pseudotyped these with S glycoprotein from multiple clinically relevant SARS-CoV-2 variants (including: Wuhan Hu-1, G614, Australia/VIC01/2020 (Aus/VIC01), B.1.1.7, B.1.351) with modifications—namely truncating the C-terminus tail by 19aa, to produce “S-LV”. Surprisingly, the S-LV of the invention can be produced at high titres, which permits expanded downstream applications, and using a range of SARS-CoV-2 variants. In particular, as exemplified herein, impressive functional titres of S-LV were achieved after transient transfection of suspension 293T/17 with eGFP or FLuc reporter-encoding HIV1 LV genome, HIV1 GagPol, HIV1 Rev plasmids, and plasmid(s) encoding the S glycoproteins of interest. S-LVs could be further concentrated and purified for expanded downstream uses including in vivo applications.

The inventors therefore provide an S-LV platform that can be used to model a library SAR-CoV-2 S glycoproteins of interest or clinical relevance, and can be used to support SARS-CoV-2-related research by providing a means to model, assess, and predict infectivity of clinically relevant VOCs or test neutralization propensities of neutralizing antibodies or convalescent plasma from COVID-19 recovered patients; all in standard laboratory containment conditions. Given its broad utility, this S-LV platform can potentially be harnessed to rapidly model emerging and novel VUI or VOCs to help assess their infectivity profiles in vitro and in vivo, and thus provide a contributing metric to pre-determine their potential impact on global health, and screen predicted variants before their potential emergence in nature, and facilitate prompt and strategic responses in the face of the on-going COVID-19/SARS-CoV-2 pandemic.

Significantly, the inventors have provided for the first time mouse (m)ACE2 adapted (ma)S-LV mediates which achieve potent in vivo gene transfer of BALB/c mice, without the need for expression of hACE2 in trans of the murine airways. The exemplified maS-LV comprise numerous mutations in the SARS-CoV-2 S protein, including the Q498Y, P499T and Δ19 mutations, and optionally also N501Y.

As exemplified herein, the inventors have provided the first demonstration of applying S-LV in vivo by intranasally dosing BALB/c mice to demonstrate potent gene transfer capabilities independent of hACE2 expression as a rapid and direct means to model SARS-CoV-2 infection in vivo. Taken all together, this S-LV platform accurately represents authentic SARS-CoV-2, in particular representative variants modelled, at the level of the SARS-CoV-2 S glycoprotein. This S-LV platform demonstrates robustness in the wide array of SARS-CoV-2 variants represented in the current study, and flexibility such that it is not constrained by high BSL requirements. The S-LV platform therefore functions as an easy to use, easy to access, and safe SARS-CoV-2 PSV resource for related research to combat the on-going COVID-19/SARS-CoV-2 pandemic.

Accordingly, the present invention provides a lentiviral vector pseudotyped with a modified severe acute respiratory syndrome coronavirus 2 (SARS-CoV-2) spike protein, which lentiviral vector comprises a transgene operably linked to a promoter; and wherein said spike protein comprises: (a) mutations at amino acid positions corresponding to, or aligning with, positions 498, 499 and 614 of SEQ ID NO: 1; and (b) a deletion of at least a portion of the cytoplasmic tail.

The invention also provides a lentiviral vector pseudotyped with a modified severe acute respiratory syndrome coronavirus 2 (SARS-CoV-2) spike protein, which lentiviral vector comprises a transgene operably linked to a promoter; and wherein said spike protein comprises: (a) a mutation at an amino acid position corresponding to, or aligning with, position 614 of SEQ ID NO: 1; (b) a deletion of at least a portion of the cytoplasmic tail; and (c) (i) mutations at amino acid positions corresponding to, or aligning with, positions 498 and 499 of SEQ ID NO: 1; and/or (ii) a mutation at an amino acid position corresponding to, or aligning with, position 501 of SEQ ID NO: 1.

The cytoplasmic tail of the spike protein may correspond to, or align with amino acid resides 1235 to 1273 of SEQ ID NO: 1. The deletion of at least a portion of the cytoplasmic tail of the spike protein may comprise: (a) deletion of at least 10 amino acids, preferably at least 15 amino acids of the cytoplasmic tail; and/or (b) deletion of the amino acid residue corresponding to, or aligning with, positions 1255 to 1273 of SEQ ID NO: 1.

One or more of the mutations of the spike protein at amino acid positions corresponding to, or aligning with, positions 498, 499 and 614 of SEQ ID NO: 1 may be amino acid substitutions, and preferably all of the mutations are amino acid substitutions. The amino acid substitutions may be non-conservative amino acid substitutions. The amino acid corresponding to, or aligning with: (a) position 498 of SEQ ID NO: 1 may be substituted by tyrosine; (b) position 499 of SEQ ID NO: 1 may be substituted by threonine; and/or (c) position 614 of SEQ ID NO: 1 may be substituted by glycine. The mutations may be Q498Y, P499T and D614G.

The modified spike protein may be capable of binding to the enzymatic domain of human angiotensin converting enzyme 2 (ACE2).

The modified SARS-CoV-2 spike protein may be derived from a SARS-CoV-2 strain selected from Wuhan-Hu-1 strain, B.1.1.7 strain, B.1.351 strain, P.1 strain, B.1.617.2 strain, B.1.427, B.1.1.529, C.37, B.1.429 or Australia/VIC01/2020 (Aus/VIC01) strain.

The modified spike protein may not be detected by anti-coronavirus spike protein antibodies, preferably, anti-coronavirus spike protein antibodies MM43 or R001.

The modified spike protein may comprise an amino acid sequence having at least 90% sequence identity with SEQ ID NO: 13. The modified spike protein may further comprise one or more additional mutation, which may optionally comprise a mutation at an amino acid position corresponding to, or aligning with, position 501 of SEQ ID NO: 1.

The mutation at an amino acid position corresponding to, or aligning with, position 501 of SEQ ID NO: 1, or the one or more additional mutation comprising a mutation at an amino acid position corresponding to, or aligning with, position 501 of SEQ ID NO: 1 may optionally: (a) be an amino acid substitution, preferably a non-conservative amino acid substitution, even more preferably a substitution by tyrosine; and/or (b) comprise N501Y.

The modified spike protein may comprise an amino acid sequence having at least 90% sequence identity with SEQ ID NO: 20.

The modified spike protein may comprise: (a) mutations at amino acid positions corresponding to, or aligning with, one or more of positions 80, 215, 417, 484, 501, 614 and 701 of SEQ ID NO: 1 wherein preferably all these residues are mutated; and (b) a deletion of at least a portion of the cytoplasmic tail. Optionally (a) said modified SARS-CoV-2 spike protein may be derived from the spike protein of the B.1.351 strain; (b) the amino acid corresponding to, or aligning with: (i) position 80 of SEQ ID NO: 1 is substituted by alanine; (ii) position 215 of SEQ ID NO: 1 is substituted by glycine; (iii) position 417 of SEQ ID NO: 1 is substituted by asparagine; (iv) position 484 of SEQ ID NO: 1 is substituted by lysine; (v) position 501 of SEQ ID NO: 1 is substituted by tyrosine; (vi) position 614 of SEQ ID NO: 1 is substituted by glycine and/or (vii) position 701 of SEQ ID NO: 1 is substituted by valine; wherein preferably all these residues are substituted; and/or (c) the deletion of at least a portion of the cytoplasmic tail comprises or consists of deletion of the amino acid residues corresponding to or aligning with positions 1255 to 1273 of SEQ ID NO: 1.

A lentiviral vector of the invention may be selected or derived from the group consisting of a Simian immunodeficiency virus (SIV) vector, a Human immunodeficiency virus (HIV) vector, a Feline immunodeficiency virus (FIV) vector, an Equine infectious anaemia virus (EIAV) vector, and a Visna/maedi virus vector.

A lentiviral vector of the invention may be capable of transducing rodent cells in vivo, preferably mouse cells in vivo.

The invention also provides a modified severe acute respiratory syndrome coronavirus 2 (SARS-CoV-2) spike protein of the invention.

The invention further provides a polynucleotide molecule encoding a modified spike protein of the invention. The invention also provides an expression construct comprising a polynucleotide of the invention, wherein optionally said polynucleotide is operably linked to a promoter.

The invention also provides a host cell comprising a lentiviral vector of the invention, a modified spike protein of the invention, a polynucleotide of the invention or an expression construct of the invention.

The invention further provides a virus-like particle (VLP) comprising a modified SARS-CoV-2 spike protein of the invention.

The invention also provides a lentiviral vector of the invention, a modified spike protein of the invention, a polynucleotide of the invention, an expression construct of the invention or a VLP of the invention, for use in therapy, wherein preferably the therapy is gene therapy.

The invention further provides the in vitro use of the lentiviral vector of the invention, a modified spike protein of the invention, a polynucleotide of the invention, an expression construct of the invention, or a VLP of the invention.

The invention also provides a method of producing a lentiviral vector of the invention, the method comprising: (a) introducing (i) a nucleic acid sequence encoding a modified SARS-CoV-2 spike protein of the invention; and (ii) one or more nucleic acid sequence encoding lentiviral packaging components, lentiviral envelope components, and a lentiviral genome, into a viral vector production cell; and (b) culturing the production cell under conditions suitable for the production of the lentiviral vector. Said method may further comprise harvesting said lentiviral vector. The nucleic acid sequence encoding the modified SARS-CoV-2 spike protein may be comprised in a polynucleotide molecule of the invention or an expression construct of the invention. The one or more nucleic acid sequence encoding the lentiviral packaging components, lentiviral envelope components, and a lentiviral genome may be comprised in (i) the same polynucleotide molecule or expression construct as the nucleic acid sequence encoding the modified SARS-CoV-2 spike protein or (ii) in one or more separate polynucleotide molecule or expression construct. SARS-CoV-2 nucleoprotein may be co-expressed during the culturing of the production cell, wherein preferably the SARS-CoV-2 nucleoprotein is from the Wuhan-Hu-1 or B.1.1.529 strain.

### BRIEF DESCRIPTION OF THE DRAWINGS

FIG. 1: A Representative schematic of full-length and Δ19aa C-terminus truncated SARS-CoV-2 S protein (top panel and bottom panel, respectively) and annotated domains. Open triangle refers to S1/S2 furin cleavage site, and closed triangle refers to the S2 cleavage site. Protein and annotated domains are not to scale. aa, amino acid(s); CT, cytoplasmic tail; FL, full length; FP, fusion peptide; HR1 and 2, heptide repeat 1 and 2, respectively; NTD, N-terminal domain; RBD, receptor binding domain; SS, signal sequence; TM, transmembrane domain; Δ, deleted. B Representative SARS-CoV-2 S pseudotype plasmid configurations and their associated S-LV version (v01-05). C Plasmid map for pGM998 (maS^{498Y,P499T,N501Y+Δ19aa}). D Plasmid map for pGM1000 (S^{1.1.7+1.351 chimera+Δ19aa}) E Plasmid map for pGM1027 (S^{B.1.617.2+Δ19aa}). F Plasmid map for pGM1028 (S^{C.37+Δ19aa}) G Plasmid map for pGM1038 (S^{B.1.617.2,Y501+Δ19aa}) H Plasmid map for pGM1039 (S^{C.37,Y501+Δ19aa}) I Plasmid map for pGM1040 (S^{B.1.617.2,Y498,T499,Y501+Δ19aa}) J Plasmid map for pGM1041 (S^{C.37,Y498,T499,Y501+Δ19aa}) Schematics depict the configurations of pseudotype plasmids generated to express the SARS-CoV-2 (co)S protein under CMVIE enhancer and chicken β-actin promoter control. The specific set of mutations that define SARS-CoV-2 S variants are annotated, with amino acid changes highlighted: blue annotated mutations refer to mutations within the N-terminus domain (NTD); red annotated mutations refer to mutations within the receptor binding domain (RBD), and black annotated mutations refer to mutations outside of the aforementioned domains (i.e. remainder S1 and S2 domains). CBA/RBG intron, chicken β-actin and Rabbit β-globin chimeric intron; co, codon optimised; CMVIE enhancer, cytomegalovirus immediate-early enhancer; UTR, untranslated region

FIG. 2: Multiple sequence alignment of cDNA sequences between Wuhan Hu-1 and SARS-CoV-2 S variants used to pseudotype rHIV1 lentiviral vectors. Underlined sequence refers to a silent introduction of an Agel restriction enzyme site into B.1.1.7+Δ19aa cDNA sequence with no impact on encoding protein sequence.

FIG. 3: Multiple sequence alignment of SARS-CoV-2 S protein sequence between Wuhan Hu-1 and SARS-CoV-2 S variants used to pseudotype rHIV1 lentiviral vectors. Highlighted and underlined amino acid residues or deletions indicate mutations/changes compared to Wuhan Hu-1 reference sequence to generate the respective SARS-CoV-2 S variant and/or +Δ19aa derivative.

FIG. 4: Co-expression of (co)hACE2 and hTMPRSS2 significantly improves the permissiveness of 293T/17 cells to, and rescues the transduction of, S-LV. The indicated LV.eGFP pseudotyped with SARS-CoV-2 (co)S, VSVg, or bald LV were produced by transient co-transfection of 293T/17 SF cells as described (see Materials and Methods), with crude LV collected 72 h post-transfection. Parental 293T/17, (co)hACE2±hTMRPSS2 expressing cell lines were transduced with a fixed dilution of (A) rHIV1.VSVg (n=4), and S-LV pseudotyped with the prototypical SARS-CoV-2 (B) SWuhan Hu-1±Δ19aa (n=4), (C) SG614±Δ19aa (n=4), and (D) SAus/VIC01±Δ19aa (n=4). Approximately 72hpi, cells were subjected to flow cytometry analysis for transduction efficiency by EGFP reporter. Individual data points reflecting transduction efficiencies are presented with groups' mean±SD indicated (*, *, * and **** represents P<0.05, <0.01, <0.001, and <0.0001, respectively). Inoculation of cell lines with rHIV1.bald material of parental and engineered cell lines was used to determine the average baseline fluorescence and the lower gating threshold for EGFP-positive cell populations by flow cytometry and is presented in each graph as a dotted line.

FIG. 5: The S-LV platform can model SARS-CoV-2 variants of concern or interest and recapitulates their inherent features in vitro. The indicated S- and maS-LV and rHIV1.bald were produced by transient co-transfection of 293T/17 SF cells, with crude LV collected 72 h post-transfection. (A) Parental 293T/17, (co)hACE2±hTMRPSS2 expressing cell lines with a fixed dilution of S-LV pseudotyped with (co)S^{B1.1.7+Δ19aa} or (co)S^{B.1.351+Δ19aa}, and maS-LV pseudotyped with maS^{Wuhan Hu-1,Y498,T499+Δ19aa} or maS^{Y498,T499,G614+Δ19aa} (n=4 each). Individual data points reflecting transduction efficiencies are presented with groups' mean±SD indicated (*, *, * and **** represents P<0.05, <0.01, <0.001, and <0.0001, respectively). On the other hand, the selected S- and maS-LVs described in (A) were also transduced on (B) (co)mACE2±hTMRPSS2 expressing cell lines, to demonstrate permissiveness of N501Y- or Q498Y & P499T-containing SARS-CoV-2 (co)S using the mACE2 cell entry receptor. Data is presented as mean±SD. Inoculation of cell lines with rHIV1.bald material of parental and engineered cell lines was used to determine the average baseline fluorescence and the lower gating threshold for eGFP-positive cell populations by flow cytometry and is presented as a dotted line. (C) Parental 293T/17 cells were seeded in 24 well plates for 70% confluency the next day. Cells were then co-transfected with 200 ng pCMV+eGFP reporter plasmid and 100 ng of the indicated SARS-CoV-2 (co)S or maS pseudotype plasmids, or pCAG eGFP as mock control, at 1:2 ratio with FuGene 6 transfection reagent. Cells were cultured and visualized for eGFP-fluorescence at the indicated time points. Cells highlighted by white arrow heads indicate cells exhibiting syncytia. Scale bar=500 μm.

FIG. 6: Δ19aa deletion of SARS-CoV-2 (co)S cytoplasmic tail and addition of D614G rescues S-LV titres on permissive 293T/17 cells co-expressing (co)hACE2 & hTMPSS2. S-LVs based on (A) (co)S^{Wuhan Hu-1±Δ19aa}, (B) (co)S^{G614±Δ19aa}, (C) (co)S^{Aus/VIC01±Δ19aa} pseudotypes were produced and harvested at 24h intervals between 72-144 h post-transfection, and then titred on 293T/17 cells co-expressing (co)hACE2 & hTMPRSS2. Titres were determined after FCS analysis of transduced cells. S-LVs that harbour a 19aa deletion of the cytoplasmic tail encompassing a putative endoplasmic retention signal demonstrated substantial improvement and rescue functional titres compared to their full-length counterparts, with peak IU/mL titres calculated for material harvested at 72 h post-transfection. Furthermore (D) (co)S^{B1.1.7+Δ19aa} and (co)S^{B.1.1351+Δ19aa}, and (E) maS^{Y498,T499±G614+Δ19aa} pseudotypes also demonstrate successful packaging and similar production kinetics, with impressive titres using FCS analysis.

FIG. 7: N501Y-containing- and mouse ACE2 adapted S-LVs are permissive for and can be titred on (co)mACE2 & hTMPRRS2 co-expressing cells. (A) 293T/17 cells co-expressing (co)mACE2 & hTMPRSS2 were transduced with LV pseudotyped with G614, N501Y, or Y498, T499-containing SARS-CoV-2 (co)S proteins. Using fluorescence microscopy LVs pseudotyped with N501Y, or Y498, T499-containing SARS-CoV-2 (co)S demonstrated impressive permissiveness for 293T/17 cells co-expressing (co)mACE2 & hTMPRSS2, compared to reduced/near-ablated permissiveness for G614-containing S-LV. (B) S^{B.1.1.7+Δ19aa}, S^{B.1.1351+Δ19aa}, and maS^{Y498,T499+Δ19aa}-LVs were produced and harvested at 24 h intervals between 72-144 h post-transfection, and then titred on 293T/17 cells co-expressing (co)mACE2 & hTMPRSS2. Titres were determined after FCS analysis of transduced cells.

FIG. 8: Mouse ACE2 adaptation via Y498 and T499 further rescues functional titres of maS-LV based on SARS-CoV-2 (co)S^{Aus/VIC01} pseudotype. S^{Aus/VIC01+Δ19aa} LV and maS^{Aus/VIC01,Y498,T499+Δ19aa}-LV were produced and harvested at 72 h post-transfection, and titred on permissive 293T/17 cells co-expressing (co)hACE2 & hTMPSS2. Transduced cells were analysed by FCS. IU/mL titres calculated for S^{Aus/VIC01+Δ19aa}-LV and maS^{Aus/VIC01,Y498,T499+Δ19aa}-LV indicate that the Y498 and T499 mutations can confer both mouse ACE2 adaptation and potentially rescue functional titres of SARS-CoV-2 S^{Aus/VIC01} pseudotyped LVs further.

FIG. 9: S-LV can be produced at high functional titre after AEX&TFF and can model infectivity of SARS-CoV-2 S VOCs. The indicated S- and maS-LV.FLuc vectors were produced, purified, diafiltrated and concentrated by combination of AEX&TFF methods. Purified and concentrated vector material was titrated on 293T/17 cells co-expressing (co)hACE2 or (co)mACE2 (as indicated) & hTMPRSS2 (seeded to achieve 30-40% confluency on day of transduction). 72hpi cells were harvested, lysed, and luciferase activity measured per dilution. (A) Luciferase activity per well was normalised to input ng p24 at transduction of (co)hACE2 & hTMPRSS2 co-expressing cells. Groups' mean±SD are indicated (Kruskal-Wallis One-Way ANOVA with Dunn's multiple comparisons test; **** represents P<0.0001). (B) Luciferase activity per well was normalised to input ng p24 at transduction of (co)mACE2 & hTMPRSS2 co-expressing cells. Groups' mean±SD are indicated (Kruskal-Wallis One-Way ANOVA with Dunn's multiple comparisons test; *** and **** represents P<0.001 and 0.0001, respectively).

FIG. 10: maS^{Y498,T499,G614+Δ19aa}-LV can be potently neutralised in vitro by commercially available neutralising antibodies raised against SARS-CoV-2 Spike protein. Permissive 293T/17 cells co-expressing (co)hACE2 & hTMPSS2 were transduced with S-LV at multiplicity of infection 1 in the presence of mouse IgG1 MM43 (SARS-CoV-2 S neutralising antibody, Sino Biological) or isotype control, and rabbit IgG R001 (SARS-CoV-2 S neutralising antibody, Sino Biological) or isotype control at the presented working concentrations. 48-72 h post-transduction, cells were subjected to FCS to calculate IC₅₀'s. (A) Transduction profiles of maS^{Y498,T499,G614+Δ19aa}-LV in the presence of MM43 or isotype control across tested range of either antibodies. (B) Neutralisation curve of maS^{Y498,T499,G614+Δ19aa}-LV by MM43; neutralisation was calculated as % transduction of mouse IgG1 isotype control. (C) Transduction profiles of maS^{Y49S,T499,G614+Δ19aa}-LV in the presence of R001 or isotype control across tested range of either antibodies. (D) Neutralisation curve of maS^{Y498,T499,G614+Δ19aa}-LV by R001; neutralisation was calculated as % transduction of rabbit IgG isotype control. (E) Neutralisation curve of G614+Δ19aa; Alpha+Δ19aa, Beta+Δ19aa and maS2+Δ19aa by R001. The dotted line refers to 50% neutralization from which average IC₅₀ were calculated. (F) Neutralisation curve of G614+Δ19aa; Alpha+Δ19aa, Beta+Δ19aa and maS2+Δ19aa by MM43. The dotted line refers to 50% neutralization from which average IC₅₀ were calculated.

FIG. 11: FIG. 8. Graph showing preliminary rSIV1.5-LV titres by flow cytometry. S-LV was produced using transient transfection of 293T/17 SF cells for VSVg pseudotyped rSIV1 lentiviral vectors (n=1) or SARS-CoV-2 (co)S^{G614+Δ19aa} and maS^{Y49S,T499,G614+Δ19aa} (n=2 each). Crude rSIV1.VSVg and rSIV1.S-LV vector material were harvested 72 h post-transfection, serially diluted and transduced on (co)hACE2 & hTMPRSS2 co-expressing cells. 72 h post transduction, cells were subjected to flow cytometry to determine IU/mL titres based on eGFP transduction.

FIG. 12: maS^{G614+Δ19aa}-LV (S-LVv03) and N501Y containing S-LV (S-LVv04) can transduce BALB/c mice in vivo without exogenous (co)hACE2 priming. (A) Bioluminescence images of BALB/c mice was captured by IVIS at the indicated time points post-i.n. dosing with the indicated dose of S-LV based on p24 quantification (40 or 166 ng). IVIS results provide evidence that maS-LV and N501Y-containing S-LV are capable of robustly transducing mouse lungs without priming and humanising airways with exogenous (co)hACE2 expression. IVIS images were further analysed to determine luminescence activity between groups in the (B) nose and (C) lungs, further demonstrating that maS-LV and N501Y-containing S-LVs are capable of transducing mouse respiratory airway independent of (co)hACE2 priming, in a dose dependent manner. Naïve mice dosed with TSSM LV formulation buffer only served as a negative control and exhibited no-to-background luminescence. i.n., intranasal; ma, mACE2-adapted

FIG. 13: Representative investigation plan to demonstrate gene therapy applications of SARS-CoV-2 S pseudotyped LV using in vitro models. S-LV (S-LV v02, 03, 05, see FIG. 1) encoding therapeutic gene of interest: (so)SFTPB or (so)CFTR2 can be produced to correct mutant SFTPB or CFTR cell models (SFTPB^{lo} or 16HBE14o-with CFTR F508A models, respectively). Rescue of protein expression profile can be demonstrated by WB.

FIG. 14: Graph showing in vivo luciferase transgene expression in the lungs using S^{Y498,T499,G613Δ19aa}-LV (S-LV maS2), S^{B.1.351+Δ19aa}-LV (S-LV Beta) and a vehicle control. (A) Lung luciferase expression was determined at the indicated days post delivery using IVIS in vivo imaging. (B) Cumulative lung luciferase expression was determined over the duration of the study by calculating the area under the curve presented in (A).

FIG. 15: Western blot (SDS PAGE under non-reducing conditions) showing that SARS-CoV-2 S permissive 293T/17 (stably co-expressing hACE2 & hTMPRSS2) transduced with mouse-adapted S-LV (maS3, SARS-CoV-2 S^{P498,T499,Y501,G614+Δ19aa}) express mature SP-B homodimer, compared to null background levels of non-transduced cells. GAPDH served as loading control.

FIG. 16: S-LV transduces Lung Bud Organoids (LBOs) to model COVID-19 infection. Concentrated S-LV (G614+Δ19aa, SARS-CoV-2 S^{G614+Δ19aa}) encoding EGFP was mixed with phenol red (2:1 ratio) and maximal feasible dose (MFD) was microinjected into LBOs embedded in Matrigel. Microinjection of rAAV8 encoding human IgG served as negative control. Fluorescence microscopy (eGFP) was performed 72 h post injection using the EVOS FL Auto. BF=bright field microscopy. Scale bar refers to 500 μm.

FIG. 17: Graphs showing that SARS-CoV-2 2 Nucleoprotein (N) boosts recombinant HIV1 lentiviral vector (LV) titres. (A) Titres from crude vector material titrated on SARS-CoV-2 permissive 293T/17 cells stably expressing hACE2 and hTMPRSS2. nt=not tested (B) Graph showing difference between matched log 10 transformed titre pairs from (A) (paired t-test; P=0.0006).

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the terms “vector”, “viral vector” and “lentiviral vector” are used interchangeably to mean a lentiviral vector pseudotyped with a modified severe acute respiratory syndrome coronavirus 2 (SARS-CoV-2) spike protein, unless otherwise stated.

As used herein, the terms “titre” and “yield” are used interchangeably to mean the amount of lentiviral (e.g. SIV) vector produced by a method of the invention. Titre is the primary benchmark characterising manufacturing efficiency, with higher titres generally indicating that more lentiviral (e.g. SIV) vector is manufactured (e.g. using the same amount of reagents). Titre or yield may relate to the number of vector genomes that have integrated into the genome of a target cell (integration titre), which is a measure of “active” virus particles, i.e. the number of particles capable of transducing a cell. Transducing units (TU/mL also referred to as TTU/mL) is a biological readout of the number of host cells that get transduced under certain tissue culture/virus dilutions conditions, and is a measure of the number of “active” virus particles. The number of “active” virus particles may be quantified in terms of the number of infectious units (IU) per unit volume, such as IU/mL. The total number of (active+inactive) virus particles may also be determined using any appropriate means, such as by measuring either how much Gag is present in the test solution or how many copies of viral RNA are in the test solution. Assumptions are then made that a lentivirus particle contains either 2000 Gag molecules or 2 viral RNA molecules. Once total particle number and a transducing titre/TU have been measured, a particle:infectivity ratio calculated. Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, lie or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, or at least about 100%, or at least about 150%, or at least about 200%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

As used herein the terms “signal peptide”, “signal sequence”, “targeting sequence”, “leader sequence” and “secretory signal” are used interchangeably to mean heterogenous peptide sequences that are found at the N-terminus of secreted proteins that are instrumental in initiating the secretion process. In particular, signal peptides are found in proteins that are targeted to the endoplasmic reticulum and eventually destined to be either secreted or retained in the cell membrane of the cell, particularly as single-pass membrane proteins. Signal peptides are typically removed to produce the mature form of the protein. Signal peptides are normally short peptides, typically about 5 to about 40 amino acids in length, such as about 5 to about 35, or about 10 to about 35 amino acids in length, preferably about 10 to about 30 or about 15 to about 30 amino acids in length. A signal peptide may comprise a core of hydrophobic amino acids, said core typically being about 4 to about 20, such as about 5 to about 20, about 5 to about 16 or about 5 to about 15 amino acids in length). When present, a signal peptide is typically present at the N-terminus of a protein.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual.

Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Lentiviral Vectors

The invention relates to the lentiviral (e.g. SIV) vectors pseudotyped with a modified severe acute respiratory syndrome coronavirus 2 (SARS-CoV-2) spike protein. The term “lentivirus” refers to a genus of the Retroviridae family of RNA viruses that encode the enzyme reverse transcriptase.

Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. A particularly preferred lentiviral vector is an HIV (including all strains and subtypes), or a SIV vector (including all strains and subtypes), such as a SIV-AGM (originally isolated from African green monkeys, Cercopithecus aethiops).

The lentiviral (e.g. HIV/SIV) vectors of the present invention are pseudotyped with a modified severe acute respiratory syndrome coronavirus 2 (SARS-CoV-2) spike protein as described herein. The pseudotyped lentiviral vectors of the invention may therefore be referred to interchangeably as S-LV vectors.

A lentiviral (e.g. HIV/SIV) vector of the invention comprises a transgene operably linked to a promoter. The transgene may encode a therapeutic protein as described herein. In embodiments where the therapeutic protein is a secreted protein, the therapeutic protein may comprise or be associated with a signal peptide. The transgene may include a nucleic acid sequence encoding for a signal peptide (such as the endogenous signal peptide of a secreted protein), or may exclude a nucleic acid sequence encoding for a signal peptide. The therapeutic protein may include a signal peptide (such as the endogenous signal peptide of a secreted protein), or may exclude a signal peptide. Where appropriate, endogenous signal peptides have been identified in the sequence information section herein. All disclosure herein relates to both transgenes and therapeutic proteins including and excluding signal peptides unless explicitly stated. By way of non-limiting example, sequence identity of variants, and/or lengths of fragments may be based on the sequence with or without a signal peptide. Suitable signal peptides are known in the art and can be selected by one of ordinary skill in the art by routine practice. Non-limiting examples of suitable signal peptides are described in UK Patent Application No. 2105277.4, which is herein incorporated by reference in its entirety.

A lentiviral (e.g. HIV/SIV) vector of the invention may have no intron positioned between the promoter and the nucleic acid encoding the signal peptide and/or the nucleic acid encoding the therapeutic protein. Similarly, there may be no intron between the promoter and the nucleic acid encoding the signal peptide and/or the nucleic acid encoding the therapeutic protein in the vector genome plasmid.

A lentiviral (e.g. HIV/SIV) vector according to the invention may be integrase-competent (IC). Alternatively, the lentiviral (e.g. HIV/SIV) vector may be integrase-deficient (ID).

The retroviral/lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO: 46.

Lentiviral vectors, such as those according to the invention, can integrate into the genome of transduced cells and lead to long-lasting expression, making them suitable for transduction of stem/progenitor cells. In the lung, several cell types with regenerative capacity have been identified as responsible for maintaining specific cell lineages in the conducting airways and alveoli. These include basal cells and submucosal gland duct cells in the upper airways, club cells and neuroendocrine cells in the bronchiolar airways, bronchioalveolar stem cells in the terminal bronchioles and type II pneumocytes in the alveoli. Therefore, and without being bound by theory, it is believed that said lentiviral (e.g. HIV/SIV) vectors bring about long term gene expression of the transgene of interest by introducing the transgene into one or more long-lived airway epithelial cells or cell types, such as basal cells and submucosal gland duct cells in the upper airways, club cells and neuroendocrine cells in the bronchiolar airways, bronchioalveolar stem cells in the terminal bronchioles and type II pneumocytes in the alveoli.

Accordingly, the lentiviral (e.g. HIV/SIV) vectors produced according to the invention may transduce one or more cells or cell lines with regenerative potential within the lung (including the airways and respiratory tract) to achieve long term gene expression. For example, the lentiviral (e.g. HIV/SIV) vectors may transduce basal cells, such as those in the upper airways/respiratory tract. Basal cells have a central role in processes of epithelial maintenance and repair following injury. In addition, basal cells are widely distributed along the human respiratory epithelium, with a relative distribution ranging from 30% (larger airways) to 6% (smaller airways).

The lentiviral (e.g. HIV/SIV) vectors according to the invention may be used to transduce isolated and expanded stem/progenitor cells ex vivo prior administration to a patient. Preferably, the lentiviral (e.g. HIV/SIV) vectors produced according to the invention are used to transduce cells within the lung (or airways/respiratory tract) in vivo.

The lentiviral (e.g. HIV/SIV) vectors of the invention demonstrate remarkable resistance to shear forces with only modest reduction in transduction ability when passaged through clinically-relevant delivery devices such as bronchoscopes, spray bottles and nebulisers.

The modified SARS-CoV-2 spike protein with which the lentivirus (e.g. HIV/SIV) vectors of the invention are pseudotyped are rodent/mouse-adapted as described herein. Accordingly, the modified SARS-CoV-2 spike protein with which the lentivirus (e.g. HIV/SIV) vectors of the invention are pseudotyped enables the vector to transduce rodent cells in vitro, ex vivo and in vivo. In particular, the modified SARS-CoV-2 spike protein with which the lentivirus (e.g. HIV/SIV) vectors of the invention are pseudotyped enables the vector to transduce mouse cells in vitro, ex vivo and in vivo. This makes the lentiviral vectors of the invention particularly useful for research, including pre-clinical research, they may be used with rodent (particularly mouse) cell lines and animal models. In contrast, the wild-type SARS-CoV-2 spike protein is not capable of facilitating entry into rodent cells. Accordingly, the pseudotyped lentiviral vectors of the invention are capable of transducing rodent cells, particularly mouse cells, in vitro, ex vivo and in vivo.

In addition, and as described herein the modified SARS-CoV-2 spike protein with which the lentivirus (e.g. HIV/SIV) vectors of the invention are pseudotyped are typically capable of binding to human ACE. Accordingly, the modified SARS-CoV-2 spike protein with which the lentivirus (e.g. HIV/SIV) vectors of the invention are pseudotyped enables the vector to transduce human cells in vitro, ex vivo and in vivo. The ability to transduce both rodent/mouse cells and human cells is advantageous, as it allows the vectors of the invention to be used both in research, including preclinical research, and also in clinical research and applications.

Typically the lentiviral (e.g. HIV/SIV) vectors of the invention comprise a transgene which can be expressed in a host cell. The transgene may encode any protein of interest, such as a therapeutic protein or a reporter protein. Typically the transgene is operably linked to a promoter as described herein.

The lentiviral (e.g. HIV/SIV) vectors of the present invention enable high levels of transgene expression, resulting in high levels (therapeutic levels) of expression of a therapeutic protein. The lentiviral (e.g. HIV/SIV) vectors of the present invention typically provide high expression levels of a transgene when administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or nM.

Expression of a transgene of interest may be given relative to the expression of the corresponding endogenous (defective) gene in a patient. Expression may be measured in terms of mRNA or protein expression. The expression of the transgene of the invention may be quantified relative to the endogenous gene. By way of non-limiting example, when the transgene comprised in a lentiviral vector of the invention is a functional CFTR gene, transgene expression may be relative to the endogenous (dysfunctional) CFTR genes in terms of mRNA copies per cell or any other appropriate unit.

When administered to a subject, in vivo expression levels of a transgene and/or the encoded (therapeutic) protein of the invention may be measured in the lung tissue, epithelial lining fluid and/or serum/plasma as appropriate. A high and/or therapeutic expression level may therefore refer to the concentration in the lung, epithelial lining fluid and/or serum/plasma.

When used in vitro or ex vivo, expression levels of a transgene and/or the encoded (therapeutic) protein of the invention may be measured in conditioned culture medium, or intracellularly (e.g. in a lysate of the cells).

The transgene included in the vector of the invention may be modified to facilitate expression. For example, the transgene sequence may be in CpG-depleted (or CpG-fee) and/or codon-optimised form to facilitate gene expression. Standard techniques for modifying the transgene sequence in this way are known in the art.

The lentiviral (e.g. HIV/SIV) vectors of the invention exhibit efficient airway cell uptake, enhanced transgene expression, and suffer no loss of efficacy upon repeated administration. Accordingly, the lentiviral (e.g. HIV/SIV) vectors of the invention are capable of producing long-lasting, repeatable, high-level expression in airway cells without inducing an undue immune response.

The lentiviral (e.g. HIV/SIV) vectors of the present invention enable long-term transgene expression, resulting in long-term expression of a therapeutic protein. As described herein, the phrases “long-term expression”, “sustained expression”, “long-lasting expression” and “persistent expression” are used interchangeably. Long-term expression according to the present invention means expression of a (therapeutic) gene and/or protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more. This long-term expression may be achieved by repeated doses or by a single dose.

Repeated doses may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated (for in vivo applications), or for the time course of an experiment (for in vitro or ex vivo applications).

The lentiviral (e.g. HIV/SIV) vector typically comprises a promoter operably linked to a transgene, enabling expression of the transgene. The promoter may be a hybrid human CMV enhancer/EF1a (hCEF) promoter. This hCEF promoter may lack the intron corresponding to nucleotides 570-709 and the exon corresponding to nucleotides 728-733 of the hCEF promoter. A preferred example of an hCEF promoter sequence of the invention is provided by SEQ ID NO: 43. The promoter may be a CMV promoter. An example of a CMV promoter sequence is provided by SEQ ID NO: 44. The promoter may be a human elongation factor 1a (EF1a) promoter. An example of a EF1a promoter is provided by SEQ ID NO: 45. Other promoters for transgene expression are known in the art and their suitability for the lentiviral (e.g. HIV/SIV) vectors of the invention determined using routine techniques known in the art. Non-limiting examples of other promoters include UbC and UCOE. As described herein, the promoter may be modified to further regulate expression of the transgene of the invention.

The promoter included in the lentiviral (e.g. HIV/SIV) vector of the invention may be specifically selected and/or modified to further refine regulation of expression of the therapeutic gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90(12): 1487-96), which is herein incorporated by reference in its entirety. Preferably, the lentiviral vectors (particularly SIV vectors) of the invention comprise a hCEF promoter having low or no CpG dinucleotide content. The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO: 43. The absence of CpG dinucleotides further improves the performance of lentiviral (e.g. HIV/SIV) vectors of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The lentiviral (e.g. HIV/SIV) vector of the invention may be modified to allow shut down of gene expression. Standard techniques for modifying the vector in this way are known in the art. As a non-limiting example, Tet-responsive promoters are widely used.

Preferably, the invention relates to lentiviral vectors comprising a promoter and a transgene, particularly HIV/SIV vectors. Pseudotyping with modified SARS-CoV-2 spike protein according to the invention is particularly efficient at targeting cells in the airway epithelium, and as such, for therapeutic applications it is typically delivered to cells of the respiratory tract, including the cells of the airway epithelium. Accordingly, the lentiviral (e.g. HIV/SIV) vectors of the invention are particularly suited for treatment of diseases or disorders of the airways, respiratory tract, or lung. Typically, the lentiviral (e.g. HIV/SIV) vectors may be used for the treatment of a genetic respiratory disease.

A lentiviral (e.g. HIV/SIV) vector of the invention may comprise a transgene that encodes a polypeptide or protein that is therapeutic for the treatment of such diseases, particularly a disease or disorder of the airways, respiratory tract, or lung.

The invention also provides a method of expressing a transgene in a target cell, comprising delivering a pharmaceutical composition or vector as defined herein into the target cell. Said delivering may comprise integrating the lentiviral (e.g. SIV) vector of the pharmaceutical composition into the genome of the target cell.

### SARS-CoV-2 Spike Protein

The SARS-CoV-2 spike protein (also referred to interchangeably herein and in the art as the SARS-CoV-2 S protein) is a class I fusion transmembrane structural glycoprotein that is composed of S1 and S2 subunits. The structure of the SARS-CoV-2 spike protein has been the subject of intense research since the emergence of the SARS-CoV-2 virus and the COVID-19 pandemic. Details of its structure and function are therefore well-known in the art, for example as described in Almehdi et al. (Infection 2021, doi: 10.1007/s15010-021-01677-8, herein incorporated by reference in its entirety). Briefly, the SARS-CoV-2 spike protein is a homotrimer with a size of 180-200 kDa, and a total length of between 1273 and 1300 amino acids.

The SARS-CoV-2 spike comprises a signal peptide (amino acid residues 1-13) and two functional subunits, the S1 and S2 subunits. The S1 subunit comprises an N-terminal domain (NTD) and a receptor binding domain (RBD). The S2 subunit comprises a fusion peptide (FP), heptad repeat 1 (HR1), central helix (CH), connector domain (CD), heptad repeat 2 (HR2), transmembrane domain (TM), and cytoplasmic tail (CT). The function of S1 subunit is bind to the receptor (ACE2) on target cells. The S2 subunit functions to fuse the membranes of viruses and target cells, and thus facilitate entry of the SARS-CoV-2 viral particles into target cells. There is a cleavage site at the border between the S1 and S2 subunits, which is termed the S1/S2 protease cleavage site. As for all coronaviruses, proteases within the target cell cleave the spike protein at the S2′ cleavage site to activate the proteins which is critical to fuse the membranes of viruses and target cells through irreversible conformational changes.

The SARS-CoV-2 spike protein protrudes from the surface of the SARS-CoV-2 virus, where it is capable of recognising and biding to human Angiotensin-converting enzyme 2 (ACE2) receptor on the surface of target cells. ACE2 is found in specific cell types in most organs. For example, ACE2 is abundant in alveolar type II (ATII) cells of the lung, enterocytes of the small intestine, arterial/venous endothelial cells, glia and cortical neurons in the brain. A SARS-CoV-2 pseudotyped lentiviral vector may reasonably be assumed to efficiently target such cells.

Cellular entry of SARS-CoV-2 relies on the viral spike protein binding to ACE2, an interaction synergised by cleavage of the spike protein by Transmembrane protease Serine 2 (TMPRSS2). The sequence for human TMPRSS2 is deposited under UniProt Accession Number 015393 (version 3 of the sequence, accessed 1 Oct. 2021).

However, the spike proteins of all SARS-CoV-2 native variants found in the wild that have been identified to-date bind to human ACE2, but not to rodent homologues. In particular, no native SARS-CoV-2 variant identified to date binds to mouse ACE2. This represents a technical hurdle when researching SARS-CoV-2 and potential treatments and vaccines for COVID-19, because standard research tools such as rodent cell line and rodent models cannot be used. Rather, it is necessary to carry out investigations solely using human cells/tissue (which adds to the costs and limits the scope of pre-clinical testing), or else develop rodent-adapted SARS-CoV-2 spike proteins (which again adds to the cost and complexity of research).

To that end, some work has been carried out on generating mouse-adapted SARS-CoV-2 spike proteins. However, to-date the primary focus of this work has been to generate SARS-CoV-2 viral variants that can be used in research into SARS-Co-V2, and into treatments and vaccines for COVID-19. To-date, despite all the research that is being conducted globally in relation to SARS-CoV-2, there has been no attempt to harness mouse-adapted SARS-CoV-2 spike protein to pseudotype viral vectors for research/therapeutic purposes beyond COVID-19.

The present inventors are the first to develop a lentiviral vector that is pseudotyped with a modified, mouse-adapted SARS-CoV-2 spike protein. Accordingly, the invention provides modified SARS-CoV-2 spike proteins as described herein, as well as lentiviral (e.g. HIV/SIV) vectors pseudotyped with said modified SARS-CoV-2 spike proteins.

The modified SARS-CoV-2 spike protein of the invention may be derived from any SARS-CoV-2 spike protein. By “derived from”, it is meant that a modified SARS-CoV-2 spike protein of the invention may comprise one or more modification, such as one, two, three, four, five, six, seven, eight, nine, 10 or more modifications, compared with the SARS-CoV-2 spike protein from which it is derived.

A modified SARS-CoV-2 spike protein of the invention may be derived from the spike protein of any SARS-CoV-2 strain or variant. For example, a modified SARS-CoV-2 spike protein of the invention may be derived from: the spike protein of any one of the Wuhan-Hu-1 strain, the B.1.1.529 (Omicron variant), the B.1.351 strain (Beta variant), the P.1 strain (Gamma variant), the B.1.617.2 strain (Delta variant), the B.1.621 strain (Mu variant), the C.37 strain (Lambda variant), the B.1.620 strain, the B.1.1.7 strain (Alpha variant), the B.1.427 or B.1.429 strain (Epsilon variant), the B.1.525 strain (Eta variant), the B.1.526 strain (Iota variant), the B.1.617 strain (Kappa variant), the G614 strain, the Australia/VIC01/2020 (Aus/VIC01) strain, the B.1.1.529 Strain (Omicron variant) or any substrain/sublineage thereof, or a SARS-CoV-2 spike protein having at least at least 95%, at least 96%, at least 97%, at least 98%, at least 99%, at least 99.5% or more sequence identity to any one of these SARS-Co-V-2 spike proteins. The spike protein of the Wuhan-Hu-1 strain of SARS-CoV-2 is SEQ ID NO: 1 herein. By way of non-limiting example, a modified SARS-CoV-2 spike protein of the invention may be derived from a spike protein having at least 90%, at least 95%, at least 96%, at least 97%, at least 98%, at least 99%, at least 99.5% or more sequence identity to SEQ ID NO: 1.

A modified SARS-CoV-2 spike protein of the invention typically comprises a modification at one or more amino acid position corresponding to, or aligning with one or more specific amino acid positions of SEQ ID NO: 1. Wherein the modified SARS-CoV-2 spike protein is derived from a SARS-CoV-2 spike protein other than that of SEQ ID NO: 1, one of ordinary skill in the art will readily be able to determine the corresponding amino acid positions within the SARS-CoV-2 spike protein from which the modified SARS-CoV-2 spike protein of the invention is to be derived. By way of non-limiting example, the SARS-CoV-2 spike protein from which the modified SARS-CoV-2 spike protein of the invention it to be derived may be aligned with the SARS-CoV-2 spike protein of SEQ ID NO: 1 using readily available alignment tools (such as the BLAST tool described herein). The amino acid residues within the SARS-CoV-2 spike protein from which the modified SARS-CoV-2 spike protein of the invention is to be derived that align with/correspond to the identified residues within SEQ ID NO: 1 (e.g. residues 498, 499 and 614 of SEQ ID NO: 1) may then readily be identified using the alignment.

An amino acid modification according to the invention may be a mutation, such as a substitution, deletion, addition, or other modification, including post-translational modification, unless the relevant disclosure explicitly says otherwise. Preferably said modifications are amino acid substitutions. In other words, the amino acid at a specified position within the SARS-CoV-2 spike protein is substituted by a naturally occurring or non-naturally occurring amino acid that is different to the amino acid present at that position in the SARS-CoV-2 spike protein from which the modified SARS-CoV-2 spike protein of the invention is derived. Alternatively, the amino acid at a specified position within the modified SARS-CoV-2 spike protein of the invention may be modified post-translationally. Post-translational modifications include glycosylations, acetylations, acylations, de-aminations, phosphorylisations, isoprenylisations, glycosyl phosphatidyl inositolisations and further modifications known to a person skilled in the art.

The modification of one or more amino acid position as described herein may be performed, for example, by specific mutagenesis, or any other method known in the art.

Wherein the one or more amino acid position is substituted relative to the corresponding SARS-CoV-2 spike protein from which the modified SARS-CoV-2 spike protein of the invention is derived, the substitution may be a conservative substitution or a non-conservative substitution, preferably a non-conservative substitution.

A modified SARS-CoV-2 spike protein of the invention typically comprises a modification at each amino acid position corresponding to, or aligning with amino acid positions 498, 499 and 614 of SEQ ID NO: 1. Typically the modifications at one or more of the amino acid positions corresponding to, or aligning with amino acid positions 498, 499 and 614 of SEQ ID NO: 1, or any combination thereof, are mutations, preferably substitutions. In some particularly preferred embodiments, a modified SARS-CoV-2 spike protein of the invention comprises a substitution at each amino acid position corresponding to, or aligning with amino acid positions 498, 499 and 614 of SEQ ID NO: 1.

The amino acid residue corresponding to or aligning with amino acid position 498 of SEQ ID NO: 1 may be substituted by a tyrosine or a histidine residue.

The amino acid residue corresponding to or aligning with amino acid position 499 of SEQ ID NO: 1 may be substituted by a threonine residue.

The amino acid residue corresponding to or aligning with amino acid position 614 of SEQ ID NO: 1 may be substituted by a glycine residue.

Any combination of these substitutions may be comprised in a modified SARS-CoV-2 spike protein of the invention. For example: (i) the amino acid residue corresponding to or aligning with amino acid position 498 of SEQ ID NO: 1 may be substituted by a tyrosine residue and the amino acid residue corresponding to or aligning with amino acid position 499 of SEQ ID NO: 1 may be substituted by a threonine residue; (ii) the amino acid residue corresponding to or aligning with amino acid position 498 of SEQ ID NO: 1 may be substituted by a tyrosine residue and the amino acid residue corresponding to or aligning with amino acid position 614 of SEQ ID NO: 1 may be substituted by a glycine residue; (iii) the amino acid residue corresponding to or aligning with amino acid position 499 of SEQ ID NO: 1 may be substituted by a threonine residue and the amino acid residue corresponding to or aligning with amino acid position 614 of SEQ ID NO: 1 may be substituted by a glycine residue; or (iv) the amino acid residue corresponding to or aligning with amino acid position 498 of SEQ ID NO: 1 may be substituted by a tyrosine residue, the amino acid residue corresponding to or aligning with amino acid position 499 of SEQ ID NO: 1 may be substituted by a threonine residue and the amino acid residue corresponding to or aligning with amino acid position 614 of SEQ ID NO: 1 may be substituted by a glycine residue. Preferably, a modified SARS-CoV-2 spike protein of the invention comprises an amino acid substitution at each amino acid position corresponding to or aligning with amino acid positions 498, 499 and 614 of SEQ ID NO: 1, wherein the amino acid residue corresponding to or aligning with amino acid position 498 of SEQ ID NO: 1 is substituted by a tyrosine residue, the amino acid residue corresponding to or aligning with amino acid position 499 of SEQ ID NO: 1 is substituted by a threonine residue and the amino acid residue corresponding to or aligning with amino acid position 614 of SEQ ID NO: 1 is substituted by a glycine residue.

A modified SARS-CoV-2 spike protein of the invention may comprise one or more of the mutations (i) Q498Y; (ii) P499T; or (iii) D614G; or any combination thereof. A modified SARS-CoV-2 spike protein of the invention may comprise the Q498Y and P499T mutations, the Q498Y and D614G mutations, the P499T and D614G, or the Q498Y, P499T and D614G mutations.

The invention also provides a modified severe acute respiratory syndrome coronavirus 2 (SARS-CoV-2) spike protein which comprises: (a) a mutation at an amino acid position corresponding to, or aligning with, position 614 of SEQ ID NO: 1; (b) a deletion of at least a portion of the cytoplasmic tail; and (c) (i) mutations at amino acid positions corresponding to, or aligning with, positions 498 and 499 of SEQ ID NO: 1; and/or (ii) a mutation at an amino acid position corresponding to, or aligning with, position 501 of SEQ ID NO: 1. For the avoidance of doubt, all disclosure herein in relation to modified SARS-CoV-2 spike proteins of the invention applies equally and without reservation to said modified SARS-CoV-2 spike proteins. By way of non-limiting example, said modified SARS-CoV-2 spike protein may comprise one or more additional mutation as described herein and/or the deletion of at least a portion of the cytoplasmic tail may be as described herein.

The present inventors are the first to successfully generate lentiviral vectors pseudotyped with mouse-adapted SARS-CoV-2 spike protein. Therefore, the invention provides lentiviral vectors comprising one or more mutation within the SARS-CoV-2 spike protein which allows cellular entry via mouse ACE2. Accordingly, the invention also provides a modified severe acute respiratory syndrome coronavirus 2 (SARS-CoV-2) spike protein which comprises: (a) a modification at each amino acid position corresponding to, or aligning with amino acid positions 498 and 499 of SEQ ID NO: 1; and (b) a deletion of at least a portion of the cytoplasmic tail. Said modified SARS-CoV-2 spike protein may comprise one or more additional mutation, as described herein. By way of non-limiting example, said modified SARS-CoV-2 spike protein may comprise one or more additional mutation as described herein and/or the deletion of at least a portion of the cytoplasmic tail may be as described herein.

A modified SARS-CoV-2 spike protein of the invention may comprise a mutation, typically a substitution at an amino acid position corresponding to or aligning with amino acid position 493 of SEQ ID NO: 1, either instead of a mutation at positions amino acid position corresponding to or aligning with amino acid positions 498 or 499 or SEQ ID NO: 1, or in addition to mutations at amino acid positions corresponding to or aligning with amino acid positions 498 and/or 498 of SEQ ID NO: 1. The amino acid residue corresponding to or aligning with amino acid position 493 of SEQ ID NO: 1 may be substituted by a lysine residue. Any disclosure herein relating to modified SARS-CoV-2 spike proteins of the invention comprising mutations at amino acid positions 498, 499 and/or 614 of SEQ ID NO: 1, or any combination thereof, apply equally and without reservation to modified SARS-CoV-2 spike proteins which comprise a mutation at an amino acid position corresponding to or aligning with amino acid position 493 of SEQ ID NO: 1. By way of non-limiting example, a modified SARS-CoV-2 spike protein of the invention may have mutations at (i) amino acid positions 493 and 498 of SEQ ID NO: 1; (ii) amino acid positions 493 and 499 of SEQ ID NO: 1; (iii) amino acid positions 493 and 614 of SEQ ID NO: 1; (iv) amino acid positions 493, 498 and 499 of SEQ ID NO: 1; (v) amino acid positions 493, 498 and 614 of SEQ ID NO: 1; (vi) amino acid positions 493, 499 and 614 of SEQ ID NO: 1; or (vii) amino acid positions 493, 498, 498 and 614 of SEQ ID NO: 1. Said combinations may also comprise a C-terminal deletion and/or any other mutation or substitution described herein.

A modified SARS-CoV-2 spike protein of the invention typically comprises a C-terminal deletion compared with the unmodified SARS-CoV-2 spike protein from which it is derived. The C-terminal deletion typically deletes at least a portion of the cytoplasmic tail of the spike protein. The cytoplasmic tail of the SARS-CoV-2 spike protein of the Wuhan-Hu-1 strain corresponds to amino acid residues 1235 to 1273 of SEQ ID NO: 1. The cytoplasmic tail of the spike protein from other SARS-CoV-2 strains typically corresponds to or aligns with the cytoplasmic tail of the SARS-CoV-2 spike protein of the Wuhan-Hu-1 strain.

A modified SARS-CoV-2 spike protein of the invention may comprise a C-terminal deletion of at least 10 amino acids, at least 15 amino acids, at least 16 amino acids, at least 17 amino acids, at least 18 amino acids, at least 19 amino acids, at least 20 amino acids or more compared with the unmodified SARS-CoV-2 spike protein from which it is derived. A modified SARS-CoV-2 spike protein of the invention may comprise a C-terminal deletion of the entire cytoplasmic tail. The C-terminal deletion may delete at least a portion of a putative endoplasmic reticulum retention signal (ERS) within the cytoplasmic tail of the SARS-CoV-2 spike protein. The C-terminal deletion may delete at the entire putative endoplasmic reticulum retention signal (ERS) within the cytoplasmic tail of the SARS-CoV-2 spike protein. In some preferred embodiments, a modified SARS-CoV-2 spike protein of the invention may comprise a C-terminal deletion of the amino acids corresponding to, or aligning with amino acid positions 1255 to 1273 of SEQ ID NO: 1.

Typically a lentiviral vector of the invention comprises a modified SARS-CoV-2 spike protein which comprises both (i) modifications (such as mutations, particularly substitutions) at amnio acid positions corresponding to or aligning with amino acid positions 498, 499 and 614 of SEQ ID NO: 1; and (ii) a deletion of at least a portion of the cytoplasmic tail. Any modifications at amnio acid positions corresponding to or aligning with amino acid positions 498, 499 and 614 of SEQ ID NO: 1 may be used in combination with any deletion or partial deletion of the cytoplasmic tail in a modified SARS-CoV-2 spike protein according to the invention. In particularly preferred embodiments, a lentiviral vector of the invention comprises substitutions at amnio acid positions corresponding to or aligning with amino acid positions 498, 499 and 614 of SEQ ID NO: 1 and a deletion of at least 15 amino acids of the cytoplasmic tail, even more preferably wherein the deletion corresponds to or aligns with positions 1255 to 1273 of SEQ ID NO: 1.

A modified SARS-CoV-2 spike protein of the invention may comprise or consist of an amino acid sequence having at least 90% identity, at least 95%, at least 96%, at least 97%, at least 98%, at least 99%, at least 99.5% or more sequence identity to SEQ ID NO: 13.

A modified SARS-CoV-2 spike protein of the invention may comprise one or more additional modification, typically one or more additional mutation, compared with the SARS-CoV-2 spike protein from which it is derived. Said one or more additional mutation may be an amino acid substitution.

Examples of additional modifications, particularly additional mutations, and even more particularly substitutions, in SARS-CoV-2 spike proteins are well-known in the art, and it is within the routine skill of one of ordinary skill in the art to select and introduce one or more additional modification into a modified SARS-CoV-2 spike protein of the invention.

Typically, when a modified SARS-CoV-2 spike protein of the invention comprises one or more additional modification, said one or more additional modification comprises or consists of a modification at an amino acid position corresponding to, or aligning with amino acid position 501 of SEQ ID NO: 1. Said modification at an amino acid position corresponding to, or aligning with amino acid position 501 of SEQ ID NO: 1 is typically a mutation. Said mutation at an amino acid position corresponding to, or aligning with amino acid position 501 of SEQ ID NO: 1 may be an amino acid substitution, either a non-conservative or conservative amino acid substitution. In some preferred embodiments, said substitution at an amino acid position corresponding to, or aligning with amino acid position 501 of SEQ ID NO: 1 is a non-conservative amino acid substitution, with substitution by tyrosine being particularly preferred. Thus, in some preferred embodiments, a modified SARS-CoV-2 spike protein of the invention comprises one or more additional amino acid modification comprising or consisting of a substitution at an amino acid position corresponding to or aligning with amino acid position 501 of SEQ ID NO: 1, wherein the amino acid residue corresponding to or aligning with amino acid position 501 of SEQ ID NO: 1 is substituted by a tyrosine residue.

A modified SARS-CoV-2 spike protein of the invention may therefore comprise one or more additional modification comprising N501Y.

Other non-limiting examples of amino acid modifications that may be comprised in a modified SARS-CoV-2 spike protein of the invention include a modification at any one of amino acid positions corresponding to or aligning with residues 5, 69, 70, 80, 95, 142, 144, 154, 157, 215, 241, 242, 243, 253, 346, 417, 452, 477, 478, 484, 494, 570, 981, 701, 716, 859, 950, 951, 957, 982, 1071, 1118, and 1191 of SEQ ID NO: 1, or any combination thereof. In particular, other non-limiting examples of amino acid modifications that may be comprised in a modified SARS-CoV-2 spike protein of the invention include: a substitution at an amino acid position corresponding to or aligning with amino acid position 5 of SEQ ID NO: 1, wherein the amino acid residue corresponding to or aligning with amino acid position 5 of SEQ ID NO: 1 is substituted by a phenylalanine residue; a deletion at an amino acid position corresponding to or aligning with amino acid position 69 of SEQ ID NO: 1; a deletion at an amino acid position corresponding to or aligning with amino acid position 70 of SEQ ID NO: 1; a substitution at an amino acid position corresponding to or aligning with amino acid position 80 of SEQ ID NO: 1, wherein the amino acid residue corresponding to or aligning with amino acid position 80 of SEQ ID NO: 1 is substituted by a glycine or alanine residue; a substitution at an amino acid position corresponding to or aligning with amino acid position 95 of SEQ ID NO: 1, wherein the amino acid residue corresponding to or aligning with amino acid position 95 of SEQ ID NO: 1 is substituted by an isoleucine residue; a substitution at an amino acid position corresponding to or aligning with amino acid position 142 of SEQ ID NO: 1, wherein the amino acid residue corresponding to or aligning with amino acid position 142 of SEQ ID NO: 1 is substituted by an aspartic acid residue; a substitution or a deletion at an amino acid position corresponding to or aligning with amino acid position 144 of SEQ ID NO: 1; a substitution at an amino acid position corresponding to or aligning with amino acid position 154 of SEQ ID NO: 1, wherein the amino acid residue corresponding to or aligning with amino acid position 154 of SEQ ID NO: 1 is substituted by a lysine residue; a substitution at an amino acid position corresponding to or aligning with amino acid position 157 of SEQ ID NO: 1, wherein the amino acid residue corresponding to or aligning with amino acid position 157 of SEQ ID NO: 1 is substituted by a serine residue; a substitution at an amino acid position corresponding to or aligning with amino acid position 215 of SEQ ID NO: 1, wherein the amino acid residue corresponding to or aligning with amino acid position 215 of SEQ ID NO: 1 is substituted by a glycine residue; a deletion at an amino acid position corresponding to or aligning with amino acid position 241 of SEQ ID NO: 1; a deletion at an amino acid position corresponding to or aligning with amino acid position 242 of SEQ ID NO: 1; a deletion at an amino acid position corresponding to or aligning with amino acid position 243 of SEQ ID NO: 1; a substitution at an amino acid position corresponding to or aligning with amino acid position 253 of SEQ ID NO: 1, wherein the amino acid residue corresponding to or aligning with amino acid position 253 of SEQ ID NO: 1 is substituted by a glycine residue; a substitution at an amino acid position corresponding to or aligning with amino acid position 346 of SEQ ID NO: 1, wherein the amino acid residue corresponding to or aligning with amino acid position 346 of SEQ ID NO: 1 is substituted by a lysine residue; a substitution at an amino acid position corresponding to or aligning with amino acid position 417 of SEQ ID NO: 1, wherein the amino acid residue corresponding to or aligning with amino acid position 417 of SEQ ID NO: 1 is substituted by an asparagine or threonine residue; a substitution at an amino acid position corresponding to or aligning with amino acid position 452 of SEQ ID NO: 1, wherein the amino acid residue corresponding to or aligning with amino acid position 452 of SEQ ID NO: 1 is substituted by an arginine residue; a substitution at an amino acid position corresponding to or aligning with amino acid position 477 of SEQ ID NO: 1, wherein the amino acid residue corresponding to or aligning with amino acid position 477 of SEQ ID NO: 1 is substituted by an asparagine residue; a substitution at an amino acid position corresponding to or aligning with amino acid position 478 of SEQ ID NO: 1, wherein the amino acid residue corresponding to or aligning with amino acid position 478 of SEQ ID NO: 1 is substituted by a lysine residue; a substitution at an amino acid position corresponding to or aligning with amino acid position 484 of SEQ ID NO: 1, wherein the amino acid residue corresponding to or aligning with amino acid position 484 of SEQ ID NO: 1 is substituted by a lysine or glutamine residue; a substitution at an amino acid position corresponding to or aligning with amino acid position 494 of SEQ ID NO: 1, wherein the amino acid residue corresponding to or aligning with amino acid position 494 of SEQ ID NO: 1 is substituted by a proline residue; a substitution at an amino acid position corresponding to or aligning with amino acid position 570 of SEQ ID NO: 1, wherein the amino acid residue corresponding to or aligning with amino acid position 570 of SEQ ID NO: 1 is substituted by an aspartic acid residue; a substitution at an amino acid position corresponding to or aligning with amino acid position 618 of SEQ ID NO: 1, wherein the amino acid residue corresponding to or aligning with amino acid position 618 of SEQ ID NO: 1 is substituted by an arginine residue; a substitution at an amino acid position corresponding to or aligning with amino acid position 681 of SEQ ID NO: 1, wherein the amino acid residue corresponding to or aligning with amino acid position 681 of SEQ ID NO: 1 is substituted by a histidine residue; a substitution at an amino acid position corresponding to or aligning with amino acid position 701 of SEQ ID NO: 1, wherein the amino acid residue corresponding to or aligning with amino acid position 701 of SEQ ID NO: 1 is substituted by a valine residue; a substitution at an amino acid position corresponding to or aligning with amino acid position 716 of SEQ ID NO: 1, wherein the amino acid residue corresponding to or aligning with amino acid position 716 of SEQ ID NO: 1 is substituted by an isoleucine residue; a substitution at an amino acid position corresponding to or aligning with amino acid position 859 of SEQ ID NO: 1, wherein the amino acid residue corresponding to or aligning with amino acid position 859 of SEQ ID NO: 1 is substituted by an asparagine residue; a substitution at an amino acid position corresponding to or aligning with amino acid position 950 of SEQ ID NO: 1, wherein the amino acid residue corresponding to or aligning with amino acid position 950 of SEQ ID NO: 1 is substituted by a histidine residue; a substitution at an amino acid position corresponding to or aligning with amino acid position 957 of SEQ ID NO: 1, wherein the amino acid residue corresponding to or aligning with amino acid position 957 of SEQ ID NO: 1 is substituted by an arginine residue; a substitution at an amino acid position corresponding to or aligning with amino acid position 982 of SEQ ID NO: 1, wherein the amino acid residue corresponding to or aligning with amino acid position 982 of SEQ ID NO: 1 is substituted by an alanine residue; a substitution at an amino acid position corresponding to or aligning with amino acid position 1071 of SEQ ID NO: 1, wherein the amino acid residue corresponding to or aligning with amino acid position 1071 of SEQ ID NO: 1 is substituted by a histidine residue; a substitution at an amino acid position corresponding to or aligning with amino acid position 1118 of SEQ ID NO: 1, wherein the amino acid residue corresponding to or aligning with amino acid position 1118 of SEQ ID NO: 1 is substituted by a histidine residue; or a substitution at an amino acid position corresponding to or aligning with amino acid position 1191 of SEQ ID NO: 1, wherein the amino acid residue corresponding to or aligning with amino acid position 1191 of SEQ ID NO: 1 is substituted by an asparagine residue; or any combination thereof.

Thus, a modified SARS-CoV-2 spike protein of the invention may therefore comprise one or more additional modification comprising one or more of L5F, T19R, 69del, V70F, 70del, G75V, T761, D80G/A, T951, G142D, Y144X or 144del, E154K, E156-, F157-, F157S, R158G, D215G, A222V, 241del, 242del, 243del, 246-252del, D253G, W258L, R346K, K417N/T, L452Q/R, S477N, T478K, E484K/Q, F490S, S494P, A570D, P618R, P681H, A701V, T7161, T859N, D950N/H, Q957R, S982A, Q1071H, D1118H or K1191N, or any combination thereof.

A lentiviral vector of the invention may comprise a modified SARS-CoV-2 spike protein which comprises (i) modifications (such as mutations, particularly substitutions) at amnio acid positions corresponding to or aligning with amino acid positions 498, 499 and 614 of SEQ ID NO: 1; (ii) a deletion of at least a portion of the cytoplasmic tail; and (iii) a modification (such as a mutation, particularly a substitution) at an amino acid position corresponding to or aligning with amino acid position 501 of SEQ ID NO: 1. Any modifications at amnio acid positions corresponding to or aligning with amino acid positions 498, 499 and 614 of SEQ ID NO: 1 may be used in combination with any deletion or partial deletion of the cytoplasmic tail and any modification at an amino acid position corresponding to or aligning with amino acid position 501 of SEQ ID NO: 1 in a modified SARS-CoV-2 spike protein according to the invention. In particularly preferred embodiments, a lentiviral vector of the invention comprises substitutions at amnio acid positions corresponding to or aligning with amino acid positions 498, 499, 501 and 614 of SEQ ID NO: 1 and a deletion of at least 15 amino acids of the cytoplasmic tail, even more preferably wherein the deletion corresponds to or aligns with positions 1255 to 1273 of SEQ ID NO: 1.

A modified SARS-CoV-2 spike protein of the invention may comprise or consist of an amino acid sequence having at least 90% identity, at least 95%, at least 96%, at least 97%, at least 98%, at least 99%, at least 99.5% or more sequence identity to SEQ ID NO: 20.

In some embodiments, a modified SARS-CoV-2 spike protein of the invention may comprise a mutation at position corresponding to or aligning with amino acid position 614 of SEQ ID NO: 1 (as described herein), and wherein said modified SARS-CoV-2 spike protein is derived from the spike protein of the B.1.351 strain (Beta variant). Preferably such modified SARS-CoV-2 spike protein of the invention further comprise a deletion of at least a portion of the cytoplasmic tail (as described herein), more preferably a deletion of at least 15 amino acids of the cytoplasmic tail, and even more preferably wherein the deletion corresponds to or aligns with positions 1255 to 1273 of SEQ ID NO: 1. The invention provides a modified SARS-CoV-2 spike protein comprising D80A, D215G, K417N, E484K, N501Y, D614G, A701V and a C-terminal deletion which corresponds to or aligns with positions 1255 to 1273 of SEQ ID NO: 1. Such a modified SARS-CoV-2 spike protein is typically derived from the spike protein of the B.1.351 strain (Beta variant). As exemplified herein, such a modified SARS-CoV-2 spike protein can provide further increases in in vivo transgene expression in the lungs Thus, the invention provides a lentiviral vector pseudotyped with a modified severe acute respiratory syndrome coronavirus 2 (SARS-CoV-2) spike protein, which lentiviral vector comprises a transgene operably linked to a promoter; and wherein said spike protein comprises: (a) mutations at amino acid positions corresponding to, or aligning with, one or more of positions 80, 215, 417, 484, 501, 614 and 701 of SEQ ID NO: 1 wherein preferably all these residues are mutated; and (b) a deletion of at least a portion of the cytoplasmic tail. Said modified SARS-CoV-2 spike protein may be derived from the spike protein of the B.1.351 strain. In some embodiments: the amino acid corresponding to, or aligning with: (i) position 80 of SEQ ID NO: 1 is substituted by alanine; (ii) position 215 of SEQ ID NO: 1 is substituted by glycine; (iii) position 417 of SEQ ID NO: 1 is substituted by asparagine; (iv) position 484 of SEQ ID NO: 1 is substituted by lysine; (v) position 501 of SEQ ID NO: 1 is substituted by tyrosine; (vi) position 614 of SEQ ID NO: 1 is substituted by glycine and/or (vii) position 701 of SEQ ID NO: 1 is substituted by valine; wherein preferably all these residues are substituted. Alternatively or in addition, the deletion of at least a portion of the cytoplasmic tail comprises or consists of deletion of the amino acid residues corresponding to or aligning with positions 1255 to 1273 of SEQ ID NO: 1.

As described herein, the modified SARS-CoV-2 spike proteins of the invention are rodent-adapted, particularly mouse-adapted. As used herein, the term “rodent-adapted” means that the modified SARS-CoV-2 spike proteins are capable of binding to rodent ACE2, and facilitating entry to a target rodent cell which expresses rodent ACE2. Similarly, “mouse-adapted” means that the modified SARS-CoV-2 spike proteins are capable of binding to mouse ACE2, and facilitating entry to a target mouse cell which expresses mouse ACE2. Methods for determining mouse-adaptation include assays for determining/quantifying transduction of mouse cells, such as those described in the Examples herein. Other suitable techniques are known in the art and could be readily selected and used by one of ordinary skill without undue burden.

Rodent/mouse adaptation of SARS-CoV-2 spike protein comprised in pseudotyped lentiviral vectors of the invention allows the pseudotyped lentiviral vectors of the invention greater flexibility for use in research, for example in in vitro and/or ex vivo assays using rodent/mouse cells or tissue, or in in vivo rodent/mouse models, as described herein.

The modified SARS-CoV-2 spike proteins of the invention are typically capable of binding to human ACE2, particularly to the enzymatic domain of human ACE2, and facilitating entry to a target human cell which expresses human ACE2. Typically, the modified SARS-CoV-2 spike proteins of the invention are capable of binding to human ACE2, particularly to the enzymatic domain of human ACE2 in addition to being rodent/mouse-adapted. Standard techniques for assessing binding are known in the art (such as surface plasmon resonance) and could be readily used by one of ordinary skill without undue burden.

The modified SARS-CoV-2 spike proteins of the invention may be detected by anti-coronavirus spike protein antibodies. In other words, the modified SARS-CoV-2 spike proteins of the invention may bind to/be recognised by antibodies that bind specifically to an unmodified SARS-CoV-2 spike protein. This may be described as the modified SARS-CoV-2 spike proteins of the invention being neutralised by neutralising antibodies that bind specifically to an unmodified SARS-CoV-2 spike protein. By way of non-limiting example, the modified SARS-CoV-2 spike proteins of the invention may bind to/be detected/recognised by the MM43 and/or R001 antibodies (both available from SinoBiological: R001=Catalogue No. 40592-R001, MM43=Catalogue No. 40591-MM43). Alternatively, the modified SARS-CoV-2 spike proteins of the invention may be not be detected by anti-coronavirus spike protein antibodies. In other words, the modified SARS-CoV-2 spike proteins of the invention may not bind to/be recognised by antibodies that bind specifically to an unmodified SARS-CoV-2 spike protein. This may be described as the modified SARS-CoV-2 spike proteins of the invention escaping neutralisation by neutralising antibodies that bind specifically to an unmodified SARS-CoV-2 spike protein. By way of non-limiting example, the modified SARS-CoV-2 spike proteins of the invention may not bind to/be detected/recognised by the MM43 and/or R001 antibodies. Preferably, the modified SARS-CoV-2 spike proteins of the invention may be not be detected by anti-coronavirus spike protein antibodies. Thus, lentiviral vectors of the invention that are pseudotyped with a modified SARS-CoV-2 spike protein of the invention may avoid the immune system of an individual following administration of the pseudotyped lentiviral vector to said individual, and be more efficacious and/or persist for longer within the individual compared with a lentiviral vector pseudotyped with the corresponding unmodified SARS-CoV-2 spike protein.

Neutralisation or neutralisation escape by a modified SARS-CoV-2 spike proteins of the invention may be specific to particular antibodies that bind specifically to an unmodified SARS-CoV-2 spike protein. By way of non-limiting example, a modified SARS-CoV-2 spike protein of the invention derived from the B.1.1.7 SARS-CoV-2 strain may escape neutralisation by MM43, but not R001. By way of a further non-limiting example, a modified SARS-CoV-2 spike protein of the invention derived from the B.1.351 SARS-CoV-2 strain may escape neutralisation by R001, but not MM43.

### Polynucleotides and Constructs

The present invention also provides a polynucleotide that encodes a modified SARS-CoV-2 spike protein of the invention. The term polynucleotide encompasses both DNA and RNA sequences.

A polynucleotide of the invention may be used for recombinant expression of the modified SARS-CoV-2 spike protein of the invention.

A polynucleotide of the invention may optionally be codon optimised for expression in a particular cell type, for example, eukaryotic cells (e.g. mammalian cells, yeast cells, insect cells or plants cells) or prokaryotic cells (e.g. E. coli). The term “codon optimised” refers to the replacement of at least one codon within a base polynucleotide sequence with a codon that is preferentially used by the host organism in which the polynucleotide is to be expressed. Typically, the most frequently used codons in the host organism are used in the codon-optimised polynucleotide sequence. Methods of codon optimisation are well known in the art.

It will be understood by a skilled person that numerous different polynucleotides can encode the same polypeptide as a result of the degeneracy of the genetic code. It is also understood that skilled persons may, using routine techniques, make nucleotide substitutions that do not affect the polypeptide sequence encoded by the nucleic acid molecules to reflect the codon usage of any particular host organism in which the polypeptides are to be expressed. Therefore, unless otherwise specified, a “polynucleotide that encodes the protein or immunogenic fragment of the invention” includes all polynucleotide sequences that are degenerate versions of each other and that encode the same amino acid sequence.

The present invention also provides an expression cassette comprising a polynucleotide of the invention operably linked to a promoter. The choice of promoter will depend on where the ultimate expression of the polynucleotide will take place. In general, constitutive promoters are preferred, but inducible promoters may likewise be used. Suitable promoter sequences are well known in the art, and examples are described herein.

The expression cassette may be DNA, such as a DNA plasmid, or an RNA vector, such as an mRNA vector or a self-amplifying RNA vector. The expression cassette of the invention may be capable of expression in eukaryotic and/or prokaryotic cells.

The polynucleotide or expression cassette may also comprise a suitable terminator sequence. Suitable terminator sequences are well known in the art.

The polynucleotides and expression cassettes of the invention may be made using any suitable process known in the art. Thus, the polynucleotides and expression cassettes may be made using chemical synthesis techniques. Alternatively, the polynucleotides and expression cassettes of the invention may be made using molecular biology techniques.

Polynucleotides and expression cassettes of the present invention may be designed in silico, and then synthesised by conventional polynucleotide synthesis techniques.

### Methods of Production

The present invention also provides methods for producing lentiviral (e.g. HIV/SIV) vectors pseudotyped with a modified SARS-CoV-2 spike protein of the invention.

Accordingly, the invention provides a method of producing a lentiviral vector as described herein, the method comprising: (a) introducing (i) a nucleic acid sequence encoding a modified SARS-CoV-2 spike protein of the invention; and (ii) one or more nucleic acid sequence encoding lentiviral packaging components, lentiviral envelope components, and a lentiviral genome, into a viral vector production cell; and (b) culturing the production cell under conditions suitable for the production of the lentiviral vector. Said method may further comprise harvesting the lentiviral vector.

The nucleic acid sequence encoding the modified SARS-CoV-2 spike protein may be comprised in a polynucleotide or expression construct of the invention.

Said method may comprise the use of codon-optimised gag-pol genes. Typically the codon-optimised gag-pol genes used in the production methods of the invention are matched to the lentiviral vector being produced. By way of non-limiting example, when the lentiviral vector is an HIV vector, the codon-optimised gag-pol genes used in the production methods of the invention are HIV gag-pol genes. By way of non-limiting example, when the lentiviral vector is an SIV vector, the codon-optimised gag-pol genes used in the production methods of the invention are SIV gag-pol genes.

In addition to codon-optimisation, the codon-optimised gag-pol genes used in the production methods of the invention may comprise other modifications, such as a translational slip (which allows translation to slip from one region to another to allow the production of both Gag and Pol). Any suitable variation of codon usage may be used in the codon-optimised gag-pol genes of the invention, provided that (i) homology between the vector genome plasmid and GagPol plasmid is reduced to minimise the risk of RCL production and (ii) after codon optimisation there is production of sufficient GagPol without the inclusion of RRE (this further reduces homology and the risk of RCL production).

The codon-optimised gag-pol genes used in the production methods of the invention may be completely (100%) or partially codon-optimised. Partial codon-optimisation encompasses at least 70%, at least 80%, at least 95%, at least 90%, at least 95%, at least 96%, at least 97%, at least 98%, at least 99% or more codon optimisation.

Preferably, the gag-pol genes themselves are completely codon-optimised, but may comprise non-contain regions of non-codon-optimised sequence (e.g. between the gag and pol genes). By way of non-limiting example, to maintain the translational slip of reading frames between the gag and pol genes, the region around the translational slip sequence may not be codon-optimised (e.g. in case the precise translational slip sequence is important for this function).

The method of the invention may be a scalable GMP-compatible method. Thus, the method of the invention typically allows the generation of high titre purified pseudotyped lentiviral (e.g. HIV/SIV) vectors.

Typically, the C-terminal deletion in a modified SARS-CoV-2 spike protein of the invention as described herein is associated with production of lentiviral (e.g. HIV/SIV) vector titre that is at least equivalent to the titre of lentiviral (e.g. HIV/SIV) vector produced using corresponding SARS-CoV-2 spike protein (modified or unmodified) which lacks the C-terminal deletion. As used herein, the term “equivalent” may be defined such that the use of the C-terminal deletion does not significantly decrease the titre of lentiviral (e.g. HIV/SIV) vector compared with the use of the corresponding SARS-CoV-2 spike protein (modified or unmodified) which lacks the C-terminal deletion. By way of non-limiting example, use of the C-terminal deletion may produce/be associated with a titre of lentiviral (e.g. HIV/SIV) vector that is no more than 2-fold lower, no more than 1.5-fold lower, no more than 1.0-fold lower, no more than 0.5-fold lower, no more than 0.25-fold lower, or less than the titre of lentiviral (e.g. HIV/SIV) vector compared with the use of a corresponding SARS-CoV-2 spike protein (modified or unmodified) which lacks the C-terminal deletion. The term “equivalent” may be defined such that titre of lentiviral (e.g. HIV/SIV) vector produced using the C-terminal deletion is statistically unchanged (e.g. p<0.05, p<0.01) compared with the titre of lentiviral (e.g. HIV/SIV) vector produced using a corresponding SARS-CoV-2 spike protein (modified or unmodified) which lacks the C-terminal deletion.

In particular, the C-terminal deletion in a modified SARS-CoV-2 spike protein of the invention as described herein may produce/be associated with an increased titre of lentiviral vectors pseudotyped with said modified SARS-CoV-2 spike protein, compared with lentiviral vectors pseudotyped with the corresponding SARS-CoV-2 spike protein (modified or unmodified) which lacks the C-terminal deletion.

Preferably, use of the C-terminal deletion produces/is associated with a titre of lentiviral (e.g. HIV/SIV) vector that is increased compared with the titre of lentiviral (e.g. HIV/SIV) vector produced using a corresponding SARS-CoV-2 spike protein (modified or unmodified) which lacks the C-terminal deletion. The titre of lentiviral (e.g. HIV/SIV) vector may be at least 2-fold, at least 3-fold, or at least 4-fold, at least 5-fold, at least 10-fold, at least 15-fold or greater than the titre of lentiviral (e.g. HIV/SIV) vector produced using a corresponding SARS-CoV-2 spike protein (modified or unmodified) which lacks the C-terminal deletion.

As described in the art, inclusion of mutations at amino acid positions corresponding to, or aligning with, positions 498 and 499 of SEQ ID NO: 1 have been associated with a decrease in manufacturing titres compared with corresponding SARS-CoV-2 spike proteins without these mutations. However, the inventors have surprisingly shown that for some S-LV with SARS-CoV-2 spike proteins, particularly those which are known in the art to be difficult to produce at reasonable titres (e.g. the AUS/VIC1 strain of SARS-CoV-2), the inclusion of mutations at amino acid positions corresponding to, or aligning with, positions 498 and 499 of SEQ ID NO: 1 can surprisingly increase S-LV titre. This rescue of S-LV titre by inclusion of mutations at amino acid positions corresponding to, or aligning with, positions 498 and 499 of SEQ ID NO: 1 can be further increased by a C-terminal deletion, as described herein. Thus, according to the invention, mutations at amino acid positions corresponding to, or aligning with, positions 498 and 499 of SEQ ID NO: 1 in a modified SARS-CoV-2 spike protein of the invention as described herein is associated with production of lentiviral (e.g. HIV/SIV) vector titre that is at least equivalent to the titre of lentiviral (e.g. HIV/SIV) vector produced using corresponding SARS-CoV-2 spike protein (modified or unmodified) which lacks mutations at one or both of these positions. As used herein, the term “equivalent” may be defined such that the use of mutations at amino acid positions corresponding to, or aligning with, positions 498 and 499 of SEQ ID NO: 1 does not significantly decrease the titre of lentiviral (e.g. HIV/SIV) vector compared with the use of the corresponding SARS-CoV-2 spike protein (modified or unmodified) which lacks the C-terminal deletion. By way of non-limiting example, use of mutations at amino acid positions corresponding to, or aligning with, positions 498 and 499 of SEQ ID NO: 1 may produce/be associated with a titre of lentiviral (e.g. HIV/SIV) vector that is no more than 2-fold lower, no more than 1.5-fold lower, no more than 1.0-fold lower, no more than 0.5-fold lower, no more than 0.25-fold lower, or less than the titre of lentiviral (e.g. HIV/SIV) vector compared with the use of a corresponding SARS-CoV-2 spike protein (modified or unmodified) which lacks these mutations. The term “equivalent” may be defined such that titre of lentiviral (e.g. HIV/SIV) vector produced using mutations at amino acid positions corresponding to, or aligning with, positions 498 and 499 of SEQ ID NO: 1 is statistically unchanged (e.g. p<0.05, p<0.01) compared with the titre of lentiviral (e.g. HIV/SIV) vector produced using a corresponding SARS-CoV-2 spike protein (modified or unmodified) which lacks these mutations.

In particular, mutations at amino acid positions corresponding to, or aligning with, positions 498 and 499 of SEQ ID NO: 1 in a modified SARS-CoV-2 spike protein of the invention as described herein may produce/be associated with an increased titre of lentiviral vectors pseudotyped with said modified SARS-CoV-2 spike protein, compared with lentiviral vectors pseudotyped with the corresponding SARS-CoV-2 spike protein (modified or unmodified) which lacks these mutations. This improvement in titre may be particularly observed when the modified SARS-CoV-2 spike protein is derived from a strain which is conventionally hard to produce at reasonable titres, such as the Australia/VIC01/2020 (Aus/VIC01) strain, as described and exemplified herein.

Preferably, use of mutations at amino acid positions corresponding to, or aligning with, positions 498 and 499 of SEQ ID NO: 1 produces/is associated with a titre of lentiviral (e.g. HIV/SIV) vector that is increased compared with the titre of lentiviral (e.g. HIV/SIV) vector produced using a corresponding SARS-CoV-2 spike protein (modified or unmodified) which lacks these mutations. The titre of lentiviral (e.g. HIV/SIV) vector may be at least 1.5-fold, at least 2-fold, at least 3-fold, or at least 4-fold, at least 5-fold, at least 10-fold, at least 15-fold or greater than the titre of lentiviral (e.g. HIV/SIV) vector produced using a corresponding SARS-CoV-2 spike protein (modified or unmodified) which lacks these mutations.

Typically a lentiviral (e.g. HIV/SIV) vector of the invention, such as obtainable by a method of the invention, or using a polynucleotide, expression cassette and/or host cell of the invention is produced at a high-titre. Particularly, a lentiviral vector of the invention comprising a C-terminal deletion as described herein may be produced at a high-titre. Titre may be measured in terms of transducing units, as defined here. As described herein, a lentiviral (e.g. SIV/HIV) vector of the invention, particularly a lentiviral vector comprising a C-terminal deletion as described herein may be produced at equivalent or higher titres than corresponding lentiviral vectors which comprise a corresponding unmodified SARS-CoV-2 spike protein. Particularly, a lentiviral vector of the invention comprising a SARS-CoV-2 spike protein with a C-terminal deletion as described herein may be produced at equivalent or higher titres than corresponding lentiviral vectors pseudotyped with the corresponding SARS-CoV-2 spike protein (modified or unmodified) which lacks the C-terminal deletion. Accordingly, a lentiviral (e.g. HIV/SIV) vector of the invention, such as obtainable by a method of the invention, or using a polynucleotide, expression cassette and/or host cell of the invention may optionally be at a titre of at least about 2.0×10⁶ IU/mL, at least about 2.5×10⁶ IU/mL, at least about 3.0×10⁶ IU/mL, at least about 3.5×10⁶ IU/mL, at least about 3.7×10⁶ IU/mL, at least about 4.0×10⁶ IU/mL, at least about 4.2×10⁶ IU/mL, at least about 4.5×10⁶ IU/mL, at least about 5.0×10⁶ IU/mL, at least about 5.5×10⁶ IU/MI, at least about 5.7×10⁶ IU/mL, at least about 6.0×10⁶ IU/mL, at least about 6.1×10⁶ IU/mL, at least about 6.2×10⁶ IU/mL, or more. Preferably the retroviral/lentiviral (e.g. SIV) vector is produced at a titre of at least about 3.0×10⁶ IU/mL, at least about 4.0×10⁶ IU/mL, or at least about 6.0×10⁶ IU/mL.

The method of the invention may comprise co-expressing a SARS-CoV-2 nucleoprotein in the production cell. As exemplified herein, co-expression of a SARS-CoV-2 nucleoprotein may further increase S-LV production titres. Preferably, co-expression of a SARS-CoV-2 nucleoprotein produces/is associated with a titre of lentiviral (e.g. HIV/SIV) vector that is increased compared with the titre of lentiviral (e.g. HIV/SIV) vector produced using a corresponding SARS-CoV-2 spike protein (modified or unmodified) wherein said method does not comprise co-expression of a SARS-CoV-2 nucleoprotein.

The titre of lentiviral (e.g. HIV/SIV) vector may be at least 1.5-fold, at least 2-fold, at least 3-fold, or at least 4-fold, at least 5-fold, or greater than the titre of lentiviral (e.g. HIV/SIV) vector produced using a corresponding SARS-CoV-2 spike protein (modified or unmodified) which is produced by a method which does not comprise co-expression of a SARS-CoV-2 nucleoprotein.

Typically the SARS-CoV-2 nucleoprotein is co-expressed during the culturing of the production cell. A nucleic acid sequence encoding the SARS-CoV-2 nucleoprotein may be comprised in (i) the same polynucleotide molecule or expression construct as the nucleic acid sequence encoding the modified SARS-CoV-2 spike protein and/or any of the other components need to produce the S-LV vector, or (ii) in one or more separate polynucleotide molecule or expression construct.

The SARS-CoV-2 nucleoprotein may be from any SARS-CoV-2 strain, such as those disclosed herein. Preferably the SARS-CoV-2 nucleoprotein is from the Wuhan-Hu-1 strain or the B.1.1.529, (Omicron) strain.

The production of high-titre lentiviral (e.g. SIV/HIV) vectors according to the invention may impart other desirable properties on the resulting vector products. For example, without being bound by theory, it is believed that production at high titres without the need for intense concentration by methods such as TFF results in a higher quality vector product than lentiviral vectors which comprise a corresponding unmodified SARS-CoV-2 spike protein, or corresponding lentiviral vectors pseudotyped with the corresponding SARS-CoV-2 spike protein (modified or unmodified) which lacks the C-terminal deletion. Without being bound by theory, it is believed that vectors produced at high-titres and without the need for intense concentration are exposed to less shear forces which can damage the viral particles and their RNA cargo.

The invention also provides a method of increasing SARS-CoV-2 spike protein pseudotyped lentiviral (e.g. SIV/HIV) vector titre comprising the use of a C-terminal deletion in the SARS-CoV-2 spike protein. Said method of increasing lentiviral (e.g. SIV/HIV) vector titre according to the invention may increase titre by at least 2-fold, at least 3-fold, or at least 4-fold, at least 5-fold, at least 10-fold, at least 15-fold or more compared with the production of corresponding lentiviral vectors pseudotyped with the corresponding SARS-CoV-2 spike protein (modified or unmodified) which lacks the C-terminal deletion. Alternatively, a method of increasing lentiviral (e.g. SIV/HIV) titre according to the invention may increase titre by at least about at least about 50%, at least about 100%, at least about 150%, at least about 200%, at least about 250%, at least about 300%, at least about 500% or more compared with production of corresponding lentiviral vectors pseudotyped with the corresponding SARS-CoV-2 spike protein (modified or unmodified) which lacks the C-terminal deletion. Preferably, a method of increasing lentiviral (e.g. SIV/HIV) titre according to the invention may increase titre by (a) by at least 5-fold or at least 10-fold; and/or (b) by at least about 100%, more preferably at least about 200%, even more preferably at least about 300%. Typically, the corresponding lentiviral vector/SARS-CoV-2 spike protein is identical to the lentiviral vector/SARS-CoV-2 spike protein of the invention, except that it lacks the C-terminal deletion. All the disclosure herein in relation to method of producing a lentiviral (e.g. SIV/HIV) vector applies equally and without reservation to the methods of increasing lentiviral (e.g. SIV/HIV) titre of the invention.

The production of lentiviral (e.g. HIV/SIV) vectors typically employs one or more plasmids which provide the elements needed for the production of the vector: the genome for the retroviral/lentiviral vector, the Gag-Pol, Rev, SARS-CoV-2 spike protein. Multiple elements can be provided on a single plasmid. Preferably each element is provided on a separate plasmid, such that there four plasmids, one for each of the vector genome, the Gag-Pol, Rev, SARS-CoV-2 spike protein, respectively.

Alternatively, a single plasmid may provide the Gag-Pol and Rev elements, and may be referred to as a packaging plasmid (pDNA2). The remaining elements (genome, SARS-CoV-2 spike protein) may be provided by separate plasmids (pDNA1 and pDNA3 respectively), such that four plasmids are used for the production of a lentiviral (e.g. HIV/SIV) vector according to the invention. In the three plasmid methods, pDNA1 and pDNA3 may be as described herein in the context of the four-plasmid method.

In the preferred four plasmid method of the invention, the vector genome plasmid encodes all the genetic material that is packaged into final lentiviral vector, including the transgene. Typically, only a portion of the genetic material found in the vector genome plasmid ends up in the virus. The vector genome plasmid may be designated herein as “pDNA1”, and typically comprises the transgene and the transgene promoter.

The other three plasmids are manufacturing plasmids encoding the Gag-Pol, Rev and SARS-CoV-2 spike proteins. These plasmids may be designated “pDNA2a”, “pDNA2b” and “pDNA3” respectively.

In the four-plasmid method of the invention all four plasmids contribute to the formation of the final lentiviral (e.g. HIV/SIV) vector. During manufacture of the lentiviral (e.g. HIV/SIV) vector, the vector genome plasmid (pDNA1) typically provides an enhancer/promoter, Psi, RRE, cPPT, mWPRE, SIN LTR, polyA (e.g. SV40 poly A), which are important for virus manufacture. The RRE, cPPT (central polypurine tract), promoter, transgene and mWPRE are typically found in the final lentiviral (e.g. HIV/SIV) vector. SIN LTR (long terminal repeats, SIN/IN self-inactivating) and Psi (packaging signal) may be found in the final lentiviral (e.g. HIV/SIV) vector.

The SARS-CoV-2 spike protein from pDNA3 is important for infection of target cells with the final lentiviral (e.g. HIV/SIV) vector, i.e. for entry of a patient's epithelial cells (typically lung or nasal cells as described herein). The products of the pDNA2a and pDNA2b plasmids are important for virus transduction, i.e. for inserting the lentiviral (e.g. HIV/SIV) DNA into the host's genome. The promoter, regulatory elements (such as WPRE) and transgene are important for transgene expression within the target cell(s).

A method of the invention may comprise or consist of the following steps: (a) growing cells in suspension; (b) transfecting the cells with one or more plasmids; (c) adding a nuclease; (d) harvesting the lentiviral (e.g. HIV/SIV); and (e) purification of the lentiviral (e.g. HIV/SIV). A method of the invention may comprise or consist of the following steps: (a) growing cells in suspension; (b) transfecting the cells with one or more plasmids; (c) adding a nuclease; (d) harvesting the lentiviral (e.g. HIV/SIV); (e) adding trypsin; and (f) purification of the lentiviral (e.g. HIV/SIV).

This method may use the four- or three-plasmid system described herein. Thus, for the preferred four-plasmid method, the one or more plasmids may comprise or consist of: a vector genome plasmid pDNA1; a galpol plasmid (which may be codon-optimised), pDNA2a; a Rev plasmid, pDNA2b; and a SARS-CoV-2 spike protein plasmid, pDNA3.

Any appropriate ratio of vector genome plasmid: gagpol plasmid: Rev plasmid: SARS-CoV-2 spike protein plasmid may be used to further optimise (increase) the lentiviral (e.g. HIV/SIV) titre produced. Suitable ratios may be readily determined by one of ordinary skill in the art. By way of non-limiting example, a ratio of 20:9:6:12 may be used.

Steps (a)-(f) of the method are typically carried out sequentially, starting at step (a) and continuing through to step (f). The method may include one or more additional step, such as additional purification steps, buffer exchange, concentration of the lentiviral (e.g. HIV/SIV) vector after purification, and/or formulation of the lentiviral (e.g. HIV/SIV) vector after purification (or concentration). Each of the steps may comprise one or more sub-steps. For example, harvesting may involve one or more steps or sub-steps, and/or purification may involve one or more steps or sub-steps.

Any appropriate cell type may be transfected with the one or more plasmids to produce a lentiviral (e.g. HIV/SIV) vector of the invention. Typically mammalian cells, particularly human cell lines are used. Non-limiting examples of cells suitable for use in the methods of the invention are HEK293 cells (such as HEK293F or HEK293T cells) and 293T/17 cells. Commercial cell lines suitable for the production of virus are also readily available (e.g. Gibco Viral Production Cells—Catalogue Number A35347 from ThermoFisher Scientific).

The cells may be grown in animal-component free media, including serum-free media. The cells may be grown in a media which contains human components. The cells may be grown in a defined media comprising or consisting of synthetically produced components.

Any appropriate transfection means may be used according to the invention. Selection of appropriate transfection means is within the routine practice of one of ordinary skill in the art. By way of non-limiting example, transfection may be carried out by the use of PEIPro™, Lipofectamine2000™ or Lipofectamine3000™.

Any appropriate nuclease may be used according to the invention. Selection of appropriate nuclease is within the routine practice of one of ordinary skill in the art. Typically the nuclease is an endonuclease. By way of non-limiting example, the nuclease may be Benzonase® or Denarase®. The addition of the nuclease may be at the pre-harvest stage or at the post-harvest stage, or between harvesting steps.

When used, the trypsin activity may preferably be provided by an animal origin free, recombinant enzyme such as TrypLE Select™. The addition of trypsin may be at the pre-harvest stage or at the post-harvest stage, or between harvesting steps.

Any appropriate purification means may be used to purify the lentiviral (e.g. HIV/SIV) vector. Non-limiting examples of suitable purification steps include depth/end filtration, tangential flow filtration (TFF) and chromatography. The purification step typically comprises at least on chromatography step. Non-limiting examples of chromatography steps that may be used in accordance with the invention include mixed-mode size exclusion chromatography (SEC) and/or anion exchange chromatography. Elution may be carried out with or without the use of a salt gradient, preferably without.

Harvesting the lentiviral (e.g. HIV/SIV) vector may be carried out after any appropriate time period following transfection, such as between about 12 to about 144 hours post transfection, between about 24 hours to about 96 hours post transfection, between about 36 hours to about 96 hours post transfection, between about 54 to about 84 hours post transfection, between about 54 hours to about 72 hours post transfection or about 72 hours to about 96 hours post transfection. In some preferred embodiments, harvesting is carried out about 72 hours post transfection.

This method may be used to produce the lentiviral (e.g. HIV/SIV) vectors of the invention.

The method of the invention may use codon-optimised gag-pol genes (or nucleic acids or plasmids comprising or consisting thereof), as they facilitate the production of high titre lentiviral (e.g. HIV/SIV) vectors. Typically said codon-optimised gag-pol genes can be used to produces a titre of lentiviral (e.g. HIV/SIV) vector that is at least equivalent to the titre of lentiviral (e.g. HIV/SIV) vector produced by a corresponding method which does not use codon-optimised gal-pol genes, as described herein. Preferably, the codon-optimised gag-pol genes allow for the production of a titre of lentiviral (e.g. HIV/SIV) vector that is increased compared with the titre of lentiviral (e.g. HIV/SIV) vector produced by a corresponding method which does not use codon-optimised gal-pol genes, as described herein.

The invention also provides host cells (also referred to as viral vector production cells) comprising (i) a lentiviral (e.g. HIV/SIV) vector of the invention, (ii) a modified SARS-CoV-2 spike protein as described herein; (iii) an expression construct of the invention and/or (iii) a polynucleotide of the invention; or any combination thereof. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein). The host cells of the invention may express human TMPRSS2 (hTMPRSS2), or a functional fragment thereof, either endogenously or following introduction of an exogenous nucleic acid, vector or plasmid. The expressed TMPRSS2 amino acid sequence may be as described herein.

The invention also provides a lentiviral (e.g. HIV/SIV) vector obtainable by a method of the invention, or using an expression construct, polynucleotide, plasmid, modified SARS-CoV-2 spike protein and/or host cell of the invention.

The one or more nucleic acid sequence encoding the lentiviral packaging components, lentiviral envelope components, and a lentiviral genome may be comprised in (i) the same polynucleotide molecule or expression construct as the nucleic acid sequence encoding the modified SARS-CoV-2 spike protein or (ii) in one or more separate polynucleotide molecule or expression construct. Exemplary plasmid systems for use in the methods of the invention are described herein.

### Virus-Like Particles

The invention also provides virus-like particles (VLP) comprising one or more modified SARS-CoV-2 spike protein of the invention as described herein.

As defined herein, a VLP of the invention is an empty lentiviral particle, i.e. a VLP lacks a lentiviral genome.

Methods for generating VLPs are known in the art (see, for example, Brune et al. Sci. Rep. (2016), 19(6):19234, which is incorporated by reference in its entirety) and can readily be applied to the present invention. References herein to vectors of the invention may apply equally to VLP of the invention.

### In Vitro and Animal Models

The invention also provides in vitro and animal models suitable for use in development of prophylaxes, therapeutics, and vaccine strategies for SARS-CoV-2 or other disease or disorders.

The invention provides an in vitro model comprising rodent cells, particularly mouse cells, that are transduced with a lentiviral (e.g. HIV/SIV) vector of the invention. Such models can be used, for example, to assess whether a given antibody is capable of preventing in vitro cell entry of the lentiviral vector.

The invention also provides an in vivo animal model, particularly a rodent model, especially a mouse model. For example, a vector expressing an antibody candidate may be administered to a mouse. Following transduction of said vector, the antibody candidate may be produced in vivo by the cells of the mouse. Subsequent challenge with a lentiviral (e.g. HIV/SIV) vector of the invention can be used, for example, to assess whether a given antibody is capable of preventing in vitro cell entry of the lentiviral vector.

### Therapeutic Indications

The nucleic acid cassettes and vectors of the invention, and particularly the pseudotyped lentiviral (e.g. HIV/SIV) vectors of the invention are typically capable of: (i) airway transduction without disruption of epithelial integrity; (ii) persistent gene expression; (iii) lack of chronic toxicity; and/or (iv) efficient repeat administration. Long term/persistent stable gene expression, preferably at a therapeutically-effective level, may be achieved using repeat doses of a nucleic acid cassette or vector of the present invention. Alternatively, a single dose may be used to achieve the desired long-term expression.

Thus, advantageously, the nucleic acid cassettes and vectors of the present invention, and particularly the lentiviral (e.g. HIV/SIV) vectors of the present invention can be used in gene therapy. Accordingly, the present invention provides a nucleic acid cassette or gene therapy vector as defined herein for use in a method of treating or preventing a disease. The disease to be treated may be chronic or acute.

The nucleic acid cassettes and vectors of the invention, particularly the lentiviral (e.g. HIV/SIV) vectors of the present invention may be used to deliver any transgene useful in gene therapy. Typically, the nucleic acid cassettes and vectors of the invention, particularly the lentiviral (e.g. HIV/SIV) vectors of the present invention are for use in gene therapy for the treatment of a disease or disorder of the airways, respiratory tract, or lung.

By way of example, efficient airway cell uptake properties of the nucleic acid cassettes and vectors of the present invention, and particularly the lentiviral (e.g. HIV/SIV) vectors of the invention make them highly suitable for treating respiratory or respiratory tract diseases, particularly genetic respiratory diseases. In particular, and without being bound by theory, given that SARS-CoV-2 pseudotyped lentiviral (e.g. HIV/SIV) vectors will bind to ACE2 that is abundant in ATII cells of the lung, the lentiviral (e.g. HIV/SIV) vectors of the present invention may be used to express therapeutic proteins within ATII cells. Non-limiting example of such therapeutic proteins include surfactant protein A1 (SP-A1, encoded by SFTPA1), surfactant protein A2 (SP-A2, encoded by SFTPA2), surfactant protein B (SP-B, encoded by SFTPB), surfactant protein C (SP-C, encoded by SFTPC), surfactant protein D (SP-D, encoded by SFTPD), ATP-binding cassette sub-family member A (ABCA3), thyroid transcription factor 1 (TITF1, encoded by NK2 homeobox 1 (NKX2-1)) and solute carrier family 34 member 2 (SLC34A2), as well as other therapeutic proteins disclosed herein. Expression of SP-A1 and/or SP-A2 may be used to treat a number of diseases including ARDS, COPD, pulmonary fibrosis (e.g. IPF) and respiratory infections. Expression of SP-B may be used to treat SP-B deficiency (also referred to as surfactant metabolism dysfunction, pulmonary, 1 (SMDP1)). Expression of SP-C may be used to treat SP-C deficiency (also referred to as surfactant metabolism dysfunction, pulmonary, 2 (SMDP2)). Expression of SP-D may be used to treat respiratory infections. Expression of ABCA3 may be used to treat ABCA3 deficiency (also referred to as surfactant metabolism dysfunction, pulmonary, 3 (SMDP3)). Expression of SLC34A2 may be used to treat pulmonary alveolar microlithiasis and IPF.

The lentiviral (e.g. HIV/SIV) vectors of the present invention may be used to express two or more (e.g. from about 2 to about 10, about 2 to about 5, 2, 3, 4 or 5) therapeutic proteins within ATII cells. The expression of two or more therapeutic proteins by ATII cells using nucleic acid cassettes and vectors of the present invention, and particularly the lentiviral (e.g. HIV/SIV) vectors of the invention may be useful in the treatment of diseases or disorders in which two or more genes (e.g. from about 2 to about 10, about 2 to about 5, 2, 3, 4 or 5 genes) are involved. For example, nucleic acid cassettes and vectors of the present invention, and particularly the lentiviral (e.g. HIV/SIV) vectors of the invention may be used to express two or more (e.g. from about 2 to about 10, about 2 to about 5, 2, 3, 4 or 5) therapeutic proteins selected from SP-A1, SP-A2, SP-B, SP-C, SP-D, ABCA3, TITF1, SLC34A2, telomerase reverse transcriptase (TERT), telomerase RNA component (TERC), decorin, TRIM72, GM-CSF, and GM-CSF receptors (including CSF2RA and CSF2RB). Non-limiting examples of such diseases and disorders include Pulmonary Alveolar Proteinosis (PAP, hereditary (hPAP) and/or acquired (aPAP)) and pulmonary fibrotic diseases (including idiopathic pulmonary fibrosis (IPF)). For example, treatment of aPAP may comprise expression of GM-CSF. Treatment of hPAP may comprise expression of CSF2RA and/or CSF2RB. Treatment of IPF may comprise expression of one or more of SP-A2, decorin, TRIM72, TERT and/or TERC.

The nucleic acid cassettes and vectors of the present invention, and particularly the lentiviral (e.g. HIV/SIV) vectors of the invention can also be used in methods of gene therapy to promote secretion of therapeutic proteins. By way of further example, the invention provides secretion of therapeutic proteins into the lumen of the respiratory tract (e.g. lung lumen) and/or the circulatory system. In such instances, transduction of ATII cells by nucleic acid cassettes and vectors of the present invention may not be essential, as the ATII cells are not the target cells of interest, but rather the therapeutic protein is secreted into the lumen of the respiratory tract (e.g. lung lumen) or the circulatory system. Without being bound by theory, expression of therapeutic proteins by ATII cells may be advantageous, as ATII cells are typically adjacent to endothelial cells within the respiratory tract, such that expression of therapeutic proteins by ATII cells may be well suited for secretion into the circulatory system. Thus, administration of a nucleic acid cassettes and vectors of the present invention, and particularly a lentiviral (e.g. HIV/SIV) vector of the invention and its uptake by airway cells may enable the use of the lungs (or nose or airways) as a “factory” to produce a therapeutic protein that is then secreted and enters the general circulation at therapeutic levels, where it can travel to cells/tissues of interest to elicit a therapeutic effect. In contrast to intracellular or membrane proteins, the production of such secreted proteins does not rely on specific disease target cells being transduced, which is a significant advantage and achieves high levels of protein expression. Thus, other diseases which are not respiratory tract diseases, such as cardiovascular diseases, particularly genetic cardiovascular diseases or blood disorders, particularly blood clotting deficiencies, can also be treated by the nucleic acid cassettes and vectors of the present invention, and particularly the lentiviral (e.g. SIV/HIV) vectors of the present invention.

Non-limiting examples of therapeutic secreted proteins that may be expressed by nucleic acid cassettes and vectors of the present invention, and particularly the lentiviral (e.g. HIV/SIV) vectors of the invention include AAT, Factor VIII, SP-A1, SP-A2, SP-B, SP-C, SP-D, Factor VII, Factor IX, Factor X, Factor XI, van Willebrand Factor, Granulocyte-Macrophage Colony-Stimulating Factor (GM-CSF), decorin, anti-inflammatory proteins (e.g. IL-10 or TGFβ) monoclonal antibodies, anti-inflammatory decoys, and monoclonal antibodies against an infectious agent.

Non-limiting examples of other therapeutic proteins that may be expressed by nucleic acid cassettes and vectors of the present invention, and particularly the lentiviral (e.g. HIV/SIV) vectors of the invention include CFTR, TRIM72, CSF2RA, ABCA3, TITF1, SLC34A2, TERT, TERC and CSF2RB.

Nucleic acid cassettes and vectors of the present invention, and particularly the lentiviral (e.g. HIV/SIV) vectors of the invention can effectively treat a disease by providing a transgene for the correction of the disease. For example, inserting a functional copy of the CFTR gene to ameliorate or prevent lung disease in CF patients, independent of the underlying mutation. Accordingly, nucleic acid cassettes and vectors of the present invention, and particularly lentiviral (e.g. HIV/SIV) vectors of the invention may be used to treat cystic fibrosis (CF), typically by gene therapy with a CFTR transgene as described herein.

As a further example, nucleic acid cassettes and vectors of the present invention, and particularly lentiviral (e.g. HIV/SIV) vectors of the invention may be used to treat SFTPB deficiency (also known as surfactant metabolism dysfunction, pulmonary 1 (SMDP1)), typically by gene therapy with a SFTPB transgene as described herein.

As another example, nucleic acid cassettes and vectors of the present invention, and particularly lentiviral (e.g. HIV/SIV) vectors of the invention may be used to treat alpha-1-antitrypsin (AAT) deficiency, typically by gene therapy with a AAT transgene (SERPINA1) as described herein. AAT is a secreted anti-protease that is produced mainly in the liver and then trafficked to the lung, with smaller amounts also being produced in the lung itself. The main function of AAT is to bind and neutralise/inhibit neutrophil elastase. Gene therapy with AAT according to the present invention is relevant to AAT deficient patient, as well as in other lung diseases such as CF or chronic obstructive pulmonary disease (COPD), and offers the opportunity to overcome some of the problems encountered by conventional enzyme replacement therapy (in which AAT isolated from human blood and administered intravenously every week), providing stable, long-lasting expression in the target tissue (lung/nasal epithelium), ease of administration and unlimited availability.

Transduction with a nucleic acid cassettes and vectors of the present invention, and particularly a lentiviral (e.g. HIV/SIV) vector of the invention may lead to secretion of the recombinant protein into the lumen of the lung as well as into the circulation. One benefit of this is that the therapeutic protein reaches the interstitium. AAT gene therapy may therefore also be beneficial in other disease indications, non-limiting examples of which include type 1 and type 2 diabetes, acute myocardial infarction, ischemic heart disease, rheumatoid arthritis, inflammatory bowel disease, transplant rejection, graft versus host (GvH) disease, multiple sclerosis, liver disease, cirrhosis, vasculitides and infections, such as bacterial and/or viral infections.

AAT has numerous other anti-inflammatory and tissue-protective effects, for example in pre-clinical models of diabetes, graft versus host disease and inflammatory bowel disease. The production of AAT in the lung and/or nose following transduction according to the present invention may, therefore, be more widely applicable, including to these indications.

Other examples of diseases that may be treated with gene therapy of a secreted protein according to the present invention include cardiovascular diseases and blood disorders, particularly blood clotting deficiencies such as haemophilia (A, B or C), von Willebrand disease and Factor VII deficiency.

Other examples of diseases or disorders to be treated include Primary Ciliary Dyskinesia (PCD), acute lung injury, Surfactant Protein B (SFTB) deficiency, Pulmonary Alveolar Proteinosis (PAP, hereditary and/or acquired), Chronic Obstructive Pulmonary Disease (COPD), pulmonary surfactant metabolism dysfunction 3 (SMDP3) or another surfactant deficiency, acute respiratory distress syndrome (ARDS), COVID-19, a pulmonary fibrotic disease (including idiopathic pulmonary fibrosis), a pulmonary allergic condition, asthma, lung cancer or a dysplastic change in the lungs, haemophilia and/or inflammatory, infectious, immune or metabolic conditions, such as lysosomal storage diseases or a pulmonary bacterial infection, or any other lung disease or disorder.

The nucleic acid cassettes and vectors of the invention, particularly the lentiviral (e.g. HIV/SIV) vectors of the present invention, typically provide high expression levels of a therapeutic protein when administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or μM.

Expression/secretion/membrane insertion of a therapeutic protein of interest may be given in absolute terms. Alternatively, expression/secretion/membrane insertion of a therapeutic protein may be given in relative terms, for example relative to the expression/secretion/membrane insertion of the therapeutic protein encoded by a corresponding nucleic acid cassette or vector of the invention without the exogenous signal peptide or with the endogenous signal peptide of the therapeutic protein or relative to the expression/secretion/membrane insertion of the corresponding endogenous (defective) gene.

Expression may be measured in terms of mRNA or protein expression. The expression of the therapeutic protein of the invention, such as a functional CFTR gene, may be quantified relative to the endogenous protein or gene, such as the endogenous (dysfunctional) CFTR genes in terms of protein concentration, mRNA copies per cell or any other appropriate unit. Secretion and/or membrane insertion of a therapeutic protein may be quantified relative to secretion/membrane insertion of the corresponding endogenous protein, or relative to the level of secretion/membrane insertion of the therapeutic protein introduced via an expression cassette lacking the exogenous signal peptide and/or comprising the endogenous signal peptide of the therapeutic protein.

Expression levels of a nucleic acid encoding a therapeutic protein and/or the expression/secretion/membrane insertion of the encoded therapeutic protein of the invention may be measured ex vivo (e.g. in the conditioned media used to culture the cells or within the cells themselves) or in vivo (e.g. in the lung tissue, epithelial lining fluid and/or serum/plasma) as appropriate. A high and/or therapeutic expression level may therefore refer to the concentration in the lung, epithelial lining fluid and/or serum/plasma.

Repeated doses of nucleic acid cassettes and vectors of the invention, particularly the lentiviral (e.g. HIV/SIV) vectors of the present invention may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

The invention also provides nucleic acid cassettes and vectors of the present invention, and particularly lentiviral (e.g. HIV/SIV) vectors of the invention as described herein for use in a method of gene therapy, wherein said method comprises the steps of: (a) transducing cells (e.g. airway cells) ex vivo to produce modified cells expressing a transgene of interest; and (b) administering the resulting modified cells.

The invention provides a method of treating a disease, the method comprising administering a nucleic acid cassette or vector of the present invention, and particularly a lentiviral (e.g. HIV/SIV) vector of the invention to a subject. Any disease described herein may be treated according to the invention. In particular, the invention provides a method of treating a lung disease using a nucleic acid cassette or vector of the present invention, and particularly a lentiviral (e.g. HIV/SIV) vector of the invention. The disease to be treated may be a chronic disease.

The invention also provides a nucleic acid cassette or vector of the present invention, and particularly a lentiviral (e.g. HIV/SIV) vector of the invention as described herein for use in a method of treating a disease. Any disease described herein may be treated according to the invention. In particular, the invention provides a nucleic acid cassette or vector of the present invention, and particularly a lentiviral (e.g. HIV/SIV) vector of the invention for use in a method of treating a lung disease. The disease to be treated may be a chronic disease.

The invention also provides the use of a nucleic acid cassette or vector of the present invention, and particularly a lentiviral (e.g. HIV/SIV) vector of the invention as described herein in the manufacture of a medicament for use in a method of treating a disease. Any disease described herein may be treated according to the invention. In particular, the invention provides the use of a nucleic acid cassette or vector of the present invention, and particularly a lentiviral (e.g. HIV/SIV) vector of the invention for the manufacture of a medicament for use in a method of treating a lung disease. The disease to be treated may be a chronic disease.

The invention also provides a cell comprising a nucleic acid cassette or vector of the present invention, and particularly a lentiviral (e.g. HIV/SIV) vector. Said cell may be an airway cell as described herein, or a host cell for the production of said nucleic acid cassette or vector of the present invention, as described herein.

Any and all disclosure herein in relation to nucleic acid cassettes or vectors of the present invention, and particularly lentiviral (e.g. HIV/SIV) vectors of the invention applies equally and without reservation to the therapeutic uses and methods described herein.

Long term/persistent stable gene expression, preferably at a therapeutically-effective level, may be achieved using repeat doses of nucleic acid cassettes or vectors of the present invention, and particularly lentiviral (e.g. HIV/SIV) vectors of the invention of the present invention. Alternatively, a single dose may be used to achieve the desired long-term expression.

### Formulation and Administration

The invention also provides a composition comprising a nucleic acid cassette or vector of the present invention, and particularly a lentiviral (e.g. HIV/SIV) vector of the invention, and optionally a pharmaceutically acceptable carrier, excipient, buffer or diluent.

The nucleic acid cassettes or vectors of the present invention, and particularly lentiviral (e.g. HIV/SIV) vectors of the invention may be administered in any dosage appropriate for achieving the desired therapeutic effect. Appropriate dosages may be determined by a clinician or other medical practitioner using standard techniques and within the normal course of their work. Non-limiting examples of suitable dosages of viral vectors of the invention include 1×10⁸ transduction units (TU), 1×10⁹ TU, 1×10¹⁰ TU, 1×10¹¹ TU or more. Non-limiting examples of suitable dosages of non-viral vectors/delivery means of the invention include a maximum of 30 mL per dose, a maximum of 25 mL per dose, a maximum of 20 mL per dose, a maximum of 15 mL per dose, a maximum of 10 mL per dose, or less, preferably a maximum of 20 mL per dose.

Non-limiting examples of pharmaceutically acceptable carriers that may be comprised in a composition of the invention include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long-term storage.

The nucleic acid cassettes or vectors of the present invention, and particularly retroviral/lentiviral (e.g. SIV) vectors of the invention may be administered by any appropriate route. It may be desired to direct the compositions of the present invention (as described above) to the respiratory system of a subject. Efficient transmission of a therapeutic/prophylactic composition or medicament to the site of a disease or disorder in the respiratory tract may be achieved by oral or intra-nasal administration, for example, as aerosols (e.g. nasal sprays), or by catheters. Typically the nucleic acid cassettes or vectors of the present invention, and particularly retroviral/lentiviral (e.g. SIV) vectors of the invention are stable in clinically relevant nebulisers, inhalers (including metered dose inhalers), catheters and aerosols, etc.

Other routes of administration, including but not limited to i.v. administration, intranasal administration and intraplural injection are also encompassed by the present invention. Suitable administration routes are known in the art.

In some embodiments the nose is a preferred production site for a therapeutic protein using nucleic acid cassettes or vectors of the present invention, and particularly retroviral/lentiviral (e.g. SIV) vectors of the invention for at least one of the following reasons: (i) extracellular barriers such as inflammatory cells and sputum are less pronounced in the nose; (ii) ease of vector administration; (iii) smaller quantities of vector required; and (iv) ethical considerations. Thus, nasal administration of nucleic acid cassettes or vectors of the present invention, and particularly retroviral/lentiviral (e.g. SIV) vectors of the invention may result in efficient (high-level) and long-lasting expression of the therapeutic protein of interest. Accordingly, nasal administration of nucleic acid cassettes or vectors of the present invention, and particularly retroviral/lentiviral (e.g. SIV) vectors of the invention may be preferred.

Formulations for intra-nasal administration may be in the form of nasal droplets or a nasal spray. An intra-nasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J. in Remington's Pharmaceutical Sciences (supra). The agents may be formulated as solution aerosols, dispersion or suspension aerosols of dry powders, emulsions or semisolid preparations. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising nucleic acid cassettes or vectors of the present invention, and particularly retroviral/lentiviral (e.g. SIV) vectors of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

In some cases, after an initial administration a subsequent administration of nucleic acid cassettes or vectors of the present invention, and particularly retroviral/lentiviral (e.g. SIV) vectors of the invention may be performed. The administration may, for instance, be at least a week, two weeks, a month, two months, six months, a year or more after the initial administration. In some instances, nucleic acid cassettes or vectors of the present invention, and particularly retroviral/lentiviral (e.g. SIV) vectors of the invention may be administered at least once a week, once a fortnight, once a month, every two months, every six months, annually or at longer intervals. Preferably, administration is every six months, more preferably annually. The nucleic acid cassettes or vectors of the present invention, and particularly retroviral/lentiviral (e.g. SIV) vectors may, for instance, be administered at intervals dictated by when the effects of the previous administration are decreasing.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY
A R N D C Q E G H I L K M F P S T W Y V

A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4
   The percent identity is then calculated as:

$$Total number of identitcal matches$$

$$\_\_\_ \_\_\_ \_\_\_ \_\_\_ \_\_\_ \_\_\_ \_\_\_ \_\_\_ \_\_\_ \_\_\_ \_\_\_ \_ \times 100$$

$$[ length of the longer sequence plus the$$

$$number of gaps introduced into the longer$$

$$sequence in order to align the two sequences ]$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### SEQUENCE INFORMATION

### Key to Sequences

SEQ ID NO: 1 Wuhan-Hu-1 spike protein amino acid sequence (encoded by pGM887 S-LV v01)
SEQ ID NO: 2 Wuhan Hu-1+Δ19aa amino acid sequence (encoded by pGM896 S-LV v02)
SEQ ID NO: 3 S:G614 amino acid sequence (encoded by pGM906 S-LV v01)
SEQ ID NO: 4 S:G614+Δ19aa amino acid sequence (encoded by pGM907 S-LV v02)
SEQ ID NO: 5 S:Aus/VIC01 amino acid sequence (encoded by pGM898 S-LV v01)
SEQ ID NO: 6 S:Aus/VIC01+Δ19aa amino acid sequence (encoded by pGM904 S-LV v02)
SEQ ID NO: 7 S:B.1.1.7+Δ19aa amino acid sequence (encoded by pGM965 S-LV v04)
SEQ ID NO: 8 S:B.1.1351+Δ19aa amino acid sequence (encoded by pGM970 S-LV v04)
SEQ ID NO: 9 S:B.1.1.7&B.1.1351 chimera+Δ19aa amino acid sequence (encoded by pGM1000 S-LV v02)
SEQ ID NO: 10 S:B.1.617.2+Δ19aa amino acid sequence (encoded by pGM1027 S-LV v02)
SEQ ID NO: 11 C.37+Δ19aa amino acid sequence (encoded by pGM1028 S-LV v02)
SEQ ID NO: 12 maS:Y498,T499+Δ19aa amino acid sequence (encoded by pGM937 S-LV v03)
SEQ ID NO: 13 maS:Y498,T499,G614+Δ19aa amino acid sequence (encoded by pGM939 S-LV v03)
SEQ ID NO: 14 maS:Aus/VIC01,Y498,T499+Δ19aa amino acid sequence (encoded by pGM938 S-LV v03)
SEQ ID NO: 15 S:B.1.617.2,Y501+Δ19aa amino acid sequence (encoded by pGM1038 S-LV v04)
SEQ ID NO: 16 C.37,Y501+Δ19aa amino acid sequence (encoded by pGM1039 S-LV v05)
SEQ ID NO: 17 S:B.1.617.2,Y489,T499,Y501+Δ19aa amino acid sequence (encoded by pGM1040 S-LVv04)
SEQ ID NO: 18 C.37,Y489,T499,Y501+Δ19aa amino acid sequence (encoded by pGM1041 S-LV v05)
SEQ ID NO: 19 maS:Y498,T499,Y501+Δ19aa amino acid sequence (encoded by pGM998 S-LV v05)
SEQ ID NO: 20 maS:Y498,T499,Y501,G614+Δ19aa amino acid sequence (encoded by pGM999 S-LV v05)
SEQ ID NO: 21 S:G614 with furin site knock out amino acid sequence (encoded by pGM1026 S-LV v05)
SEQ ID NO: 22 Wuhan-Hu-1 spike protein nucleic acid sequence (comprised in pGM887 S-LV v01)
SEQ ID NO: 23 Wuhan Hu-1+Δ19aa nucleic acid sequence (comprised in pGM896 S-LV v02)
SEQ ID NO: 24 S:G614 nucleic acid nucleic (comprised in pGM906 S-LV v01)
SEQ ID NO: 25 S:G614+Δ19aa nucleic acid sequence (comprised in pGM907 S-LV v02)
SEQ ID NO: 26 S:Aus/VIC01 nucleic acid sequence (comprised in pGM898 S-LV v01)
SEQ ID NO: 27 S:Aus/VIC01+Δ19aa nucleic acid sequence (comprised in pGM904 S-LV v02)
SEQ ID NO: 28 S:B.1.1.7+Δ19aa nucleic acid sequence (comprised in pGM965 S-LV v04)
SEQ ID NO: 29 S:B.1.1351+Δ19aa nucleic acid sequence (comprised in pGM970 S-LV v04)
SEQ ID NO: 30 S:B.1.1.7&B.1.1351 chimera+Δ19aa nucleic acid sequence (comprised in pGM1000 S-LV v02)
SEQ ID NO: 31 S:B.1.617.2+Δ19aa nucleic acid sequence (comprised in pGM1027 S-LV v02)
SEQ ID NO: 32 C.37+Δ19aa nucleic acid sequence (comprised in pGM1028 S-LV v02)
SEQ ID NO: 33 maS:Y498,T499+Δ19aa nucleic acid sequence (comprised in pGM937 S-LV v03)
SEQ ID NO: 34 maS:Y498,T499,G614+Δ19aa nucleic acid sequence (comprised in pGM939 S-LV v03)
SEQ ID NO: 35 maS:Aus/VIC01,Y498,T499+Δ19aa nucleic acid sequence (comprised in pGM938 S-LV v03)
SEQ ID NO: 36 S:B.1.617.2,Y501+Δ19aa nucleic acid sequence (comprised in pGM1038 S-LV v04)
SEQ ID NO: 37 C.37,Y501+Δ19aa nucleic acid sequence (comprised in pGM1039 S-LV v05)
SEQ ID NO: 38 S:B.1.617.2,Y489,T499,Y501+Δ19aa nucleic acid sequence (comprised in pGM1040 S-LV v04)
SEQ ID NO: 39 C.37,Y489,T499,Y501+Δ19aa nucleic acid sequence (comprised in pGM1041 S-LVv05)
SEQ ID NO: 40 maS:Y498,T499,Y501+Δ19aa nucleic acid sequence (comprised in pGM998 S-LV v05)
SEQ ID NO: 41 maS:Y498,T499,Y501,G614+Δ19aa nucleic acid sequence (comprised in pGM999 S-LV v05)
SEQ ID NO: 42 S:G614 with furin site knock out nucleic acid sequence (comprised in pGM1026 S-LV v05)
SEQ ID NO: 43 hCEF promoter
SEQ ID NO: 44 CMV promoter
SEQ ID NO: 45 human elongation factor 1a (EF1a) promoter.
SEQ ID NO: 46 WPRE component (mWPRE)
SEQ ID NO: 47 forward primer for (co)hACE2
SEQ ID NO: 48 reverse primer for (co)hACE2
SEQ ID NO: 49 forward primer
SEQ ID NO: 50 reverse primer
SEQ ID NO: 51 forward primer for hTMPRSS2
SEQ ID NO: 52 reverse primer for hTMPRSS2
SEQ ID NO: 53 forward primer
SEQ ID NO: 54 reverse primer
SEQ ID NO: 55 forward primer for SARS-CoV-2 (co)S (Wuhan Hu-1)
SEQ ID NO: 56 reverse primer for SARS-CoV-2 (co)S (Wuhan Hu-1)
SEQ ID NO: 57 forward primer for SARS-CoV-2 (co)S (Wuhan Hu-1) Δ19
SEQ ID NO: 58 reverse primer for SARS-CoV-2 (co)S (Wuhan Hu-1) Δ19
SEQ ID NO: 59 forward primer for SARS-CoV-2 S Australia/VIC01/2020
SEQ ID NO: 60 reverse primer for SARS-CoV-2 S Australia/VIC01/2020
SEQ ID NO: 61 forward primer for SARS-CoV-2 D614G variant
SEQ ID NO: 62 reverse primer for SARS-CoV-2 D614G variant
SEQ ID NO: 63 forward primer for SARS-CoV-2 S mACE2 adaptation
SEQ ID NO: 64 reverse primer for SARS-CoV-2 S mACE2 adaptation

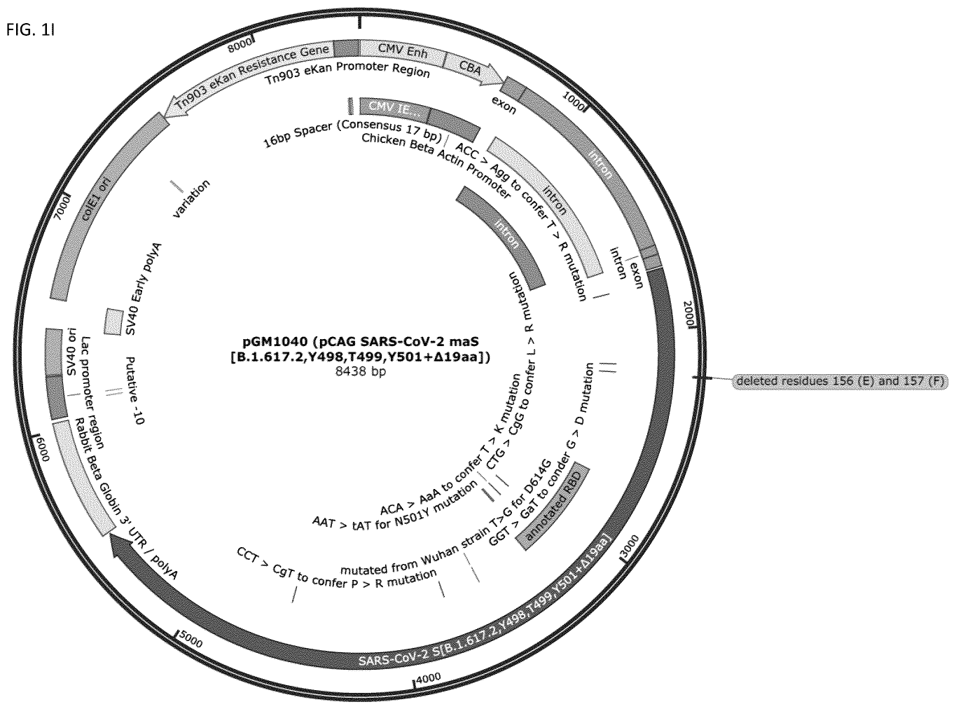

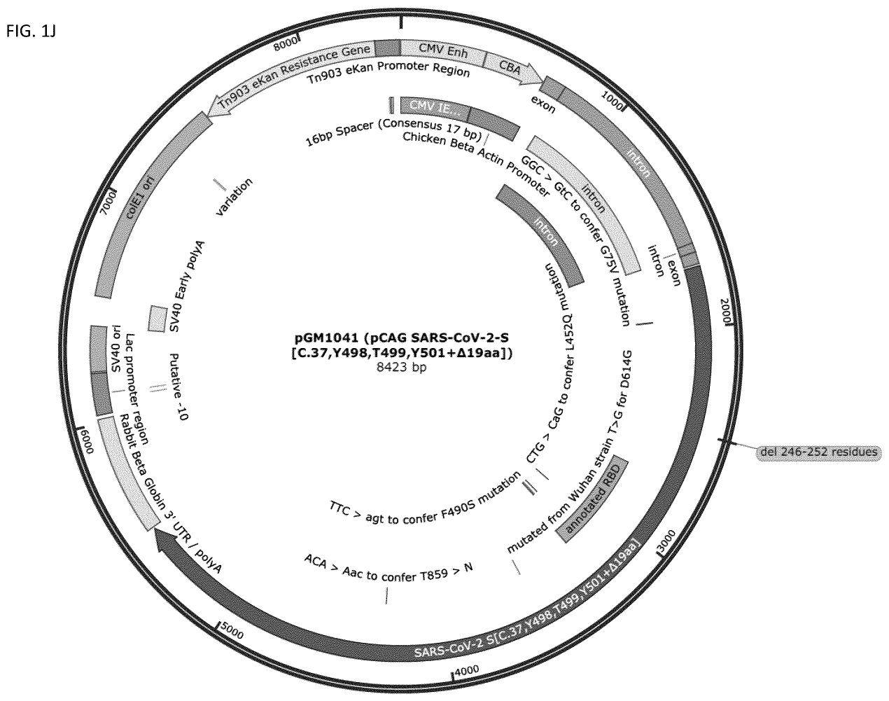

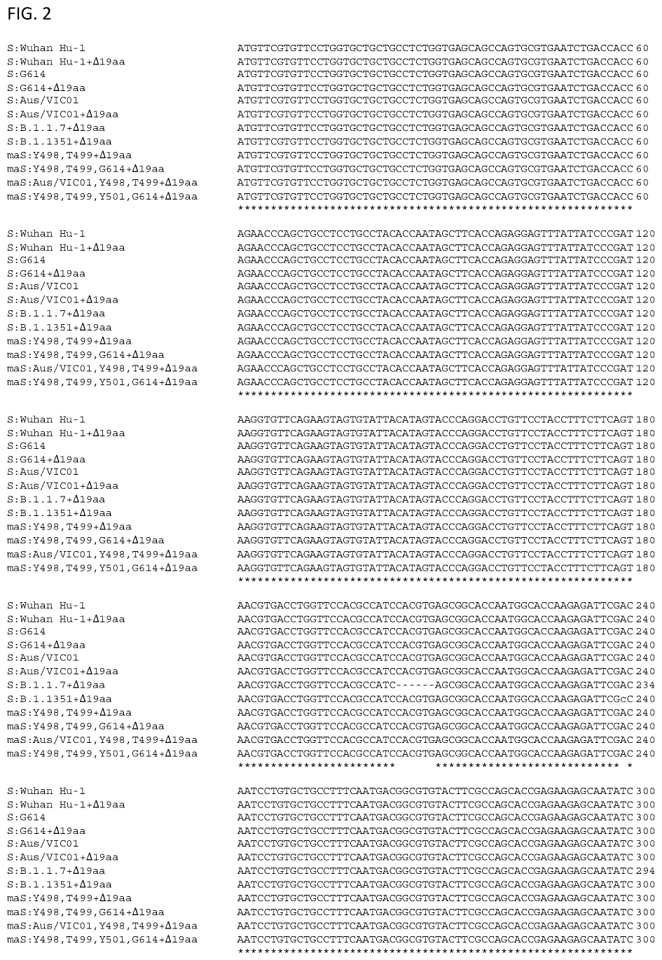

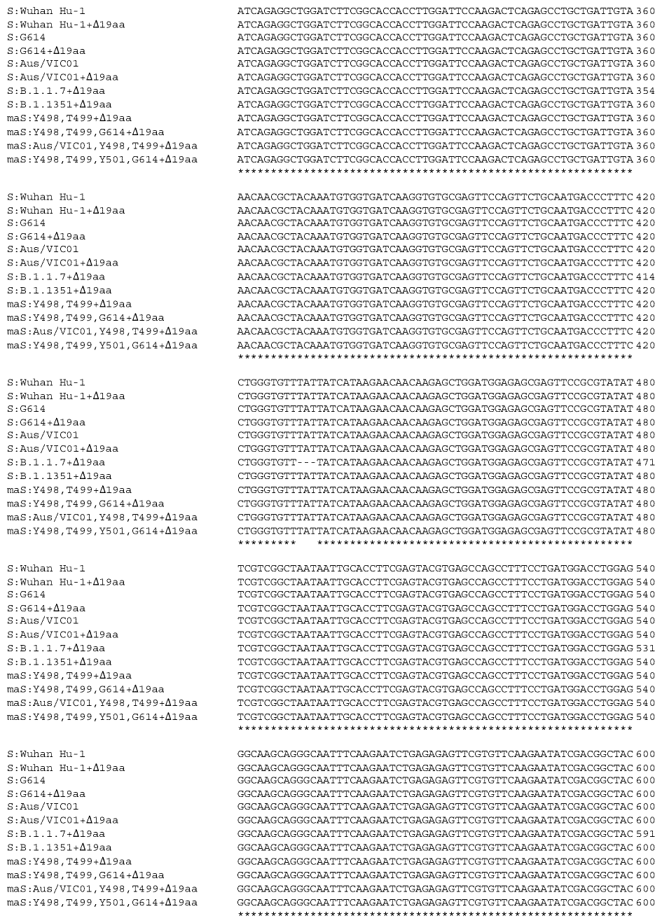

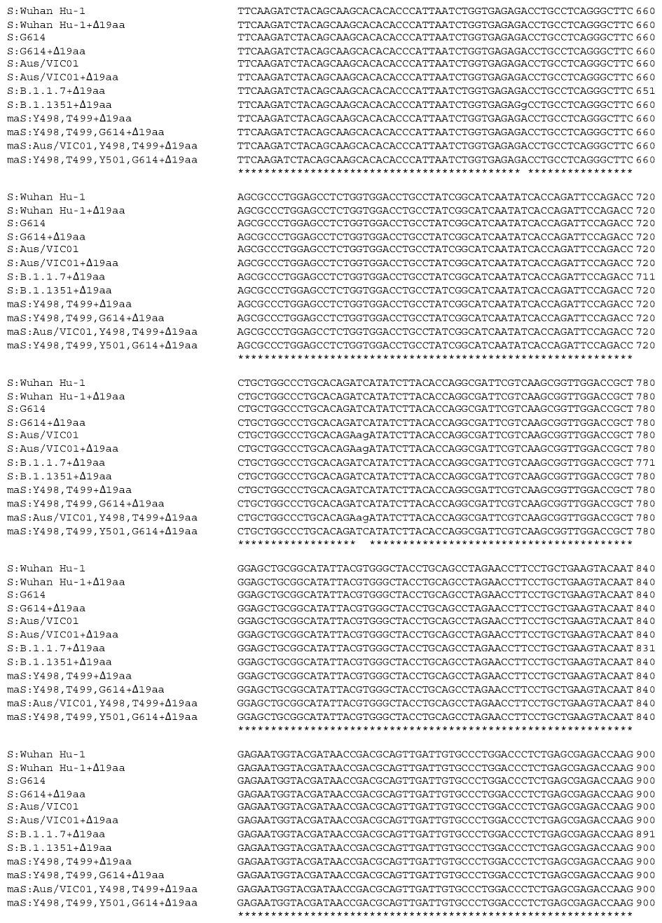

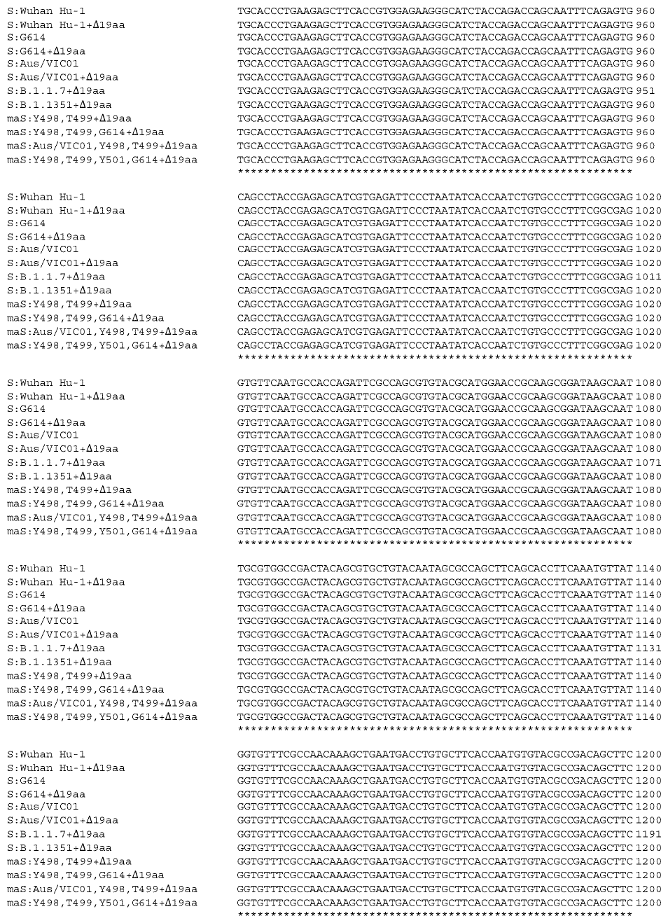

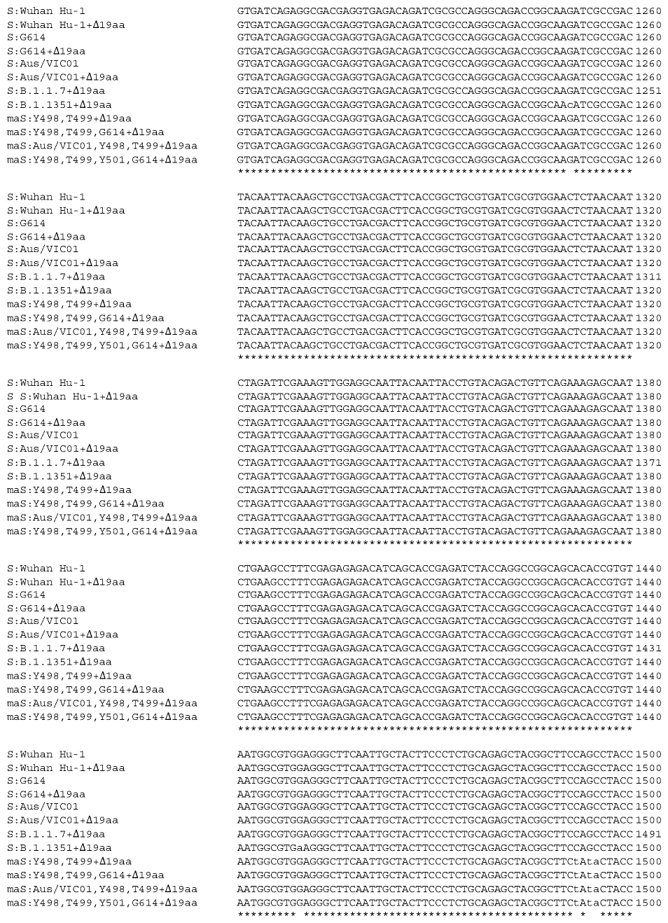

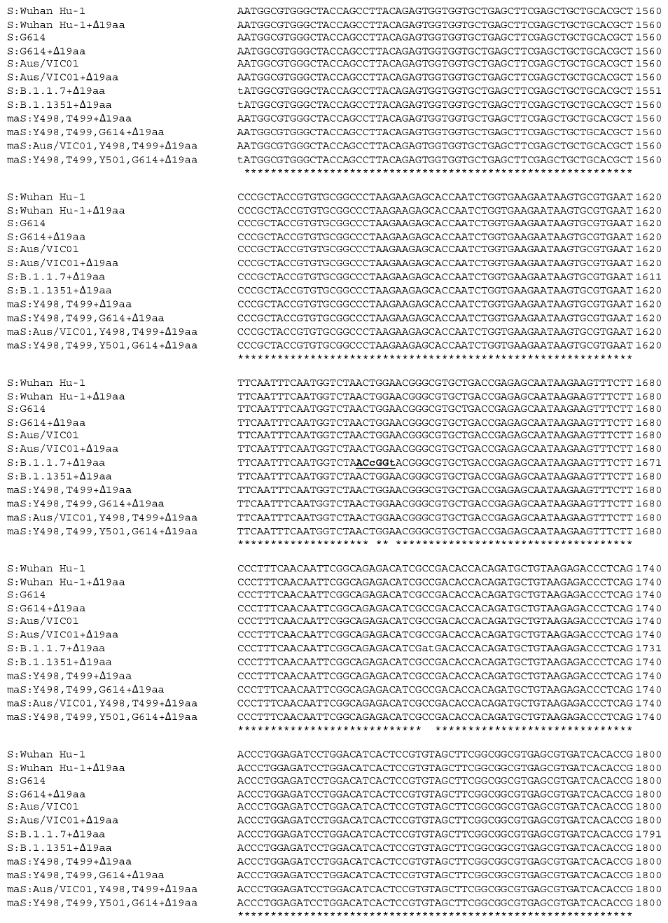

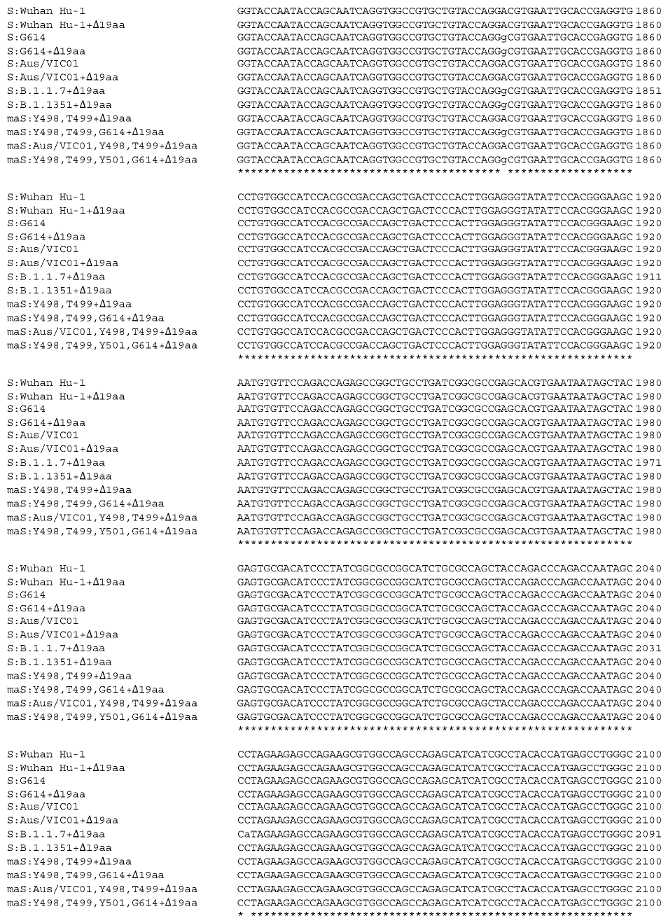

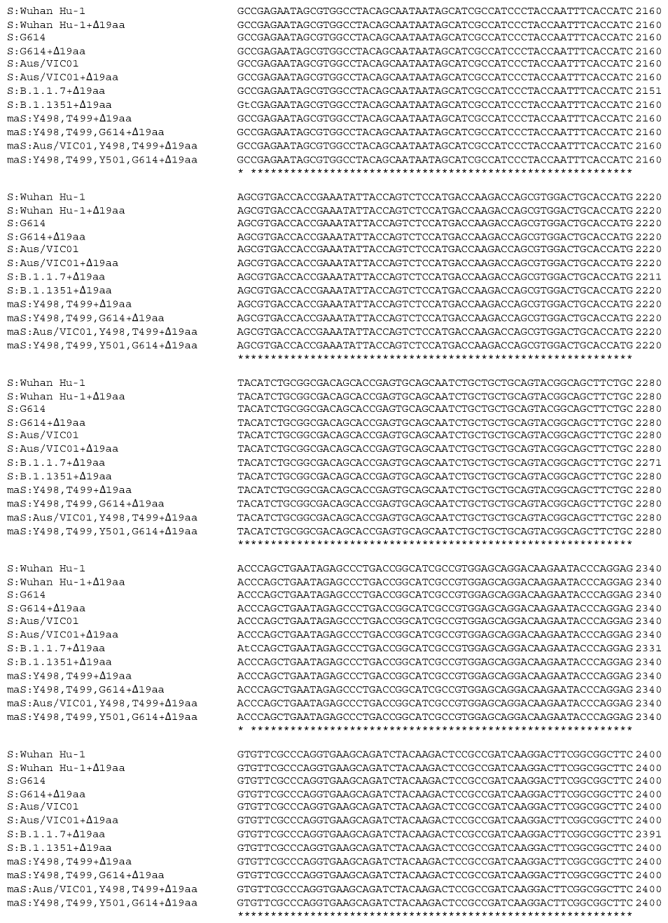

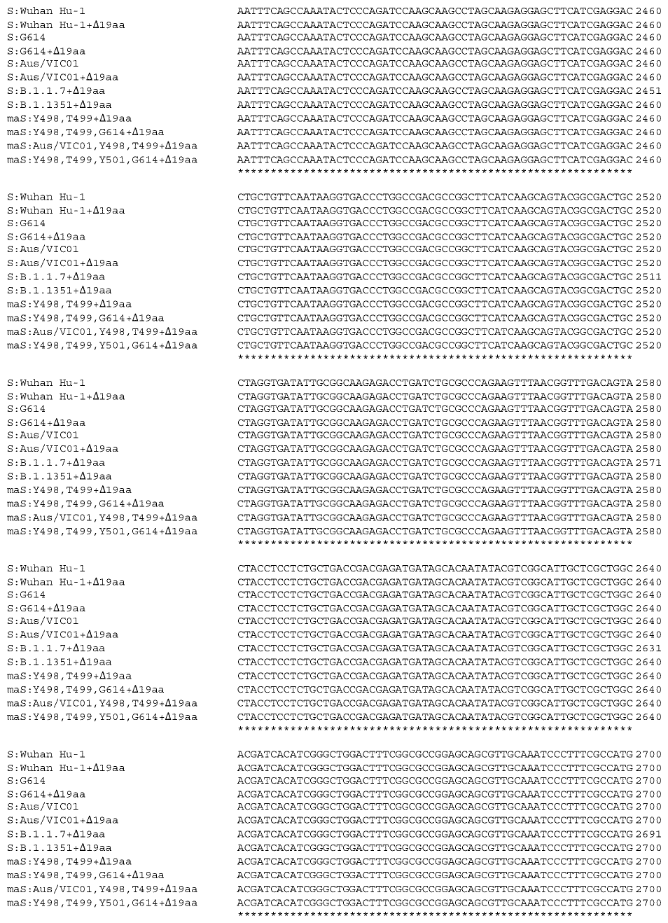

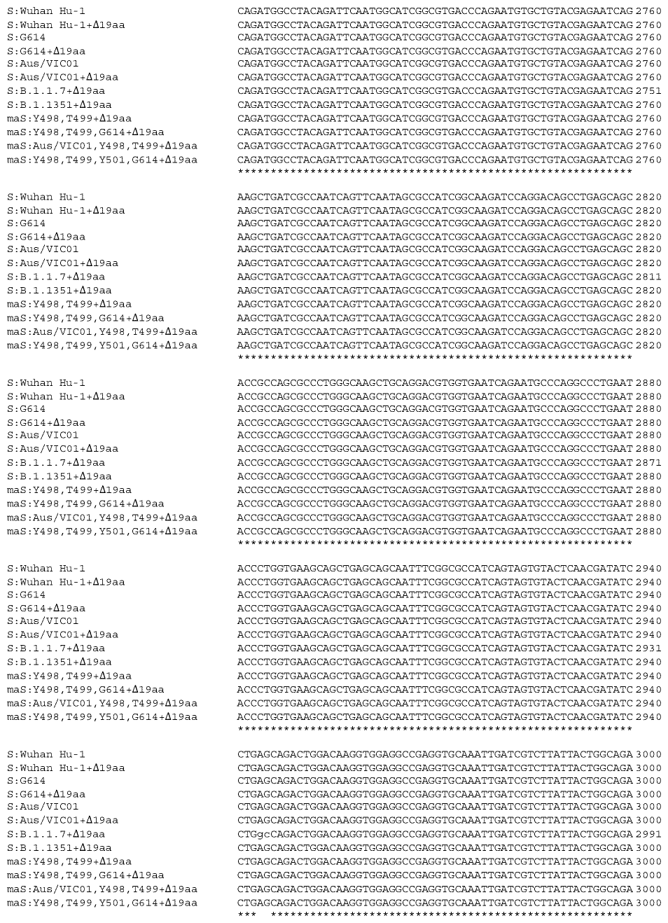

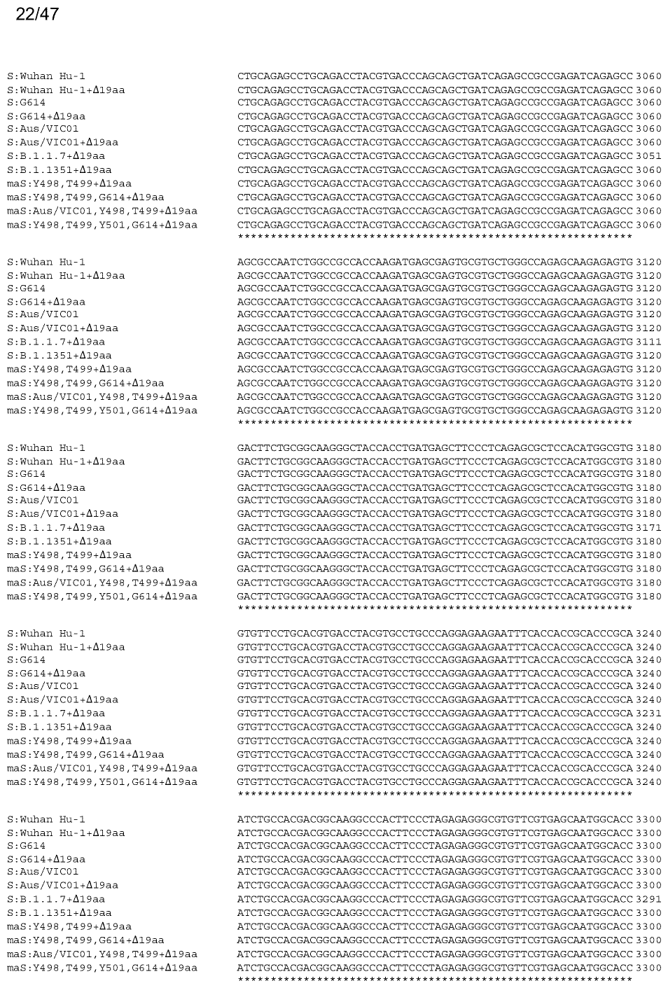

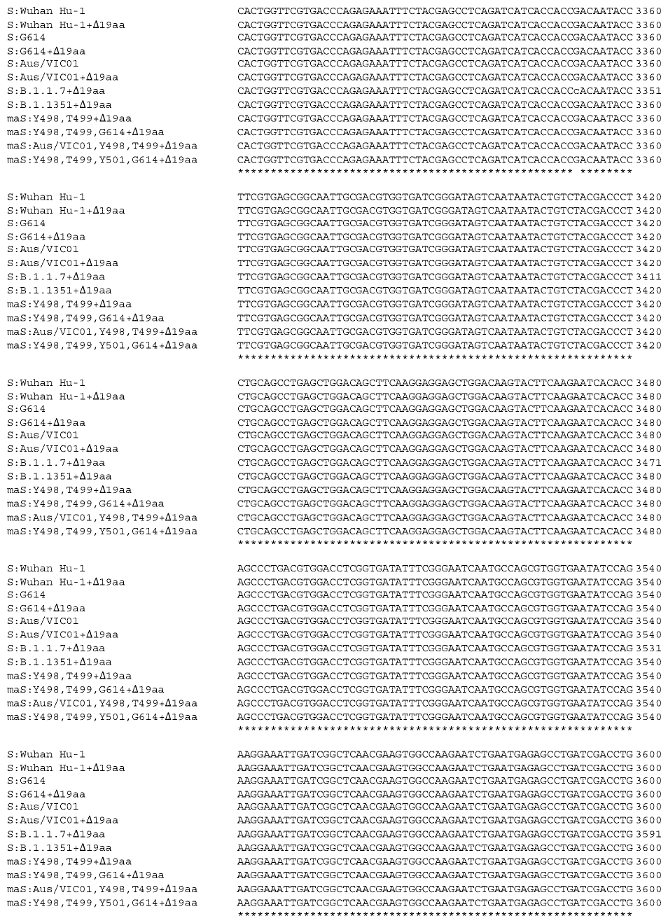

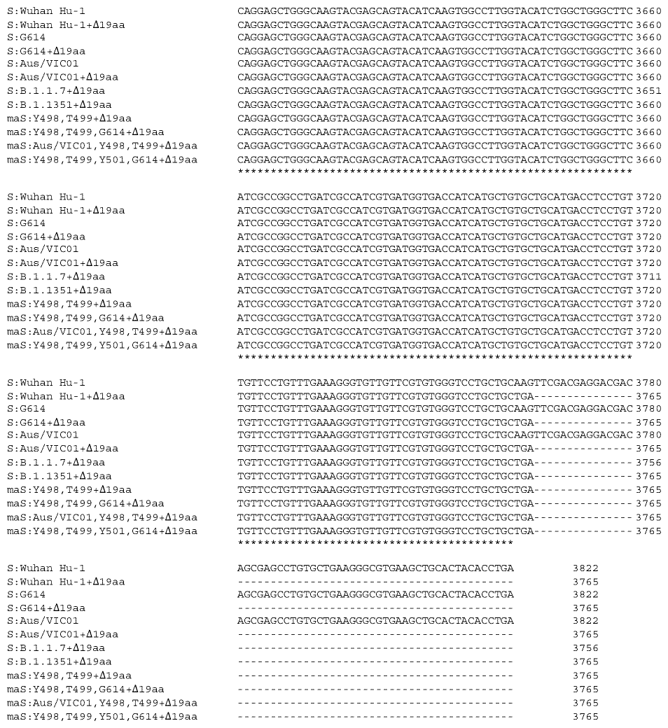

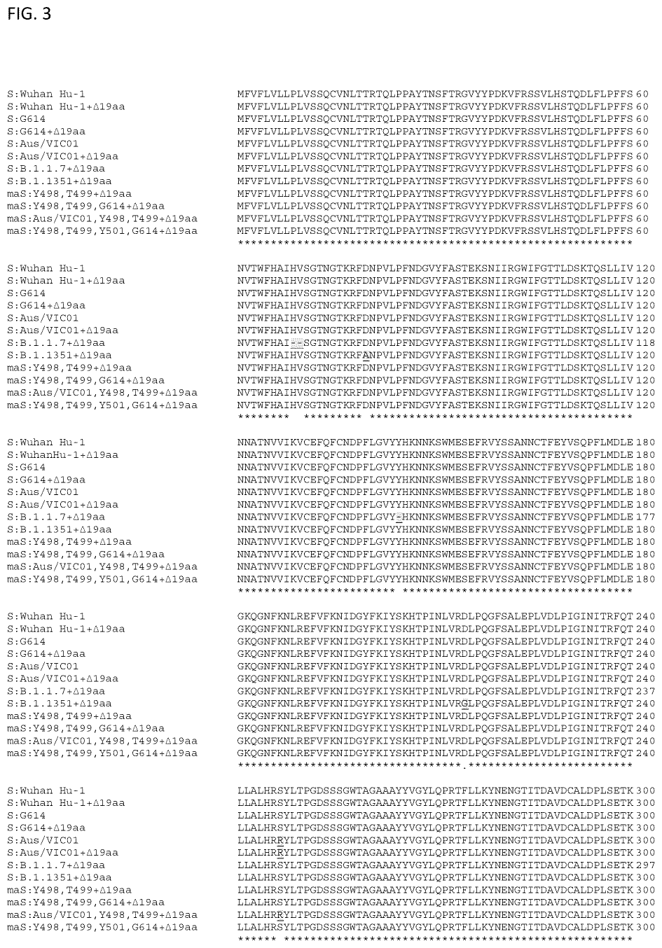

### Sequences

| Wuhan-Hu-1 spike protein amino acid sequence | |
|---|---|
| (encoded by pGM887 S-LV v01) | |
| SEQ ID NO: 1 | |
| See FIG. 3 | |
| Wuhan Hu-1 + Δ19aa amino acid sequence | |
| (encoded by pGM896 S-LV v02) | |
| SEQ ID NO: 2 | |
| See FIG. 3 | |
| S:G614 amino acid sequence | |
| (encoded by pGM906 S-LV v01) | |
| SEQ ID NO: 3 | |
| See FIG. 3 | |
| S:G614 + Δ19aa amino acid sequence | |
| (encoded by pGM907 S-LV v02) | |
| SEQ ID NO: 4 | |
| See FIG. 3 | |
| S:Aus/VIC01 amino acid sequence | |
| (encoded by pGM898 S-LV v01) | |
| SEQ ID NO: 5 | |
| See FIG. 3 | |
| S:Aus/VIC01 + Δ19aa amino acid sequence | |
| (encoded by pGM904 S-LV v02) | |
| SEQ ID NO: 6 | |
| See FIG. 3 | |
| S:B.1.1.7 + Δ19aa amino acid sequence | |
| (encoded by pGM965 S-LV v04) | |
| SEQ ID NO: 7 | |
| See FIG. 3 | |
| S: B.1.1351 + Δ19aa amino acid sequence | |
| (encoded by pGM970 S-LV v04) | |
| SEQ ID NO: 8 | |
| See FIG. 3 | |
| S:B.1.1.7 & B.1.1351 chimera + Δ19aa amino acid sequence | |
| (encoded by pGM1000 S-LV v02) | |
| SEQ ID NO: 9 | |
| MFVFLVLLPLVSSQCVNLTTRTQLPPAYTNSFTRGVYYPDKVFRSSVLHSTQDLFLPFFSNVTWFHAISGTNGTK | |
| RFDNPVLPFNDGVYFASTEKSNIIRGWIFGTTLDSKTQSLLIVNNATNVVIKVCEFQFCNDPFLGVYHKNNKSWM | |
| ESEFRVYSSANNCTFEYVSQPFLMDLEGKQGNFKNLREFVFKNIDGYFKIYSKHTPINLVRDLPQGFSALEPLVD | |
| LPIGINITRFQTLLALHRSYLTPGDSSSGWTAGAAAYYVGYLQPRTFLLKYNENGTITDAVDCALDPLSETKCTL | |
| KSFTVEKGIYQTSNFRVQPTESIVRFPNITNLCPFGEVFNATRFASVYAWNRKRISNCVADYSVLYNSASFSTFK | |
| CYGVSPTKLNDLCFTNVYADSFVIRGDEVRQIAPGQTGKIADYNYKLPDDFTGCVIAWNSNNLDSKVGGNYNYLY | |
| RLFRKSNLKPFERDISTEIYQAGSTPCNGVKGFNCYFPLQSYGFQPTYGVGYQPYRVVVLSFELLHAPATVCGPK | |
| KSTNLVKNKCVNFNFNGLTGTGVLTESNKKFLPFQQFGRDIADTTDAVRDPQTLEILDITPCSFGGVSVITPGTN | |
| TSNQVAVLYQGVNCTEVPVAIHADQLTPTWRVYSTGSNVFQTRAGCLIGAEHVNNSYECDIPIGAGICASYQTQT | |
| NSPRRARSVASQSIIAYTMSLGVENSVAYSNNSIAIPTNFTISVTTEILPVSMTKTSVDCTMYICGDSTECSNLL | |
| LQYGSFCTQLNRALTGIAVEQDKNTQEVFAQVKQIYKTPPIKDFGGFNFSQILPDPSKPSKRSFIEDLLFNKVTL | |
| ADAGFIKQYGDCLGDIAARDLICAQKFNGLTVLPPLLTDEMIAQYTSALLAGTITSGWTFGAGAALQIPFAMQMA | |
| YRFNGIGVTQNVLYENQKLIANQFNSAIGKIQDSLSSTASALGKLQDVVNQNAQALNTLVKQLSSNFGAISSVLN | |
| DILSRLDKVEAEVQIDRLITGRLQSLQTYVTQQLIRAAEIRASANLAATKMSECVLGQSKRVDFCGKGYHLMSFP | |
| QSAPHGVVFLHVTYVPAQEKNFTTAPAICHDGKAHFPREGVFVSNGTHWFVTQRNFYEPQIITTDNTFVSGNCDV | |
| VIGIVNNTVYDPLQPELDSFKEELDKYFKNHTSPDVDLGDISGINASVVNIQKEIDRLNEVAKNLNESLIDLQEL | |
| GKYEQYIKWPWYIWLGFIAGLIAIVMVTIMLCCMTSCCSCLKGCCSCGSCC* | |
| S:B.1.617.2 + Δ19aa amino acid sequence | |
| (encoded by pGM1027 S-LV v02) | |
| SEQ ID NO: 10 | |
| MFVFLVLLPLVSSQCVNLTTRTQLPPAYTNSFTRGVYYPDKVFRSSVLHSTQDLFLPFFSNVTWFHAIHVSGTNV | |
| IKRFDNPVLPFNDGVYFASTEKSNIIRGWIFGTTLDSKTQSLLIVNNATNVVIKVCEFQFCNDPFLGVYYHKNNK | |
| SWMESEFRVYSSANNCTFEYVSQPFLMDLEGKQGNFKNLREFVFKNIDGYFKIYSKHTPINLVRDLPQGFSALEP | |
| LVDLPIGINITRFQTLLALHDSSSGWTAGAAAYYVGYLQPRTFLLKYNENGTITDAVDCALDPLSETKCTLKSFT | |
| VEKGIYQTSNFRVQPTESIVRFPNITNLCPFGEVFNATRFASVYAWNRKRISNCVADYSVLYNSASFSTFKCYGV | |
| SPTKLNDLCFTNVYADSFVIRGDEVRQIAPGQTGKIADYNYKLPDDFTGCVIAWNSNNLDSKVGGNYNYQYRLFR | |
| KSNLKPFERDISTEIYQAGSTPCNGVEGFNCYSPLQSYGFQPTNGVGYQPYRVVVLSFELLHAPATVCGPKKSTN | |
| LVKNKCVNFNFNGLTGTGVLTESNKKFLPFQQFGRDIADTTDAVRDPQTLEILDITPCSFGGVSVITPGTNTSNQ | |
| VAVLYQGVNCTEVPVAIHADQLTPTWRVYSTGSNVFQTRAGCLIGAEHVNNSYECDIPIGAGICASYQTQTNSPR | |
| RARSVASQSIIAYTMSLGAENSVAYSNNSIAIPTNFTISVTTEILPVSMTKTSVDCTMYICGDSTECSNLLLQYG | |
| SFCTQLNRALTGIAVEQDKNTQEVFAQVKQIYKTPPIKDFGGFNFSQILPDPSKPSKRSFIEDLLFNKVTLADAG | |
| FIKQYGDCLGDIAARDLICAQKFNGLNVLPPLLTDEMIAQYTSALLAGTITSGWTFGAGAALQIPFAMQMAYRFN | |
| GIGVTQNVLYENQKLIANQFNSAIGKIQDSLSSTASALGKLQDVVNQNAQALNTLVKQLSSNFGAISSVLNDILS | |
| RLDKVEAEVQIDRLITGRLQSLQTYVTQQLIRAAEIRASANLAATKMSECVLGQSKRVDFCGKGYHLMSFPQSAP | |
| HGVVFLHVTYVPAQEKNFTTAPAICHDGKAHFPREGVFVSNGTHWFVTQRNFYEPQIITTDNTFVSGNCDVVIGI | |
| VNNTVYDPLQPELDSFKEELDKYFKNHTSPDVDLGDISGINASVVNIQKEIDRLNEVAKNLNESLIDLQELGKYE | |
| QYIKWPWYIWLGFIAGLIAIVMVTIMLCCMTSCCSCLKGCCSCGSCC* | |
| C.37 + Δ19aa amino acid sequence | |
| (encoded by pGM1028 S-LV v02) | |
| SEQ ID NO: 11 | |
| MFVFLVLLPLVSSQCVNLTTRTQLPPAYTNSFTRGVYYPDKVFRSSVLHSTQDLFLPFFSNVTWFHAIHVSGTNV | |
| IKRFDNPVLPFNDGVYFASTEKSNIIRGWIFGTTLDSKTQSLLIVNNATNVVIKVCEFQFCNDPFLGVYYHKNNK | |
| SWMESEFRVYSSANNCTFEYVSQPFLMDLEGKQGNFKNLREFVFKNIDGYFKIYSKHTPINLVRDLPQGFSALEP | |
| LVDLPIGINITRFQTLLALHDSSSGWTAGAAAYYVGYLQPRTFLLKYNENGTITDAVDCALDPLSETKCTLKSFT | |
| VEKGIYQTSNFRVQPTESIVRFPNITNLCPFGEVFNATRFASVYAWNRKRISNCVADYSVLYNSASFSTFKCYGV | |
| SPTKLNDLCFTNVYADSFVIRGDEVRQIAPGQTGKIADYNYKLPDDFTGCVIAWNSNNLDSKVGGNYNYQYRLFR | |
| KSNLKPFERDISTEIYQAGSTPCNGVEGFNCYSPLQSYGFQPTNGVGYQPYRVVVLSFELLHAPATVCGPKKSTN | |
| LVKNKCVNFNFNGLTGTGVLTESNKKFLPFQQFGRDIADTTDAVRDPQTLEILDITPCSFGGVSVITPGTNTSNQ | |
| VAVLYQGVNCTEVPVAIHADQLTPTWRVYSTGSNVFQTRAGCLIGAEHVNNSYECDIPIGAGICASYQTQTNSPR | |
| RARSVASQSIIAYTMSLGAENSVAYSNNSIAIPTNFTISVTTEILPVSMTKTSVDCTMYICGDSTECSNLLLQYG | |
| SFCTQLNRALTGIAVEQDKNTQEVFAQVKQIYKTPPIKDFGGFNFSQILPDPSKPSKRSFIEDLLFNKVTLADAG | |
| FIKQYGDCLGDIAARDLICAQKFNGLNVLPPLLTDEMIAQYTSALLAGTITSGWTFGAGAALQIPFAMQMAYRFN | |
| GIGVTQNVLYENQKLIANQFNSAIGKIQDSLSSTASALGKLQDVVNQNAQALNTLVKQLSSNFGAISSVLNDILS | |
| RLDKVEAEVQIDRLITGRLQSLQTYVTQQLIRAAEIRASANLAATKMSECVLGQSKRVDFCGKGYHLMSFPQSAP | |
| HGVVFLHVTYVPAQEKNFTTAPAICHDGKAHFPREGVFVSNGTHWFVTQRNFYEPQIITTDNTFVSGNCDVVIGI | |
| VNNTVYDPLQPELDSFKEELDKYFKNHTSPDVDLGDISGINASVVNIQKEIDRLNEVAKNLNESLIDLQELGKYE | |
| QYIKWPWYIWLGFIAGLIAIVMVTIMLCCMTSCCSCLKGCCSCGSCC* | |
| maS:Y498, T499 + A19aa amino acid sequence | |
| (encoded by pGM937 S-LV v03) | |
| SEQ ID NO: 12 | |
| See FIG. 3 | |
| maS:Y498, T499, G614 + Δ19aa amino acid sequence | |
| (encoded by pGM939 S-LV v03) | |
| SEQ ID NO: 13 | |
| See FIG. 3 | |
| maS: Aus/VIC01, Y498, T499 + Δ19aa amino acid sequence | |
| (encoded by pGM938 S-LV v03) | |
| SEQ ID NO: 14 | |
| See FIG. 3 | |
| S:B.1.617.2, Y501 + Δ19aa amino acid sequence | |
| (encoded by pGM1038 S-LV v04) | |
| SEQ ID NO: 15 | |
| MFVFLVLLPLVSSQCVNLRTRTQLPPAYTNSFTRGVYYPDKVFRSSVLHSTQDLFLPFFSNVTWFHAIHVSGING | |
| TKRFDNPVLPFNDGVYFASTEKSNIIRGWIFGTTLDSKTQSLLIVNNATNVVIKVCEFQFCNDPFLDVYYHKNNK | |
| SWMESGVYSSANNCTFEYVSQPFLMDLEGKQGNFKNLREFVFKNIDGYFKIYSKHTPINLVRDLPQGFSALEPLV | |
| DLPIGINITRFQTLLALHRSYLTPGDSSSGWTAGAAAYYVGYLQPRTFLLKYNENGTITDAVDCALDPLSETKCT | |
| LKSFTVEKGIYQTSNFRVQPTESIVRFPNITNLCPFGEVFNATRFASVYAWNRKRISNCVADYSVLYNSASFSTF | |
| KCYGVSPTKLNDLCFTNVYADSFVIRGDEVRQIAPGQTGKIADYNYKLPDDFTGCVIAWNSNNLDSKVGGNYNYR | |
| YRLFRKSNLKPFERDISTEIYQAGSKPCNGVEGFNCYFPLQSYGFQPTYGVGYQPYRVVVLSFELLHAPATVCGP | |
| KKSTNLVKNKCVNFNFNGLTGTGVLTESNKKFLPFQQFGRDIADTTDAVRDPQTLEILDITPCSFGGVSVITPGT | |
| NTSNQVAVLYQGVNCTEVPVAIHADQLTPTWRVYSTGSNVFQTRAGCLIGAEHVNNSYECDIPIGAGICASYQTQ | |
| TNSRRRARSVASQSIIAYTMSLGAENSVAYSNNSIAIPTNFTISVTTEILPVSMTKTSVDCTMYICGDSTECSNL | |
| LLQYGSFCTQLNRALTGIAVEQDKNTQEVFAQVKQIYKTPPIKDFGGFNFSQILPDPSKPSKRSFIEDLLFNKVT | |
| LADAGFIKQYGDCLGDIAARDLICAQKFNGLTVLPPLLTDEMIAQYTSALLAGTITSGWTFGAGAALQIPFAMQM | |
| AYRFNGIGVTQNVLYENQKLIANQFNSAIGKIQDSLSSTASALGKLQNVVNQNAQALNTLVKQLSSNFGAISSVL | |
| NDILSRLDKVEAEVQIDRLITGRLQSLQTYVTQQLIRAAEIRASANLAATKMSECVLGQSKRVDFCGKGYHLMSF | |
| PQSAPHGVVFLHVTYVPAQEKNFTTAPAICHDGKAHFPREGVFVSNGTHWFVTQRNFYEPQIITTDNTFVSGNCD | |
| VVIGIVNNTVYDPLQPELDSFKEELDKYFKNHTSPDVDLGDISGINASVVNIQKEIDRLNEVAKNLNESLIDLQE | |
| LGKYEQYIKWPWYIWLGFIAGLIAIVMVTIMLCCMTSCCSCLKGCCSCGSCC* | |
| C.37, Y501 + Δ19aa amino acid sequence | |
| (encoded by pGM1039 S-LV v05) | |
| SEQ ID NO: 16 | |
| MFVFLVLLPLVSSQCVNLTTRTQLPPAYTNSFTRGVYYPDKVFRSSVLHSTQDLFLPFFSNVTWFHAIHVSGTNV | |
| IKRFDNPVLPFNDGVYFASTEKSNIIRGWIFGTTLDSKTQSLLIVNNATNVVIKVCEFQFCNDPFLGVYYHKNNK | |
| SWMESEFRVYSSANNCTFEYVSQPFLMDLEGKQGNFKNLREFVFKNIDGYFKIYSKHTPINLVRDLPQGFSALEP | |
| LVDLPIGINITRFQTLLALHDSSSGWTAGAAAYYVGYLQPRTFLLKYNENGTITDAVDCALDPLSETKCTLKSFT | |
| VEKGIYQTSNFRVQPTESIVRFPNITNLCPFGEVFNATRFASVYAWNRKRISNCVADYSVLYNSASFSTFKCYGV | |
| SPTKLNDLCFTNVYADSFVIRGDEVRQIAPGQTGKIADYNYKLPDDFTGCVIAWNSNNLDSKVGGNYNYQYRLFR | |
| KSNLKPFERDISTEIYQAGSTPCNGVEGFNCYSPLQSYGFQPTYGVGYQPYRVVVLSFELLHAPATVCGPKKSTN | |
| LVKNKCVNFNFNGLTGTGVLTESNKKFLPFQQFGRDIADTTDAVRDPQTLEILDITPCSFGGVSVITPGTNTSNQ | |
| VAVLYQGVNCTEVPVAIHADQLTPTWRVYSTGSNVFQTRAGCLIGAEHVNNSYECDIPIGAGICASYQTQTNSPR | |
| RARSVASQSIIAYTMSLGAENSVAYSNNSIAIPTNFTISVTTEILPVSMTKTSVDCTMYICGDSTECSNLLLQYG | |
| SFCTQLNRALTGIAVEQDKNTQEVFAQVKQIYKTPPIKDFGGFNFSQILPDPSKPSKRSFIEDLLFNKVTLADAG | |
| FIKQYGDCLGDIAARDLICAQKFNGLNVLPPLLTDEMIAQYTSALLAGTITSGWTFGAGAALQIPFAMQMAYRFN | |
| GIGVTQNVLYENQKLIANQFNSAIGKIQDSLSSTASALGKLQDVVNQNAQALNTLVKQLSSNFGAISSVLNDILS | |
| RLDKVEAEVQIDRLITGRLQSLQTYVTQQLIRAAEIRASANLAATKMSECVLGQSKRVDFCGKGYHLMSFPQSAP | |
| HGVVFLHVTYVPAQEKNFTTAPAICHDGKAHFPREGVFVSNGTHWFVTQRNFYEPQIITTDNTFVSGNCDVVIGI | |
| VNNTVYDPLQPELDSFKEELDKYFKNHTSPDVDLGDISGINASVVNIQKEIDRLNEVAKNLNESLIDLQELGKYE | |
| QYIKWPWYIWLGFIAGLIAIVMVTIMLCCMTSCCSCLKGCCSCGSCC* | |
| S:B.1.617.2, Y498, T499, Y501 + Δ19aa amino acid sequence | |
| (encoded by pGM1040 SLV v04) | |
| SEQ ID NO: 17 | |
| MFVFLVLLPLVSSQCVNLRTRTQLPPAYTNSFTRGVYYPDKVFRSSVLHSTQDLFLPFFSNVTWFHAIHVSGING | |
| TKRFDNPVLPFNDGVYFASTEKSNIIRGWIFGTTLDSKTQSLLIVNNATNVVIKVCEFQFCNDPFLDVYYHKNNK | |
| SWMESGVYSSANNCTFEYVSQPFLMDLEGKQGNFKNLREFVFKNIDGYFKIYSKHTPINLVRDLPQGFSALEPLV | |
| DLPIGINITRFQTLLALHRSYLTPGDSSSGWTAGAAAYYVGYLQPRTFLLKYNENGTITDAVDCALDPLSETKCT | |
| LKSFTVEKGIYQTSNFRVQPTESIVRFPNITNLCPFGEVFNATRFASVYAWNRKRISNCVADYSVLYNSASFSTF | |
| KCYGVSPTKLNDLCFTNVYADSFVIRGDEVRQIAPGQTGKIADYNYKLPDDFTGCVIAWNSNNLDSKVGGNYNYR | |
| YRLFRKSNLKPFERDISTEIYQAGSKPCNGVEGFNCYFPLQSYGFYTTYGVGYQPYRVVVLSFELLHAPATVCGP | |
| KKSTNLVKNKCVNFNFNGLTGTGVLTESNKKFLPFQQFGRDIADTTDAVRDPQTLEILDITPCSFGGVSVITPGT | |
| NTSNQVAVLYQGVNCTEVPVAIHADQLTPTWRVYSTGSNVFQTRAGCLIGAEHVNNSYECDIPIGAGICASYQTQ | |
| TNSRRRARSVASQSIIAYTMSLGAENSVAYSNNSIAIPTNFTISVTTEILPVSMTKTSVDCTMYICGDSTECSNL | |
| LLQYGSFCTQLNRALTGIAVEQDKNTQEVFAQVKQIYKTPPIKDFGGFNFSQILPDPSKPSKRSFIEDLLFNKVT | |
| LADAGFIKQYGDCLGDIAARDLICAQKFNGLTVLPPLLTDEMIAQYTSALLAGTITSGWTFGAGAALQIPFAMQM | |
| AYRFNGIGVTQNVLYENQKLIANQFNSAIGKIQDSLSSTASALGKLQNVVNQNAQALNTLVKQLSSNFGAISSVL | |
| NDILSRLDKVEAEVQIDRLITGRLQSLQTYVTQQLIRAAEIRASANLAATKMSECVLGQSKRVDFCGKGYHLMSF | |
| PQSAPHGVVFLHVTYVPAQEKNFTTAPAICHDGKAHFPREGVFVSNGTHWFVTQRNFYEPQIITTDNTFVSGNCD | |
| VVIGIVNNTVYDPLQPELDSFKEELDKYFKNHTSPDVDLGDISGINASVVNIQKEIDRLNEVAKNLNESLIDLQE | |
| LGKYEQYIKWPWYIWLGFIAGLIAIVMVTIMLCCMTSCCSCLKGCCSCGSCC* | |
| C.37, Y489, T499, Y501 + Δ19aa amino acid sequence | |
| (encoded by pGM1041 S-LV v05) | |
| SEQ ID NO: 18 | |
| MFVFLVLLPLVSSQCVNLTTRTQLPPAYTNSFTRGVYYPDKVFRSSVLHSTQDLFLPFFSNVTWFHAIHVSGTNV | |
| IKRFDNPVLPFNDGVYFASTEKSNIIRGWIFGTTLDSKTQSLLIVNNATNVVIKVCEFQFCNDPFLGVYYHKNNK | |
| SWMESEFRVYSSANNCTFEYVSQPFLMDLEGKQGNFKNLREFVFKNIDGYFKIYSKHTPINLVRDLPQGFSALEP | |
| LVDLPIGINITRFQTLLALHDSSSGWTAGAAAYYVGYLQPRTFLLKYNENGTITDAVDCALDPLSETKCTLKSFT | |
| VEKGIYQTSNFRVQPTESIVRFPNITNLCPFGEVFNATRFASVYAWNRKRISNCVADYSVLYNSASFSTFKCYGV | |
| SPTKLNDLCFTNVYADSFVIRGDEVRQIAPGQTGKIADYNYKLPDDFTGCVIAWNSNNLDSKVGGNYNYQYRLFR | |
| KSNLKPFERDISTEIYQAGSTPCNGVEGFNCYSPLQSYGFYTTYGVGYQPYRVVVLSFELLHAPATVCGPKKSTN | |
| LVKNKCVNFNFNGLTGTGVLTESNKKFLPFQQFGRDIADTTDAVRDPQTLEILDITPCSFGGVSVITPGTNTSNQ | |
| VAVLYQGVNCTEVPVAIHADQLTPTWRVYSTGSNVFQTRAGCLIGAEHVNNSYECDIPIGAGICASYQTQTNSPR | |
| RARSVASQSIIAYTMSLGAENSVAYSNNSIAIPTNFTISVTTEILPVSMTKTSVDCTMYICGDSTECSNLLLQYG | |
| SFCTQLNRALTGIAVEQDKNTQEVFAQVKQIYKTPPIKDFGGFNFSQILPDPSKPSKRSFIEDLLFNKVTLADAG | |
| FIKQYGDCLGDIAARDLICAQKFNGLNVLPPLLTDEMIAQYTSALLAGTITSGWTFGAGAALQIPFAMQMAYRFN | |
| GIGVTQNVLYENQKLIANQFNSAIGKIQDSLSSTASALGKLQDVVNQNAQALNTLVKQLSSNFGAISSVLNDILS | |
| RLDKVEAEVQIDRLITGRLQSLQTYVTQQLIRAAEIRASANLAATKMSECVLGQSKRVDFCGKGYHLMSFPQSAP | |
| HGVVFLHVTYVPAQEKNFTTAPAICHDGKAHFPREGVFVSNGTHWFVTQRNFYEPQIITTDNTFVSGNCDVVIGI | |
| VNNTVYDPLQPELDSFKEELDKYFKNHTSPDVDLGDISGINASVVNIQKEIDRLNEVAKNLNESLIDLQELGKYE | |
| QYIKWPWYIWLGFIAGLIAIVMVTIMLCCMTSCCSCLKGCCSCGSCC* | |
| maS:Y498, T499, Y501 + Δ19aa amino acid sequence | |
| (encoded by pGM998 S-LV v05) | |
| SEQ ID NO: 19 | |
| MFVFLVLLPLVSSQCVNLTTRTQLPPAYTNSFTRGVYYPDKVFRSSVLHSTQDLFLPFFSNVTWFHAIHVSGING | |
| TKRFDNPVLPFNDGVYFASTEKSNIIRGWIFGTTLDSKTQSLLIVNNATNVVIKVCEFQFCNDPFLGVYYHKNNK | |
| SWMESEFRVYSSANNCTFEYVSQPFLMDLEGKQGNFKNLREFVFKNIDGYFKIYSKHTPINLVRDLPQGFSALEP | |
| LVDLPIGINITRFQTLLALHRSYLTPGDSSSGWTAGAAAYYVGYLQPRTFLLKYNENGTITDAVDCALDPLSETK | |
| CTLKSFTVEKGIYQTSNFRVQPTESIVRFPNITNLCPFGEVFNATRFASVYAWNRKRISNCVADYSVLYNSASFS | |
| TFKCYGVSPTKLNDLCFTNVYADSFVIRGDEVRQIAPGQTGKIADYNYKLPDDFTGCVIAWNSNNLDSKVGGNYN | |
| YLYRLFRKSNLKPFERDISTEIYQAGSTPCNGVEGFNCYFPLQSYGFYTTYGVGYQPYRVVVLSFELLHAPATVC | |
| GPKKSTNLVKNKCVNFNFNGLTGTGVLTESNKKFLPFQQFGRDIADTTDAVRDPQTLEILDITPCSFGGVSVITP | |
| GTNTSNQVAVLYQDVNCTEVPVAIHADQLTPTWRVYSTGSNVFQTRAGCLIGAEHVNNSYECDIPIGAGICASYQ | |
| TQTNSPRRARSVASQSIIAYTMSLGAENSVAYSNNSIAIPTNFTISVTTEILPVSMTKTSVDCTMYICGDSTECS | |
| NLLLQYGSFCTQLNRALTGIAVEQDKNTQEVFAQVKQIYKTPPIKDFGGFNFSQILPDPSKPSKRSFIEDLLENK | |
| VTLADAGFIKQYGDCLGDIAARDLICAQKFNGLTVLPPLLTDEMIAQYTSALLAGTITSGWTFGAGAALQIPFAM | |
| QMAYRFNGIGVTQNVLYENQKLIANQFNSAIGKIQDSLSSTASALGKLQDVVNQNAQALNTLVKQLSSNFGAISS | |
| VLNDILSRLDKVEAEVQIDRLITGRLQSLQTYVTQQLIRAAEIRASANLAATKMSECVLGQSKRVDFCGKGYHLM | |
| SFPQSAPHGVVFLHVTYVPAQEKNFTTAPAICHDGKAHFPREGVFVSNGTHWFVTQRNFYEPQIITTDNTFVSGN | |
| CDVVIGIVNNTVYDPLQPELDSFKEELDKYFKNHTSPDVDLGDISGINASVVNIQKEIDRLNEVAKNLNESLIDL | |
| QELGKYEQYIKWPWYIWLGFIAGLIAIVMVTIMLCCMTSCCSCLKGCCSCGSCC* | |
| maS:Y498, T499, Y501, G614 + Δ19aa amino acid sequence | |
| (encoded by pGM999 S-LV v05) | |
| SEQ ID NO: 20 | |
| See FIG. 3 | |
| S:G614 with furin site knock out amino acid sequence | |
| (encoded by pGM1026 S-LV v05) | |
| SEQ ID NO: 21 | |
| MFVFLVLLPLVSSQCVNLTTRTQLPPAYTNSFTRGVYYPDKVFRSSVLHSTQDLFLPFFSNVTWFHAIHVSGTNG | |
| TKRFDNPVLPFNDGVYFASTEKSNIIRGWIFGTTLDSKTQSLLIVNNATNVVIKVCEFQFCNDPFLGVYYHKNNK | |
| SWMESEFRVYSSANNCTFEYVSQPFLMDLEGKQGNFKNLREFVFKNIDGYFKIYSKHTPINLVRDLPQGFSALEP | |
| LVDLPIGINITRFQTLLALHRSYLTPGDSSSGWTAGAAAYYVGYLQPRTFLLKYNENGTITDAVDCALDPLSETK | |
| CTLKSFTVEKGIYQTSNFRVQPTESIVRFPNITNLCPFGEVFNATRFASVYAWNRKRISNCVADYSVLYNSASFS | |
| TFKCYGVSPTKLNDLCFTNVYADSFVIRGDEVRQIAPGQTGKIADYNYKLPDDFTGCVIAWNSNNLDSKVGGNYN | |
| YLYRLFRKSNLKPFERDISTEIYQAGSTPCNGVEGFNCYFPLQSYGFQPTNGVGYQPYRVVVLSFELLHAPATVC | |
| GPKKSTNLVKNKCVNFNFNGLTGTGVLTESNKKFLPFQQFGRDIADTTDAVRDPQTLEILDITPCSFGGVSVITP | |
| GTNTSNQVAVLYQGVNCTEVPVAIHADQLTPTWRVYSTGSNVFQTRAGCLIGAEHVNNSYECDIPIGAGICASYQ | |
| TQTNSPSRASSVASQSIIAYTMSLGAENSVAYSNNSIAIPTNFTISVTTEILPVSMTKTSVDCTMYICGDSTECS | |
| NLLLQYGSFCTQLNRALTGIAVEQDKNTQEVFAQVKQIYKTPPIKDFGGFNFSQILPDPSKPSKRSFIEDLLFNK | |
| VTLADAGFIKQYGDCLGDIAARDLICAQKFNGLTVLPPLLTDEMIAQYTSALLAGTITSGWTFGAGAALQIPFAM | |
| QMAYRFNGIGVTQNVLYENQKLIANQFNSAIGKIQDSLSSTASALGKLQDVVNQNAQALNTLVKQLSSNFGAISS | |
| VLNDILSRLDKVEAEVQIDRLITGRLQSLQTYVTQQLIRAAEIRASANLAATKMSECVLGQSKRVDFCGKGYHLM | |
| SFPQSAPHGVVFLHVTYVPAQEKNFTTAPAICHDGKAHFPREGVFVSNGTHWFVTQRNFYEPQIITTDNTFVSGN | |
| CDVVIGIVNNTVYDPLQPELDSFKEELDKYFKNHTSPDVDLGDISGINASVVNIQKEIDRLNEVAKNLNESLIDL | |
| QELGKYEQYIKWPWYIWLGFIAGLIAIVMVTIMLCCMTSCCSCLKGCCSCGSCC* | |
| Wuhan-Hu-1 spike protein nucleic acid sequence | |
| (comprised in pGM887 S-LV v01) | |
| SEQ ID NO: 22 | |
| See FIG. 2 | |
| Wuhan Hu-1 + Δ19aa nucleic acid sequence | |
| (comprised in pGM896 S-LV v02) | |
| SEQ ID NO: 23 | |
| See FIG. 2 | |
| S:G614 nucleic acid nucleic | |
| (comprised in pGM906 S-LV v01) | |
| SEQ ID NO: 24 | |
| See FIG. 2 | |
| S:G614 + A19aa nucleic acid sequence | |
| (comprised in pGM907 S-LV v02) | |
| SEQ ID NO: 25 | |
| See FIG. 2 | |
| S:Aus/VIC01 nucleic acid sequence | |
| (comprised in pGM898 S-LV v01) | |
| SEQ ID NO: 26 | |
| See FIG. 2 | |
| S:Aus/VIC01 + Δ19aa nucleic acid sequence | |
| (comprised in pGM904 S-LV v02) | |
| SEQ ID NO: 27 | |
| See FIG. 2 | |
| S:B.1.1.7 + Δ19aa nucleic acid sequence | |
| (comprised in pGM965 S-LV v04) | |
| SEQ ID NO: 28 | |
| See FIG. 2 | |
| S:B.1.1351 + Δ19aa nucleic acid sequence | |
| (comprised in pGM970 S-LV v04) | |
| SEQ ID NO: 29 | |
| See FIG. 2 | |
| S:B.1.1.7 & B.1.1351 chimera + A19aa nucleic acid sequence | |
| (comprised in pGM1000 S-LV v02) | |
| SEQ ID NO: 30 | |
| ATGTTCGTGTTCCTGGTGCTGCTGCCTCTGGTGAGCAGCCAGTGCGTGAATCTGACCACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCAGCGGCACCAATGGCACCAAG | |
| AGATTCGACAATCCTGTGCTGCCTTTCAATGACGGCGTGTACTTCGCCAGCACCGAGAAGAGCAATATCATCAGA | |
| GGCTGGATCTTCGGCACCACCTTGGATTCCAAGACTCAGAGCCTGCTGATTGTAAACAACGCTACAAATGTGGTG | |
| ATCAAGGTGTGCGAGTTCCAGTTCTGCAATGACCCTTTCCTGGGTGTTTATCATAAGAACAACAAGAGCTGGATG | |
| GAGAGCGAGTTCCGCGTATATTCGTCGGCTAATAATTGCACCTTCGAGTACGTGAGCCAGCCTTTCCTGATGGAC | |
| CTGGAGGGCAAGCAGGGCAATTTCAAGAATCTGAGAGAGTTCGTGTTCAAGAATATCGACGGCTACTTCAAGATC | |
| TACAGCAAGCACACACCCATTAATCTGGTGAGAGACCTGCCTCAGGGCTTCAGCGCCCTGGAGCCTCTGGTGGAC | |
| CTGCCTATCGGCATCAATATCACCAGATTCCAGACCCTGCTGGCCCTGCACAGATCATATCTTACACCAGGCGAT | |
| TCGTCAAGCGGTTGGACCGCTGGAGCTGCGGCATATTACGTGGGCTACCTGCAGCCTAGAACCTTCCTGCTGAAG | |
| TACAATGAGAATGGTACGATAACCGACGCAGTTGATTGTGCCCTGGACCCTCTGAGCGAGACCAAGTGCACCCTG | |
| AAGAGCTTCACCGTGGAGAAGGGCATCTACCAGACCAGCAATTTCAGAGTGCAGCCTACCGAGAGCATCGTGAGA | |
| TTCCCTAATATCACCAATCTGTGCCCTTTCGGCGAGGTGTTCAATGCCACCAGATTCGCCAGCGTGTACGCATGG | |
| AACCGCAAGCGGATAAGCAATTGCGTGGCCGACTACAGCGTGCTGTACAATAGCGCCAGCTTCAGCACCTTCAAA | |
| TGTTATGGTGTTTCGCCAACAAAGCTGAATGACCTGTGCTTCACCAATGTGTACGCCGACAGCTTCGTGATCAGA | |
| GGCGACGAGGTGAGACAGATCGCGCCAGGGCAGACCGGCAAGATCGCCGACTACAATTACAAGCTGCCTGACGAC | |
| TTCACCGGCTGCGTGATCGCGTGGAACTCTAACAATCTAGATTCGAAAGTTGGAGGCAATTACAATTACCTGTAC | |
| AGACTGTTCAGAAAGAGCAATCTGAAGCCTTTCGAGAGAGACATCAGCACCGAGATCTACCAGGCCGGCAGCACA | |
| CCGTGTAATGGCGTGaAGGGCTTCAATTGCTACTTCCCTCTGCAGAGCTACGGCTTCCAGCCTACCtATGGCGTG | |
| GGCTACCAGCCTTACAGAGTGGTGGTGCTGAGCTTCGAGCTGCTGCACGCTCCCGCTACCGTGTGCGGCCCTAAG | |
| AAGAGCACCAATCTGGTGAAGAATAAGTGCGTGAATTTCAATTTCAATGGTCTAACTGGLACGGGCGTGCTGACC | |
| GAGAGCAATAAGAAGTTTCTTCCCTTTCAACAATTCGGCAGAGACATCGCCGACACCACAGATGCTGTAAGAGAC | |
| CCTCAGACCCTGGAGATCCTGGACATCACTCCGTGTAGCTTCGGCGGCGTGAGCGTGATCACACCGGGTACCAAT | |
| ACCAGCAATCAGGTGGCCGTGCTGTACCAGGgCGTGAATTGCACCGAGGTGCCTGTGGCCATCCACGCCGACCAG | |
| CTGACTCCCACTTGGAGGGTATATTCCACGGGAAGCAATGTGTTCCAGACCAGAGCCGGCTGCCTGATCGGCGCC | |
| GAGCACGTGAATAATAGCTACGAGTGCGACATCCCTATCGGCGCCGGCATCTGCGCCAGCTACCAGACCCAGACC | |
| AATAGCCCTAGAAGAGCCAGAAGCGTGGCCAGCCAGAGCATCATCGCCTACACCATGAGCCTGGGCGtCGAGAAT | |
| AGCGTGGCCTACAGCAATAATAGCATCGCCATCCCTACCAATTTCACCATCAGCGTGACCACCGAAATATTACCA | |
| GTCTCCATGACCAAGACCAGCGTGGACTGCACCATGTACATCTGCGGCGACAGCACCGAGTGCAGCAATCTGCTG | |
| CTGCAGTACGGCAGCTTCTGCACCCAGCTGAATAGAGCCCTGACCGGCATCGCCGTGGAGCAGGACAAGAATACC | |
| CAGGAGGTGTTCGCCCAGGTGAAGCAGATCTACAAGACTCCGCCGATCAAGGACTTCGGCGGCTTCAATTTCAGC | |
| CAAATACTCCCAGATCCAAGCAAGCCTAGCAAGAGGAGCTTCATCGAGGACCTGCTGTTCAATAAGGTGACCCTG | |
| GCCGACGCCGGCTTCATCAAGCAGTACGGCGACTGCCTAGGTGATATTGCGGCAAGAGACCTGATCTGCGCCCAG | |
| AAGTTTAACGGTTTGACAGTACTACCTCCTCTGCTGACCGACGAGATGATAGCACAATATACGTCGGCATTGCTC | |
| GCTGGCACGATCACATCGGGCTGGACTTTCGGCGCCGGAGCAGCGTTGCAAATCCCTTTCGCCATGCAGATGGCC | |
| TACAGATTCAATGGCATCGGCGTGACCCAGAATGTGCTGTACGAGAATCAGAAGCTGATCGCCAATCAGTTCAAT | |
| AGCGCCATCGGCAAGATCCAGGACAGCCTGAGCAGCACCGCCAGCGCCCTGGGCAAGCTGCAGGACGTGGTGAAT | |
| CAGAATGCCCAGGCCCTGAATACCCTGGTGAAGCAGCTGAGCAGCAATTTCGGCGCCATCAGTAGTGTACTCAAC | |
| GATATCCTGAGCAGACTGGACAAGGTGGAGGCCGAGGTGCAAATTGATCGTCTTATTACTGGCAGACTGCAGAGC | |
| CTGCAGACCTACGTGACCCAGCAGCTGATCAGAGCCGCCGAGATCAGAGCCAGCGCCAATCTGGCCGCCACCAAG | |
| ATGAGCGAGTGCGTGCTGGGCCAGAGCAAGAGAGTGGACTTCTGCGGCAAGGGCTACCACCTGATGAGCTTCCCT | |
| CAGAGCGCTCCACATGGCGTGGTGTTCCTGCACGTGACCTACGTGCCTGCCCAGGAGAAGAATTTCACCACCGCA | |
| CCCGCAATCTGCCACGACGGCAAGGCCCACTTCCCTAGAGAGGGCGTGTTCGTGAGCAATGGCACCCACTGGTTC | |
| GTGACCCAGAGAAATTTCTACGAGCCTCAGATCATCACCACCGACAATACCTTCGTGAGCGGCAATTGCGACGTG | |
| GTGATCGGGATAGTCAATAATACTGTCTACGACCCTCTGCAGCCTGAGCTGGACAGCTTCAAGGAGGAGCTGGAC | |
| AAGTACTTCAAGAATCACACCAGCCCTGACGTGGACCTCGGTGATATTTCGGGAATCAATGCCAGCGTGGTGAAT | |
| ATCCAGAAGGAAATTGATCGGCTCAACGAAGTGGCCAAGAATCTGAATGAGAGCCTGATCGACCTGCAGGAGCTG | |
| GGCAAGTACGAGCAGTACATCAAGTGGCCTTGGTACATCTGGCTGGGCTTCATCGCCGGCCTGATCGCCATCGTG | |
| ATGGTGACCATCATGCTGTGCTGCATGACCTCCTGTTGTTCCTGTTTGAAAGGGTGTTGTTCGTGTGGGTCCTGC | |
| TGCTGA | |
| S:B.1.617.2 + Δ19aa nucleic acid sequence | |
| (comprised in pGM1027 S-LV v02) | |
| SEQ ID NO: 31 | |
| ATGTTCGTGTTCCTGGTGCTGCTGCCTCTGGTGAGCAGCCAGTGCGTGAATCTGAggACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGGC | |
| ACCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGaTGTTTATTATCATAAGAACAACAAG | |
| AGCTGGATGGAGAGCgGCGTATATTCGTCGGCTAATAATTGCACCTTCGAGTACGTGAGCCAGCCTTTCCTGATG | |
| GACCTGGAGGGCAAGCAGGGCAATTTCAAGAATCTGAGAGAGTTCGTGTTCAAGAATATCGACGGCTACTTCAAG | |
| ATCTACAGCAAGCACACACCCATTAATCTGGTGAGAGACCTGCCTCAGGGCTTCAGCGCCCTGGAGCCTCTGGTG | |
| GACCTGCCTATCGGCATCAATATCACCAGATTCCAGACCCTGCTGGCCCTGCACAGATCATATCTTACACCAGGC | |
| GATTCGTCAAGCGGTTGGACCGCTGGAGCTGCGGCATATTACGTGGGCTACCTGCAGCCTAGAACCTTCCTGCTG | |
| AAGTACAATGAGAATGGTACGATAACCGACGCAGTTGATTGTGCCCTGGACCCTCTGAGCGAGACCAAGTGCACC | |
| CTGAAGAGCTTCACCGTGGAGAAGGGCATCTACCAGACCAGCAATTTCAGAGTGCAGCCTACCGAGAGCATCGTG | |
| AGATTCCCTAATATCACCAATCTGTGCCCTTTCGGCGAGGTGTTCAATGCCACCAGATTCGCCAGCGTGTACGCA | |
| TGGAACCGCAAGCGGATAAGCAATTGCGTGGCCGACTACAGCGTGCTGTACAATAGCGCCAGCTTCAGCACCTTC | |
| AAATGTTATGGTGTTTCGCCAACAAAGCTGAATGACCTGTGCTTCACCAATGTGTACGCCGACAGCTTCGTGATC | |
| AGAGGCGACGAGGTGAGACAGATCGCGCCAGGGCAGACCGGCAAGATCGCCGACTACAATTACAAGCTGCCTGAC | |
| GACTTCACCGGCTGCGTGATCGCGTGGAACTCTAACAATCTAGATTCGAAAGTTGGAGGCAATTACAATTACCgG | |
| TACAGACTGTTCAGAAAGAGCAATCTGAAGCCTTTCGAGAGAGACATCAGCACCGAGATCTACCAGGCCGGCAGC | |
| AaACCGTGTAATGGCGTGGAGGGCTTCAATTGCTACTTCCCTCTGCAGAGCTACGGCTTCCAGCCTACCAATGGC | |
| GTGGGCTACCAGCCTTACAGAGTGGTGGTGCTGAGCTTCGAGCTGCTGCACGCTCCCGCTACCGTGTGCGGCCCT | |
| AAGAAGAGCACCAATCTGGTGAAGAATAAGTGCGTGAATTTCAATTTCAATGGTCTAACTGGAACGGGCGTGCTG | |
| ACCGAGAGCAATAAGAAGTTTCTTCCCTTTCAACAATTCGGCAGAGACATCGCCGACACCACAGATGCTGTAAGA | |
| GACCCTCAGACCCTGGAGATCCTGGACATCACTCCGTGTAGCTTCGGCGGCGTGAGCGTGATCACACCGGGTACC | |
| AATACCAGCAATCAGGTGGCCGTGCTGTACCAGGgCGTGAATTGCACCGAGGTGCCTGTGGCCATCCACGCCGAC | |
| CAGCTGACTCCCACTTGGAGGGTATATTCCACGGGAAGCAATGTGTTCCAGACCAGAGCCGGCTGCCTGATCGGC | |
| GCCGAGCACGTGAATAATAGCTACGAGTGCGACATCCCTATCGGCGCCGGCATCTGCGCCAGCTACCAGACCCAG | |
| ACCAATAGCCgTAGAAGAGCCAGAAGCGTGGCCAGCCAGAGCATCATCGCCTACACCATGAGCCTGGGCGCCGAG | |
| AATAGCGTGGCCTACAGCAATAATAGCATCGCCATCCCTACCAATTTCACCATCAGCGTGACCACCGAAATATTA | |
| CCAGTCTCCATGACCAAGACCAGCGTGGACTGCACCATGTACATCTGCGGCGACAGCACCGAGTGCAGCAATCTG | |
| CTGCTGCAGTACGGCAGCTTCTGCACCCAGCTGAATAGAGCCCTGACCGGCATCGCCGTGGAGCAGGACAAGAAT | |
| ACCCAGGAGGTGTTCGCCCAGGTGAAGCAGATCTACAAGACTCCGCCGATCAAGGACTTCGGCGGCTTCAATTTC | |
| AGCCAAATACTCCCAGATCCAAGCAAGCCTAGCAAGAGGAGCTTCATCGAGGACCTGCTGTTCAATAAGGTGACC | |
| CTGGCCGACGCCGGCTTCATCAAGCAGTACGGCGACTGCCTAGGTGATATTGCGGCAAGAGACCTGATCTGCGCC | |
| CAGAAGTTTAACGGTTTGACAGTACTACCTCCTCTGCTGACCGACGAGATGATAGCACAATATACGTCGGCATTG | |
| CTCGCTGGCACGATCACATCGGGCTGGACTTTCGGCGCCGGAGCAGCGTTGCAAATCCCTTTCGCCATGCAGATG | |
| GCCTACAGATTCAATGGCATCGGCGTGACCCAGAATGTGCTGTACGAGAATCAGAAGCTGATCGCCAATCAGTTC | |
| AATAGCGCCATCGGCAAGATCCAGGACAGCCTGAGCAGCACCGCCAGCGCCCTGGGCAAGCTGCAGaACGTGGTG | |
| AATCAGAATGCCCAGGCCCTGAATACCCTGGTGAAGCAGCTGAGCAGCAATTTCGGCGCCATCAGTAGTGTACTC | |
| AACGATATCCTGAGCAGACTGGACAAGGTGGAGGCCGAGGTGCAAATTGATCGTCTTATTACTGGCAGACTGCAG | |
| AGCCTGCAGACCTACGTGACCCAGCAGCTGATCAGAGCCGCCGAGATCAGAGCCAGCGCCAATCTGGCCGCCACC | |
| AAGATGAGCGAGTGCGTGCTGGGCCAGAGCAAGAGAGTGGACTTCTGCGGCAAGGGCTACCACCTGATGAGCTTC | |
| CCTCAGAGCGCTCCACATGGCGTGGTGTTCCTGCACGTGACCTACGTGCCTGCCCAGGAGAAGAATTTCACCACC | |
| GCACCCGCAATCTGCCACGACGGCAAGGCCCACTTCCCTAGAGAGGGCGTGTTCGTGAGCAATGGCACCCACTGG | |
| TTCGTGACCCAGAGAAATTTCTACGAGCCTCAGATCATCACCACCGACAATACCTTCGTGAGCGGCAATTGCGAC | |
| GTGGTGATCGGGATAGTCAATAATACTGTCTACGACCCTCTGCAGCCTGAGCTGGACAGCTTCAAGGAGGAGCTG | |
| GACAAGTACTTCAAGAATCACACCAGCCCTGACGTGGACCTCGGTGATATTTCGGGAATCAATGCCAGCGTGGTG | |
| AATATCCAGAAGGAAATTGATCGGCTCAACGAAGTGGCCAAGAATCTGAATGAGAGCCTGATCGACCTGCAGGAG | |
| CTGGGCAAGTACGAGCAGTACATCAAGTGGCCTTGGTACATCTGGCTGGGCTTCATCGCCGGCCTGATCGCCATC | |
| GTGATGGTGACCATCATGCTGTGCTGCATGACCTCCTGTTGTTCCTGTTTGAAAGGGTGTTGTTCGTGTGGGTCC | |
| TGCTGCTGA | |
| C.37 + Δ19aa nucleic acid sequence | |
| (comprised in pGM1028 S-LV v02) | |
| SEQ ID NO: 32 | |
| ATGTTCGTGTTCCTGGTGCTGCTGCCTCTGGTGAGCAGCCAGTGCGTGAATCTGACCACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGtC | |
| AtCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGGTGTTTATTATCATAAGAACAACAAG | |
| AGCTGGATGGAGAGCGAGTTCCGCGTATATTCGTCGGCTAATAATTGCACCTTCGAGTACGTGAGCCAGCCTTTC | |
| CTGATGGACCTGGAGGGCAAGCAGGGCAATTTCAAGAATCTGAGAGAGTTCGTGTTCAAGAATATCGACGGCTAC | |
| TTCAAGATCTACAGCAAGCACACACCCATTAATCTGGTGAGAGACCTGCCTCAGGGCTTCAGCGCCCTGGAGCCT | |
| CTGGTGGACCTGCCTATCGGCATCAATATCACCAGATTCCAGACCCTGCTGGCCCTGCACGATTCGTCAAGCGGT | |
| TGGACCGCTGGAGCTGCGGCATATTACGTGGGCTACCTGCAGCCTAGAACCTTCCTGCTGAAGTACAATGAGAAT | |
| GGTACGATAACCGACGCAGTTGATTGTGCCCTGGACCCTCTGAGCGAGACCAAGTGCACCCTGAAGAGCTTCACC | |
| GTGGAGAAGGGCATCTACCAGACCAGCAATTTCAGAGTGCAGCCTACCGAGAGCATCGTGAGATTCCCTAATATC | |
| ACCAATCTGTGCCCTTTCGGCGAGGTGTTCAATGCCACCAGATTCGCCAGCGTGTACGCATGGAACCGCAAGCGG | |
| ATAAGCAATTGCGTGGCCGACTACAGCGTGCTGTACAATAGCGCCAGCTTCAGCACCTTCAAATGTTATGGTGTT | |
| TCGCCAACAAAGCTGAATGACCTGTGCTTCACCAATGTGTACGCCGACAGCTTCGTGATCAGAGGCGACGAGGTG | |
| AGACAGATCGCGCCAGGGCAGACCGGCAAGATCGCCGACTACAATTACAAGCTGCCTGACGACTTCACCGGCTGC | |
| GTGATCGCGTGGAACTCTAACAATCTAGATTCGAAAGTTGGAGGCAATTACAATTACCaGTACAGACTGTTCAGA | |
| AAGAGCAATCTGAAGCCTTTCGAGAGAGACATCAGCACCGAGATCTACCAGGCCGGCAGCACACCGTGTAATGGC | |
| GTGGAGGGCTTCAATTGCTACagtCCTCTGCAGAGCTACGGCTTCCAGCCTACCAATGGCGTGGGCTACCAGCCT | |
| TACAGAGTGGTGGTGCTGAGCTTCGAGCTGCTGCACGCTCCCGCTACCGTGTGCGGCCCTAAGAAGAGCACCAAT | |
| CTGGTGAAGAATAAGTGCGTGAATTTCAATTTCAATGGTCTAACTGGAACGGGCGTGCTGACCGAGAGCAATAAG | |
| AAGTTTCTTCCCTTTCAACAATTCGGCAGAGACATCGCCGACACCACAGATGCTGTAAGAGACCCTCAGACCCTG | |
| GAGATCCTGGACATCACTCCGTGTAGCTTCGGCGGCGTGAGCGTGATCACACCGGGTACCAATACCAGCAATCAG | |
| GTGGCCGTGCTGTACCAGGgCGTGAATTGCACCGAGGTGCCTGTGGCCATCCACGCCGACCAGCTGACTCCCACT | |
| TGGAGGGTATATTCCACGGGAAGCAATGTGTTCCAGACCAGAGCCGGCTGCCTGATCGGCGCCGAGCACGTGAAT | |
| AATAGCTACGAGTGCGACATCCCTATCGGCGCCGGCATCTGCGCCAGCTACCAGACCCAGACCAATAGCCCTAGA | |
| AGAGCCAGAAGCGTGGCCAGCCAGAGCATCATCGCCTACACCATGAGCCTGGGCGCCGAGAATAGCGTGGCCTAC | |
| AGCAATAATAGCATCGCCATCCCTACCAATTTCACCATCAGCGTGACCACCGAAATATTACCAGTCTCCATGACC | |
| AAGACCAGCGTGGACTGCACCATGTACATCTGCGGCGACAGCACCGAGTGCAGCAATCTGCTGCTGCAGTACGGC | |
| AGCTTCTGCACCCAGCTGAATAGAGCCCTGACCGGCATCGCCGTGGAGCAGGACAAGAATACCCAGGAGGTGTTC | |
| GCCCAGGTGAAGCAGATCTACAAGACTCCGCCGATCAAGGACTTCGGCGGCTTCAATTTCAGCCAAATACTCCCA | |
| GATCCAAGCAAGCCTAGCAAGAGGAGCTTCATCGAGGACCTGCTGTTCAATAAGGTGACCCTGGCCGACGCCGGC | |
| TTCATCAAGCAGTACGGCGACTGCCTAGGTGATATTGCGGCAAGAGACCTGATCTGCGCCCAGAAGTTTAACGGT | |
| TTGAacGTACTACCTCCTCTGCTGACCGACGAGATGATAGCACAATATACGTCGGCATTGCTCGCTGGCACGATC | |
| ACATCGGGCTGGACTTTCGGCGCCGGAGCAGCGTTGCAAATCCCTTTCGCCATGCAGATGGCCTACAGATTCAAT | |
| GGCATCGGCGTGACCCAGAATGTGCTGTACGAGAATCAGAAGCTGATCGCCAATCAGTTCAATAGCGCCATCGGC | |
| AAGATCCAGGACAGCCTGAGCAGCACCGCCAGCGCCCTGGGCAAGCTGCAGGACGTGGTGAATCAGAATGCCCAG | |
| GCCCTGAATACCCTGGTGAAGCAGCTGAGCAGCAATTTCGGCGCCATCAGTAGTGTACTCAACGATATCCTGAGC | |
| AGACTGGACAAGGTGGAGGCCGAGGTGCAAATTGATCGTCTTATTACTGGCAGACTGCAGAGCCTGCAGACCTAC | |
| GTGACCCAGCAGCTGATCAGAGCCGCCGAGATCAGAGCCAGCGCCAATCTGGCCGCCACCAAGATGAGCGAGTGC | |
| GTGCTGGGCCAGAGCAAGAGAGTGGACTTCTGCGGCAAGGGCTACCACCTGATGAGCTTCCCTCAGAGCGCTCCA | |
| CATGGCGTGGTGTTCCTGCACGTGACCTACGTGCCTGCCCAGGAGAAGAATTTCACCACCGCACCCGCAATCTGC | |
| CACGACGGCAAGGCCCACTTCCCTAGAGAGGGCGTGTTCGTGAGCAATGGCACCCACTGGTTCGTGACCCAGAGA | |
| AATTTCTACGAGCCTCAGATCATCACCACCGACAATACCTTCGTGAGCGGCAATTGCGACGTGGTGATCGGGATA | |
| GTCAATAATACTGTCTACGACCCTCTGCAGCCTGAGCTGGACAGCTTCAAGGAGGAGCTGGACAAGTACTTCAAG | |
| AATCACACCAGCCCTGACGTGGACCTCGGTGATATTTCGGGAATCAATGCCAGCGTGGTGAATATCCAGAAGGAA | |
| ATTGATCGGCTCAACGAAGTGGCCAAGAATCTGAATGAGAGCCTGATCGACCTGCAGGAGCTGGGCAAGTACGAG | |
| CAGTACATCAAGTGGCCTTGGTACATCTGGCTGGGCTTCATCGCCGGCCTGATCGCCATCGTGATGGTGACCATC | |
| ATGCTGTGCTGCATGACCTCCTGTTGTTCCTGTTTGAAAGGGTGTTGTTCGTGTGGGTCCTGCTGCTGA | |
| maS:Y498, T499 + Δ19aa nucleic acid sequence | |
| (comprised in pGM937 S-LV v03) | |
| SEQ ID NO: 33 | |
| See FIG. 2 | |
| maS:Y498, T499, G614 + Δ19aa nucleic acid sequence | |
| (comprised in pGM939 S-LV v03) | |
| SEQ ID NO: 34 | |
| See FIG. 2 | |
| maS: Aus/VIC01, Y498, T499 + Δ19aa nucleic acid sequence | |
| (comprised in pGM938 S-LV v03) | |
| SEQ ID NO: 35 | |
| See FIG. 2 | |
| S:B.1.617.2, Y501 + Δ19aa nucleic acid sequence | |
| (comprised in pGM1038 S-LV v04) | |
| SEQ ID NO: 36 | |
| ATGTTCGTGTTCCTGGTGCTGCTGCCTCTGGTGAGCAGCCAGTGCGTGAATCTGAggACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGGC | |
| ACCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGaTGTTTATTATCATAAGAACAACAAG | |
| AGCTGGATGGAGAGCgGCGTATATTCGTCGGCTAATAATTGCACCTTCGAGTACGTGAGCCAGCCTTTCCTGATG | |
| GACCTGGAGGGCAAGCAGGGCAATTTCAAGAATCTGAGAGAGTTCGTGTTCAAGAATATCGACGGCTACTTCAAG | |
| ATCTACAGCAAGCACACACCCATTAATCTGGTGAGAGACCTGCCTCAGGGCTTCAGCGCCCTGGAGCCTCTGGTG | |
| GACCTGCCTATCGGCATCAATATCACCAGATTCCAGACCCTGCTGGCCCTGCACAGATCATATCTTACACCAGGC | |
| GATTCGTCAAGCGGTTGGACCGCTGGAGCTGCGGCATATTACGTGGGCTACCTGCAGCCTAGAACCTTCCTGCTG | |
| AAGTACAATGAGAATGGTACGATAACCGACGCAGTTGATTGTGCCCTGGACCCTCTGAGCGAGACCAAGTGCACC | |
| CTGAAGAGCTTCACCGTGGAGAAGGGCATCTACCAGACCAGCAATTTCAGAGTGCAGCCTACCGAGAGCATCGTG | |
| AGATTCCCTAATATCACCAATCTGTGCCCTTTCGGCGAGGTGTTCAATGCCACCAGATTCGCCAGCGTGTACGCA | |
| TGGAACCGCAAGCGGATAAGCAATTGCGTGGCCGACTACAGCGTGCTGTACAATAGCGCCAGCTTCAGCACCTTC | |
| AAATGTTATGGTGTTTCGCCAACAAAGCTGAATGACCTGTGCTTCACCAATGTGTACGCCGACAGCTTCGTGATC | |
| AGAGGCGACGAGGTGAGACAGATCGCGCCAGGGCAGACCGGCAAGATCGCCGACTACAATTACAAGCTGCCTGAC | |
| GACTTCACCGGCTGCGTGATCGCGTGGAACTCTAACAATCTAGATTCGAAAGTTGGAGGCAATTACAATTACCgG | |
| TACAGACTGTTCAGAAAGAGCAATCTGAAGCCTTTCGAGAGAGACATCAGCACCGAGATCTACCAGGCCGGCAGC | |
| AaACCGTGTAATGGCGTGGAGGGCTTCAATTGCTACTTCCCTCTGCAGAGCTACGGCTTCCAGCCTACCtATGGC | |
| GTGGGCTACCAGCCTTACAGAGTGGTGGTGCTGAGCTTCGAGCTGCTGCACGCTCCCGCTACCGTGTGCGGCCCT | |
| AAGAAGAGCACCAATCTGGTGAAGAATAAGTGCGTGAATTTCAATTTCAATGGTCTAACTGGAACGGGCGTGCTG | |
| ACCGAGAGCAATAAGAAGTTTCTTCCCTTTCAACAATTCGGCAGAGACATCGCCGACACCACAGATGCTGTAAGA | |
| GACCCTCAGACCCTGGAGATCCTGGACATCACTCCGTGTAGCTTCGGCGGCGTGAGCGTGATCACACCGGGTACC | |
| AATACCAGCAATCAGGTGGCCGTGCTGTACCAGGqCGTGAATTGCACCGAGGTGCCTGTGGCCATCCACGCCGAC | |
| CAGCTGACTCCCACTTGGAGGGTATATTCCACGGGAAGCAATGTGTTCCAGACCAGAGCCGGCTGCCTGATCGGC | |
| GCCGAGCACGTGAATAATAGCTACGAGTGCGACATCCCTATCGGCGCCGGCATCTGCGCCAGCTACCAGACCCAG | |
| ACCAATAGCCgTAGAAGAGCCAGAAGCGTGGCCAGCCAGAGCATCATCGCCTACACCATGAGCCTGGGCGCCGAG | |
| AATAGCGTGGCCTACAGCAATAATAGCATCGCCATCCCTACCAATTTCACCATCAGCGTGACCACCGAAATATTA | |
| CCAGTCTCCATGACCAAGACCAGCGTGGACTGCACCATGTACATCTGCGGCGACAGCACCGAGTGCAGCAATCTG | |
| CTGCTGCAGTACGGCAGCTTCTGCACCCAGCTGAATAGAGCCCTGACCGGCATCGCCGTGGAGCAGGACAAGAAT | |
| ACCCAGGAGGTGTTCGCCCAGGTGAAGCAGATCTACAAGACTCCGCCGATCAAGGACTTCGGCGGCTTCAATTTC | |
| AGCCAAATACTCCCAGATCCAAGCAAGCCTAGCAAGAGGAGCTTCATCGAGGACCTGCTGTTCAATAAGGTGACC | |
| CTGGCCGACGCCGGCTTCATCAAGCAGTACGGCGACTGCCTAGGTGATATTGCGGCAAGAGACCTGATCTGCGCC | |
| CAGAAGTTTAACGGTTTGACAGTACTACCTCCTCTGCTGACCGACGAGATGATAGCACAATATACGTCGGCATTG | |
| CTCGCTGGCACGATCACATCGGGCTGGACTTTCGGCGCCGGAGCAGCGTTGCAAATCCCTTTCGCCATGCAGATG | |
| GCCTACAGATTCAATGGCATCGGCGTGACCCAGAATGTGCTGTACGAGAATCAGAAGCTGATCGCCAATCAGTTC | |
| AATAGCGCCATCGGCAAGATCCAGGACAGCCTGAGCAGCACCGCCAGCGCCCTGGGCAAGCTGCAGaACGTGGTG | |
| AATCAGAATGCCCAGGCCCTGAATACCCTGGTGAAGCAGCTGAGCAGCAATTTCGGCGCCATCAGTAGTGTACTC | |
| AACGATATCCTGAGCAGACTGGACAAGGTGGAGGCCGAGGTGCAAATTGATCGTCTTATTACTGGCAGACTGCAG | |
| AGCCTGCAGACCTACGTGACCCAGCAGCTGATCAGAGCCGCCGAGATCAGAGCCAGCGCCAATCTGGCCGCCACC | |
| AAGATGAGCGAGTGCGTGCTGGGCCAGAGCAAGAGAGTGGACTTCTGCGGCAAGGGCTACCACCTGATGAGCTTC | |
| CCTCAGAGCGCTCCACATGGCGTGGTGTTCCTGCACGTGACCTACGTGCCTGCCCAGGAGAAGAATTTCACCACC | |
| GCACCCGCAATCTGCCACGACGGCAAGGCCCACTTCCCTAGAGAGGGCGTGTTCGTGAGCAATGGCACCCACTGG | |
| TTCGTGACCCAGAGAAATTTCTACGAGCCTCAGATCATCACCACCGACAATACCTTCGTGAGCGGCAATTGCGAC | |
| GTGGTGATCGGGATAGTCAATAATACTGTCTACGACCCTCTGCAGCCTGAGCTGGACAGCTTCAAGGAGGAGCTG | |
| GACAAGTACTTCAAGAATCACACCAGCCCTGACGTGGACCTCGGTGATATTTCGGGAATCAATGCCAGCGTGGTG | |
| AATATCCAGAAGGAAATTGATCGGCTCAACGAAGTGGCCAAGAATCTGAATGAGAGCCTGATCGACCTGCAGGAG | |
| CTGGGCAAGTACGAGCAGTACATCAAGTGGCCTTGGTACATCTGGCTGGGCTTCATCGCCGGCCTGATCGCCATC | |
| GTGATGGTGACCATCATGCTGTGCTGCATGACCTCCTGTTGTTCCTGTTTGAAAGGGTGTTGTTCGTGTGGGTCC | |
| TGCTGCTGA | |
| C.37, Y501 + Δ19aa nucleic acid sequence | |
| (comprised in pGM1039 S-LV v05) | |
| SEQ ID NO: 37 | |
| ATGTTCGTGTTCCTGGTGCTGCTGCCTCTGGTGAGCAGCCAGTGCGTGAATCTGACCACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGtC | |
| AtCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGGTGTTTATTATCATAAGAACAACAAG | |
| AGCTGGATGGAGAGCGAGTTCCGCGTATATTCGTCGGCTAATAATTGCACCTTCGAGTACGTGAGCCAGCCTTTC | |
| CTGATGGACCTGGAGGGCAAGCAGGGCAATTTCAAGAATCTGAGAGAGTTCGTGTTCAAGAATATCGACGGCTAC | |
| TTCAAGATCTACAGCAAGCACACACCCATTAATCTGGTGAGAGACCTGCCTCAGGGCTTCAGCGCCCTGGAGCCT | |
| CTGGTGGACCTGCCTATCGGCATCAATATCACCAGATTCCAGACCCTGCTGGCCCTGCACGATTCGTCAAGCGGT | |
| TGGACCGCTGGAGCTGCGGCATATTACGTGGGCTACCTGCAGCCTAGAACCTTCCTGCTGAAGTACAATGAGAAT | |
| GGTACGATAACCGACGCAGTTGATTGTGCCCTGGACCCTCTGAGCGAGACCAAGTGCACCCTGAAGAGCTTCACC | |
| GTGGAGAAGGGCATCTACCAGACCAGCAATTTCAGAGTGCAGCCTACCGAGAGCATCGTGAGATTCCCTAATATC | |
| ACCAATCTGTGCCCTTTCGGCGAGGTGTTCAATGCCACCAGATTCGCCAGCGTGTACGCATGGAACCGCAAGCGG | |
| ATAAGCAATTGCGTGGCCGACTACAGCGTGCTGTACAATAGCGCCAGCTTCAGCACCTTCAAATGTTATGGTGTT | |
| TCGCCAACAAAGCTGAATGACCTGTGCTTCACCAATGTGTACGCCGACAGCTTCGTGATCAGAGGCGACGAGGTG | |
| AGACAGATCGCGCCAGGGCAGACCGGCAAGATCGCCGACTACAATTACAAGCTGCCTGACGACTTCACCGGCTGC | |
| GTGATCGCGTGGAACTCTAACAATCTAGATTCGAAAGTTGGAGGCAATTACAATTACCaGTACAGACTGTTCAGA | |
| AAGAGCAATCTGAAGCCTTTCGAGAGAGACATCAGCACCGAGATCTACCAGGCCGGCAGCACACCGTGTAATGGC | |
| GTGGAGGGCTTCAATTGCTACagtCCTCTGCAGAGCTACGGCTTCCAGCCTACCtATGGCGTGGGCTACCAGCCT | |
| TACAGAGTGGTGGTGCTGAGCTTCGAGCTGCTGCACGCTCCCGCTACCGTGTGCGGCCCTAAGAAGAGCACCAAT | |
| CTGGTGAAGAATAAGTGCGTGAATTTCAATTTCAATGGTCTAACTGGAACGGGCGTGCTGACCGAGAGCAATAAG | |
| AAGTTTCTTCCCTTTCAACAATTCGGCAGAGACATCGCCGACACCACAGATGCTGTAAGAGACCCTCAGACCCTG | |
| GAGATCCTGGACATCACTCCGTGTAGCTTCGGCGGCGTGAGCGTGATCACACCGGGTACCAATACCAGCAATCAG | |
| GTGGCCGTGCTGTACCAGGgCGTGAATTGCACCGAGGTGCCTGTGGCCATCCACGCCGACCAGCTGACTCCCACT | |
| TGGAGGGTATATTCCACGGGAAGCAATGTGTTCCAGACCAGAGCCGGCTGCCTGATCGGCGCCGAGCACGTGAAT | |
| AATAGCTACGAGTGCGACATCCCTATCGGCGCCGGCATCTGCGCCAGCTACCAGACCCAGACCAATAGCCCTAGA | |
| AGAGCCAGAAGCGTGGCCAGCCAGAGCATCATCGCCTACACCATGAGCCTGGGCGCCGAGAATAGCGTGGCCTAC | |
| AGCAATAATAGCATCGCCATCCCTACCAATTTCACCATCAGCGTGACCACCGAAATATTACCAGTCTCCATGACC | |
| AAGACCAGCGTGGACTGCACCATGTACATCTGCGGCGACAGCACCGAGTGCAGCAATCTGCTGCTGCAGTACGGC | |
| AGCTTCTGCACCCAGCTGAATAGAGCCCTGACCGGCATCGCCGTGGAGCAGGACAAGAATACCCAGGAGGTGTTC | |
| GCCCAGGTGAAGCAGATCTACAAGACTCCGCCGATCAAGGACTTCGGCGGCTTCAATTTCAGCCAAATACTCCCA | |
| GATCCAAGCAAGCCTAGCAAGAGGAGCTTCATCGAGGACCTGCTGTTCAATAAGGTGACCCTGGCCGACGCCGGC | |
| TTCATCAAGCAGTACGGCGACTGCCTAGGTGATATTGCGGCAAGAGACCTGATCTGCGCCCAGAAGTTTAACGGT | |
| TTGAacGTACTACCTCCTCTGCTGACCGACGAGATGATAGCACAATATACGTCGGCATTGCTCGCTGGCACGATC | |
| ACATCGGGCTGGACTTTCGGCGCCGGAGCAGCGTTGCAAATCCCTTTCGCCATGCAGATGGCCTACAGATTCAAT | |
| GGCATCGGCGTGACCCAGAATGTGCTGTACGAGAATCAGAAGCTGATCGCCAATCAGTTCAATAGCGCCATCGGC | |
| AAGATCCAGGACAGCCTGAGCAGCACCGCCAGCGCCCTGGGCAAGCTGCAGGACGTGGTGAATCAGAATGCCCAG | |
| GCCCTGAATACCCTGGTGAAGCAGCTGAGCAGCAATTTCGGCGCCATCAGTAGTGTACTCAACGATATCCTGAGC | |
| AGACTGGACAAGGTGGAGGCCGAGGTGCAAATTGATCGTCTTATTACTGGCAGACTGCAGAGCCTGCAGACCTAC | |
| GTGACCCAGCAGCTGATCAGAGCCGCCGAGATCAGAGCCAGCGCCAATCTGGCCGCCACCAAGATGAGCGAGTGC | |
| GTGCTGGGCCAGAGCAAGAGAGTGGACTTCTGCGGCAAGGGCTACCACCTGATGAGCTTCCCTCAGAGCGCTCCA | |
| CATGGCGTGGTGTTCCTGCACGTGACCTACGTGCCTGCCCAGGAGAAGAATTTCACCACCGCACCCGCAATCTGC | |
| CACGACGGCAAGGCCCACTTCCCTAGAGAGGGCGTGTTCGTGAGCAATGGCACCCACTGGTTCGTGACCCAGAGA | |
| AATTTCTACGAGCCTCAGATCATCACCACCGACAATACCTTCGTGAGCGGCAATTGCGACGTGGTGATCGGGATA | |
| GTCAATAATACTGTCTACGACCCTCTGCAGCCTGAGCTGGACAGCTTCAAGGAGGAGCTGGACAAGTACTTCAAG | |
| AATCACACCAGCCCTGACGTGGACCTCGGTGATATTTCGGGAATCAATGCCAGCGTGGTGAATATCCAGAAGGAA | |
| ATTGATCGGCTCAACGAAGTGGCCAAGAATCTGAATGAGAGCCTGATCGACCTGCAGGAGCTGGGCAAGTACGAG | |
| CAGTACATCAAGTGGCCTTGGTACATCTGGCTGGGCTTCATCGCCGGCCTGATCGCCATCGTGATGGTGACCATC | |
| ATGCTGTGCTGCATGACCTCCTGTTGTTCCTGTTTGAAAGGGTGTTGTTCGTGTGGGTCCTGCTGCTGA | |
| S:B.1.617.2, Y484, T499, Y501 + Δ19aa nucleic acid sequence | |
| (comprised in pGM1040 S-LV v04) | |
| SEQ ID NO: 38 | |
| ATGTTCGTGTTCCTGGTGCTGCTGCCTCTGGTGAGCAGCCAGTGCGTGAATCTGAggACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGGC | |
| ACCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGaTGTTTATTATCATAAGAACAACAAG | |
| AGCTGGATGGAGAGCgGCGTATATTCGTCGGCTAATAATTGCACCTTCGAGTACGTGAGCCAGCCTTTCCTGATG | |
| GACCTGGAGGGCAAGCAGGGCAATTTCAAGAATCTGAGAGAGTTCGTGTTCAAGAATATCGACGGCTACTTCAAG | |
| ATCTACAGCAAGCACACACCCATTAATCTGGTGAGAGACCTGCCTCAGGGCTTCAGCGCCCTGGAGCCTCTGGTG | |
| GACCTGCCTATCGGCATCAATATCACCAGATTCCAGACCCTGCTGGCCCTGCACAGATCATATCTTACACCAGGC | |
| GATTCGTCAAGCGGTTGGACCGCTGGAGCTGCGGCATATTACGTGGGCTACCTGCAGCCTAGAACCTTCCTGCTG | |
| AAGTACAATGAGAATGGTACGATAACCGACGCAGTTGATTGTGCCCTGGACCCTCTGAGCGAGACCAAGTGCACC | |
| CTGAAGAGCTTCACCGTGGAGAAGGGCATCTACCAGACCAGCAATTTCAGAGTGCAGCCTACCGAGAGCATCGTG | |
| AGATTCCCTAATATCACCAATCTGTGCCCTTTCGGCGAGGTGTTCAATGCCACCAGATTCGCCAGCGTGTACGCA | |
| TGGAACCGCAAGCGGATAAGCAATTGCGTGGCCGACTACAGCGTGCTGTACAATAGCGCCAGCTTCAGCACCTTC | |
| AAATGTTATGGTGTTTCGCCAACAAAGCTGAATGACCTGTGCTTCACCAATGTGTACGCCGACAGCTTCGTGATC | |
| AGAGGCGACGAGGTGAGACAGATCGCGCCAGGGCAGACCGGCAAGATCGCCGACTACAATTACAAGCTGCCTGAC | |
| GACTTCACCGGCTGCGTGATCGCGTGGAACTCTAACAATCTAGATTCGAAAGTTGGAGGCAATTACAATTACCgG | |
| TACAGACTGTTCAGAAAGAGCAATCTGAAGCCTTTCGAGAGAGACATCAGCACCGAGATCTACCAGGCCGGCAGC | |
| AaACCGTGTAATGGCGTGGAGGGCTTCAATTGCTACTTCCCTCTGCAGAGCTACGGCTTCtataCTACCtATGGC | |
| GTGGGCTACCAGCCTTACAGAGTGGTGGTGCTGAGCTTCGAGCTGCTGCACGCTCCCGCTACCGTGTGCGGCCCT | |
| AAGAAGAGCACCAATCTGGTGAAGAATAAGTGCGTGAATTTCAATTTCAATGGTCTAACTGGAACGGGCGTGCTG | |
| ACCGAGAGCAATAAGAAGTTTCTTCCCTTTCAACAATTCGGCAGAGACATCGCCGACACCACAGATGCTGTAAGA | |
| GACCCTCAGACCCTGGAGATCCTGGACATCACTCCGTGTAGCTTCGGCGGCGTGAGCGTGATCACACCGGGTACC | |
| AATACCAGCAATCAGGTGGCCGTGCTGTACCAGGgCGTGAATTGCACCGAGGTGCCTGTGGCCATCCACGCCGAC | |
| CAGCTGACTCCCACTTGGAGGGTATATTCCACGGGAAGCAATGTGTTCCAGACCAGAGCCGGCTGCCTGATCGGC | |
| GCCGAGCACGTGAATAATAGCTACGAGTGCGACATCCCTATCGGCGCCGGCATCTGCGCCAGCTACCAGACCCAG | |
| ACCAATAGCCgTAGAAGAGCCAGAAGCGTGGCCAGCCAGAGCATCATCGCCTACACCATGAGCCTGGGCGCCGAG | |
| AATAGCGTGGCCTACAGCAATAATAGCATCGCCATCCCTACCAATTTCACCATCAGCGTGACCACCGAAATATTA | |
| CCAGTCTCCATGACCAAGACCAGCGTGGACTGCACCATGTACATCTGCGGCGACAGCACCGAGTGCAGCAATCTG | |
| CTGCTGCAGTACGGCAGCTTCTGCACCCAGCTGAATAGAGCCCTGACCGGCATCGCCGTGGAGCAGGACAAGAAT | |
| ACCCAGGAGGTGTTCGCCCAGGTGAAGCAGATCTACAAGACTCCGCCGATCAAGGACTTCGGCGGCTTCAATTTC | |
| AGCCAAATACTCCCAGATCCAAGCAAGCCTAGCAAGAGGAGCTTCATCGAGGACCTGCTGTTCAATAAGGTGACC | |
| CTGGCCGACGCCGGCTTCATCAAGCAGTACGGCGACTGCCTAGGTGATATTGCGGCAAGAGACCTGATCTGCGCC | |
| CAGAAGTTTAACGGTTTGACAGTACTACCTCCTCTGCTGACCGACGAGATGATAGCACAATATACGTCGGCATTG | |
| CTCGCTGGCACGATCACATCGGGCTGGACTTTCGGCGCCGGAGCAGCGTTGCAAATCCCTTTCGCCATGCAGATG | |
| GCCTACAGATTCAATGGCATCGGCGTGACCCAGAATGTGCTGTACGAGAATCAGAAGCTGATCGCCAATCAGTTC | |
| AATAGCGCCATCGGCAAGATCCAGGACAGCCTGAGCAGCACCGCCAGCGCCCTGGGCAAGCTGCAGaACGTGGTG | |
| AATCAGAATGCCCAGGCCCTGAATACCCTGGTGAAGCAGCTGAGCAGCAATTTCGGCGCCATCAGTAGTGTACTC | |
| AACGATATCCTGAGCAGACTGGACAAGGTGGAGGCCGAGGTGCAAATTGATCGTCTTATTACTGGCAGACTGCAG | |
| AGCCTGCAGACCTACGTGACCCAGCAGCTGATCAGAGCCGCCGAGATCAGAGCCAGCGCCAATCTGGCCGCCACC | |
| AAGATGAGCGAGTGCGTGCTGGGCCAGAGCAAGAGAGTGGACTTCTGCGGCAAGGGCTACCACCTGATGAGCTTC | |
| CCTCAGAGCGCTCCACATGGCGTGGTGTTCCTGCACGTGACCTACGTGCCTGCCCAGGAGAAGAATTTCACCACC | |
| GCACCCGCAATCTGCCACGACGGCAAGGCCCACTTCCCTAGAGAGGGCGTGTTCGTGAGCAATGGCACCCACTGG | |
| TTCGTGACCCAGAGAAATTTCTACGAGCCTCAGATCATCACCACCGACAATACCTTCGTGAGCGGCAATTGCGAC | |
| GTGGTGATCGGGATAGTCAATAATACTGTCTACGACCCTCTGCAGCCTGAGCTGGACAGCTTCAAGGAGGAGCTG | |
| GACAAGTACTTCAAGAATCACACCAGCCCTGACGTGGACCTCGGTGATATTTCGGGAATCAATGCCAGCGTGGTG | |
| AATATCCAGAAGGAAATTGATCGGCTCAACGAAGTGGCCAAGAATCTGAATGAGAGCCTGATCGACCTGCAGGAG | |
| CTGGGCAAGTACGAGCAGTACATCAAGTGGCCTTGGTACATCTGGCTGGGCTTCATCGCCGGCCTGATCGCCATC | |
| GTGATGGTGACCATCATGCTGTGCTGCATGACCTCCTGTTGTTCCTGTTTGAAAGGGTGTTGTTCGTGTGGGTCC | |
| TGCTGCTGA | |
| C.37, Y489, T499, Y501 + Δ19aa nucleic acid sequence | |
| (comprised in pGM1041 S-LV v05) | |
| SEQ ID NO: 39 | |
| ATGTTCGTGTTCCTGGTGCTGCTGCCTCTGGTGAGCAGCCAGTGCGTGAATCTGACCACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGtC | |
| AtCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGGTGTTTATTATCATAAGAACAACAAG | |
| AGCTGGATGGAGAGCGAGTTCCGCGTATATTCGTCGGCTAATAATTGCACCTTCGAGTACGTGAGCCAGCCTTTC | |
| CTGATGGACCTGGAGGGCAAGCAGGGCAATTTCAAGAATCTGAGAGAGTTCGTGTTCAAGAATATCGACGGCTAC | |
| TTCAAGATCTACAGCAAGCACACACCCATTAATCTGGTGAGAGACCTGCCTCAGGGCTTCAGCGCCCTGGAGCCT | |
| CTGGTGGACCTGCCTATCGGCATCAATATCACCAGATTCCAGACCCTGCTGGCCCTGCACGATTCGTCAAGCGGT | |
| TGGACCGCTGGAGCTGCGGCATATTACGTGGGCTACCTGCAGCCTAGAACCTTCCTGCTGAAGTACAATGAGAAT | |
| GGTACGATAACCGACGCAGTTGATTGTGCCCTGGACCCTCTGAGCGAGACCAAGTGCACCCTGAAGAGCTTCACC | |
| GTGGAGAAGGGCATCTACCAGACCAGCAATTTCAGAGTGCAGCCTACCGAGAGCATCGTGAGATTCCCTAATATC | |
| ACCAATCTGTGCCCTTTCGGCGAGGTGTTCAATGCCACCAGATTCGCCAGCGTGTACGCATGGAACCGCAAGCGG | |
| ATAAGCAATTGCGTGGCCGACTACAGCGTGCTGTACAATAGCGCCAGCTTCAGCACCTTCAAATGTTATGGTGTT | |
| TCGCCAACAAAGCTGAATGACCTGTGCTTCACCAATGTGTACGCCGACAGCTTCGTGATCAGAGGCGACGAGGTG | |
| AGACAGATCGCGCCAGGGCAGACCGGCAAGATCGCCGACTACAATTACAAGCTGCCTGACGACTTCACCGGCTGC | |
| GTGATCGCGTGGAACTCTAACAATCTAGATTCGAAAGTTGGAGGCAATTACAATTACCaGTACAGACTGTTCAGA | |
| AAGAGCAATCTGAAGCCTTTCGAGAGAGACATCAGCACCGAGATCTACCAGGCCGGCAGCACACCGTGTAATGGC | |
| GTGGAGGGCTTCAATTGCTACagtCCTCTGCAGAGCTACGGCTTCtataCTACCtATGGCGTGGGCTACCAGCCT | |
| TACAGAGTGGTGGTGCTGAGCTTCGAGCTGCTGCACGCTCCCGCTACCGTGTGCGGCCCTAAGAAGAGCACCAAT | |
| CTGGTGAAGAATAAGTGCGTGAATTTCAATTTCAATGGTCTAACTGGAACGGGCGTGCTGACCGAGAGCAATAAG | |
| AAGTTTCTTCCCTTTCAACAATTCGGCAGAGACATCGCCGACACCACAGATGCTGTAAGAGACCCTCAGACCCTG | |
| GAGATCCTGGACATCACTCCGTGTAGCTTCGGCGGCGTGAGCGTGATCACACCGGGTACCAATACCAGCAATCAG | |
| GTGGCCGTGCTGTACCAGGqCGTGAATTGCACCGAGGTGCCTGTGGCCATCCACGCCGACCAGCTGACTCCCACT | |
| TGGAGGGTATATTCCACGGGAAGCAATGTGTTCCAGACCAGAGCCGGCTGCCTGATCGGCGCCGAGCACGTGAAT | |
| AATAGCTACGAGTGCGACATCCCTATCGGCGCCGGCATCTGCGCCAGCTACCAGACCCAGACCAATAGCCCTAGA | |
| AGAGCCAGAAGCGTGGCCAGCCAGAGCATCATCGCCTACACCATGAGCCTGGGCGCCGAGAATAGCGTGGCCTAC | |
| AGCAATAATAGCATCGCCATCCCTACCAATTTCACCATCAGCGTGACCACCGAAATATTACCAGTCTCCATGACC | |
| AAGACCAGCGTGGACTGCACCATGTACATCTGCGGCGACAGCACCGAGTGCAGCAATCTGCTGCTGCAGTACGGC | |
| AGCTTCTGCACCCAGCTGAATAGAGCCCTGACCGGCATCGCCGTGGAGCAGGACAAGAATACCCAGGAGGTGTTC | |
| GCCCAGGTGAAGCAGATCTACAAGACTCCGCCGATCAAGGACTTCGGCGGCTTCAATTTCAGCCAAATACTCCCA | |
| GATCCAAGCAAGCCTAGCAAGAGGAGCTTCATCGAGGACCTGCTGTTCAATAAGGTGACCCTGGCCGACGCCGGC | |
| TTCATCAAGCAGTACGGCGACTGCCTAGGTGATATTGCGGCAAGAGACCTGATCTGCGCCCAGAAGTTTAACGGT | |
| TTGAacGTACTACCTCCTCTGCTGACCGACGAGATGATAGCACAATATACGTCGGCATTGCTCGCTGGCACGATC | |
| ACATCGGGCTGGACTTTCGGCGCCGGAGCAGCGTTGCAAATCCCTTTCGCCATGCAGATGGCCTACAGATTCAAT | |
| GGCATCGGCGTGACCCAGAATGTGCTGTACGAGAATCAGAAGCTGATCGCCAATCAGTTCAATAGCGCCATCGGC | |
| AAGATCCAGGACAGCCTGAGCAGCACCGCCAGCGCCCTGGGCAAGCTGCAGGACGTGGTGAATCAGAATGCCCAG | |
| GCCCTGAATACCCTGGTGAAGCAGCTGAGCAGCAATTTCGGCGCCATCAGTAGTGTACTCAACGATATCCTGAGC | |
| AGACTGGACAAGGTGGAGGCCGAGGTGCAAATTGATCGTCTTATTACTGGCAGACTGCAGAGCCTGCAGACCTAC | |
| GTGACCCAGCAGCTGATCAGAGCCGCCGAGATCAGAGCCAGCGCCAATCTGGCCGCCACCAAGATGAGCGAGTGC | |
| GTGCTGGGCCAGAGCAAGAGAGTGGACTTCTGCGGCAAGGGCTACCACCTGATGAGCTTCCCTCAGAGCGCTCCA | |
| CATGGCGTGGTGTTCCTGCACGTGACCTACGTGCCTGCCCAGGAGAAGAATTTCACCACCGCACCCGCAATCTGC | |
| CACGACGGCAAGGCCCACTTCCCTAGAGAGGGCGTGTTCGTGAGCAATGGCACCCACTGGTTCGTGACCCAGAGA | |
| AATTTCTACGAGCCTCAGATCATCACCACCGACAATACCTTCGTGAGCGGCAATTGCGACGTGGTGATCGGGATA | |
| GTCAATAATACTGTCTACGACCCTCTGCAGCCTGAGCTGGACAGCTTCAAGGAGGAGCTGGACAAGTACTTCAAG | |
| AATCACACCAGCCCTGACGTGGACCTCGGTGATATTTCGGGAATCAATGCCAGCGTGGTGAATATCCAGAAGGAA | |
| ATTGATCGGCTCAACGAAGTGGCCAAGAATCTGAATGAGAGCCTGATCGACCTGCAGGAGCTGGGCAAGTACGAG | |
| CAGTACATCAAGTGGCCTTGGTACATCTGGCTGGGCTTCATCGCCGGCCTGATCGCCATCGTGATGGTGACCATC | |
| ATGCTGTGCTGCATGACCTCCTGTTGTTCCTGTTTGAAAGGGTGTTGTTCGTGTGGGTCCTGCTGCTGA | |
| maS:Y498, T499, Y501 + Δ19aa nucleic acid sequence | |
| (comprised in pGM998 S-LV v05) | |
| SEQ ID NO: 40 | |
| ATGTTCGTGTTCCTGGTGCTGCTGCCTCTGGTGAGCAGCCAGTGCGTGAATCTGACCACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGGC | |
| ACCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGGTGTTTATTATCATAAGAACAACAAG | |
| AGCTGGATGGAGAGCGAGTTCCGCGTATATTCGTCGGCTAATAATTGCACCTTCGAGTACGTGAGCCAGCCTTTC | |
| CTGATGGACCTGGAGGGCAAGCAGGGCAATTTCAAGAATCTGAGAGAGTTCGTGTTCAAGAATATCGACGGCTAC | |
| TTCAAGATCTACAGCAAGCACACACCCATTAATCTGGTGAGAGACCTGCCTCAGGGCTTCAGCGCCCTGGAGCCT | |
| CTGGTGGACCTGCCTATCGGCATCAATATCACCAGATTCCAGACCCTGCTGGCCCTGCACAGATCATATCTTACA | |
| CCAGGCGATTCGTCAAGCGGTTGGACCGCTGGAGCTGCGGCATATTACGTGGGCTACCTGCAGCCTAGAACCTTC | |
| CTGCTGAAGTACAATGAGAATGGTACGATAACCGACGCAGTTGATTGTGCCCTGGACCCTCTGAGCGAGACCAAG | |
| TGCACCCTGAAGAGCTTCACCGTGGAGAAGGGCATCTACCAGACCAGCAATTTCAGAGTGCAGCCTACCGAGAGC | |
| ATCGTGAGATTCCCTAATATCACCAATCTGTGCCCTTTCGGCGAGGTGTTCAATGCCACCAGATTCGCCAGCGTG | |
| TACGCATGGAACCGCAAGCGGATAAGCAATTGCGTGGCCGACTACAGCGTGCTGTACAATAGCGCCAGCTTCAGC | |
| ACCTTCAAATGTTATGGTGTTTCGCCAACAAAGCTGAATGACCTGTGCTTCACCAATGTGTACGCCGACAGCTTC | |
| GTGATCAGAGGCGACGAGGTGAGACAGATCGCGCCAGGGCAGACCGGCAAGATCGCCGACTACAATTACAAGCTG | |
| CCTGACGACTTCACCGGCTGCGTGATCGCGTGGAACTCTAACAATCTAGATTCGAAAGTTGGAGGCAATTACAAT | |
| TACCTGTACAGACTGTTCAGAAAGAGCAATCTGAAGCCTTTCGAGAGAGACATCAGCACCGAGATCTACCAGGCC | |
| GGCAGCACACCGTGTAATGGCGTGGAGGGCTTCAATTGCTACTTCCCTCTGCAGAGCTACGGCTTCtAtaCTACC | |
| tATGGCGTGGGCTACCAGCCTTACAGAGTGGTGGTGCTGAGCTTCGAGCTGCTGCACGCTCCCGCTACCGTGTGC | |
| GGCCCTAAGAAGAGCACCAATCTGGTGAAGAATAAGTGCGTGAATTTCAATTTCAATGGTCTAACTGGAACGGGC | |
| GTGCTGACCGAGAGCAATAAGAAGTTTCTTCCCTTTCAACAATTCGGCAGAGACATCGCCGACACCACAGATGCT | |
| GTAAGAGACCCTCAGACCCTGGAGATCCTGGACATCACTCCGTGTAGCTTCGGCGGCGTGAGCGTGATCACACCG | |
| GGTACCAATACCAGCAATCAGGTGGCCGTGCTGTACCAGGACGTGAATTGCACCGAGGTGCCTGTGGCCATCCAC | |
| GCCGACCAGCTGACTCCCACTTGGAGGGTATATTCCACGGGAAGCAATGTGTTCCAGACCAGAGCCGGCTGCCTG | |
| ATCGGCGCCGAGCACGTGAATAATAGCTACGAGTGCGACATCCCTATCGGCGCCGGCATCTGCGCCAGCTACCAG | |
| ACCCAGACCAATAGCCCTAGAAGAGCCAGAAGCGTGGCCAGCCAGAGCATCATCGCCTACACCATGAGCCTGGGC | |
| GCCGAGAATAGCGTGGCCTACAGCAATAATAGCATCGCCATCCCTACCAATTTCACCATCAGCGTGACCACCGAA | |
| ATATTACCAGTCTCCATGACCAAGACCAGCGTGGACTGCACCATGTACATCTGCGGCGACAGCACCGAGTGCAGC | |
| AATCTGCTGCTGCAGTACGGCAGCTTCTGCACCCAGCTGAATAGAGCCCTGACCGGCATCGCCGTGGAGCAGGAC | |
| AAGAATACCCAGGAGGTGTTCGCCCAGGTGAAGCAGATCTACAAGACTCCGCCGATCAAGGACTTCGGCGGCTTC | |
| AATTTCAGCCAAATACTCCCAGATCCAAGCAAGCCTAGCAAGAGGAGCTTCATCGAGGACCTGCTGTTCAATAAG | |
| GTGACCCTGGCCGACGCCGGCTTCATCAAGCAGTACGGCGACTGCCTAGGTGATATTGCGGCAAGAGACCTGATC | |
| TGCGCCCAGAAGTTTAACGGTTTGACAGTACTACCTCCTCTGCTGACCGACGAGATGATAGCACAATATACGTCG | |
| GCATTGCTCGCTGGCACGATCACATCGGGCTGGACTTTCGGCGCCGGAGCAGCGTTGCAAATCCCTTTCGCCATG | |
| CAGATGGCCTACAGATTCAATGGCATCGGCGTGACCCAGAATGTGCTGTACGAGAATCAGAAGCTGATCGCCAAT | |
| CAGTTCAATAGCGCCATCGGCAAGATCCAGGACAGCCTGAGCAGCACCGCCAGCGCCCTGGGCAAGCTGCAGGAC | |
| GTGGTGAATCAGAATGCCCAGGCCCTGAATACCCTGGTGAAGCAGCTGAGCAGCAATTTCGGCGCCATCAGTAGT | |
| GTACTCAACGATATCCTGAGCAGACTGGACAAGGTGGAGGCCGAGGTGCAAATTGATCGTCTTATTACTGGCAGA | |
| CTGCAGAGCCTGCAGACCTACGTGACCCAGCAGCTGATCAGAGCCGCCGAGATCAGAGCCAGCGCCAATCTGGCC | |
| GCCACCAAGATGAGCGAGTGCGTGCTGGGCCAGAGCAAGAGAGTGGACTTCTGCGGCAAGGGCTACCACCTGATG | |
| AGCTTCCCTCAGAGCGCTCCACATGGCGTGGTGTTCCTGCACGTGACCTACGTGCCTGCCCAGGAGAAGAATTTC | |
| ACCACCGCACCCGCAATCTGCCACGACGGCAAGGCCCACTTCCCTAGAGAGGGCGTGTTCGTGAGCAATGGCACC | |
| CACTGGTTCGTGACCCAGAGAAATTTCTACGAGCCTCAGATCATCACCACCGACAATACCTTCGTGAGCGGCAAT | |
| TGCGACGTGGTGATCGGGATAGTCAATAATACTGTCTACGACCCTCTGCAGCCTGAGCTGGACAGCTTCAAGGAG | |
| GAGCTGGACAAGTACTTCAAGAATCACACCAGCCCTGACGTGGACCTCGGTGATATTTCGGGAATCAATGCCAGC | |
| GTGGTGAATATCCAGAAGGAAATTGATCGGCTCAACGAAGTGGCCAAGAATCTGAATGAGAGCCTGATCGACCTG | |
| CAGGAGCTGGGCAAGTACGAGCAGTACATCAAGTGGCCTTGGTACATCTGGCTGGGCTTCATCGCCGGCCTGATC | |
| GCCATCGTGATGGTGACCATCATGCTGTGCTGCATGACCTCCTGTTGTTCCTGTTTGAAAGGGTGTTGTTCGTGT | |
| GGGTCCTGCTGCTGA | |
| maS:Y498, T499, Y501, G614 + Δ19aa nucleic acid sequence | |
| (comprised in pGM999 S-LV v05) | |
| SEQ ID NO: 41 | |
| See FIG. 2 | |
| S:G614 with furin site knock out nucleic acid sequence | |
| (comprised in pGM1026 S-LV v05) | |
| SEQ ID NO: 42 | |
| ATGTTCGTGTTCCTGGTGCTGCTGCCTCTGGTGAGCAGCCAGTGCGTGAATCTGACCACCAGAACCCAGCTGCCT | |
| CCTGCCTACACCAATAGCTTCACCAGAGGAGTTTATTATCCCGATAAGGTGTTCAGAAGTAGTGTATTACATAGT | |
| ACCCAGGACCTGTTCCTACCTTTCTTCAGTAACGTGACCTGGTTCCACGCCATCCACGTGAGCGGCACCAATGGC | |
| ACCAAGAGATTCGACAATCCTGTGCTGCCTTTCAATGACGGCGTGTACTTCGCCAGCACCGAGAAGAGCAATATC | |
| ATCAGAGGCTGGATCTTCGGCACCACCTTGGATTCCAAGACTCAGAGCCTGCTGATTGTAAACAACGCTACAAAT | |
| GTGGTGATCAAGGTGTGCGAGTTCCAGTTCTGCAATGACCCTTTCCTGGGTGTTTATTATCATAAGAACAACAAG | |
| AGCTGGATGGAGAGCGAGTTCCGCGTATATTCGTCGGCTAATAATTGCACCTTCGAGTACGTGAGCCAGCCTTTC | |
| CTGATGGACCTGGAGGGCAAGCAGGGCAATTTCAAGAATCTGAGAGAGTTCGTGTTCAAGAATATCGACGGCTAC | |
| TTCAAGATCTACAGCAAGCACACACCCATTAATCTGGTGAGAGACCTGCCTCAGGGCTTCAGCGCCCTGGAGCCT | |
| CTGGTGGACCTGCCTATCGGCATCAATATCACCAGATTCCAGACCCTGCTGGCCCTGCACAGATCATATCTTACA | |
| CCAGGCGATTCGTCAAGCGGTTGGACCGCTGGAGCTGCGGCATATTACGTGGGCTACCTGCAGCCTAGAACCTTC | |
| CTGCTGAAGTACAATGAGAATGGTACGATAACCGACGCAGTTGATTGTGCCCTGGACCCTCTGAGCGAGACCAAG | |
| TGCACCCTGAAGAGCTTCACCGTGGAGAAGGGCATCTACCAGACCAGCAATTTCAGAGTGCAGCCTACCGAGAGC | |
| ATCGTGAGATTCCCTAATATCACCAATCTGTGCCCTTTCGGCGAGGTGTTCAATGCCACCAGATTCGCCAGCGTG | |
| TACGCATGGAACCGCAAGCGGATAAGCAATTGCGTGGCCGACTACAGCGTGCTGTACAATAGCGCCAGCTTCAGC | |
| ACCTTCAAATGTTATGGTGTTTCGCCAACAAAGCTGAATGACCTGTGCTTCACCAATGTGTACGCCGACAGCTTC | |
| GTGATCAGAGGCGACGAGGTGAGACAGATCGCGCCAGGGCAGACCGGCAAGATCGCCGACTACAATTACAAGCTG | |
| CCTGACGACTTCACCGGCTGCGTGATCGCGTGGAACTCTAACAATCTAGATTCGAAAGTTGGAGGCAATTACAAT | |
| TACCTGTACAGACTGTTCAGAAAGAGCAATCTGAAGCCTTTCGAGAGAGACATCAGCACCGAGATCTACCAGGCC | |
| GGCAGCACACCGTGTAATGGCGTGGAGGGCTTCAATTGCTACTTCCCTCTGCAGAGCTACGGCTTCCAGCCTACC | |
| AATGGCGTGGGCTACCAGCCTTACAGAGTGGTGGTGCTGAGCTTCGAGCTGCTGCACGCTCCCGCTACCGTGTGC | |
| GGCCCTAAGAAGAGCACCAATCTGGTGAAGAATAAGTGCGTGAATTTCAATTTCAATGGTCTAACTGGAACGGGC | |
| GTGCTGACCGAGAGCAATAAGAAGTTTCTTCCCTTTCAACAATTCGGCAGAGACATCGCCGACACCACAGATGCT | |
| GTAAGAGACCCTCAGACCCTGGAGATCCTGGACATCACTCCGTGTAGCTTCGGCGGCGTGAGCGTGATCACACCG | |
| GGTACCAATACCAGCAATCAGGTGGCCGTGCTGTACCAGGgCGTGAATTGCACCGAGGTGCCTGTGGCCATCCAC | |
| GCCGACCAGCTGACTCCCACTTGGAGGGTATATTCCACGGGAAGCAATGTGTTCCAGACCAGAGCCGGCTGCCTG | |
| ATCGGCGCCGAGCACGTGAATAATAGCTACGAGTGCGACATCCCTATCGGCGCCGGCATCTGCGCCAGCTACCAG | |
| ACCCAGACCAATAGCCCTAGtAGAGCCAGLAGCGTGGCCAGCCAGAGCATCATCGCCTACACCATGAGCCTGGGC | |
| GCCGAGAATAGCGTGGCCTACAGCAATAATAGCATCGCCATCCCTACCAATTTCACCATCAGCGTGACCACCGAA | |
| ATATTACCAGTCTCCATGACCAAGACCAGCGTGGACTGCACCATGTACATCTGCGGCGACAGCACCGAGTGCAGC | |
| AATCTGCTGCTGCAGTACGGCAGCTTCTGCACCCAGCTGAATAGAGCCCTGACCGGCATCGCCGTGGAGCAGGAC | |
| AAGAATACCCAGGAGGTGTTCGCCCAGGTGAAGCAGATCTACAAGACTCCGCCGATCAAGGACTTCGGCGGCTTC | |
| AATTTCAGCCAAATACTCCCAGATCCAAGCAAGCCTAGCAAGAGGAGCTTCATCGAGGACCTGCTGTTCAATAAG | |
| GTGACCCTGGCCGACGCCGGCTTCATCAAGCAGTACGGCGACTGCCTAGGTGATATTGCGGCAAGAGACCTGATC | |
| TGCGCCCAGAAGTTTAACGGTTTGACAGTACTACCTCCTCTGCTGACCGACGAGATGATAGCACAATATACGTCG | |
| GCATTGCTCGCTGGCACGATCACATCGGGCTGGACTTTCGGCGCCGGAGCAGCGTTGCAAATCCCTTTCGCCATG | |
| CAGATGGCCTACAGATTCAATGGCATCGGCGTGACCCAGAATGTGCTGTACGAGAATCAGAAGCTGATCGCCAAT | |
| CAGTTCAATAGCGCCATCGGCAAGATCCAGGACAGCCTGAGCAGCACCGCCAGCGCCCTGGGCAAGCTGCAGGAC | |
| GTGGTGAATCAGAATGCCCAGGCCCTGAATACCCTGGTGAAGCAGCTGAGCAGCAATTTCGGCGCCATCAGTAGT | |
| GTACTCAACGATATCCTGAGCAGACTGGACAAGGTGGAGGCCGAGGTGCAAATTGATCGTCTTATTACTGGCAGA | |
| CTGCAGAGCCTGCAGACCTACGTGACCCAGCAGCTGATCAGAGCCGCCGAGATCAGAGCCAGCGCCAATCTGGCC | |
| GCCACCAAGATGAGCGAGTGCGTGCTGGGCCAGAGCAAGAGAGTGGACTTCTGCGGCAAGGGCTACCACCTGATG | |
| AGCTTCCCTCAGAGCGCTCCACATGGCGTGGTGTTCCTGCACGTGACCTACGTGCCTGCCCAGGAGAAGAATTTC | |
| ACCACCGCACCCGCAATCTGCCACGACGGCAAGGCCCACTTCCCTAGAGAGGGCGTGTTCGTGAGCAATGGCACC | |
| CACTGGTTCGTGACCCAGAGAAATTTCTACGAGCCTCAGATCATCACCACCGACAATACCTTCGTGAGCGGCAAT | |
| TGCGACGTGGTGATCGGGATAGTCAATAATACTGTCTACGACCCTCTGCAGCCTGAGCTGGACAGCTTCAAGGAG | |
| GAGCTGGACAAGTACTTCAAGAATCACACCAGCCCTGACGTGGACCTCGGTGATATTTCGGGAATCAATGCCAGC | |
| GTGGTGAATATCCAGAAGGAAATTGATCGGCTCAACGAAGTGGCCAAGAATCTGAATGAGAGCCTGATCGACCTG | |
| CAGGAGCTGGGCAAGTACGAGCAGTACATCAAGTGGCCTTGGTACATCTGGCTGGGCTTCATCGCCGGCCTGATC | |
| GCCATCGTGATGGTGACCATCATGCTGTGCTGCATGACCTCCTGTTGTTCCTGTTTGAAAGGGTGTTGTTCGTGT | |
| GGGTCCTGCTGCTGA | |
| hCEF promoter | |
| SEQ ID NO: 43 | |
| 1 | AGATCTGTTA CATAACTTAT GGTAAATGGC CTGCCTGGCT GACTGCCCAA TGACCCCTGC |
| 61 | CCAATGATGT CAATAATGAT GTATGTTCCC ATGTAATGCC AATAGGGACT TTCCATTGAT |
| 121 | GTCAATGGGT GGAGTATTTA TGGTAACTGC CCACTTGGCA GTACATCAAG TGTATCATAT |
| 181 | GCCAAGTATG CCCCCTATTG ATGTCAATGA TGGTAAATGG CCTGCCTGGC ATTATGCCCA |
| 241 | GTACATGACC TTATGGGACT TTCCTACTTG GCAGTACATC TATGTATTAG TCATTGCTAT |
| 301 | TACCATGGGA ATTCACTAGT GGAGAAGAGC ATGCTTGAGG GCTGAGTGCC CCTCAGTGGG |
| 361 | CAGAGAGCAC ATGGCCCACA GTCCCTGAGA AGTTGGGGGG AGGGGTGGGC AATTGAACTG |
| 421 | GTGCCTAGAG AAGGTGGGGC TTGGGTAAAC TGGGAAAGTG ATGTGGTGTA CTGGCTCCAC |
| 481 | CTTTTTCCCC AGGGTGGGGG AGAACCATAT ATAAGTGCAG TAGTCTCTGT GAACATTCAA |
| 541 | GCTTCTGCCT TCTCCCTCCT GTGAGTTTGC TAGC |
| CMV promoter | |
| SEQ ID NO: 44 | |
| CCGCGGAGATCTCAATATTGGCCATTAGCCATATTATTCATTGGTTATATAGCATAAATCAATATTGGCTATTGG | |
| CCATTGCATACGTTGTATCTATATCATAATATGTACATTTATATTGGCTCATGTCCAATATGACCGCCATGTTGG | |
| CATTGATTATTGACTAGTTATTAATAGTAATCAATTACGGGGTCATTAGTTCATAGCCCATATATGGAGTTCCGC | |
| GTTACATAACTTACGGTAAATGGCCCGCCTGGCTGACCGCCCAACGACCCCCGCCCATTGACGTCAATAATGACG | |
| TATGTTCCCATAGTAACGCCAATAGGGACTTTCCATTGACGTCAATGGGTGGAGTATTTACGGTAAACTGCCCAC | |
| TTGGCAGTACATCAAGTGTATCATATGCCAAGTCCGCCCCCTATTGACGTCAATGACGGTAAATGGCCCGCCTGG | |
| CATTATGCCCAGTACATGACCTTACGGGACTTTCCTACTTGGCAGTACATCTACGTATTAGTCATCGCTATTACC | |
| ATGGTGATGCGGTTTTGGCAGTACACCAATGGGCGTGGATAGCGGTTTGACTCACGGGGATTTCCAAGTCTCCAC | |
| CCCATTGACGTCAATGGGAGTTTGTTTTGGCACCAAAATCAACGGGACTTTCCAAAATGTCGTAATAACCCCGCC | |
| CCGTTGACGCAAATGGGCGGTAGGCGTGTACGGTGGGAGGTCTATATAAGCAGAGCTCGTTTAGTGAACCGTCAG | |
| ATCACTAGAAGCTTTATTGCGGTAGTTTATCACAGTTAAATTGCTAACGCAGTCAGTGCTTCTGACACAACAGTC | |
| TCGAACTTAAGCTGCAGAAGTTGGTCGTGAGGCACTGGGCAGGCTAGC | |
| human elongation factor 1a (EF1a) promoter. | |
| SEQ ID NO: 45 | |
| AGATCCATATCCGCGGCAATTTTAAAAGAAAGGGAGGAATAGGGGGACAGACTTCAGCAGAGAGACTAATTAATA | |
| TAATAACAACACAATTAGAAATACAACATTTACAAACCAAAATTCAAAAAATTTTAAATTTTAGAGCCGCGGAGA | |
| TCCCGTGAGGCTCCGGTGCCCGTCAGTGGGCAGAGCGCACATCGCCCACAGTCCCCGAGAAGTTGGGGGGAGGGG | |
| TCGGCAATTGAACCGGTGCCTAGAGAAGGTGGCGCGGGGTAAACTGGGAAAGTGATGTCGTGTACTGGCTCCGCC | |
| TTTTTCCCGAGGGTGGGGGAGAACCGTATATAAGTGCAGTAGTCGCCGTGAACGTTCTTTTTCGCAACGGGTTTG | |
| CCGCCAGAACACAGGCTAGC | |
| WPRE component (mWPRE) | |
| SEQ ID NO: 46 | |
| 1 | GGGCCCAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCCGCAAGCT |

forward primer for (co)hACE2
SEQ ID NO: 47
CGA TCT AGA GAA ACT TGT TTG CAC GT
reverse primer for (co)hACE2
SEQ ID NO: 48
CGA TCT AGA GAA ACT TGT TTG CAC GT
forward primer
SEQ ID NO: 49
CGA CTC TAG AGT CGA CGG TT
reverse primer
SEQ ID NO: 50
GAT GCG GCC GCT TCA GGC ACC GGG CTT G
forward primer for hTMPRSS2
SEQ ID NO: 51
AAT GCT AGC CAC CAT GGC TTT GAA CTC AG
reverse primer for hTMPRSS2
SEQ ID NO: 52
GTC TCT AGA GCC GTC TGC CCT CAT TT
forward primer
SEQ ID NO: 53
ACG TCT AGA GCA ACA AAC TTC TCT CTG CT
reverse primer
SEQ ID NO: 54
TAT GCG GCC GCT TAG CCC TCC CAC ACA TAA C
forward primer for SARS-COV-2 (co)S (Wuhan Hu-1)
SEQ ID NO: 55
CAG GCT AGC CAC CAT GTT CGT GTT CCT GGT
reverse primer for SARS-COV-2 (co)S (Wuhan Hu-1)
SEQ ID NO: 56
ATA GCG GCC GCT CAG GTG TAG TGC AGC TTC AC
forward primer for SARS-COV-2 (co)S (Wuhan Hu-1) A19
SEQ ID NO: 57
CAG GCT AGC CAC CAT GTT CGT GTT CCT GGT
reverse primer for SARS-COV-2 (co)S (Wuhan Hu-1) A19
SEQ ID NO: 58
ATA GCG GCC GCT TCA GCA GCA GGA CCC ACA CGA ACA
forward primer for SARS-COV-2 S Australia/VIC01/2020
SEQ ID NO: 59
CCT GCA CAG AAA GTA TCT TAC ACC AGG CGA TTC
reverse primer for SARS-COV-2 S Australia/VIC01/2020
SEQ ID NO: 60
GCC AGC AGG GTC TGG AAT
forward primer for SARS-COV-2 D614G variant
SEQ ID NO: 61
GCT GTA CCA GGG CGT GAA TTG C
reverse primer for SARS-COV-2 D614G variant
SEQ ID NO: 62
ACG GCC ACC TGA TTG CTG
forward primer for SARS-COV-2 S mACE2 adaptation
SEQ ID NO: 63
CTA CGG CTT CTA TAC TAC CAA TGG CG
reverse primer for SARS-COV-2 S mACE2 adaptation
SEQ ID NO: 64
CTC TGC AGA GGG AAG TAG

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Materials and Methods

Synthesised cDNA Sequences, Plasmids, Cloning, and Site Directed Mutagenesis

Human (h)ACE2 cDNA (NM_021804.2) was codon optimised ((co)hACE2) for expression in H. sapiens using Java Codon Adaptation Tool (JCat; http://www.jcat.de/CAICalculation.jsp) and synthesised as a cloning plasmid with 5′ Nhel and 3′ PspOMI restriction enzyme sites (pGM845) by Twist Bioscience (https://www.twistbioscience.com/products/genes). Mouse (m)ACE2 (NM_001130513.1) was codon optimised ((co)mACE2) for expression in H. sapiens and synthesised with deletion of its canonical stop codon and addition of 5′ Nhel and 3′ Xbal restriction sites as a cloning plasmid (pGM942), using GeneArt Gene Synthesis services (https://www.thermofisher.com/order/geneartgenes). The hTMPRSS2 cDNA was encoded by pCSDest-hTMPRSS2, and was a gift from Roger Reeves (Addgene, 53887; http://n2t.net/addgene:53887; RRID:Addgene_53887). Plasmids OCT4-F2A-Puromycin resistance (puroR) and pHIV1.EF1a-spCas9-FLAG-P2A-Blasticidin resistance (BSR)-WPRE were provided as gifts from Jacob Hanna (Addgene, 52379; http://n2t.net/addgene:52379; RRID:Addgene_52379), and Feng Zhang (Addgene, 52962; http://n2t.net/addgene:52962; RRID:Addgene_52962), respectively. Codon optimised SARS-CoV-2 Spike ((co)S) derived from the Wuhan Hu-1 strain sequence was provided as an expression vector pcDNA3.1(+)SARS-CoV-2 (co)SWuhanHu-1-Strep tag II, and was a kind gift from Nigel Temperton, University of Kent. Partial SARS-CoV-2 (co)S sequences (from 5′ Nhel and 3′ Sbfl inclusive) encompassing the S protein mutations that define the Alpha (B.1.1.7: A69-70, A145, N501Y, A570D, D614G, 9681H, T7161, S982A, D1118H) and Beta (B.1.351: D80A, D215G, K417N, E484K, N501Y, D614G, A701V) variants of concern were synthesised as cloning plasmids (pGM964 and pGM969, respectively) by Twist Bioscience.

Third generation, self-inactivating (SIN) LV vector plasmids included LV genomes pGM285 (pHIV1 CMV eGFP WPRE), pGM836 (HIV1 CMV Firefly Luciferase [FLuc] WPRE), pGM849 (pHIV1 CMV (co)hACE2-F2A-puroR WPRE), pGM889 (pHIV1 CMV hTMPRSS2-P2A-BSR WPRE), and pGM943 (pHIV1 CMV (co)mACE2-F2A-puroR WPRE) as described below. Additionally, HIV1 packaging plasmids included pMDLg/pRRE (HIV1 GagPol) and pRSV-Rev (HIV1 Rev), which were gifts from Didier Trono (Addgene: 12251; http://n2t.net/addgene:12251; RRID:Addgene_12251, and Addgene: 12253; http://n2t.net/addgene:12253; RRID:Addgene_12253, respectively). Finally, envelope plasmids included: pMD2-G encoding VSVg, which was a gift from Didier Trono (Addgene: 12259; http://623 n2t.net/addgene:12259; RRID:Addgene_12259), and pGM887, pGM896, pGM898, pGM904, pGM906, pGM907, pGM937, pGM939, pGM965, and pGM970 are as described below.

The (co)hACE2 cDNAwas PCR amplified from pGM845 as an Nhel-Xbal fragment, with deletion of its wildtype stop codon (using primer pairs: 5′ CGA TCT AGA GAA ACT TGT TTG CAC GT 3′ (SEQ ID NO: 47) and 5′ CGA TCT AGA GAA ACT TGT TTG CAC GT 3′ (SEQ ID NO: 48)), and ligated with PCR amplified F2A-puroR sequence as an Xbal-Notl fragment from OCT4-F2A-puroR template (using primer pairs: 5′ CGA CTC TAG AGT CGA CGG TT 3′ (SEQ ID NO: 49) and 5′ GAT GCG GCC GCT TCA GGC ACC GGG CTT G 3′ (SEQ ID NO: 50)) with T4 DNA Ligase (NEB) to assemble (co)hACE2-F2A-puroR as a final Nhel-Notl fragment. The hTMPRSS2 cDNA was PCR amplified from pCSDest-hTMPRSS2 as an Nhel-Xbal fragment, with deletion of its wildtype stop codon (using primer pairs: 5′ AAT GCT AGC CAC CAT GGC TTT GAA CTC AG 3′ (SEQ ID NO: 51) and 5′ GTC TCT AGA GCC GTC TGC CCT CAT TT 3′ (SEQ ID NO: 52)), and ligated with PCR amplified P2A-BSR sequence as an Xbal-Notl fragment from pHIV1.EF1a-spCas9-FLAG-P2A-BSR-WPRE (using primer pairs: 5′ ACG TCT AGA GCA ACA AAC TTC TCT CTG CT 3′ (SEQ ID NO: 53) and 5′ TAT GCG GCC GCT TAG CCC TCC CAC ACA TAA C 3′ (SEQ ID NO: 54)) with T4 DNA Ligase to assemble hTMPRSS2-P2A-BSR as a final Nhel-Notl fragment. Assembled cDNAs were gel purified and cloned into HIV lentiviral vector genome plasmid pGM836 via Nhel and Notl restriction enzyme sites, using T4 DNA Ligase (NEB) to generate pGM849 (pHIV1 CMV (co)hACE2-F2A-puroR WPRE) and pGM889 (pHIV1 CMV hTMPRSS2-P2A-BSR WPRE) LV genome plasmids. The (co)mACE2 sequence was cloned from pGM942 into pGM849 backbone as a Nhel-Xbal fragment using T4 DNA Ligase to keep (co)mACE2 sequence in frame with F2A-puroR open reading frame to generate pGM943 LV genome plasmid (pHIV1 CMV (co)mACE2-F2A-puroR-WPRE). The SARS-CoV-2 (co)S (Wuhan Hu-1) sequence was PCR amplified as Nhel-Notl cloning fragments using pcDNA3.1-SARS-CoV-2 (co)S^{Wuhan Hu-1-Strep tag II} template as 647 full-length cDNA (to recover the SARS-CoV-2 S cDNA based on YP_009724390.1; using primer pairs: 5′ CAG GCT AGC CAC CAT GTT CGT GTT CCT GGT 3′ (SEQ ID NO: 55) and 5′ ATA GCG GCC GCT CAG GTG TAG TGC AGC TTC AC 3′ (SEQ ID NO: 56)) or deletion of the 3′ end corresponding to the C-terminus-most 19aa and endoplasmic retention signal (+Δ19aa; using primer pairs: 5′ CAG GCT AGC CAC CAT GTT CGT GTT CCT GGT 3′ (SEQ ID NO: 57) and 5′ ATA GCG GCC GCT TCA GCA GGA CCC ACA CGA ACA 3′ (SEQ ID NO: 58)), and cloned into envelope plasmid pCAGG-coFct4.1 backbone (pGM321) to generate pCAG.SARS-CoV-2 (co)S^{Wuhan Hu-1} (pGM887) and (co)S^{Wuhan Hu-1+19aa} (pGM896) envelope plasmids. Using pGM887 as template the SARS-CoV-2 S Australia/VIC01/2020 (Aus/VIC01; S247R) and D614G variants were modelled by Q5® Site Directed Mutagenesis (SDM; NEB) using primer pairs 5′ CCT GCA CAG AAA GTA TCT TAC ACC AGG CGA TTC 3′ (SEQ ID NO: 59) and 5′ GCC AGC AGG GTC TGG AAT 3′ (SEQ ID NO: 60), or 5′ GCT GTA CCA GGG CGT GAA TTG C 3′ (SEQ ID NO: 61) and 5′ ACG GCC ACC TGA TTG CTG 3′, (SEQ ID NO: 62) respectively; and sub-cloned as Nhel-BstBI or Kpnl-Sbfl fragments, respectively (encompassing the mutations to confer either S247R and D614G) back into pGM887 and pGM896 using T4 DNA Ligase to generate envelope plasmids pCAGG-SARS-CoV-2 (co)S^{Aus/VIC01} (pGM898) and pCAGG-SARS-CoV-2 (co)S^{Aus/VIC01+Δ19aa}(pGM904), respectively, or pCAGG-SARS-CoV-2 (co)S^{G614} (pGM906) and pCAGG-SARS-CoV-2 (co)S^{G614+Δ19aa} (pGM907), respectively. Sequences encompassing the SARS-CoV-2 variants of concern, B.1.1.7 and B.1.351, were sub-cloned from plasmids pGM964 and pGM969 (respectively) into pGM907 backbone as Nhel-Sbfl fragments to generate pCAGG-SARS-CoV-2 (co)S^{B1.1.7+Δ19aa} (pGM965) and (co)S^{B.1.351+Δ19aa} (pGM970) envelope plasmids, respectively. Finally, Q498Y and P499T mutations to confer SARS-CoV-2 S mACE2 adaptation (maS) was mediated by Q5® SDM using 5′ CTA CGG CTT CTA TAC CAA TGG CG 3′ (SEQ ID NO: 63) and 5′ CTC TGC AGA GGG AAG TAG 3′ (SEQ ID NO: 64) primer pairs with pGM896 and pGM907 plasmids as template, and then sub-cloned back into pGM896 and pGM907 backbones, respectively, as BstBI and Kpnl fragments using T4 DNA Ligase to generate pCAGG-SARS-CoV-2 maS^{Wuhan Hu-1,Y498,T499+Δ19aa} (pGM937) and pCAGG-SARS-CoV-2 maS^{Y498,T499,G614+Δ19aa} (pGM939) envelope plasmids.

### Cell Lines and Culture Conditions

Human Embryonic Kidney (HEK) 293T/17 cells (ATC® CRL-11268™) and derivatives described herein were cultured in Dulbecco's Modified Eagle's Medium (Gibco) supplemented with 10% Foetal Bovine Serum (Sigma-Aldrich), 1% penicillin-streptomycin (Gibco) and GlutaMAX™ (Gibco), and cultured at 37° C., 5% CO₂ atmosphere. Where applicable, stable (co)hACE2±hTMPRSS2 cell lines were cultured in complete DMEM as above, with the addition of 3 μg/mL Puromycin (MP Biomedicals) ±5 μg/mL Blasticidin S (Gibco). (HEK)293T/17 SF (ATCC® ACS-4500™) were cultured in FreeStyle™ 293 Expression Medium (Gibco) at 37° C., 8% CO₂ atmosphere and 125 rpm orbital shaking.

Production of Third Generation SIN rHIV1 LVs

The production of S-LV and rHIV1.VSVg LV was performed by co-transfection of human embryonic kidney (HEK) 293T/17 SF suspension cells with third generation, SIN rHIV1 LV vector genome plasmid encoding reporter gene of interest (enhanced green fluorescent protein, eGFP, or Firefly Luciferase, FLuc, as indicated), HIV1 GagPol, pMDLg/pRRE, and HIV1 Rev, pRSV-Rev, using PEIpro® (Polyplus). VSVg pseudotyped LV was achieved by co-transfection with pMD2-G; and in the case of SARS-CoV-2 S-LV pseudotypes pMD2-G plasmid was substituted for SARS-CoV-2 S-LV pseudotype plasmids described in synthesised cDNA sequences, plasmids, cloning, and site directed mutagenesis. After approximately 16 h post-transfection cells were fed with FreeStyle™ 293 Expression Medium supplemented with final 5 mM concentration Sodium Butyrate (Sigma-Aldrich). Transfected cells were cultured for a total of 72 h (or as otherwise stated) at which point LV-containing culture media was harvested. The LV-containing supernatant was then treated with 50 U/mL Benzonase (Merck) in the presence of 1 mM MgCl₂ (Gibco) for 1 h at 37° C. Where indicated LV-containing supernatant was further subjected to centrifugation at 4,650×g for 24 h at 4° C., or purified by Anion Exchange Chromatography (AEX) and Tangential Flow Filtration (TFF). In all cases LV material was filter sterilised and stored at −80° C.

### Sars-CoV-2 S Pseudotype Expression and Syncytia Study

293T/17 cells were seeded into 24 well plates for approximately 70% confluency the next day. Cells were transfected with 200 ng each well with pCMVIE+-eGFP plasmid and 100 ng of the indicated SARS-CoV-2 S pseudotype plasmid. Mock transfection involved co-transfection with 200 ng of pCAGG-eGFP plasmid. Transfections were performed using FuGENE 6 at 1:2 ratio of plasmid to FuGENE 6 reagent. Cells were visualised at the indicated time points post-transfection and eGFP fluorescence images captured using the EVOS™ FL Auto 2 fluorescence microscope (Thermo Scientific™)

### Establishing SARS-CoV-2 S-LV Permissive Cell Lines

293T/17 cells were seeded into 6 well plates for approximately 30-40% confluency the next day. Cells were transduced with crude rHIV1(VSVg) CMV (co)mACE2- or (co)hACE2-F2A-puroR WPRE LV, diluted in OptiMEM-I (Gibco) in the presence of 8 μg/mL polybrene (Sigma-Aldrich) for 6 h at 37° C., 5% CO₂, following which media was replaced with culture media. Transduced cells were cultured for a total of 72 h total at 37° C., 5% CO², and puromycin resistant cells were selected for with the addition of 3 μg/mL puromycin (MP Biomedicals) in culture media. Cells were kept in culture media with 3 μg/mL puromycin for several passages to establish (co)hACE2 or (co)mACE2 only cell lines. After which either cell line was seeded and transduced with rHIV1(VSVg) CMV hTMPRSS2-P2A-BSR WPRE LV as above.

Cells were selected with culture media supplemented with 3 μg/mL Puromycin and 5 ug/mL Blasticidin (Gibco) to select for puromycin- and blasticidin-resistant cells. Cells were kept in culture media with 3 μg/mL puromycin and 5 μg/mL blasticidin for several passages to establish (co)hACE2 or (co)mACE2 & hTMPRSS2 co-expressing cell lines.

### LV Transduction and Titrations

For transduction and LV titration experiments the indicated cell line was seeded into 24 well plates for approximately 30-40% confluency the next day. On the day of transduction cells were counted and media was aspirated. Cells were transduced with inoculum of LV (pseudotypes as indicated) diluted in OptiMEM-I in the presence of 8 μg/mL polybrene to achieve the required multiplicity of infection (MOI) or dilution series. Cells were incubated with diluted vector inoculum for 6h, following which the transduction mix was replaced with culture media. After a total of 48-72 h cells were subjected to flow cytometry as described below. Transductions between 2-20% inclusive were only analysed for titre calculations. Titres were calculated as per equation: N is the number of cells counted at transduction, P is the percentage of eGFP-positive cells, DF is the dilution factor applied at transduction, V is the total volume of vector inoculum per well (mL), and IU is infectious units. Alternatively, with respects to Firefly luciferase encoding vectors, LV material was subjected to HIV1 p24 ELISA (SEK11695, SinoBiological) according to manufacturer's instructions to measure p24 concentrations, with the minor modification of lysing vector material in minimal volume of assay dilution buffer with 0.5% Triton™ X-100 for 15 mins at RT before serial dilutions.

### In Vitro S-LV Neutralisation Assays

293T/17 cells co-expressing (co)hACE2 & hTMPRSS2 were seeded into 24 well plates for approximately 30-40% confluency the next day. On the day of transduction the culture media was aspirated and cells were transduced with MOI 1 (based on cell counts) of S-LV (variants as indicated) diluted in OptiMEM-I with final 8 μg/mL polybrene with nAb (40591-MM43, SinoBiological or 40592-R001, SinoBiological), or corresponding human (GTX35068, GeneTex), mouse (MAB002, R&D Systems), rabbit (AB-105-C, R&D Systems) IgG isotype controls. The titration of IgG is indicated in figure legends. Cells were incubated with antibody:S-LV pre-mixes for 6 h at 37° C., 5% CO₂. Following which the transduction mix was replaced with culture media, and cells cultured for up to 72h. Cells were then subjected to flow cytometry as described below.

### Western Blotting (WB)

Purified S-LV or transduced/transfected cells and their appropriate controls (as indicated in figure legends) were briefly lysed at the indicated timepoints in ice-cold 1× RIPA buffer (150 mM NaCl, 1% Triton™ X-100 [v/v], 0.5% sodium deoxycholate, 0.1% SDS [w/v], 50 mM Tris-HCl [pH 8.0], supplemented with protease inhibitor cocktail (Roche)). Insoluble debris was pelleted at max speed for 30 mins, 4° C., and supernatant measured for protein concentration by DC Protein Assay (Bio-Rad). Protein lysates (or approximately 300 ng worth of p24 for purified S-LV) were separated via 10% SDS-PAGE after boiling samples in Laemlli Buffer with or without final 550 mM @3-Mercaptoethanol (Sigma-Aldrich) as indicated. Proteins were then transferred onto 0.45 μm nitrocellulose membranes, and blocked with TBS-Tween® 20 (0.1%) and 5% milk for 2h. Blots were sequentially incubated with the primary antibodies overnight at RT for hACE2 (R&D Systems, AF933; 1:650), mACE2 (R&D Systems, AF3437; 1:1000), hTMPRSS2 (Abcam, ab242384; 1:2000), SARS-CoV-2 S2 (Thermo Fisher Scientific, MA5-35946 [1A9]; 1:4000), VSVg (Santa Cruz, sc-365019 [F-6]; 1:1000), HIV1 p24 (R&D Systems, MAB7360; 1:700), or GAPDH (Merck; CB1001, 1:20,000), and then with αMouse (Abcam; ab6789, 1:20,000), αRabbit (Abcam; ab6721, 1:20,000), or αGoat (Abcam; ab6885 1:20,000)-HRP conjugated secondary antibodies, accordingly, for 2 h at RT. All antibodies were diluted in TBS-Tween 20 (0.1%) and 5% milk. Blotted proteins were then detected by chemiluminescence with Clarity™ Western ECL Substrate (Bio-Rad). Chemiluminescence signals were visualised and captured on an iBright™ FL1000 (Invitrogen™).

### Immunocytochemistry (ICC)

Cells lines were seeded onto sterile No. 1.0 coverslips in 24 well plates. At confluency cells and their appropriate controls (as indicated in figure legends) were fixed in final 4% formaldehyde solution for 15 mins at RT, and then permeabilised with 0.1% Triton™ X-100 [v/v]solution for 20 mins at RT. Cells were then blocked using blocking solution (5 mg/mL BSA [w/v], 5% FBS [v/v]in D-PBS) for 2 h with gentle agitation. Cells were then incubated overnight at 4° C. with primary antibodies: αACE2 (AF933, 791 1:100) or αTMPRSS2 (ab242384, 1:100). After which, cells were then incubated with secondary αGoat-(A11078, Invitrogen, 1:500) or αRabbit-(A11008, Invitrogen, 1:500) Alexa-Fluor 488-conjugated secondary antibodies accordingly for 2 h at RT with gentle agitation. In between steps cells were washed with D-PBS five times. Parental 293T/17 cells were also completely stained with either αACE2 or αTMPRSS2 and corresponding secondary antibodies to threshold against non-specific primary antibody staining. Cells were washed five times with D-PBS and then coverslips were mounted onto microscope slides with DAPI-containing mounting media (Fluoroshield™; Sigma-Aldrich). Images were captured on the EVOS™ FL Auto 2 fluorescence microscope (Thermo Scientific™).

### Flow Cytometry

Cells were harvested by washing cells with D-PBS (Gibco), and trypsinised to dissociate cells from well plates using TypLE Express (Gibco). Cells were then prepared with D-PBS washes with centrifugation at 500×g for 5 mins and fixed in final 2% formaldehyde solution for 15 mins at RT. Fixative was removed, cells washed and resuspended in D-PBS, and then subjected to flow cytometry using BD™ LSR II Flow Cytometer System. Data were analysed using FlowJo™ v10.7 software.

### Animal Studies

All procedures involving laboratory mice were carried out in accordance with UK Home Office approved project and personal licenses under the terms of the Animals Scientific Procedures Act 1986 (ASPA 1986). All animal procedures were performed at the University of Oxford's Biomedical Services (BMS) Unit situated at the John Radcliffe Hospital, Oxford, UK.

S- and maS-LV.FLuc Administration In Vivo.

Female BALB/c mice (6 weeks old; Envigo RMS, UK) were dosed with the indicated S- or maS-LV.FLuc vectors (corresponding to the indicated dose based on p24 quantification (40 or 166 ng) via nasal sniffing under light isoflurane anaesthesia. S- and maS-LV.FLuc were delivered by intranasal instillation (i.n.) directly onto the nares via a single and continuous droplet (total 100 μL). Control mice were dosed with 100 μL LV formulation buffer (TSSM) by i.n., instead.

### In Vivo Bioluminescence Imaging and Quantitative Analyses

At the indicated time points after S-, maS-LV.FLuc, or control TSSM dosing described in S- and maS-LV.FLuc administration in vivo, mice were dosed with 100 μL of 15 mg/mL D-Luciferin (Xenogen Corporation Alameda) via the i.n. method under light anaesthesia with isoflurane. After 10 mins incubation bioluminescence imaging was performed using the IVIS spectrum imaging system (IVIS Lumina LT, Series III, PerkinElmer). Average bioluminescence (photons/sec/cm2/sr) values were visualized and quantified using a pseudocolor range representing light intensity within standardised tissue areas for the murine nose and lung.

### Statistics

Post-hoc statistical analysis was performed using Prism 8.4.3 (GraphPad Software). Where appropriate comparisons between multiple groups were performed using One-Way ANOVA followed by Dunn's multiple comparisons test to a chosen comparator group or Tukey's comparison of all groups as appropriate. Where the assumptions of One-way ANOVA were violated, the non-parametric Kruskal-Wallis test followed by Dunn's multiple comparisons test to a chosen comparator group was used, or, where appropriate, data was log 10 transformed and analysed by One-Way ANOVA followed by Tukey's comparison of all groups. Data is presented as group mean and/or with individual data points plotted, and where appropriate±standard deviation of the mean (SD). In all cases, P value <0.05 was considered statistically significant. ns, *, *, * and **** indicate P-values of >0.05, <0.05, <0.01, <0.001, and <0.0001, respectively.

### Example 1—Deletion of SARS-CoV-2 S Putative ERS and Transducing Permissive (Co)hACE2 and hTMPRSS2 Co-Expressing Cells Rescues S-LV Transduction

To test and examine the function of in-house and BSL1-compliant SARS-CoV-2 S pseudotyped LV (S-LV) for subsequent titrations and in vitro neutralisation, permissive cell lines expressing the human ACE2 receptor±TMPRSS2 protease were first engineered. Stable cell lines co-expressing codon optimized (co)hACE2±hTMPRSS2 were generated from the parental 293T/17 cell line using VSVg pseudotyped SIN rHIV1 LVs encoding (co)hACE2-F2A-puroR or hTMPRSS2-P2A-BSR (not shown), with both transgenes under CMV promoter control.

Stable cell lines were selected and established and the expression of (co)hACE2±hTMPRSS2 was confirmed by immunocytochemistry (ICC) and Western Blotting (WB) (data not shown). As intended, detectable levels of either ACE2 or TMPRSS2 were appreciably above that of parental 293T/17 in their respective cell lines. The engineered (co)hACE2 cell line demonstrated significantly elevated (co)hACE2 expression compared to its (co)hACE2 & hTMPRSS2 co-expressing counterpart (data not shown, P=0.0173, unpaired t-test with Welch's correction) and corresponded to approximately 42.8% reduction in signal intensity. Expression of hTMPRSS2 expression was appreciably well above that of parental 293T/17 and (co)hACE2 only expressing cell line (data not shown). Regardless of the (co)hACE2-expression disparity between stable (co)hACE2 cell lines, the fact that the expression of was lower in engineered (co)hACE2 & hTMPRSS2 co-expressing cell line still allowed us to subsequently make informative comparisons between permissive cell lines in the context and function of hTMPRSS2 co-expression.

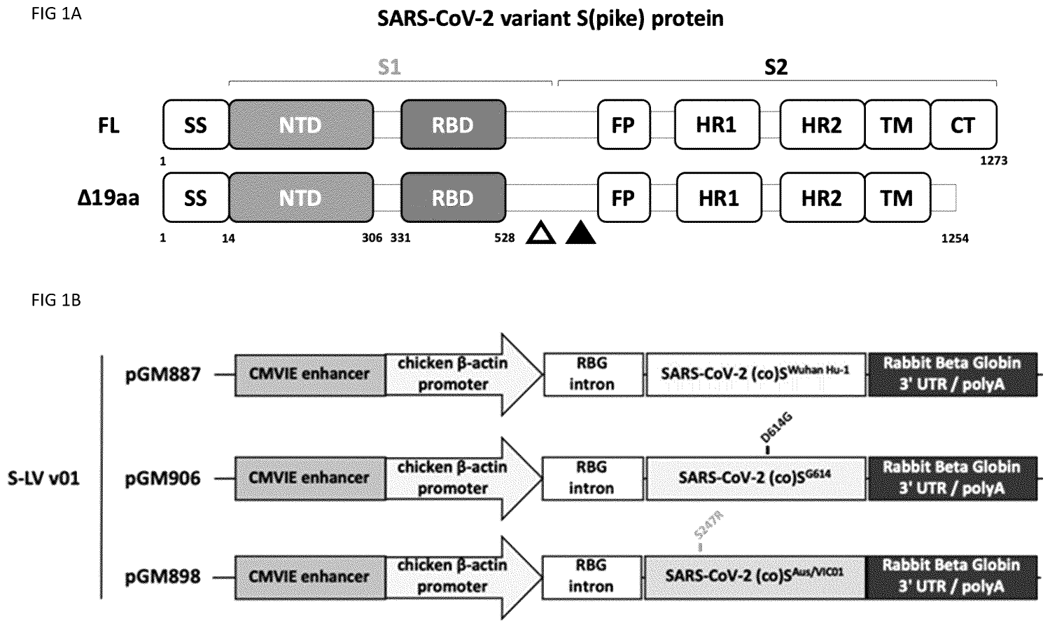

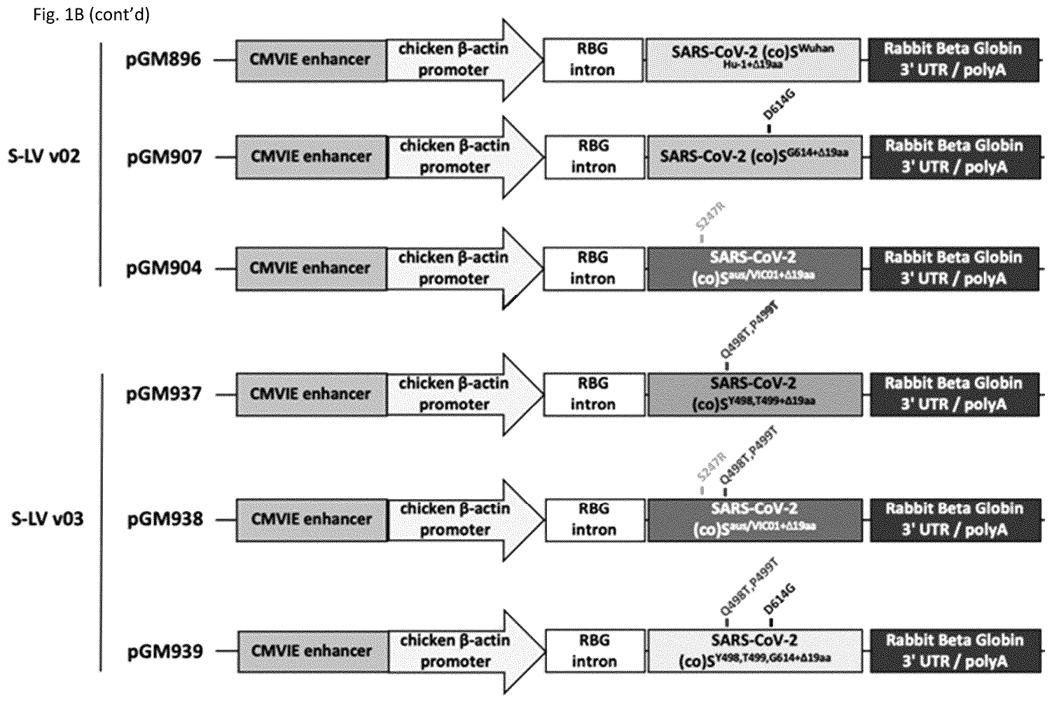

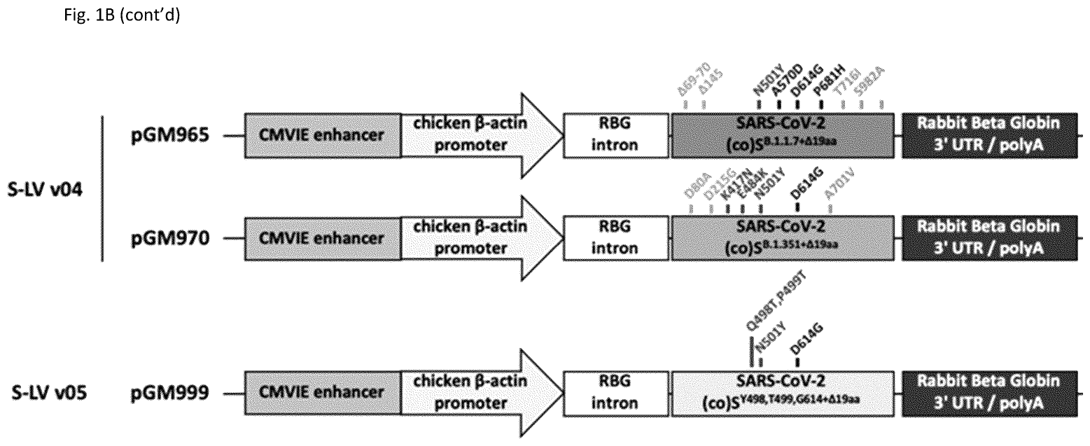

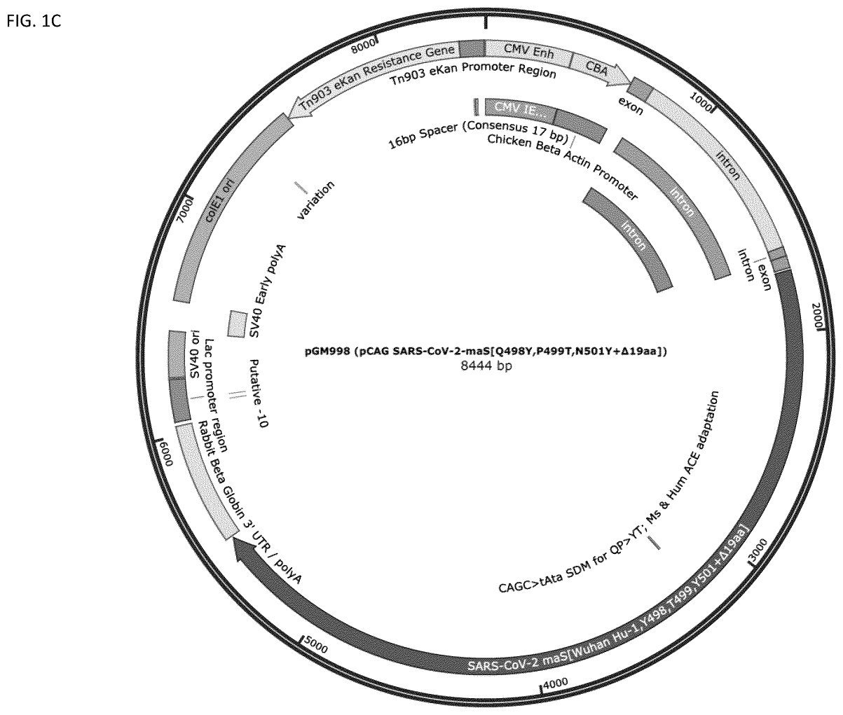

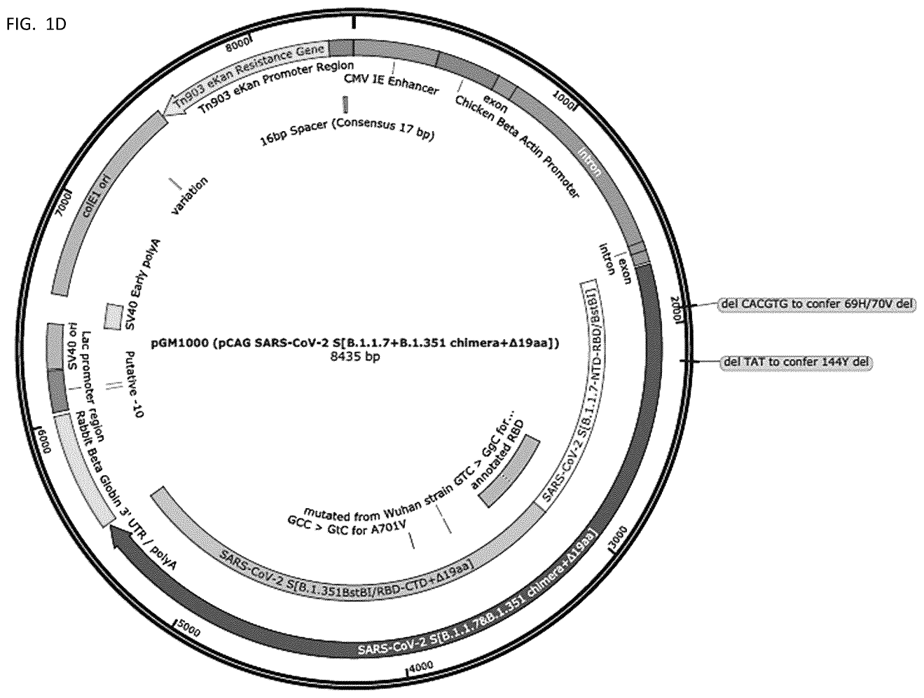

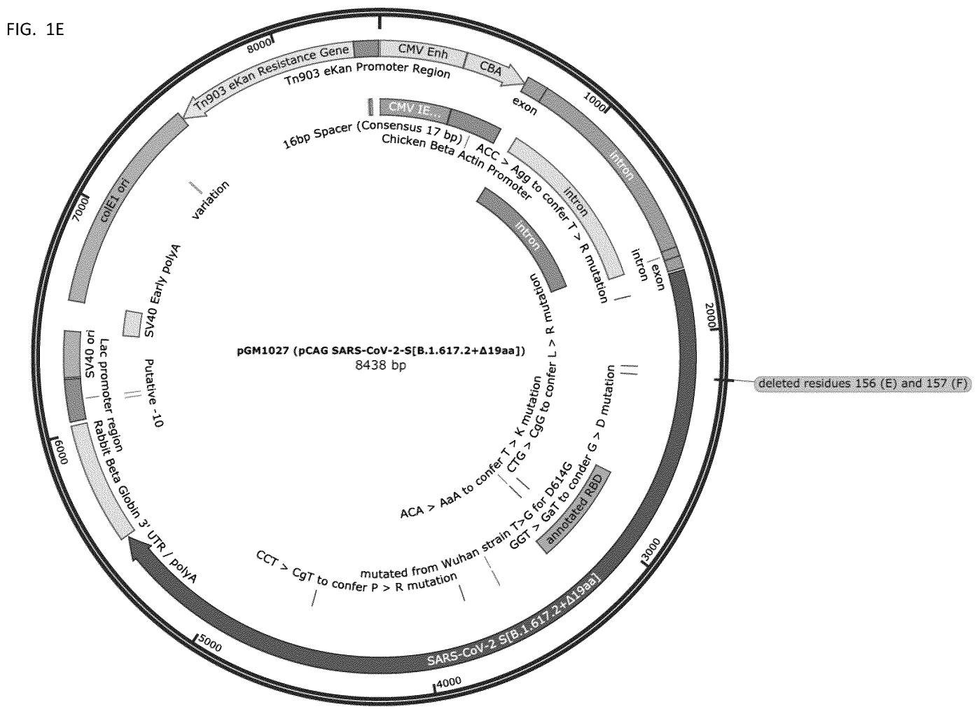

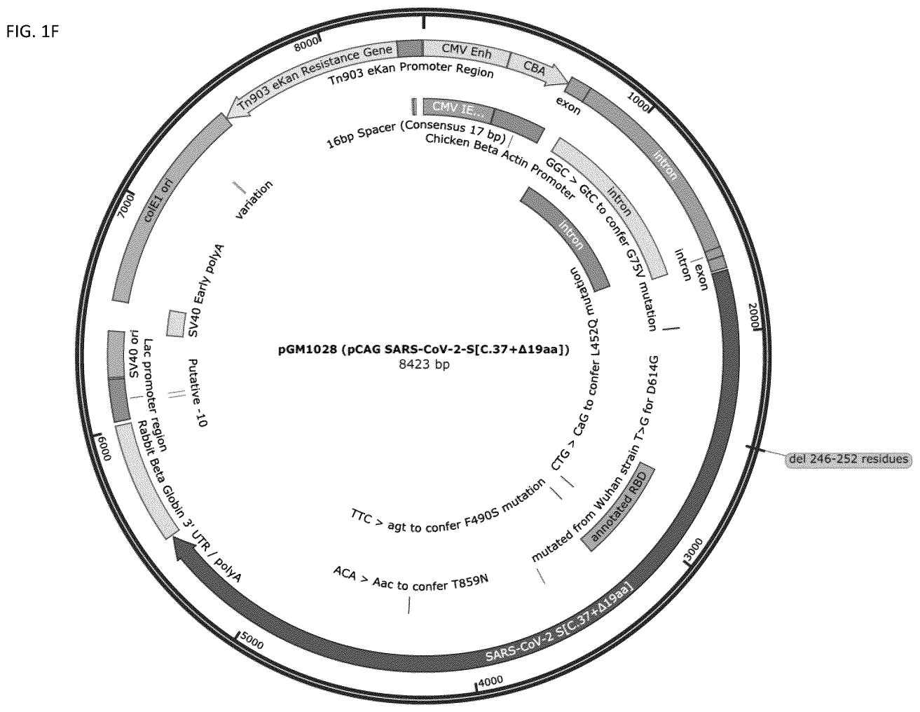

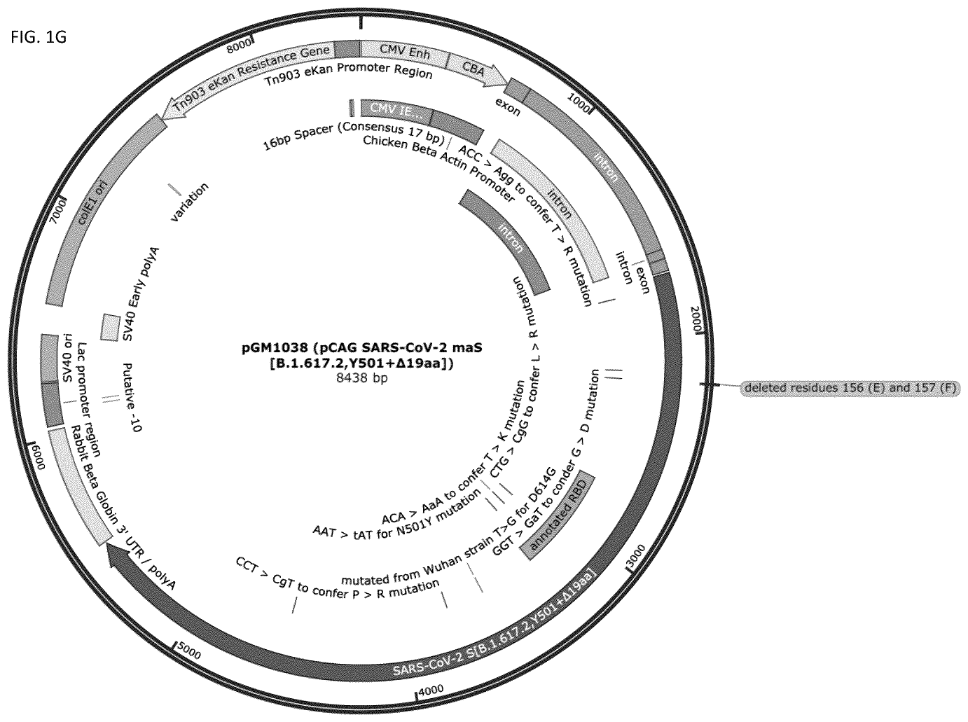

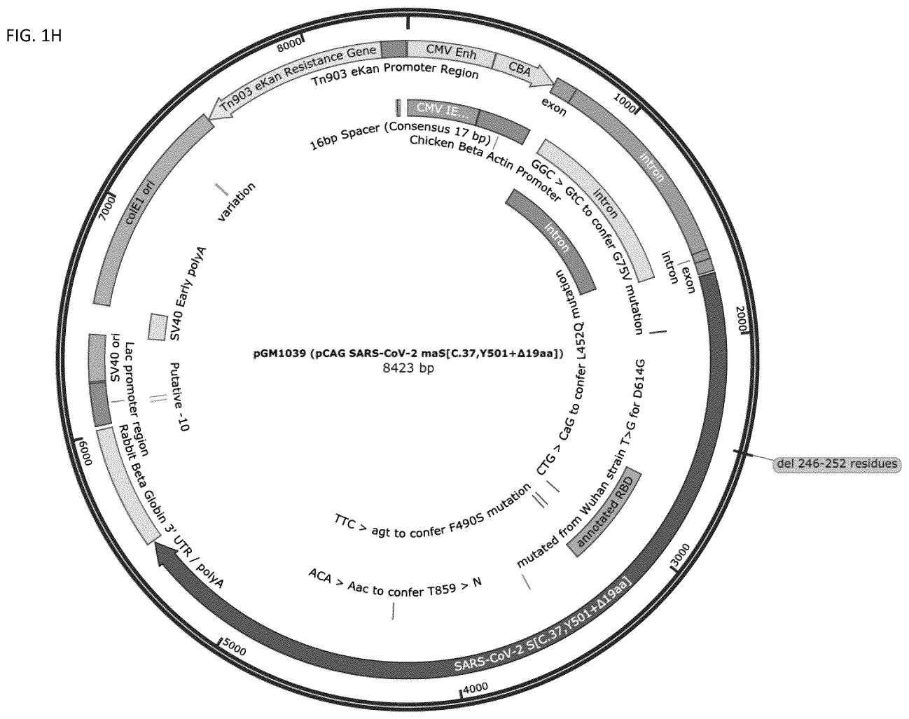

Moving forward and as proof of principle the following SARS-CoV-2 S variants were modelled: Wuhan Hu-1, G614, Aus/VIC01±deletion of 19aa residues of the C-terminal tail putatively harbouring an endoplasmic reticulum retention signal (+Δ19aa; see FIG. 1A for a representative graphic comparing full length and +Δ19aa SARS-CoV-2 S proteins relative to annotated domains). The complete mutation profiles defining modelled SARS-CoV-2 S variants (compared to Wuhan Hu-1 reference) used to pseudotype LV in the present study is presented in Table 1. Sequences were cloned

TABLE 1
Mutation profiles of SARS-CoV-2 S variants modelled
relative to Wuhan Hu-1 sequence as baseline reference

| | S1 | | | | | | | | | | |
|---|---|---|---|---|---|---|---|---|---|---|---|
| | NTD | | | | | | RBD | | | | |
| variant\aa residue | 69 | 70 | 80 | 145 | 215 | 247 | 417 | 484 | 496 | 499 | 501 |
| (co)S^{Wuhan Hu-1±Δ19aa} | H | V | D | Y | D | S | K | E | Q | P | N |
| (co)S^{G614±Δ19aa} | | | | | | | | | | | |
| (co)S^{Aus/VIC01±Δ19aa} | | | | | | R | | | | | |
| (co)S^{B.1.1.7+Δ19aa} | Δ | Δ | | Δ | | | | | | | Y |
| (co)S^{B.1.1351+Δ19aa} | | | A | | G | | N | K | | | Y |
| ma(co)S^{Wuhan Hu-1, Y498, T499+Δ19aa} | | | | | | | | | Y | T | |
| ma(co)S^{Y498, T499, G614+Δ19aa} | | | | | | | | | Y | T | |
| ma(co)S^{Aus/VIC01, Y498, T499+419aa} | | | | | | R | | | Y | T | |
| ma(co)S^{Y498, T499, G614, Y501+Δ19aa} | | | | | | | | | Y | T | Y |

| | | | S2 | | | | | |
|---|---|---|---|---|---|---|---|---|
| | S1 | | | | | | | 1255- |
| variant\aa residue | 570 | 614 | 701 | 681 | 716 | 982 | 1118 | 1273 |
| (co)S^{Wuhan Hu-1±Δ19aa} | A | D | A | P | T | S | D | ±Δ |
| (co)S^{G614±Δ19aa} | | G | | | | | | ±Δ |
| (co)S^{Aus/VIC01±Δ19aa} | | | | | | | | ±Δ |
| (co)S^{B.1.1.7+Δ19aa} | D | G | | H | I | A | H | Δ |
| (co)S^{B.1.1351+Δ19aa} | | G | V | | | | | Δ |
| ma(co)S^{Wuhan Hu-1, Y498, T499+Δ19aa} | | | | | | | | Δ |
| ma(co)S^{Y498, T499, G614+Δ19aa} | | G | | | | | | Δ |
| ma(co)S^{Aus/VIC01, Y498, T499+419aa} | | | | | | | | Δ |
| ma(co)S^{Y498, T499, G614, Y501+Δ19aa} | | G | | | | | | Δ |

Blank cells indicate no change in aa residue compared to Wuhan Hu-1 reference aa, amino acid residue(s); co, codon optimised; ma, mouse-ACE2 adapted; NTD, N-terminal domain; RBD, receptor binding domain; Δ, deleted

TABLE 2
The effect of the deletion on S-LV and maS-LV titres

| | SARS- | | | | | | |
|---|---|---|---|---|---|---|---|
| | CoV-2 | Average IU/mL (±SD) | | | Average RLU/mL (±SD) | | |
| vGM | variant | Full length S | (+Δ19aa) | | batch 1 | batch 2 | batch 3 |
| vGM258&259 | Wuhan | 3.65E+05 (±2.17E+04) | 1.88E+06 | (±3.07E+05) | — | — | — |
| | Hu-1 | | | | | | |
| vGM262&263 | G614 | 3.68E+05 (±5.71E+04) | 6.3E+06 | (±2.72E+05) | 3.45E+12 (±1.78E+12) | 2.81E+12 (±5.94E+11) | 3.34E+12 (±5.78E+11) |
| vGM288 | B.1.1.7 | — | 7.64+06 | (±1.51E+05) | 2.81E+12 (±5.94E+11) | — | — |
| | (Alpha) | | | | | | |
| vGM289 | B.1.351 | — | 4.42E+06 | (±7.62E+05) | 1.72E+12 (±7.38E+11) | — | — |
| | (Beta) | | | | | | |
| vGM273 | ma Wuhan | — | 4.01E+06 | (±7.44E+05) | — | — | — |
| | Hu-1 | | | | | | |
| vGM275 | ma G614 | — | 6.25E+06 | (±9.76E+05) | 1.49E+12 (±6.95E+11) | — | — |

as codon optimized cDNA into expression plasmids under CAG promoter control (see FIG. 1B for a schematic overview on pseudotype plasmid configurations and annotated mutations that confer each SARS-CoV-2 S variants). The sequences of the modified SARS-CoV-2 S variants are aligned in FIG. 2.

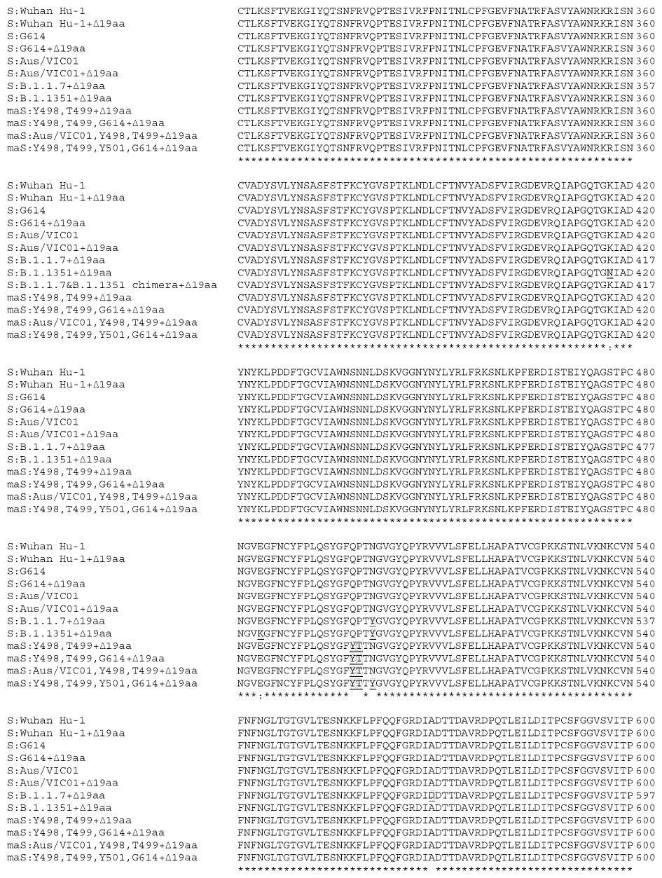

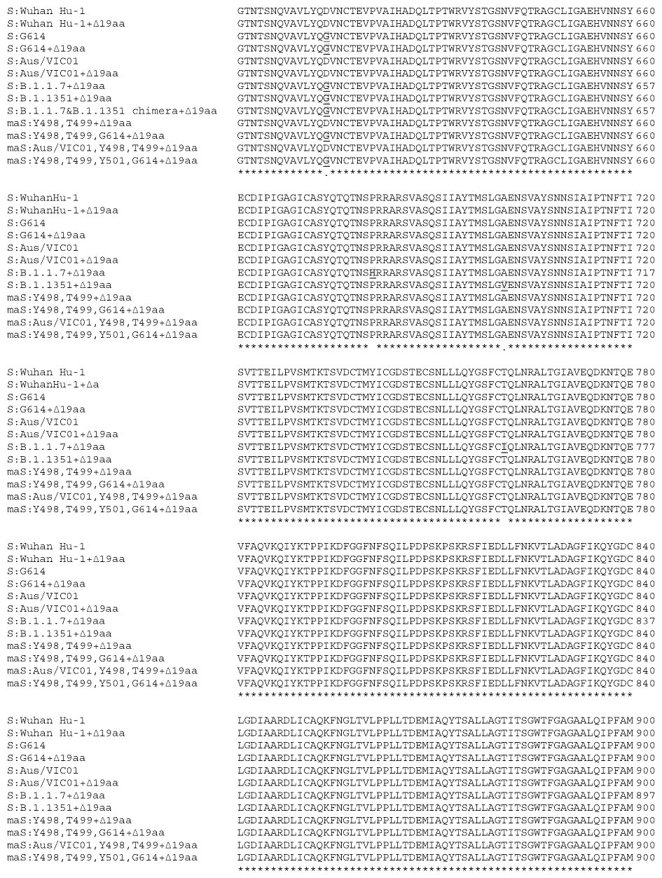

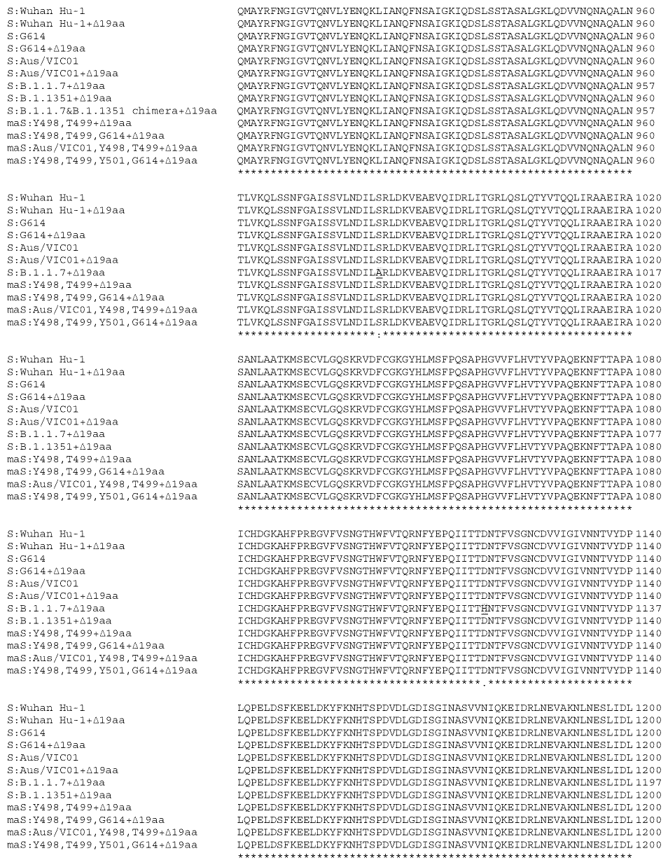

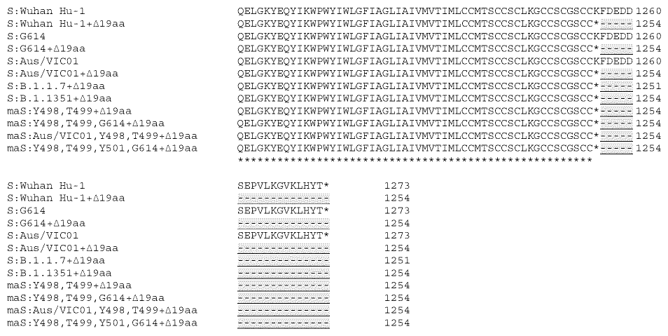

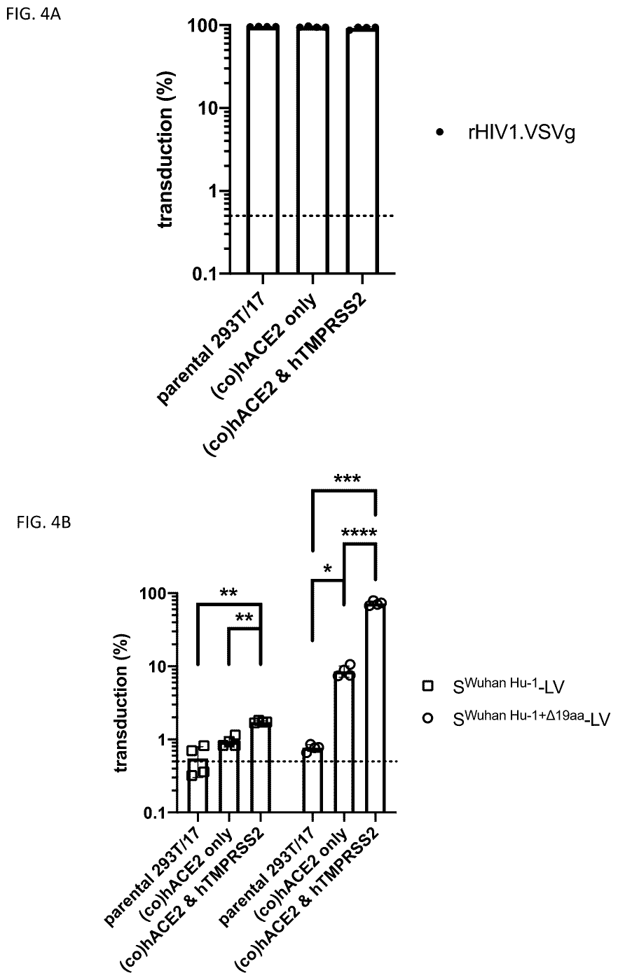

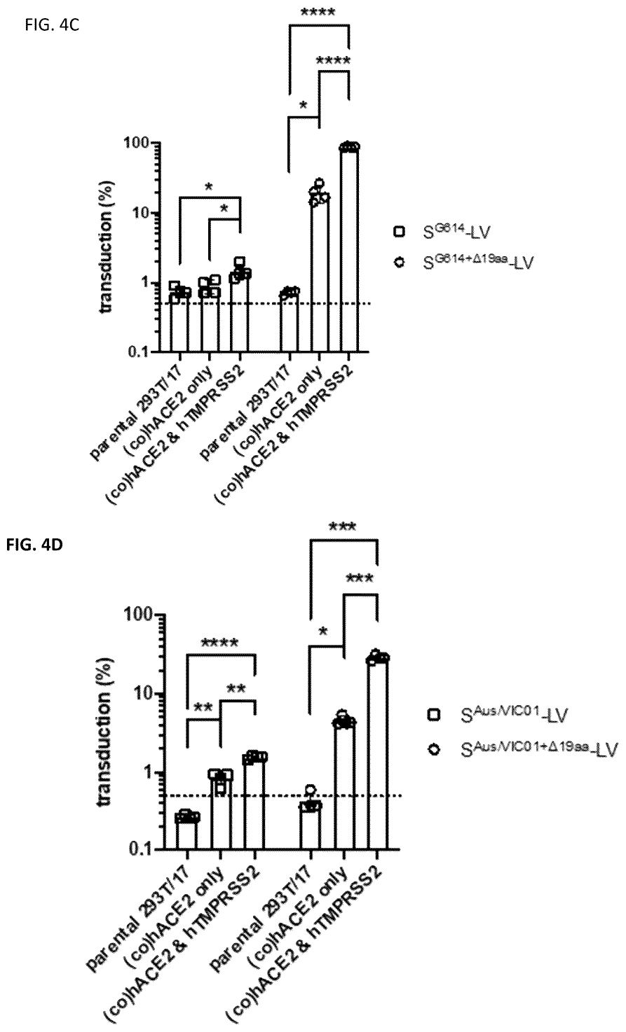

SARS-CoV-2(co)S (Wuhan Hu-1±Δ19aa, G614±Δ19aa, and Aus/VIC01±Δ19aa) pseudotyped rHIV1 LV (encoding eGFP: S-LV.eGFP) transduction was examined across novel engineered (co)hACE2 ±hTMPRSS2 cell lines or parental 293T/17 (FIG. 4).

As expected, rHIV1.VSVg LV transduced similarly between parental 293T/17 and engineered cell lines (FIG. 4A; P >0.05, Repeat Measure One-Way ANOVA with Geisser-Greenhouse correction, and Tukey's multiple comparisons test). In contrast, the modelled non-mouse adapted S-LV.eGFP showed a clear minimum requirement for (co)hACE2 receptor-dependent transduction with transduction efficiencies generally showing most improvements on (co)hACE2 only expressing cells in the context of +Δ19aa (FIG. 4B-D; P<0.05, Repeat Measure One-Way ANOVA with Geisser-Greenhouse correction, and Tukey's multiple comparisons test). Furthermore, transductions were prominently further rescued with the co-expression of hTMPRSS2 by permissive cell lines, and especially in the context of S-LVs with +Δ19aa C-terminus truncation of the SARS-CoV-2 S glycoprotein (FIG. 4B-D; P<0.001, Repeat Measure One-Way ANOVA with Geisser-Greenhouse correction, and Tukey's multiple comparisons test). Generally, and with regards to the repertoire of +Δ19aa truncated S-LV.eGFP tested, up to a log improvement in transduction was measured between parental, (co)hACE2 only, and (co)hACE2 & hTMPRSS2 expressing cell lines. 293T/17 expressing only hTMPRSS2 were not permissive for S-LV (data not shown), and therefore, without being bound by theory, the substantial improvement and rescue in S-LV transduction may likely be attributed to the complex interplay between ACE2 entry receptor and hTMPRSS2.

### Example 2—the S-LV Platform can Rapidly Model 5 Glycoproteins Derived from Variants of Concern or of Interest

The COVID-19 and SARS-CoV-2 pandemic remains an on-going dilemma with the repeated surge in novel and emerging SARS-CoV-2 S VOCs that are occurring globally. These VOCs show increased transmissibility potentials and thus a further concern to public health. The B.1.1.7 and B.1.351 VOCs S glycoprotein (+Δ19aa) were modelled for rapid LV pseudotyping. In addition to this a mouse (m)ACE2-adapted SARS-CoV-2 S harbouring the Q498Y and P499T mutations was also modelled, in order to capture the ability for the S-LV platform to be pseudotyped with a wide array of SARS-CoV-2 S glycoproteins and thus function as an extensive resource to model alternative SARS-CoV-2 (see FIG. 1B schematic overview for these pseudotype plasmid configurations and annotated mutations that confer each SARS-CoV-2 S variant). The complete mutation profiles defining these additional SARS-CoV-2 S and maS variants (compared to Wuhan Hu-1 reference) are also presented in Table 1. Nucleic acid and amino acid alignments of the SARS-CoV-2 S and maS variants are shown in FIGS. 2 and 3 respectively.

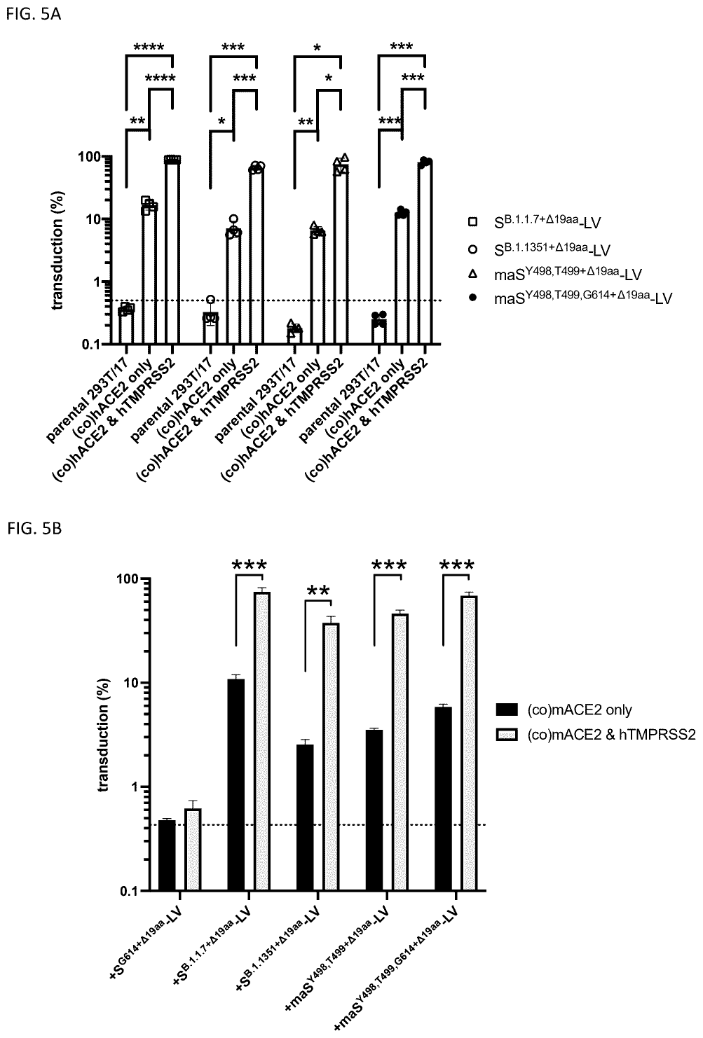

S-LVs pseudotyped with B.1.1.7 (S^{B1.1.7+Δ19aa}-LV.eGFP), B.1.351 (S^{B.1.351+Δ19aa-}LV.eGFP), and maS (maS^{Wuhan Hu-1,Y498,T499+Δ19aa}-LV.eGFP and maS^{Y498,T499,G614+Δ19aa}-LV.eGFP) with +Δ19aa C-terminus truncation performed similarly to S^{Wuhan Hu-1+Δ19aa}-LV and S^{G614+Δ19aa}-LV; and expectedly transduced (co)hACE2±hTMPRSS2 cell lines and parental 293T/17, displaying a minimum requirement for (co)hACE2 receptor dependent transduction, and with transduction substantially rescued further with hTMPRSS2 co-expression (FIG. 5A, in all cases P<0.05 within each S-LV.eGFP or maS-LV.eGFP transduction group, Repeat Measure One-Way ANOVA with Geisser-Greenhouse correction, and Tukey's multiple comparisons test).

The maS (Q498Y and P499T) and N501Y-containing S (particularly concerning VOCs B.1.1.7 and B.1.351 pseudotypes)-LV.eGFP were further able to transduce mACE2 expressing cells (FIG. 5B). Low to negligible transduction of mACE2-expressing cell lines with S^{G614+Δ19aa}-LV.eGFP was statistically insignificant to that measured from rHIV1.bald treated cells (FIG. 5B; P >0.05, Repeat Measure 193 One-Way ANOVA with Geisser-Greenhouse correction, and Tukey's multiple comparisons test).Transductions of mACE2 co-permissive S- and maS-LVs were found significantly rescued further (up to 1 log) by the co-expression of hTMPRSS2 (FIG. 5B; in all cases P<0.01 within each S-LV.eGFP or maS-LV.eGFP transduction group between (co)mACE2 and (co)mACE2 & hTMPRSS2 co-expressing cell lines, Repeat Measure One-Way ANOVA with Geisser-Greenhouse correction, and Tukey's multiple comparisons test). Lastly, the expressed S glycoprotein retained their function to mediate cell-to-cell fusion and syncytia, post transfection of parental 293T/17 cells, in a time and SARS-CoV-2 S G614-containing-dependent fashion FIG. 5C). Representative fluorescence imaging of co-transfected cells co-expressing eGFP and SARS-CoV-2 S^{Wuhan Hu-1+Δ19aa}, S^{G614+Δ19aa}, or S^{B.1.1.7+Δ19aa} demonstrated that, relative to eGFP mock co-transfected cells as control, S^{Wuhan Hu-1+Δ19aa} was potently capable of mediating syncytia by 72 h post transfection. Cell death and cytotoxicity was notable for S^{Wuhan Hu-1+Δ19aa} co-expressing cells at an extended period in culture (144 h post-transfection), whereas G614-containing S glycoproteins co-expressing cells exhibited notably diminished syncytia at 72 h post-transfection with delayed onset by 144 h post-transfection (FIG. 5C, see white arrow heads highlighting syncytia formation in 72 h eGFP panels).

Overall, these data show the robust and rapid ability for this S-LV platform to model functional SARS-CoV-2 S glycoproteins from modelled VOCs and VOIs, and recapitulate inherent functions as per their S defining mutation profiles, including mACE2 co-permissiveness and mediating syncytia formation. In particular the Q498Y, P499T, D614G and N501Y substitutions were found to be particularly effective in achieving mouse adaptation, both in terms of facilitating transduction and mediating syncytia formation.

### Example 3—the S-LV Platform Achieves High Titre PSV

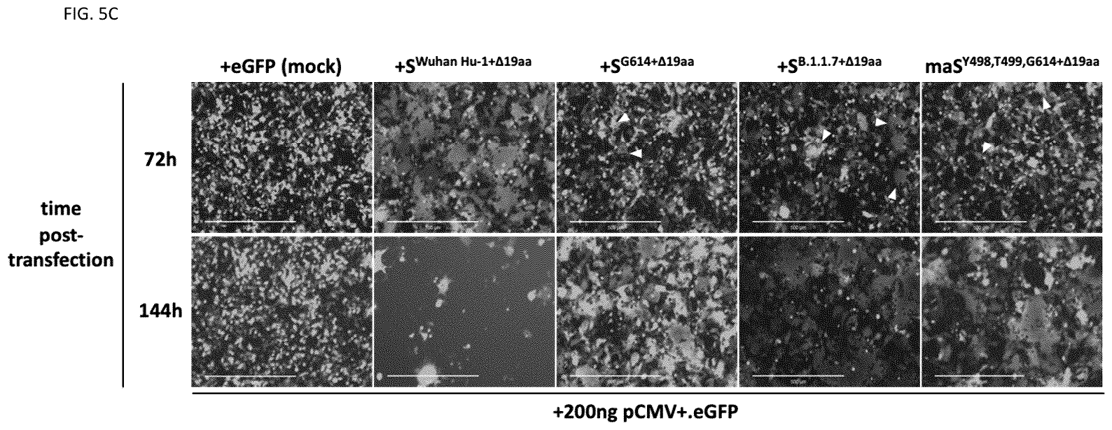

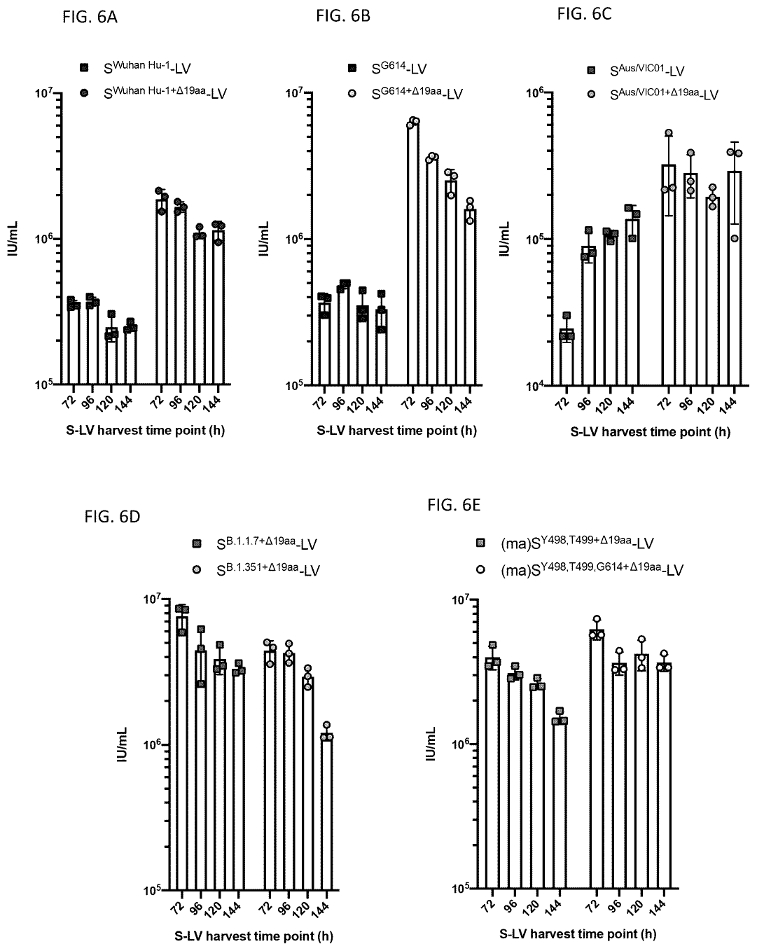

Optimal transduction for non-mouse adapted S-LVs was achieved using (co)hACE2 & hTMPRSS2 co-expressing cells, and in turn were used to examine functional titres of S-LVs. The optimal harvest point of S-LV was first determined by examining S-LV.eGFP titres over production time courses of 72-144 h with crude LV collected at 24 h intervals (FIG. 6), given preliminary titres suggesting that 48 h post-transfection was not the optimal harvest window for S-LV (data not shown). With benchmark rHIV1.VSVg titres of approximately 2E7 IU/mL at 72 h post-transfection (data not shown), S-LVs derived from Wuhan Hu-1, G614, Aus/VIC01, VOC strains B.1.1.7 and B.1.351, and the variants of interest harbouring mACE2-permissive mutations, and including the Δ19aa C-terminus truncation, were generally found to generate peak functional titres at 72 h post-transfection, achieving in excess of >1E6 IU/mL (FIGS. 6A-E, respectively), with as high as 5-7E6 IU/mL for S^{G614+Δ19aa}-S^{B.1.1.7+Δ19aa}-S^{B.1.351+Δ19aa}, and maS^{Y498,T488,±G614+Δ19aa}-LVs (FIGS. 3B, D, and E, respectively). The exception to this rule was the S^{Aus/VIC01+Δ19aa} pseudotype, which achieved lower titres of approximately 3E5 IU/mL (FIG. 3D). S^{Wuhan}, S^{G614}, and S^{Aus/VIC01} without the Δ19aa C-terminus truncation performed poorly across the production time course (FIGS. 3A-C, respectively), and was in line with initial transduction propensities for these S pseudotyped derivatives described in FIG. 4.

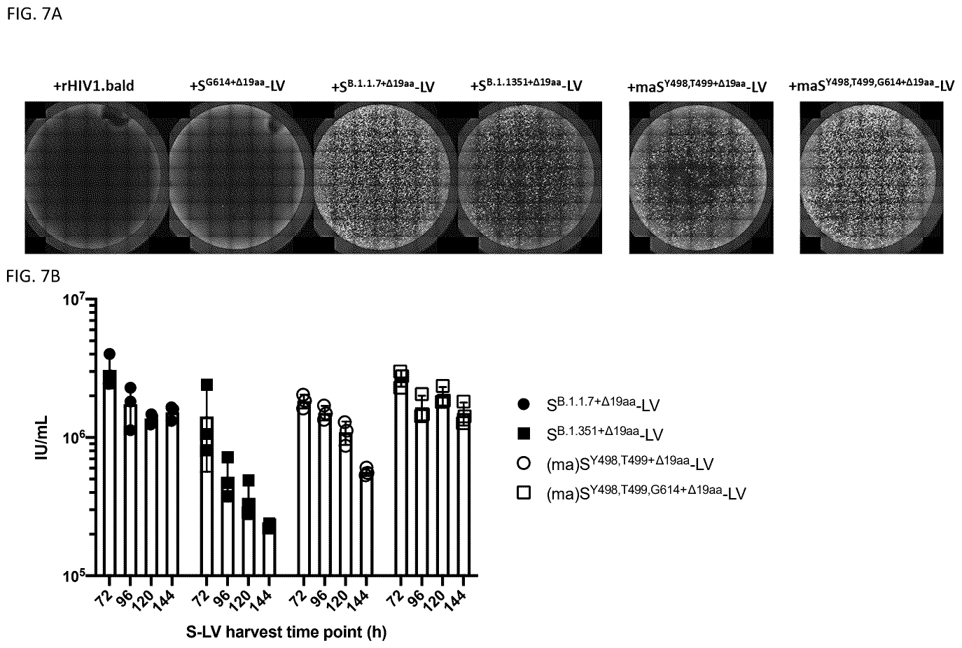

No additive benefit in functional titres was measured for extended periods in culture post-transfection; in fact, functional titres generally started to decline from 96 h post-transfection onwards (FIG. 7). Furthermore, functional titres correlated with the expression profiles of monomeric SARS-CoV-2 S and HIV1 p24 proteins over the 144 h time courses, with peak expression of both measured by WB generally at 72 h post transfection (data not shown). Concentration of S- and maS-LVs by slow spin centrifugation was found to be a viable protocol and was also performed to permit commitment to more translationally relevant means to purify LVs, including more traditional anion exchange chromatography (AEX) and tangential flow filtration (TFF) combinations. Centrifugation and volumetric concentration of select S- and maS-LVs (S^{Wuhan Hu-1+Δ19aa}-LV S^{G614+Δ19aa}-LV, S^{Aus/VIC01+Δ19aa}-LV S^{B.1.1.7+Δ19aa}-LV S^{B.1.351+Δ19aa}-LV, maS^{Y498,T499+Δ19aa}-LV, and maS^{Y498,T499,G614+Δ19aa}-LV) were successfully achieved as indicated by improved functional titres (≥1E8 IU/mL) for selected variants, especially when compared against functional titres of corresponding crude S-LV material collected at harvest (>1E6 IU/mL for all variants except 6.75E5 IU/mL for S^{Aus/VIC01+Δ19aa}LV). An average of 138.8±35.22-fold increase in functional titres was demonstrated when up to 100-fold volumetric concentration of centrifuged S-LV was applied across the S-LV library generated.

Overall, this S-LV platform, especially with the inclusion of Δ19aa C-terminus truncation in SARS-CoV-2 S glycoproteins demonstrated the capacity to achieve high functional titres, with the added ability to model VOCs that have profound clinical relevance, and particularly enables the high-titre production of mouse-adapted S-LV, making this a potentially valuable research tool. Exemplary titres are set out in Table 2.

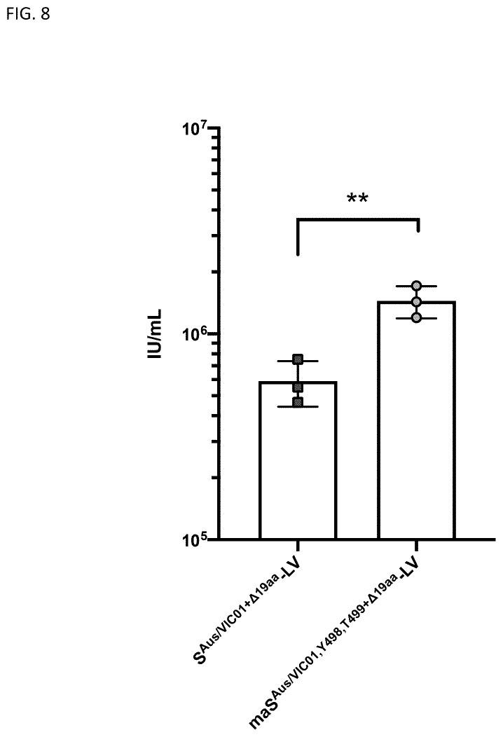

It was then investigated whether the lower titre observed for S^{Aus/VIC01+Δ19aa}-LV could be further rescued using the mouse adaption mutations. Thus, a further variant S-LV was generated in which the Y498 and T499 substitutions were introduced into S^{Aus/VIC01+Δ19aa}-LV. As shown in FIG. 8, compared with the functional titres reported in FIG. 6, initial experiments indicate that the Y498 and T499 substitutions can further rescue the functional titres of the S^{Aus/VIC01+Δ19aa}-LV in addition to the Δ19 C-terminal deletion.

### Example 4—S-LV can Model and Infer SARS-CoV-2 S VOCs' Infectivity In Vitro

Having established the feasibility of producing high functional titres of crude S-LV derived from a myriad of clinically relevant or modified SARS-CoV-2 S variants, the utility of the S-LV library was expanded for additional downstream applications. Given significant volumetric concentration of S-LV was achieved after centrifugation, it was examined if S-LV could be produced to sub-clinically relevant standards for use in vivo. To achieve this S-LV encoding Firefly luciferase pseudotyped with codon optimised (co)S^{G614+Δ19aa}, (co)S^{B.1.1.7+Δ19 aa}, (co)S^{B.1.351+Δ19aa}, ma(co)S^{Y498,T499,G614+Δ19aa} were produced and subjected to concentration and purification by a combination of AEX and TFF. In addition, given that N501Y-containing S (particularly concerning VOCs B.1.1.7 and B.1.351 pseudotypes)-LV.eGFP were able to transduce mACE2 expressing cells as observed for the maS-LV comprising the Q498Y and P499T substitutions (see Example 2 and FIG. 5), it was decided to introduce the N501Y substitution into the maS-LV to investigate whether the addition of N501Y would further improve the function/functional titre of maS-LV. Cloning was conducted to introduce the N501Y substitution into the maS-LV v3 pseudotype cDNA (maS^{Y498,T499,G614+Δ19aa}-LV, pGM939) to generate maS-LV v5 (pGM999, see FIG. 1). Ma(co)S^{Y498,T499,Y501,G614+Δ19aa} encoding Firefly luciferase was produced and subjected to concentration and purification by a combination of AEX and TFF. The resultant S-LV.FLuc vectors were used to transduce 293T/17 cells co-expressing (co)hACE2 and hTMPRSS2 and lysates examined for functional titres by luciferase activity.

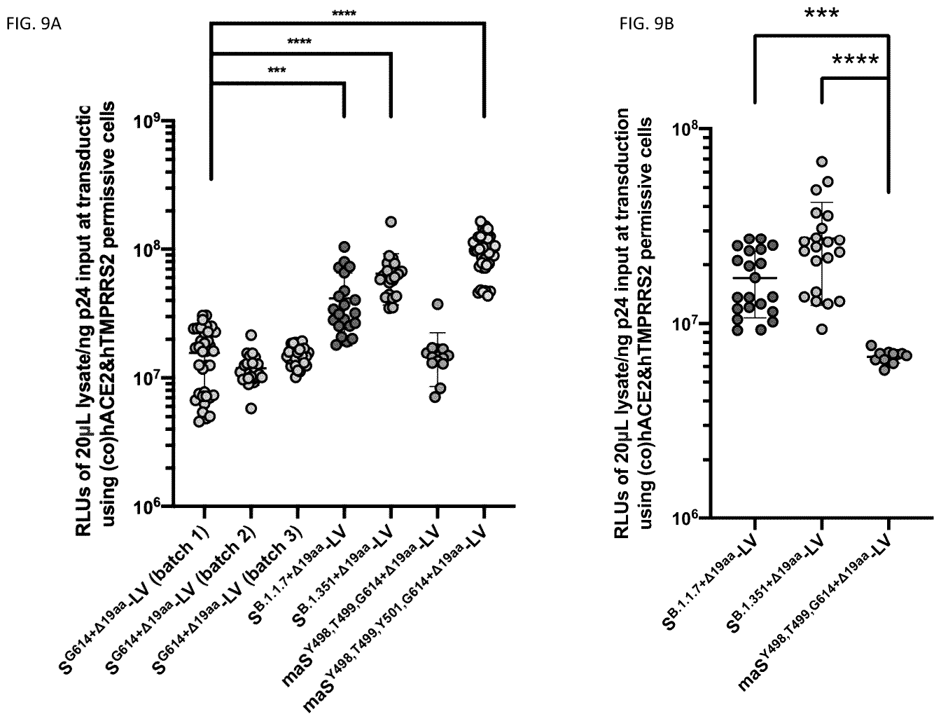

RLUs captured from (co)hACE2 and hTMPRSS2 co-expressing permissive cells were normalised by a function of input p24 at transduction, and the functional quality of each purified S-LV (1.57E7 ±8.01E6, 1.19E7±3.07E6, 1.49E7±2.44E6, 4.15E7±2.31E7, 1.38E7±3.06E6 RLUs of 20 μL lysate per ng p24 input for S^{G614+Δ19aa} batches 1-3, and maS^{Y498,T499,G614+Δ19aa}-LV, respectively) were largely comparable and not statistically significant (FIG. 9A; P >0.05 each compared to SG614+Δ19aa-LV batch 1, Mann Whitney t-test). Of note, the modelled SARS-CoV-2 S VOCs (B.1.1.7 and B.1.351) and ma(co)S^{Y498,T499,Y501,G614+Δ19aa} all exhibited statistically significant increase in their functionality per ng p24 input when compared to S^{G614+Δ19aa}-LV (4.15E7±2.27E7, 6.45E7+2.77E7, and 9.92E7±3.17E7 RLUs of 20 μL lysate per ng p24 input, respectively; P<0.001 and P<0.0001, Mann Whitney t-test). Impressively, an improved functional quality of approximately 7-fold was calculated between maS^{Y498,T499,Y501,G614+Δ19aa}-LV (i.e. with the addition of the N501Y mutation) and G614-containing S variants. Alternatively, 3-4-fold improved transduction per ng p24 input between S-LV modelling SARS-CoV-2 S VOCs and G614-containing S variants were calculated. Similar was calculated with RLUs captured from (co)mACE2 & hTMPRSS2 co-expressing cells—the functional quality of S-LVs per ng input of p24 also reflected that the S-LVs modelling VOCs showed improved transduction of permissive cells by a factor of 2-3-fold (FIG. 9B; P<0.001 each compared to ma(co)S^{Y498,T499,G614+Δ19aa}-LV, Mann Whitney t-test).

Overall, these data further highlight successful purification and concentration of S- and maS-LVs after concentration and purification with AEX and TFF, and that modelled VOCs can be assessed further to qualify and ascertain their infectivity profiles relative to S^{G614+Δ19aa}-LV or suitable PSV reference, in vitro. The data also demonstrate that the N501Y mutation improves the functional transduction of S-LVs, particularly maS-LVs.

### Example 5—maS-LVs can be Neutralised by Commercially Available SARS-CoV-2 Neutralising Antibodies

It was next investigated whether the maS-LV could prove a useful resource for pre-clinical/clinical use by testing maS^{Y49S,T499,G614+Δ19aa}-LV's susceptibility for neutralisation. maS-LV also serves to model the prospective neutralisation propensity of potential S variants of interest. After characterizing the optimal MOI to transduce (co)hACE2 & hTMPRSS2 co-expressing cell lines without saturating transduction efficiency (MOI 1 demonstrated high transduction efficiency without saturating upper transduction sensitivity; data not shown), maS^{Y498,T499,G614+Δ19aa}-LV was subjected to in vitro neutralisation assays with commercially available neutralising antibodies (nAbs).

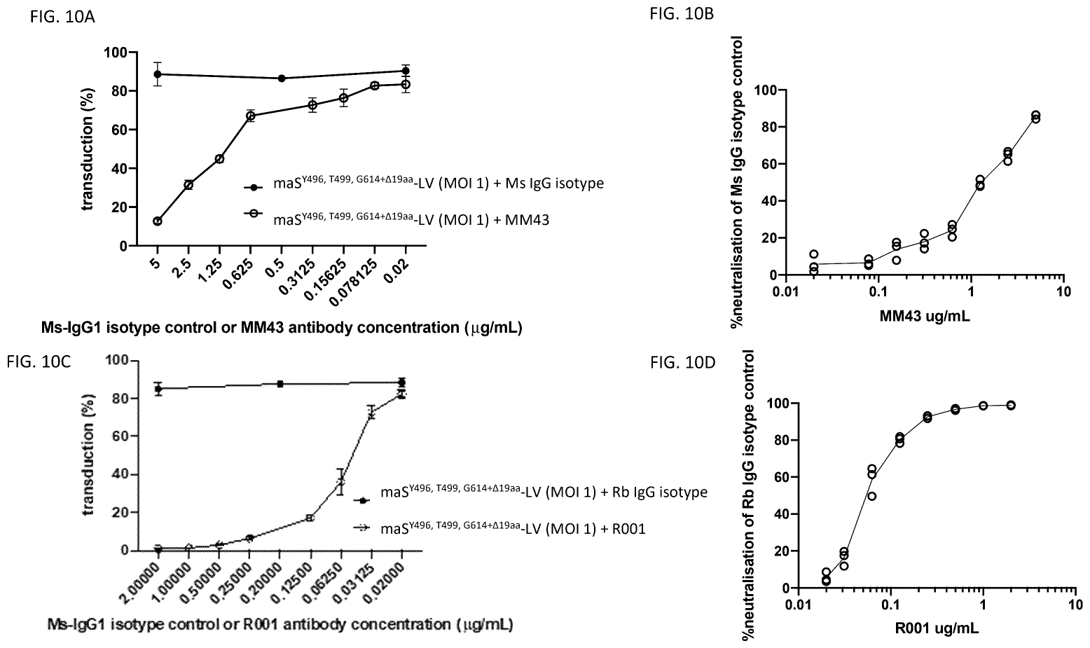

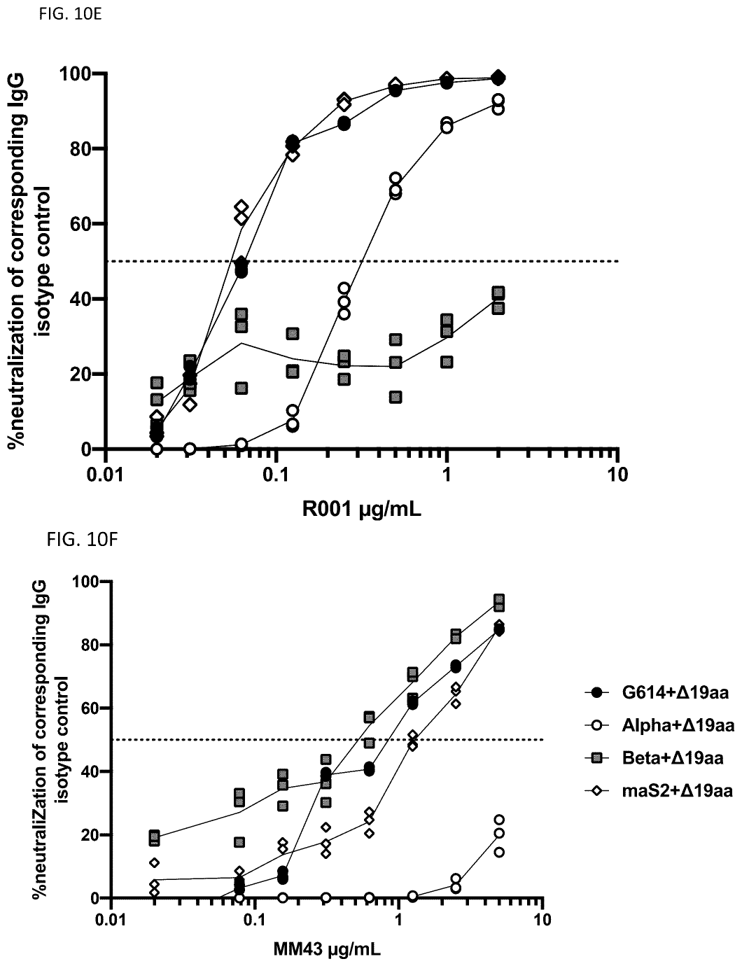

As shown in FIG. 10C and D, the RBD-targeting R001 nAb showed potent neutralisation of m^{aSY498,T499,G614+Δ19aa}-LVs (IC50=0.05 μg/mL). As shown in FIG. 10A and B, the S1-targeting MM43 was also capable of neutralising maS^{Y49S,T499,G614+Δ19aa}-LV (IC50=1.2 μg/mL).

However, as shown in FIG. 10E and F, S-LVs can be neutralized in a SARS-CoV-2 S variant-dependent manner by neutralizing antibodies. 293T/17 cells co-expressing (co)hACE2 or (co)mACE2 (as indicated) & hTMPRSS2 (seeded to achieve 30-40% confluency on day of transduction) were transduced with premix of the indicated S or maS-LV.eGFP (SARS-CoV-2 S^{G614+Δ19aa} (G614+Δ19); B.1.1.7+Δ19 (Alpha+Δ19); B.1.351+Δ19 (Beta+Δ19); and SARS-CoV-2 S^{G614+Y498+T499+Δ19aa} (maS2+Δ19)) and (E) R001 nAb targeting the SARS-CoV-2 S RBD, or (F) MM43 nAb targeting SARS-CoV-2 S1 domain, at the indicated final working concentrations. 72hpi transduction efficiencies were measured by flow cytometry and % neutralization was calculated as the % of average transduction of the corresponding IgG isotype control. The dotted line refers to 50% neutralization from which average IC₅₀ were calculated.

Measured R001 IC50 (ug/mL) were as follows: for G614+19del S-LV=0.06; for Alpha+19del S-LV=0.32; for Beta+19del S-LV=escapes neutralization; for maS2+19del (S-LV v02)S-LV=0.05

Measured MM43 IC50 (ug/mL) for G614+19del S-LV=0.8; for Alpha+19del S-LV=escapes neutralization; for Beta+19del S-LV=0.5; for maS2+19del (S-LV v02)S-LV=1.25.

Overall, these data help demonstrate that the S-LV platform is capable of recapitulating neutralization or neutralization escape profiles using nAbs.

### Example 6—Mouse-Adapted SARS-CoV-2 Spike Proteins can be Used to Pseudotype SIV1 Vectors at Acceptable Functional Titres

Next, it was investigated whether maS^{Y498,T499,G614+Δ19aa} could be used to pseudotype other lentiviral vectors. SIV1 was used for proof-of-concept studies.

S-LV was produced using transient transfection of 293T/17 SF cells for VSVg pseudotyped rSIV1 lentiviral vectors (n=1) or SARS-CoV-2 (co)S^{G614+Δ19aa} and maS^{Y498,T499,G614+Δ19aa} (n=2 each). Crude rSIV1.VSVg and rSIV1.S-LV vector material were harvested 72 h post-transfection, serially diluted and transduced on (co)hACE2 & hTMPRSS2 co-expressing cells. 72 h post transduction, cells were subjected to flow cytometry to determine IU/mL titres based on eGFP transduction.

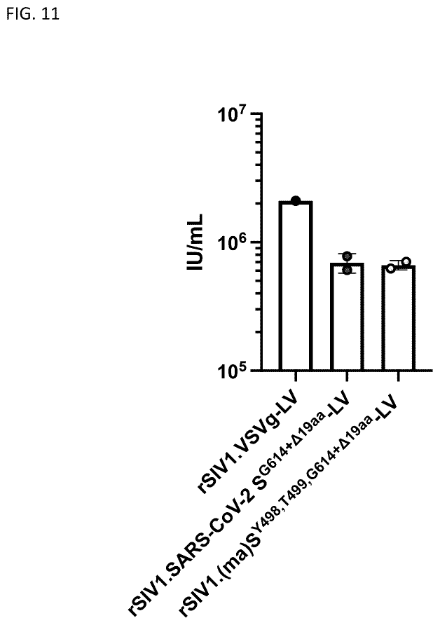

As shown in FIG. 11, rSIV1 vector genome can be packaged with SARS-CoV-2 (co)S pseudotypes. Thus, S-LV pseudotypes, including maS^{Y498,T499,G614+Δ19aa}, incorporate into envelopes independent of SIV/HIV LV platform (e.g. compared to SIVctHN pseudotype), with titres in the region of 6-7E5 IU/mL.

### Example 7—mACE2-Permissive S-LVs Prove a Potent Gene Transfer Tool In Vivo Independent of hACE2 Expression in Trans

To further substantiate the in vitro mACE2 and hACE2 co-permissiveness of maS-LV transduction, purified and concentrated maS^{Y498,T499,G614+Δ19aa}-LV.FLuc was tested in vivo to assess its translation in gene transfer capabilities. Additionally, given observed transduction advantages exhibited by VOC-derived S-LVs, it was investigated if S-LVs could also be modelled in vivo as a predictive tool to screen SARS-CoV-2 S variants as an extension of the in vitro findings reported in Example 4 (FIG. 9). A dose titration study involved dosing non-hACE2 expressing BALB/c mice with increasing % of the maximum feasible dose (MFD) and the luciferase expression kinetics monitored.

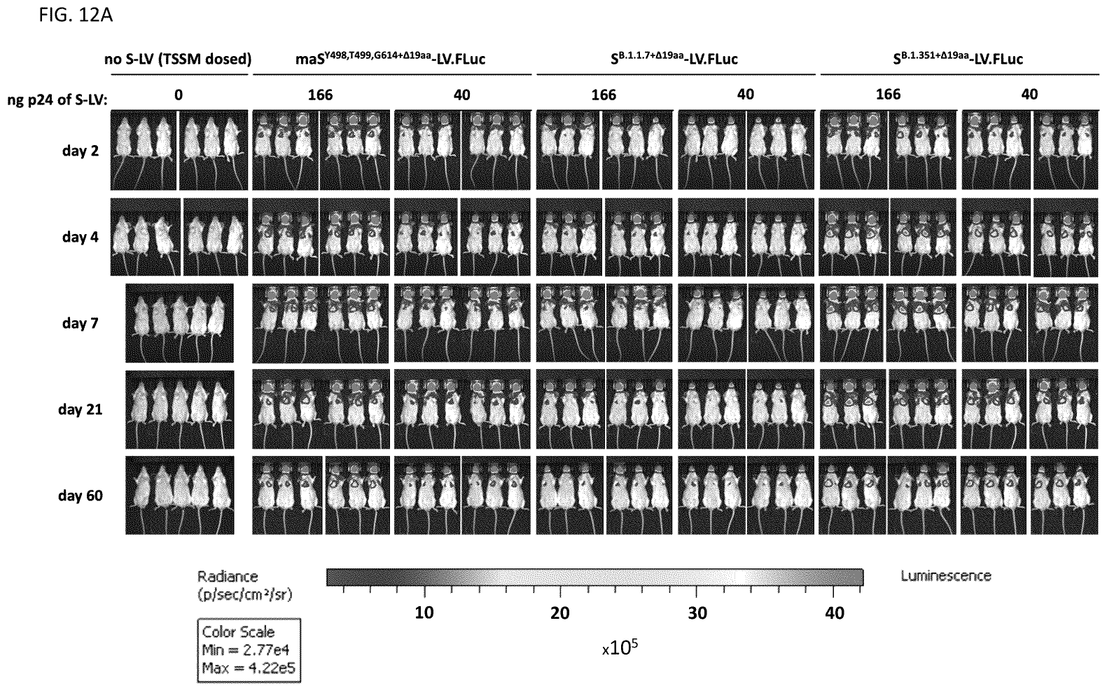

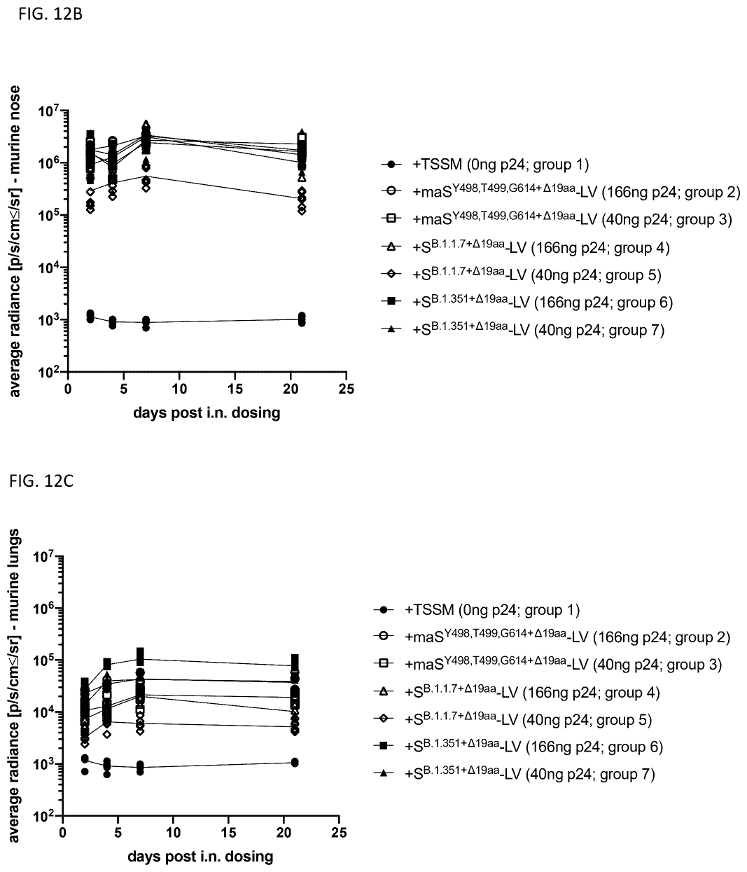

A dose-dependent increase in maS-LV.FLuc transduction was observed, inferred by in vivo bioluminescence over time, independent of hACE2 expression in trans (FIG. 12). Especially, as per representative FIG. 12A, maS-LV transduction was measurable in the murine lungs but more prominently in the murine nose by mACE2 co-permissive maS-LVs tested. Luciferase expression was well detectable above the background, as inferred by naïve animals for both the murine nose (FIG. 12B) and lungs (FIG. 12C), with signal intensity detected as early as 2 days post i.n. dosing, peaking approximately 7 days post dosing, and becoming persistent (with a modest decline) up to 21 days post dosing for either the murine nose or lungs (FIGS. 12B&C, respectively). Further to this, similar transduction kinetics over the 21-day time course was measured in the murine nose (approximately 1000-fold improved signal intensities above background) between maS^{Y49S,T499,G614+Δ19aa}-LVs; the exception being mice dosed with 40 ng S^{B.1.1.7+Δ19aa}-LV.FLuc that showed approximately 100-fold improved signal intensities above background. In the murine lungs, the maS^{Y49S,T499,G614+Δ19aa}-LV-signal intensities ranged between approximately 10-fold increase (e.g. regarding 40 ng p24 dose of maS^{Y498,T499,G614+Δ19aa}-LV.FLuc) to approximately 100-fold increase in signal intensities above the background for S^{B.1.351+Δ19aa}-LV.FLuc doses.

These observations were further appreciated given dosed mice did not show substantial weight loss, and generally were comparable with TSSM dosed control mice (data not shown), and in turn infers sufficient tolerability of S-LV at the tested ng doses as an in vivo gene transfer tool.

Overall, the presented data supports that the S-LV platform can be administered in vivo without the need of hACE2 expression in trans in murine lungs and airways.

### Example 8—mACE2-Permissive S-LVs as Gene Therapy Agents

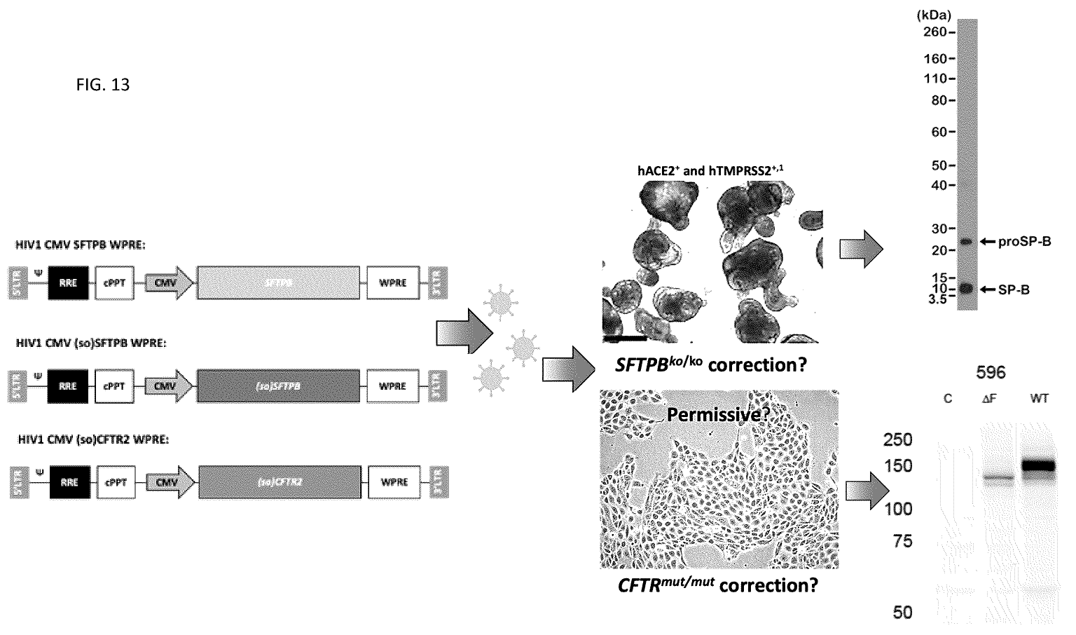

maS-LV vectors, such as those exemplified herein and illustrated in FIG. 1 are used as gene therapy vectors. S-LV (S-LV v02, 03, 05) encoding therapeutic gene of interest: (so)SFTPB or (so)CFTR2 are produced to correct mutant SFTPB or CFTR cell models (SFTPB^{ko} or 16HBE14o-with CFTR F508Δ models, respectively). Rescue of protein expression profile is demonstrated by WB. A representative plan is set out in FIG. 13.

### Example 9—S^{B.1.351+Δ19aa}-LV Directs Higher Lung Transgene Expression than S^{Y498, T499, G613 Δ19aa}-LV

In vivo experiments were conducted to compare transgene expression in the lungs of mice. In particular, S^{Y498,T499,G613 Δ19aa}-LV (S-LV maS2) was compared with S^{B.1.351+Δ19aa}-LV (S-LV Beta).

S^{Y498,T499, G613 Δ19aa}-LV (S-LV maS2), S^{B.1.351+Δ19aa}-LV (S-LV Beta) or vehicle was delivered to the lungs of BALB/c mice (n=6/group 166 ng p24 S-LV delivered).

Compared to the Wuhan isolate of SARS-CoV-2, S^{B.1.351+Δ19aa}-LV (S-LV Beta) contains the following sequence variations: D80A, D215G, K417N, E484K, N501Y, D614G, A701V and the Δ19 amino acid deletion at the C-terminus. Similarly, S^{Y498,T499, G613 Δ19aa}-LV (S-LV maS2) contains Q498Y, P498T, D614G, and the Δ19 amino acid deletion at the C-terminus. Both S-LV vectors contained a firefly luciferase transgene under the control of the CMV promoter.

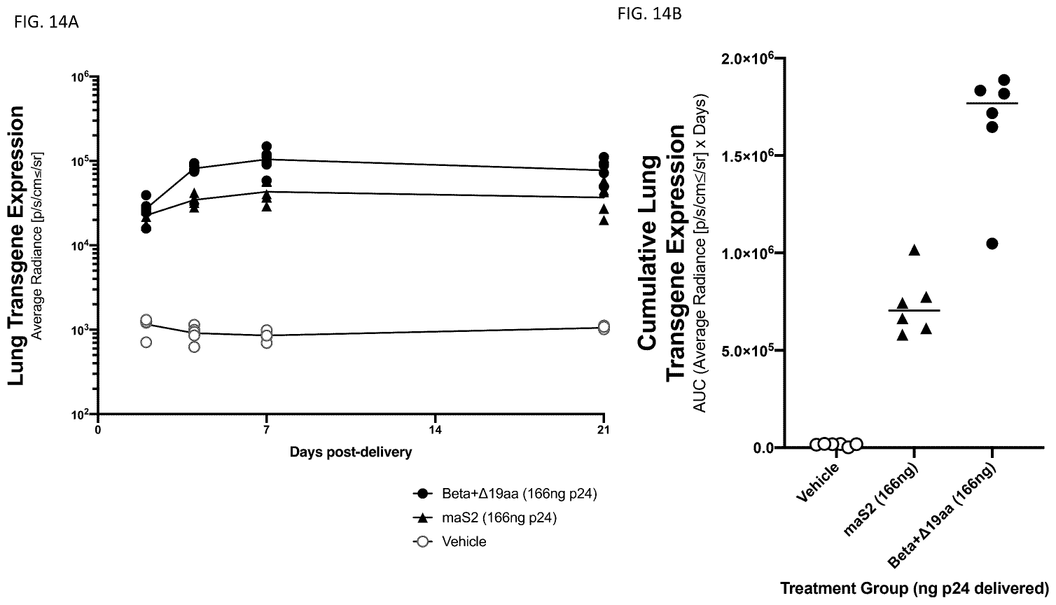

As shown in FIG. 14B, both S^{Y498, T499, G613 Δ19aa}-LV (S-LV maS2) and S^{B.1.351+Δ19aa}-LV (S-LV Beta) directed significantly greater luciferase activity than vehicle treated animals (47.5-fold and 107.6-fold respectively, p<0.0001, one-way ANOVA). Further, S^{B.1.351+Δ19aa}-LV (S-LV Beta) directs approximately 2.3-fold greater in vivo lung transgene expression than S^{Y498,T499,G613 Δ19aa}-LV (S-LV maS2) (p<0.0001, one-way ANOVA).

### Example 10—S-LVs can Deliver Therapeutic Transgenes to SARS-CoV-2 Permissive Human Cells

As per Example 8, a mACE2-permisive S-LV (maS3, SARS-CoV-2 S^{P498,T499,Y501,G614+Δ19aa}) was produced with a Surfactant Protein B (SF-B) transgene and used to transduce SARS-CoV-2 permissive 293T/17 (stably co-expressing hACE2 & hTMPRSS2), either in crude or concentrated S-LV forms at concentrations of 100 μl, 250 μl or 500 μl (crude) or 1 μl, 10 μl or 50 μl (concentrated). Post transduction, cell lysates were subjected to SDS-PAGE and Western blot analysis for mature SP-B homodimer expression (under non-reducing conditions), compared to null background levels of non-transduced cells. GAPDH served as loading control.

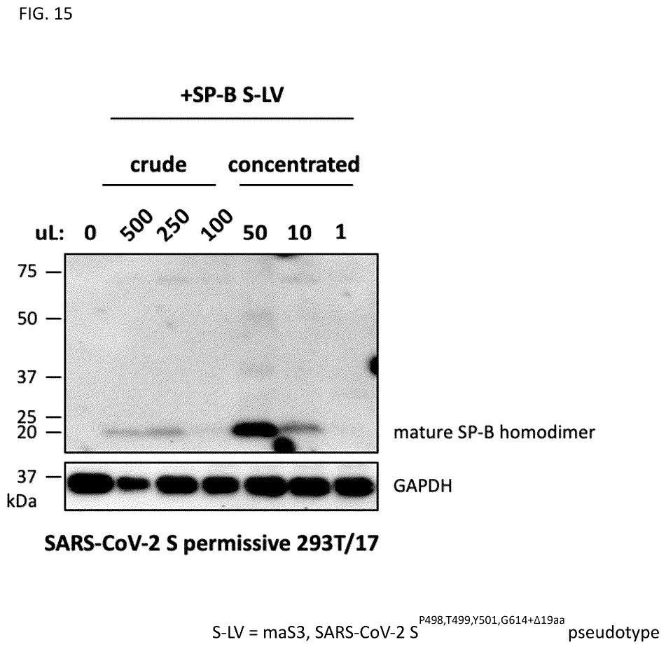

As shown in FIG. 15, this mACE2-permisive S-LV (maS3, SARS-CoV-2 S^{P498,T499,Y501,G614+Δ19aa}) successfully drives therapeutic transgene (human SF-B) expression SARS-CoV-2 permissive human 293T/17 cells, when delivered as either crude or concentrated form, even at the lowest volumes administered.

Additionally, (G614+Δ19aa, SARS-CoV-2 S^{G614+Δ19aa}) was produced with an EGFP transgene and microinjected into human lung bud organoids (LBO). LBO were generated from the human RUES2 embryonic stem cell line, and model alveolar type II cells in 3D organoids. Concentrated S-LV encoding EGFP was mixed with phenol red (2:1 ratio) and maximal feasible dose (MFD) was microinjected into LBOs embedded in Matrigel. Microinjection of rAAV8 encoding human IgG served as negative control. Fluorescence microscopy was performed 72 h post injection using the EVOS FL Auto.

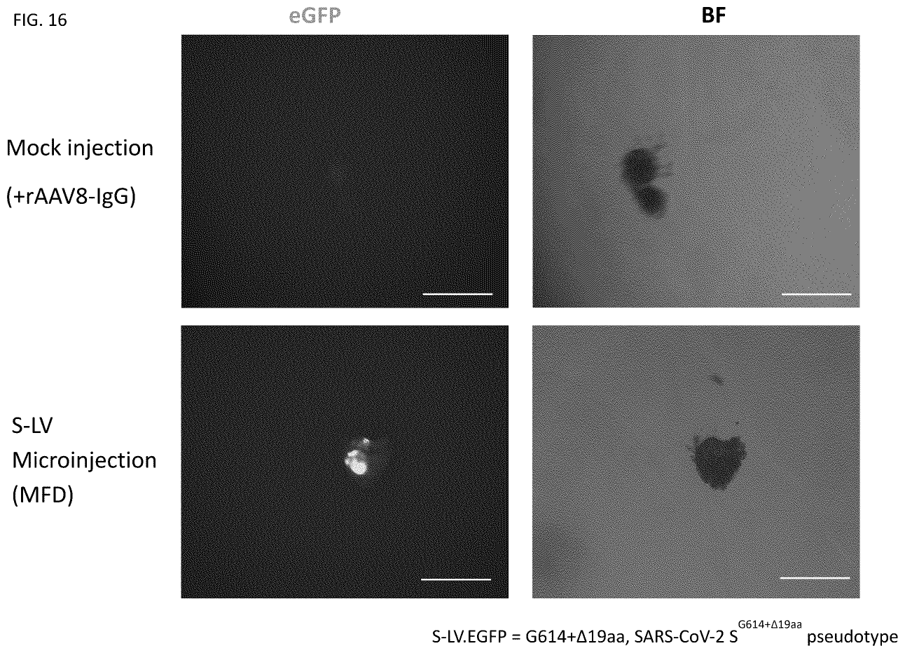

As shown in FIG. 16, EGFP was expressed in LBO post-delivery of (G614+Δ19aa, SARS-CoV-2 S^{G614+Δ19aa}).

### Example 11—SARS-CoV-2 2 Nucleoprotein (N) Boosts Recombinant HIV1 Lentiviral Vector (LV) Titres

The following S-LV vectors were tested:

$$SARS - Cov - 2 {S}^{G 614 + Δ 19 aa} = G6 \; 14 + Δ 19$$

$$SARS - Cov - 2 {S}^{G 614 + FKO + Δ 19 aa} = G 614 + FKO + Δ 19$$

$$B \text{.1} . 617.2 + Δ 19 = Delta + Δ 19$$

$$C \text{.37} + Δ 19 = Lambda + Δ 19$$

These S-LV vectors encoding EGFP were produced as described in the materials and methods section above, with and without SARS-CoV-2 nucleoprotein derived from Wuhan or Omicron variants, included at co-transfection. Both SARS-CoV-2 nucleoprotein derived from Wuhan or Omicron variants were tested with B.1.617.2+Δ19 (Delta+Δ19) and C.37+Δ19 (Lambda+Δ19). Only SARS-CoV-2 nucleoprotein derived from Wuhan variant was tested with SARS-CoV-2 S^{G614+Δ19aa} (G614+Δ19) and SARS-CoV-2 S^{G614+FKO+Δ19aa} (G614+FKO+Δ19, where FKO=furin knock out).

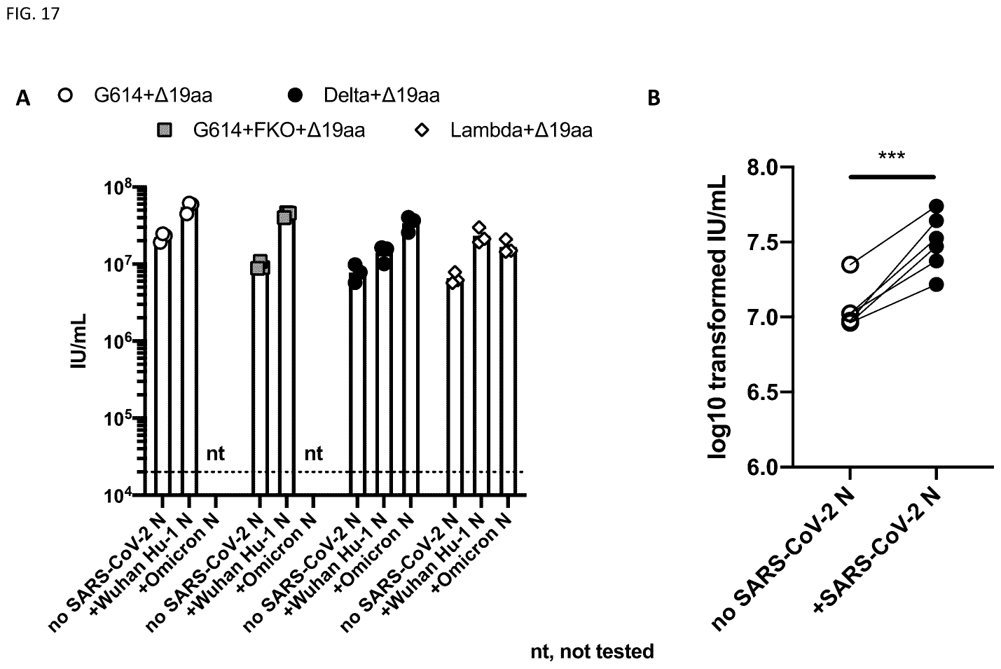

Crude vector material were titrated on SARS-CoV-2 permissive 293T/17 cells stably expressing hACE2 and hTMPRSS2, and titres calculated after flow cytometry analysis (FIG. 17A). These titres were log 10 transformed and the difference between matched titre pairs analysed. As shown in FIG. 17B, a significant improvement in yields and titres was observed when SARS-CoV-2 N (Wuhan or Omicron) was co-transfected (paired t-test; P=0.0006).

### DISCUSSION

In response to the demand for high titre SARS-CoV-2 PSV resource a S-LV platform has been established that can be readily harnessed to model a library of clinically relevant SARS-CoV-2 S glycoproteins. This included S derived from VOCs (namely G614, B.1.1.7, and B.1.351) or S variants of interest as surrogates to model potentially emerging SARS-CoV-2 variants. In particular, this has been exemplified by using modified S proteins that comprise the Q498Y and P499T mutations, which have been shown herein to confer mACE2 co-permissiveness.

A comprehensive examination of the S-LV platform and the ability to model SARS-CoV-2 is provided. We achieved impressively high functional titres of mouse adapted S-LVs. This was achieved using scalable 293T/17 suspension cells and incorporating a truncation of the SARS-CoV-2 S cytoplasmic tail encompassing a putative ERS (+Δ19aa), which improved SARS-CoV-2 S packaging and titres after its deletion. Despite this Δ19aa truncation of the cytoplasmic tail, the encoded SARS-CoV-2 S in our S-LV library demonstrably retain their natural tertiary structure as per natural SARS-CoV-2 S and recapitulates functional SARS-CoV-2 S protein (given evidence of ACE2-dependent cell entry, ability to mediate syncytia, co-purification of SARS-CoV-2 S trimer with S-LV PSV preps exemplified above, and using predictive 3D modelling and structure alignments of our encoded SARS-CoV-2 S sequences (for example, predicted 3D models resolved with confidence to the Wuhan Hu-1 S protein as per PDBs: 7czpA, 7cwl, and 7a93A) using the IntFOLD tool30 (data not shown)). Interestingly, using our S-LV platform, incorporating the Δ19aa truncation, and by employing the use of permissive 293T/17 cells that expressed both the ACE2 entry receptor and hTMPRSS2 serine protease we observed significant rescue of SARS-CoV-2 S pseudotyped LV—by up to a log in functional titres in a given prep. This was especially true for difficult to pseudotype SARS-CoV-2 variants—the Aus/VIC01 strain, especially as full-length S protein, was found particularly difficult to pseudotype to comparable titres for other S-LVs modelled.

Further interrogation of the SARS-CoV-2 S glycoproteins and S-LV platform was performed to substantiate the impressive yields and titres achieved. If particular, it was examined if the modified SARS-CoV-2 S glycoproteins of the invention recapitulated well known and characterised inherent features to improve upon the potential translational benefits of the S-LV platform. For example, demonstrable neutralization to well established and commercially available neutralizing antibodies was achieved. Neutralization escape to monoclonal nAbs, was demonstrable in a variant dependent manner as expected given the individual mutation profiles that confer escape from nAbs. Furthermore, the expressed SARS-CoV-2 S glycoprotein demonstrated potent ability to induce fusion and syncytium between cells as is inherent to the fusion peptide located within the S2 domain. The S^{G614} mutant was found to improve S glycoprotein stability, reduced premature S1 cleavage and shedding, and promote more dynamically open prefusion conformations to facilitate ACE2 binding relative to the S^{D614}. This was observed in the present study as substantially delayed onset of syncytium in transfection studies.

Via additional representative S-LVs we were able to further present the ability to successfully model SARS-CoV-2 VOC strains' (B.1.1.7 and B.1.351) aptitudes to impressively infect permissive cells relative to a benchmark reference (in this case PSVs representing the G614 variant) in vitro. Importantly, the measured 3-4-fold improved transduction capabilities inherent to N501Y-containing VOCs were successfully recapitulated as per authentic SARS-CoV-2 variants suggested in the literature. All together, and with S^{Aus/VIC01} in mind also, we present perhaps the first example in which modelling SARS-CoV-2 as a PSV can infer much about the associated S glycoprotein's ability to infect permissive cells via the ACE2 entry receptor. This S-LV tool offers a potential means to rapidly examine the extent of which VUIs, VOCs, emergent, and predicted SARS-CoV-2 variants can infect permissive cells in context of its S glycoprotein to enhance surveillance efforts.

Promisingly, these results also demonstrate that select SARS-CoV-2 PSV can function directly in vivo without the need for hACE2 expression supplied in trans after taking advantage of the SARS-CoV-2 S Q498Y and P499T19, and optionally also N501Y, mutations. The ability for select S-LVs (and by extension their corresponding authentic SARS-CoV-2 counterpart) to infect via the endogenous mACE2 entry receptor has useful implications, especially in contexts of examining the efficacy of prophylaxes, therapeutics, and vaccine strategies. We note that currently available mouse models for authentic and non-mouse-adapted SARS-CoV-2 infection are difficult to engineer with significant costs and animal wastage associated with supplying hACE2 transgenic52 or CRISPR/Cas9-mediated knock-in animals as significant primary examples. Even simplifying the humanization and sensitization process of mouse models to SARS-CoV-2, for example by introducing hACE2 to the murine lungs and airway in trans by replication incompetent Adenoviral (AdV), recombinant Adenovirus-Associated Viral (rAAV), or LV vectors, results in non-physiologically relevant biodistribution of hACE2 in the murine lungs as dictated by the tropism of the selected gene transfer vector. Additionally, the complexity of challenging animal models with multiple vectors (assumingly at least two different viral-based materials are necessary—i. the vector of choice for gene transfer to achieve hACE2-humanization, and ii. the challenging PSV or authentic SARS-CoV-2) can be alleviated by mACE2 co-permissive SARS-CoV-2.

Further, individual variants of the S-LV provide further advantages, particularly in terms of in vivo transgene expression, over and above the advantageous properties of the S-LV of the invention as a whole In particular, S^{B.1.351+Δ19aa}-LV has been shown to provide a significant increase in in vivo lung transgene expression compared with S^{Y498, T499, G613Δ19aa}-LV.

Also, mouse-adapted S-LV of the invention are still able to efficiently transduce SARS-CoV-2 permissive human cells to drive therapeutic transgene expression, and SARS-CoV-2 S^{G614+Δ19aa} was able to transduce human cells in LBO, an accepted model for alveolar type II cells. As such, these data suggest that the S-LV of the invention can be used to deliver therapeutic transgenes to human alveolar type II cells in vivo. As alveolar type II cells are the target cell type for numerous respiratory diseases and disorders, such as surfactant deficiency (ABCA3 or SP-B deficiencies) and idiopathic pulmonary fibrosis, the S-LV of the invention have potential as gene therapy vectors.

To conclude, the Examples herein demonstrate the ability to pseudotype third generation, SIN HIV1 LVs with SARS-CoV-2 S—referred to as S-LV. A wide S-LV library was established, encompassing and modelling S glycoproteins from VOCs, variants of clinical relevance, or of particular interest to function as a novel PSV resource that can be used in standard laboratory containment conditions. Impressive functional titres of S-LV were achieved after transient transfection of suspension 293T/17 with eGFP or FLuc reporter-encoding HIV1 LV genome, HIV1 GagPol, HIV1 Rev plasmids, and plasmid(s) encoding the S glycoproteins of interest. S-LVs could be further concentrated and purified for expanded downstream uses including in vivo applications. Importantly, this S-LV platform and library can potentially support the progress of COVID-19/SARS-CoV-2-related research by modelling VOCs and variants of interest in order to interrogate the SARS-CoV-2 S variants' neutralization potential to nAbs or convalescent plasma from COVID-19 recovered patients, or function to help determine a SARS-CoV-2 variant's potential in infectivity through the strain defining S glycoprotein. Finally, these Examples provide the first demonstration of applying S-LV in vivo by intranasally dosing BALB/c mice to demonstrate potent gene transfer capabilities independent of hACE2 expression as a rapid and direct means to model SARS-CoV-2 infection in vivo. Taken all together, this S-LV platform accurately represents authentic SARS-CoV-2, in particular representative variants modelled, at the level of the SARS-CoV-2 S glycoprotein. This S-LV platform demonstrates robustness in the wide array of SARS-CoV-2 variants represented in the current study, and flexibility such that it is not constrained by high BSL requirements. The S-LV platform therefore functions as an easy to use, easy to access, and safe SARS-CoV-2 PSV resource for related research to combat the on-going COVID-19/SARS-CoV-2 pandemic. Furthermore, these S-LV vectors are also capable of transducing SARS-CoV-2 permissive human cells to drive therapeutic transgene expression, indicating that these S-LV may be useful as gene therapy vectors in the clinic, as well as a SARS-CoV-2 PSV resource.

1. A lentiviral vector pseudotyped with a modified severe acute respiratory syndrome coronavirus 2 (SARS-CoV-2) spike protein, which lentiviral vector comprises a transgene operably linked to a promoter; and wherein said spike protein comprises:
a) mutations at amino acid positions corresponding to, or aligning with, positions 498, 499 and 614 of SEQ ID NO: 1; and
b) a deletion of at least a portion of the cytoplasmic tail.

2. A lentiviral vector pseudotyped with a modified severe acute respiratory syndrome coronavirus 2 (SARS-CoV-2) spike protein, which lentiviral vector comprises a transgene operably linked to a promoter; and wherein said spike protein comprises:
a) a mutation at an amino acid position corresponding to, or aligning with, position 614 of SEQ ID NO: 1;
b) a deletion of at least a portion of the cytoplasmic tail; and
c) (i) mutations at amino acid positions corresponding to, or aligning with, positions 498 and 499 of SEQ ID NO: 1; and/or (ii) a mutation at an amino acid position corresponding to, or aligning with, position 501 of SEQ ID NO: 1.

3. The lentiviral vector of claim 1 or 2, wherein the cytoplasmic tail of the spike protein corresponds to, or aligns with amino acid resides 1235 to 1273 of SEQ ID NO: 1.

4. The lentiviral vector of any one of the preceding claim 1, wherein the deletion of at least a portion of the cytoplasmic tail of the spike protein comprises:
a) deletion of at least 10 amino acids, preferably at least 15 amino acids of the cytoplasmic tail; and/or
b) deletion of the amino acid residues corresponding to, or aligning with, positions 1255 to 1273 of SEQ ID NO: 1.

5. The lentiviral vector of any one of the preceding claims, wherein one or more of the mutations of the spike protein at amino acid positions corresponding to, or aligning with, positions 498, 499 and 614 of SEQ ID NO: 1 are amino acid substitutions, and preferably wherein all of the mutations are amino acid substitutions.

6. The lentiviral vector of claim 5, wherein the amino acid substitutions are non-conservative amino acid substitutions.

7. The lentiviral vector of any one of the preceding claims, wherein the amino acid corresponding to, or aligning with:
a) position 498 of SEQ ID NO: 1 is substituted by tyrosine;
b) position 499 of SEQ ID NO: 1 is substituted by threonine; and/or
c) position 614 of SEQ ID NO: 1 is substituted by glycine.

8. The lentiviral vector of any one of the preceding claims, wherein the mutations are Q498Y, P499T and/or D614G.

9. The lentiviral vector of any one of the preceding claims, wherein the modified spike protein is capable of binding to the enzymatic domain of human angiotensin converting enzyme 2 (ACE2).

10. The lentiviral vector of any one of the preceding claims, wherein the modified SARS-CoV-2 spike protein is derived from a SARS-CoV-2 strain selected from Wuhan-Hu-1 strain, B.1.1.7 strain, B.1.351 strain, P.1 strain, B.1.617.2 strain, B.1.427, C.37,B.1.429 or Australia/VIC01/2020 (Aus/VIC01) strain.

11. The lentiviral vector of any one of the preceding claims, wherein the modified spike protein is not detected by anti-coronavirus spike protein antibodies, preferably, anti-coronavirus spike protein antibodies MM43 or R001.

12. The lentiviral vector of any one of the preceding claims, wherein the modified spike protein comprises an amino acid sequence having at least 90% sequence identity with SEQ ID NO: 13.

13. The lentiviral vector of any one of the preceding claims, wherein the modified spike protein further comprises one or more additional mutation.

14. The lentiviral vector of any one of the preceding claims, which comprises a mutation at an amino acid position corresponding to, or aligning with, position 501 of SEQ ID NO: 1, or wherein said one or more additional mutation comprises a mutation at amino acid position corresponding to, or aligning with, position 501 of SEQ ID NO: 1, wherein optionally:
a) the mutation at amino acid position corresponding to, or aligning with, position 501 of SEQ ID NO: 1, is an amino acid substitution, preferably a non-conservative amino acid substitution, even more preferably a substitution by tyrosine; and/or
b) the one or more additional mutation comprises N501Y.

15. The lentiviral vector of any one of the preceding claims, wherein the modified spike protein comprises an amino acid sequence having at least 90% sequence identity with SEQ ID NO: 20.

16. The lentiviral vector of any one of claims 2 to 15, wherein said spike protein comprises:
a) mutations at amino acid positions corresponding to, or aligning with, one or more of positions 80, 215, 417, 484, 501, 614 and 701 of SEQ ID NO: 1 wherein preferably all these residues are mutated; and
b) a deletion of at least a portion of the cytoplasmic tail.

17. The lentiviral vector of claim 16, wherein:
a) said modified SARS-CoV-2 spike protein is derived from the spike protein of the B.1.351 strain;
b) the amino acid corresponding to, or aligning with: (i) position 80 of SEQ ID NO: 1 is substituted by alanine; (ii) position 215 of SEQ ID NO: 1 is substituted by glycine; (iii) position 417 of SEQ ID NO: 1 is substituted by asparagine; (iv) position 484 of SEQ ID NO: 1 is substituted by lysine; (v) position 501 of SEQ ID NO: 1 is substituted by tyrosine; (vi) position 614 of SEQ ID NO: 1 is substituted by glycine and/or (vii) position 701 of SEQ ID NO: 1 is substituted by valine; wherein preferably all these residues are substituted; and/or
c) the deletion of at least a portion of the cytoplasmic tail comprises or consists of deletion of the amino acid residues corresponding to or aligning with positions 1255 to 1273 of SEQ ID NO: 1.

18. The lentiviral vector of any one of claims 1 to 17, which is selected from the group consisting of a Simian immunodeficiency virus (SIV) vector, a Human immunodeficiency virus (HIV) vector, a Feline immunodeficiency virus (FIV) vector, an Equine infectious anaemia virus (EIAV) vector, and a Visna/maedi virus vector.

19. The lentiviral vector of any one of claims 1 to 18, wherein the vector is capable of transducing rodent cells in vivo, preferably mouse cells in vivo.

20. A modified severe acute respiratory syndrome coronavirus 2 (SARS-CoV-2) spike protein as defined in any one of claims 1 to 16.

21. A polynucleotide molecule encoding a modified spike protein as defined in claim 20.

22. An expression construct comprising the polynucleotide of claim 21, operably linked to a promoter.

23. A host cell comprising the lentiviral vector of any one of claims 1 to 19, the modified spike protein of claim 20, the polynucleotide of claim 21 or the expression construct of claim 22.

24. A virus-like particle (VLP) comprising a modified SARS-CoV-2 spike protein of claim 20.

25. The lentiviral vector of any one of claims 1 to 19, the modified spike protein of claim 20, the polynucleotide of claim 21, the expression construct of claim 22 or the VLP of claim 24, for use in therapy, wherein preferably the therapy is gene therapy.

26. In vitro use of the lentiviral vector of any one of claims 1 to 19, the modified spike protein of claim 20, the polynucleotide of claim 21, the expression construct of claim 22, or the VLP of claim 24.

27. A method of producing a lentiviral vector according to any of claims 1 to 19, the method comprising:
a) introducing (i) a nucleic acid sequence encoding a modified SARS-CoV-2 spike protein as defined in claim 20; and (ii) one or more nucleic acid sequence encoding lentiviral packaging components, lentiviral envelope components, and a lentiviral genome, into a viral vector production cell; and
b) culturing the production cell under conditions suitable for the production of the lentiviral vector.

28. The method of claim 27, wherein:
a) the method further comprises harvesting said lentiviral vector;
b) the nucleic acid sequence encoding the modified SARS-CoV-2 spike protein is comprised in a polynucleotide molecule as defined in claim 21 or an expression construct as defined in claim 22;
c) the one or more nucleic acid sequence encoding the lentiviral packaging components, lentiviral envelope components, and a lentiviral genome are comprised in (i) the same polynucleotide molecule or expression construct as the nucleic acid sequence encoding the modified SARS-CoV-2 spike protein or (ii) in one or more separate polynucleotide molecule or expression construct; and/or
d) SARS-CoV-2 nucleoprotein is co-expressed during the culturing of the production cell, wherein preferably the SARS-CoV-2 nucleoprotein is from the Wuhan-Hu-1 or B.1.1.529 strain.


SEQUENCE LISTING (64 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1254 aa, synthetic construct
  REGION 1..1254: note = Wuhan Hu-1+del19aa (encoded by pGM896 S-LV v02)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 3 - protein, 1273 aa, synthetic construct
  REGION 1..1273: note = S:G614 (encoded by pGM906 S-LV v01)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1254 aa, synthetic construct
  REGION 1..1254: note = S:G614+del19aa (encoded by pGM907 S-LV v02)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 5 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRRYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1254 aa, synthetic construct
  REGION 1..1254: note = S:Aus/VIC01+del19aa (encoded by pGM904 S-LV v02)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRRYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 7 - protein, 1251 aa, synthetic construct
  REGION 1..1251: note = S:B.1.1.7+del19aa(encoded by pGM965 S-LV v04)
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCIQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 8 - protein, 1254 aa, synthetic construct
  REGION 1..1254: note = S:B.1.1351+del19aa(encoded by pGM970 S-LV v04)
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 9 - protein, 1251 aa, synthetic construct
  REGION 1..1251: note = S:B.1.1.7&B.1.1351 chimera+del19aa (encoded by pGM1000 S-LV v02)
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 10 - protein, 1247 aa, synthetic construct
  REGION 1..1247: note = S:B.1.617.2+del19aa (encoded by pGM1027 S-LV v02)
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 11 - protein, 1247 aa, synthetic construct; identical to SEQ ID NO: 10
  REGION 1..1247: note = C.37+del19aa (encoded by pGM1028 S-LV v02)

SEQ ID NO: 12 - protein, 1254 aa, synthetic construct
  REGION 1..1254: note = maS:Y498,T499+del19aa (encoded by pGM937 S-LV v03)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFYTTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 13 - protein, 1254 aa, synthetic construct
  REGION 1..1254: note = maS:Y498,T499,G614+del19aa (encoded by pGM939 S-LV v03)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFYTTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 14 - protein, 1254 aa, synthetic construct
  REGION 1..1254: note = maS:Aus/VIC01,Y498,T499+del19aa (encoded by pGM938 S-LV v03)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRRYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFYTTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 15 - protein, 1252 aa, synthetic construct
  REGION 1..1252: note = S:B.1.617.2,Y501+del19aa (encoded by pGM1038 S-LV v04)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 16 - protein, 1247 aa, synthetic construct
  REGION 1..1247: note = C.37,Y501+del19aa (encoded by pGM1039 S-LV v05)
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 17 - protein, 1252 aa, synthetic construct
  REGION 1..1252: note = S:B.1.617.2,Y498,T499,Y501+del19aa (encoded by pGM1040 S-LV v04)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFYTTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 18 - protein, 1247 aa, synthetic construct
  REGION 1..1247: note = C.37,Y489,T499,Y501+del19aa (encoded by pGM1041 S-LV v05)
MFVFLVLLPLVSSQCVNLTTRTQLPPAYTNSFTRGVYYPDKVFRSSVLHSTQDLFLPFFSNVTWFHAIHVSGTNVIKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYQYRLFRKSNLKPFERDISTEIYQAGSTPCNGVEGFNCYSPLQSYGFYTTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLNVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 19 - protein, 1254 aa, synthetic construct
  REGION 1..1254: note = maS:Y498,T499,Y501+del19aa (encoded by pGM998 S-LV v05)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFYTTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 20 - protein, 1254 aa, synthetic construct
  REGION 1..1254: note = maS:Y498,T499,Y501,G614+del19aa (encoded by pGM999 S-LV v05)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFYTTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 21 - protein, 1254 aa, synthetic construct
  REGION 1..1254: note = S:G614 with furin site knock out (encoded by pGM1026 S-LV v05)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPSRASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 22 - DNA, 3822 bp, SARS-CoV-2
  source 1..3822: mol_type = genomic DNA
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgcaagttcgacgaggacgacagcgagcctgtgctgaagggcgtgaagctgcactacacctga

SEQ ID NO: 23 - DNA, 3765 bp, synthetic construct
  source 1..3765: mol_type = other DNA
  misc_feature 1..3765: note = Wuhan Hu-1+del19aa (comprised in pGM896 S-LV v02)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 24 - DNA, 3822 bp, synthetic construct
  source 1..3822: mol_type = other DNA
  misc_feature 1..3822: note = S:G614 (comprised in pGM906 S-LV v01)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgcaagttcgacgaggacgacagcgagcctgtgctgaagggcgtgaagctgcactacacctga

SEQ ID NO: 25 - DNA, 3765 bp, synthetic construct
  source 1..3765: mol_type = other DNA
  misc_feature 1..3765: note = S:G614+del19aa (comprised in pGM907 S-LV v02)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 26 - DNA, 3822 bp, SARS-CoV-2
  source 1..3822: mol_type = genomic DNA
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagaagatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgcaagttcgacgaggacgacagcgagcctgtgctgaagggcgtgaagctgcactacacctga

SEQ ID NO: 27 - DNA, 3765 bp, synthetic construct
  source 1..3765: mol_type = other DNA
  misc_feature 1..3765: note = S:Aus/VIC01+del19aa (comprised in pGM904 S-LV v02)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagaagatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 28 - DNA, 3756 bp, synthetic construct
  source 1..3756: mol_type = other DNA
  misc_feature 1..3756: note = S:B.1.1.7+del19aa (comprised in pGM965 S-LV v04)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatcagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaaccggtacgggcgtgctgaccgagagcaataagaagtttcttccctttcaacaattcggcagagacatcgatgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccatagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcatccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctggccagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccacccacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 29 - DNA, 3765 bp, synthetic construct
  source 1..3765: mol_type = other DNA
  misc_feature 1..3765: note = S:B.1.1351+del19aa (comprised in pGM970 S-LV v04)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgccaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagaggcctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaacatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtgaagggcttcaattgctacttccctctgcagagctacggcttccagcctacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgtcgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 30 - DNA, 3756 bp, synthetic construct
  source 1..3756: mol_type = other DNA
  misc_feature 1..3756: note = S:B.1.1.7&B.1.1351 chimera+del19aa (comprised in pGM1000 S-LVv02)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatcagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtgaagggcttcaattgctacttccctctgcagagctacggcttccagcctacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggtacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgtcgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 31 - DNA, 3759 bp, synthetic construct
  source 1..3759: mol_type = other DNA
  misc_feature 1..3759: note = S:B.1.617.2+del19aa (comprised in pGM1027 S-LV v02)
atgttcgtgttcctggtgctgctgcctctggtgagcagccagtgcgtgaatctgaggaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctggatgtttattatcataagaacaacaagagctggatggagagcggcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattaccggtacagactgttcagaaagagcaatctgaagcctttcgagagagacatcagcaccgagatctaccaggccggcagcaaaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccgtagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcagaacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 32 - DNA, 3744 bp, synthetic construct
  source 1..3744: mol_type = other DNA
  misc_feature 1..3744: note = C.37+del19aa (comprised in pGM1028 S-LV v02)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatgtcatcaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattaccagtacagactgttcagaaagagcaatctgaagcctttcgagagagacatcagcaccgagatctaccaggccggcagcacaccgtgtaatggcgtggagggcttcaattgctacagtcctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgaacgtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 33 - DNA, 3765 bp, synthetic construct
  source 1..3765: mol_type = other DNA
  misc_feature 1..3765: note = maS:Y498,T499+del19aa (comprised in pGM937 S-LV v03)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttctatactaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 34 - DNA, 3765 bp, synthetic construct
  source 1..3765: mol_type = other DNA
  misc_feature 1..3765: note = maS:Y498,T499,G614+del19aa (comprised in pGM939 S-LV v03)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttctatactaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 35 - DNA, 3765 bp, synthetic construct
  source 1..3765: mol_type = other DNA
  misc_feature 1..3765: note = maS:Aus/VIC01,Y498,T499+del19aa (comprised in pGM938 S-LV v03)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagaagatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttctatactaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 36 - DNA, 3759 bp, synthetic construct
  source 1..3759: mol_type = other DNA
  misc_feature 1..3759: note = S:B.1.617.2,Y501+del19aa (comprised in pGM1038 S-LV v04)
atgttcgtgttcctggtgctgctgcctctggtgagcagccagtgcgtgaatctgaggaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctggatgtttattatcataagaacaacaagagctggatggagagcggcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattaccggtacagactgttcagaaagagcaatctgaagcctttcgagagagacatcagcaccgagatctaccaggccggcagcaaaccgtgtaatggcgtggagggcttcaattgctacttccctctgcagagctacggcttccagcctacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccgtagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcagaacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 37 - DNA, 3744 bp, synthetic construct
  source 1..3744: mol_type = other DNA
  misc_feature 1..3744: note = C.37,Y501+del19aa (comprised in pGM1039 S-LV v05)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatgtcatcaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattaccagtacagactgttcagaaagagcaatctgaagcctttcgagagagacatcagcaccgagatctaccaggccggcagcacaccgtgtaatggcgtggagggcttcaattgctacagtcctctgcagagctacggcttccagcctacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgaacgtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 38 - DNA, 3759 bp, synthetic construct
  source 1..3759: mol_type = other DNA
  misc_feature 1..3759: note = S:B.1.617.2,Y484,T499,Y501+del19aa (comprised in pGM1040 S-LVv04)
atgttcgtgttcctggtgctgctgcctctggtgagcagccagtgcgtgaatctgaggaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctggatgtttattatcataagaacaacaagagctggatggagagcggcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattaccggtacagactgttcagaaagagcaatctgaagcctttcgagagagacatcagcaccgagatctaccaggccggcagcaaaccgtgtaatggcgtggagggcttcaattgctacttccctctgcagagctacggcttctatactacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccgtagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcagaacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 39 - DNA, 3744 bp, synthetic construct
  source 1..3744: mol_type = other DNA
  misc_feature 1..3744: note = C.37,Y489,T499,Y501+del19aa (comprised in pGM1041 S-LV v05)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatgtcatcaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattaccagtacagactgttcagaaagagcaatctgaagcctttcgagagagacatcagcaccgagatctaccaggccggcagcacaccgtgtaatggcgtggagggcttcaattgctacagtcctctgcagagctacggcttctatactacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgaacgtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 40 - DNA, 3765 bp, synthetic construct
  source 1..3765: mol_type = other DNA
  misc_feature 1..3765: note = maS:Y498,T499,Y501+del19aa (comprised in pGM998 S-LV v05)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttctatactacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccaggacgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 41 - DNA, 3765 bp, synthetic construct
  source 1..3765: mol_type = other DNA
  misc_feature 1..3765: note = maS:Y498,T499,Y501,G614+del19aa (comprised in pGM999 S-LV v05)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttctatactacctatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagaagagccagaagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 42 - DNA, 3765 bp, synthetic construct
  source 1..3765: mol_type = other DNA
  misc_feature 1..3765: note = S:G614 with furin site knock out (comprised in pGM1026 S-LV v05)
atgttcgtgttcctggtgctgctgcctctggtgagcagccagtgcgtgaatctgaccaccagaacccagctgcctcctgcctacaccaatagcttcaccagaggagtttattatcccgataaggtgttcagaagtagtgtattacatagtacccaggacctgttcctacctttcttcagtaacgtgacctggttccacgccatccacgtgagcggcaccaatggcaccaagagattcgacaatcctgtgctgcctttcaatgacggcgtgtacttcgccagcaccgagaagagcaatatcatcagaggctggatcttcggcaccaccttggattccaagactcagagcctgctgattgtaaacaacgctacaaatgtggtgatcaaggtgtgcgagttccagttctgcaatgaccctttcctgggtgtttattatcataagaacaacaagagctggatggagagcgagttccgcgtatattcgtcggctaataattgcaccttcgagtacgtgagccagcctttcctgatggacctggagggcaagcagggcaatttcaagaatctgagagagttcgtgttcaagaatatcgacggctacttcaagatctacagcaagcacacacccattaatctggtgagagacctgcctcagggcttcagcgccctggagcctctggtggacctgcctatcggcatcaatatcaccagattccagaccctgctggccctgcacagatcatatcttacaccaggcgattcgtcaagcggttggaccgctggagctgcggcatattacgtgggctacctgcagcctagaaccttcctgctgaagtacaatgagaatggtacgataaccgacgcagttgattgtgccctggaccctctgagcgagaccaagtgcaccctgaagagcttcaccgtggagaagggcatctaccagaccagcaatttcagagtgcagcctaccgagagcatcgtgagattccctaatatcaccaatctgtgccctttcggcgaggtgttcaatgccaccagattcgccagcgtgtacgcatggaaccgcaagcggataagcaattgcgtggccgactacagcgtgctgtacaatagcgccagcttcagcaccttcaaatgttatggtgtttcgccaacaaagctgaatgacctgtgcttcaccaatgtgtacgccgacagcttcgtgatcagaggcgacgaggtgagacagatcgcgccagggcagaccggcaagatcgccgactacaattacaagctgcctgacgacttcaccggctgcgtgatcgcgtggaactctaacaatctagattcgaaagttggaggcaattacaattacctgtacagactgttcagaaagagcaatctgaagcctttcgagagagacatcagcaccgagatctaccaggccggcagcacaccgtgtaatggcgtggagggcttcaattgctacttccctctgcagagctacggcttccagcctaccaatggcgtgggctaccagccttacagagtggtggtgctgagcttcgagctgctgcacgctcccgctaccgtgtgcggccctaagaagagcaccaatctggtgaagaataagtgcgtgaatttcaatttcaatggtctaactggaacgggcgtgctgaccgagagcaataagaagtttcttccctttcaacaattcggcagagacatcgccgacaccacagatgctgtaagagaccctcagaccctggagatcctggacatcactccgtgtagcttcggcggcgtgagcgtgatcacaccgggtaccaataccagcaatcaggtggccgtgctgtaccagggcgtgaattgcaccgaggtgcctgtggccatccacgccgaccagctgactcccacttggagggtatattccacgggaagcaatgtgttccagaccagagccggctgcctgatcggcgccgagcacgtgaataatagctacgagtgcgacatccctatcggcgccggcatctgcgccagctaccagacccagaccaatagccctagtagagccagtagcgtggccagccagagcatcatcgcctacaccatgagcctgggcgccgagaatagcgtggcctacagcaataatagcatcgccatccctaccaatttcaccatcagcgtgaccaccgaaatattaccagtctccatgaccaagaccagcgtggactgcaccatgtacatctgcggcgacagcaccgagtgcagcaatctgctgctgcagtacggcagcttctgcacccagctgaatagagccctgaccggcatcgccgtggagcaggacaagaatacccaggaggtgttcgcccaggtgaagcagatctacaagactccgccgatcaaggacttcggcggcttcaatttcagccaaatactcccagatccaagcaagcctagcaagaggagcttcatcgaggacctgctgttcaataaggtgaccctggccgacgccggcttcatcaagcagtacggcgactgcctaggtgatattgcggcaagagacctgatctgcgcccagaagtttaacggtttgacagtactacctcctctgctgaccgacgagatgatagcacaatatacgtcggcattgctcgctggcacgatcacatcgggctggactttcggcgccggagcagcgttgcaaatccctttcgccatgcagatggcctacagattcaatggcatcggcgtgacccagaatgtgctgtacgagaatcagaagctgatcgccaatcagttcaatagcgccatcggcaagatccaggacagcctgagcagcaccgccagcgccctgggcaagctgcaggacgtggtgaatcagaatgcccaggccctgaataccctggtgaagcagctgagcagcaatttcggcgccatcagtagtgtactcaacgatatcctgagcagactggacaaggtggaggccgaggtgcaaattgatcgtcttattactggcagactgcagagcctgcagacctacgtgacccagcagctgatcagagccgccgagatcagagccagcgccaatctggccgccaccaagatgagcgagtgcgtgctgggccagagcaagagagtggacttctgcggcaagggctaccacctgatgagcttccctcagagcgctccacatggcgtggtgttcctgcacgtgacctacgtgcctgcccaggagaagaatttcaccaccgcacccgcaatctgccacgacggcaaggcccacttccctagagagggcgtgttcgtgagcaatggcacccactggttcgtgacccagagaaatttctacgagcctcagatcatcaccaccgacaataccttcgtgagcggcaattgcgacgtggtgatcgggatagtcaataatactgtctacgaccctctgcagcctgagctggacagcttcaaggaggagctggacaagtacttcaagaatcacaccagccctgacgtggacctcggtgatatttcgggaatcaatgccagcgtggtgaatatccagaaggaaattgatcggctcaacgaagtggccaagaatctgaatgagagcctgatcgacctgcaggagctgggcaagtacgagcagtacatcaagtggccttggtacatctggctgggcttcatcgccggcctgatcgccatcgtgatggtgaccatcatgctgtgctgcatgacctcctgttgttcctgtttgaaagggtgttgttcgtgtgggtcctgctgctga

SEQ ID NO: 43 - DNA, 574 bp, synthetic construct
  source 1..574: mol_type = other DNA
  misc_feature 1..574: note = hCEF promoter
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 44 - DNA, 873 bp, Cytomegalovirus
  source 1..873: mol_type = genomic DNA
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 45 - DNA, 395 bp, Homo sapiens
  source 1..395: mol_type = genomic DNA
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 46 - DNA, 600 bp, Woodchuck hepatitis virus
  source 1..600: mol_type = genomic DNA
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct

SEQ ID NO: 47 - DNA, 26 bp, synthetic construct
  source 1..26: mol_type = other DNA
  misc_feature 1..26: note = forward primer for (co)hACE2
cgatctagagaaacttgtttgcacgt

SEQ ID NO: 48 - DNA, 26 bp, synthetic construct
  source 1..26: mol_type = other DNA
  misc_feature 1..26: note = reverse primer for (co)hACE2
cgatctagagaaacttgtttgcacgt

SEQ ID NO: 49 - DNA, 20 bp, synthetic construct
  source 1..20: mol_type = other DNA
  misc_feature 1..20: note = forward primer
cgactctagagtcgacggtt

SEQ ID NO: 50 - DNA, 28 bp, synthetic construct
  source 1..28: mol_type = other DNA
  misc_feature 1..28: note = reverse primer
gatgcggccgcttcaggcaccgggcttg

SEQ ID NO: 51 - DNA, 29 bp, synthetic construct
  source 1..29: mol_type = other DNA
  misc_feature 1..29: note = forward primer for hTMPRSS2
aatgctagccaccatggctttgaactcag

SEQ ID NO: 52 - DNA, 26 bp, synthetic construct
  source 1..26: mol_type = other DNA
  misc_feature 1..26: note = reverse primer for hTMPRSS2
gtctctagagccgtctgccctcattt

SEQ ID NO: 53 - DNA, 29 bp, synthetic construct
  source 1..29: mol_type = other DNA
  misc_feature 1..29: note = forward primer
acgtctagagcaacaaacttctctctgct

SEQ ID NO: 54 - DNA, 31 bp, synthetic construct
  source 1..31: mol_type = other DNA
  misc_feature 1..31: note = reverse primer
tatgcggccgcttagccctcccacacataac

SEQ ID NO: 55 - DNA, 30 bp, synthetic construct
  source 1..30: mol_type = other DNA
  misc_feature 1..30: note = forward primer for SARS-CoV-2 (co)S (Wuhan Hu-1)
caggctagccaccatgttcgtgttcctggt

SEQ ID NO: 56 - DNA, 32 bp, synthetic construct
  source 1..32: mol_type = other DNA
  misc_feature 1..32: note = reverse primer for SARS-CoV-2 (co)S (Wuhan Hu-1)
atagcggccgctcaggtgtagtgcagcttcac

SEQ ID NO: 57 - DNA, 30 bp, synthetic construct; identical to SEQ ID NO: 55
  source 1..30: mol_type = other DNA
  misc_feature 1..30: note = forward primer for SARS-CoV-2 (co)S (Wuhan Hu-1) del19

SEQ ID NO: 58 - DNA, 36 bp, synthetic construct
  source 1..36: mol_type = other DNA
  misc_feature 1..36: note = reverse primer for SARS-CoV-2 (co)S (Wuhan Hu-1) del19
atagcggccgcttcagcagcaggacccacacgaaca

SEQ ID NO: 59 - DNA, 33 bp, synthetic construct
  source 1..33: mol_type = other DNA
  misc_feature 1..33: note = forward primer for SARS-CoV-2 S Australia/VIC01/2020
cctgcacagaaagtatcttacaccaggcgattc

SEQ ID NO: 60 - DNA, 18 bp, synthetic construct
  source 1..18: mol_type = other DNA
  misc_feature 1..18: note = reverse primer for SARS-CoV-2 S Australia/VIC01/2020
gccagcagggtctggaat

SEQ ID NO: 61 - DNA, 22 bp, synthetic construct
  source 1..22: mol_type = other DNA
  misc_feature 1..22: note = forward primer for SARS-CoV-2 D614G variant
gctgtaccagggcgtgaattgc

SEQ ID NO: 62 - DNA, 18 bp, synthetic construct
  source 1..18: mol_type = other DNA
  misc_feature 1..18: note = reverse primer for SARS-CoV-2 D614G variant
acggccacctgattgctg

SEQ ID NO: 63 - DNA, 26 bp, synthetic construct
  source 1..26: mol_type = other DNA
  misc_feature 1..26: note = forward primer for SARS-CoV-2 S mACE2 adaptation
ctacggcttctatactaccaatggcg

SEQ ID NO: 64 - DNA, 18 bp, synthetic construct
  misc_feature 1..18: note = reverse primer for SARS-CoV-2 S mACE2 adaptation
  source 1..18: mol_type = other DNA
ctctgcagagggaagtag